(19) United States
(12) Patent Application Publication
(10) Pub. No.: US 2023/0279029 A1
(43) Pub. Date: Sep. 7, 2023

(54) ORGANIC ELECTROLUMINESCENT MATERIALS AND DEVICES

(71) Applicant: Universal Display Corporation, Ewing, NJ (US)

(72) Inventors: Tyler FLEETHAM, Yardley, PA (US); Nicholas J. THOMPSON, New Hope, PA (US)

(73) Assignee: Universal Display Corporation, Ewing, NJ (US)

(21) Appl. No.: 18/316,380

(22) Filed: May 12, 2023

Related U.S. Application Data
(63) Continuation-in-part of application No. 17/672,934, filed on Feb. 16, 2022.
Application No. 17/672,934 is a continuation-in-part of application No. 17/672,895, filed on Feb. 16, 2022.
Application No. 17/672,934 is a continuation-in-part of application No. 17/063,884, filed on Oct. 6, 2020.
Application No. 17/672,895 is a continuation-in-part of application No. 17/063,884, filed on Oct. 6, 2020.
Application No. 17/063,884 is a continuation-in-part of application No. 17/864,455, filed on Jul. 14, 2022.
Application No. 17/864,455 is a continuation-in-part of application No. 17/672,895, filed on Feb. 16, 2022.
Application No. 17/672,895 is a continuation-in-part of application No. 17/672,934, filed on Feb. 16, 2022.
(60) Provisional application No. 63/341,499, filed on May 13, 2022.
(60) Provisional application No. 63/396,356, filed on Aug. 9, 2022.
(60) Provisional application No. 63/220,429, filed on Jul. 9, 2021.
(60) Provisional application No. 63/154,320, filed on Feb. 26, 2021.
(60) Provisional application No. 63/229,748, filed on Aug. 5, 2021.
(60) Provisional application No. 62/982,883, filed on Feb. 28, 2020.
(60) Provisional application No. 62/971,295, filed on Feb. 7, 2020.
(60) Provisional application No. 62/926,035, filed on Oct. 25, 2019.
(60) Provisional application No. 63/313,545, filed on Feb. 24, 2022.

(51) Int. Cl. C07F 7/08; C07D 487/14; C07D 209/86; C07C 211/54; C07F 15/00; C07F 1/02; C07D 495/04; H10K 85/60

(52) U.S. Cl. CPC C07F 7/0812; C07D 487/14; C07D 209/86; C07C 211/54; C07F 15/0086; C07F 1/02; C07D 495/04; H10K 85/6572; H10K 85/6576; H10K 85/6574; H10K 85/654; H10K 50/121

(57) ABSTRACT

Provided are compounds consisting of a first group and a second group; and the first group does not overlap with the second group; the compounds have a HOMO and a LUMO; the second group consists of at least 70% of the electron densities of the HOMO and the LUMO; and the first group is at least 30% deuterated. Also provided are their uses in OLED related electronic devices.

### CROSS-REFERENCE TO RELATED APPLICATIONS

This application is a continuation-in-part of co-pending U.S. patent application Ser. No. 17/672,934, filed on Feb. 16, 2022, which claims benefit of U.S. Provisional applications No. 63/220,429, filed on Jul. 9, 2021, No. 63/154,320, filed on Feb. 26, 2021, and No. 63/229,748, filed on Aug. 5, 2021. Application Ser. No. 17/672,934 is also a continuation-in-part of U.S. patent application Ser. No. 17/063,884, filed on Oct. 6, 2020, which claims benefit of U.S. Provisional applications No. 62/926,035, filed on Oct. 25, 2019, No. 62/971,295, filed on Feb. 7, 2020, and No. 62/982,883, filed on Feb. 28, 2022. This application is also a continuation-in-part of co-pending U.S. patent application Ser. No. 17/672,895, filed on Feb. 16, 2022, which claims benefit of U.S. Provisional applications No. 63/229,748, filed on Aug. 5, 2021, No. 63/220,429, filed on Jul. 9, 2021, and No. 63/154,320, filed on Feb. 26, 2021. Application Ser. No. 17/672,895 is also a continuation-in-part of U.S. patent application Ser. No. 17/063,884, filed on Oct. 6, 2020, which claims benefit of U.S. Provisional applications No. 62/982,883, filed on Feb. 28, 2020, No. 62/971,295, filed on Feb. 7, 2020, and No. 62/926,035, filed on Oct. 25, 2019. This application is also a continuation-in-part of co-pending U.S. patent application Ser. No. 17/864,455, filed on Jul. 14, 2022, which is a continuation-in-part of U.S. patent application Ser. No. 17/672,895, filed on Feb. 16, 2022. The patent application Ser. No. 17/864,455 is also a continuation-in-part of U.S. patent application Ser. No. 17/672,934, filed on Feb. 16, 2022, and claims benefit of U.S. Provisional application No. 63/313,545, filed on Feb. 24, 2022. This application claims priority under 35 U.S.C. § 119(e) to U.S. Provisional Applications No. 63/396,356, filed on Aug. 9, 2022, and No. 63/341,499, filed on May 13, 2022. The entire contents of all the above identified applications are incorporated herein by reference.

### FIELD

The present disclosure generally relates to compounds having a first group and a second group with the first group not overlapping with the second group and the second group consisting of at least 70% of the electron densities of the HOMO (highest occupied molecular orbital) and the LUMO (lowest unoccupied molecular orbital) and the first group being at least 30% deuterated, and their use in OLED related electronic devices including consumer products.

### BACKGROUND

Opto-electronic devices that make use of organic materials are becoming increasingly desirable for various reasons. Many of the materials used to make such devices are relatively inexpensive, so organic opto-electronic devices have the potential for cost advantages over inorganic devices. In addition, the inherent properties of organic materials, such as their flexibility, may make them well suited for particular applications such as fabrication on a flexible substrate. Examples of organic opto-electronic devices include organic light emitting diodes/devices (OLEDs), organic phototransistors, organic photovoltaic cells, and organic photodetectors. For OLEDs, the organic materials may have performance advantages over conventional materials.

OLEDs make use of thin organic films that emit light when voltage is applied across the device. OLEDs are becoming an increasingly interesting technology for use in applications such as flat panel displays, illumination, and backlighting.

One application for phosphorescent emissive molecules is a full color display. Industry standards for such a display call for pixels adapted to emit particular colors, referred to as “saturated” colors. In particular, these standards call for saturated red, green, and blue pixels. Alternatively, the OLED can be designed to emit white light. In conventional liquid crystal displays emission from a white backlight is filtered using absorption filters to produce red, green and blue emission. The same technique can also be used with OLEDs. The white OLED can be either a single emissive layer (EML) device or a stack structure. Color may be measured using CIE coordinates, which are well known to the art.

### SUMMARY

Disclosed are organic materials for use as host materials in organic electroluminescent devices. The host materials comprise bulky groups such as triaryl silanes and tetraphenylenes which are deuterated to substantially improve the device lifetime compared to non-deuterated host materials or deuteration of other components of the host materials.

In one aspect, the present disclosure provides a compound consisting of a first group and a second group; wherein each of the first group and the second group independently comprises at least one continuous moiety where each of the at least one continuous moiety independently comprising at least three rings; atoms of the first group do not overlap with atoms of the second group and no fused ring system is part of both the first group and the second group; where the compound has a HOMO and a LUMO; the second group comprises at least 70% of the electron density of the HOMO and at least 70% of the electron density of the LUMO; and the first group is at least 30% deuterated.

In another aspect, the present disclosure provides a formulation of the compound of the present disclosure.

In yet another aspect, the present disclosure provides an OLED having an organic layer comprising the compound of the present disclosure.

In yet another aspect, the present disclosure provides a consumer product comprising an OLED with an organic layer comprising the compound of the present disclosure.

### BRIEF DESCRIPTION OF THE DRAWINGS

FIG. 1 shows an organic light emitting device.

FIG. 2 shows an inverted organic light emitting device that does not have a separate electron transport layer.

### DETAILED DESCRIPTION

### A. Terminology

Unless otherwise specified, the below terms used herein are defined as follows:

As used herein, the term “organic” includes polymeric materials as well as small molecule organic materials that may be used to fabricate organic opto-electronic devices. “Small molecule” refers to any organic material that is not a polymer, and “small molecules” may actually be quite large. Small molecules may include repeat units in some circumstances. For example, using a long chain alkyl group as a substituent does not remove a molecule from the “small molecule” class. Small molecules may also be incorporated into polymers, for example as a pendent group on a polymer backbone or as a part of the backbone. Small molecules may also serve as the core moiety of a dendrimer, which consists of a series of chemical shells built on the core moiety. The core moiety of a dendrimer may be a fluorescent or phosphorescent small molecule emitter. A dendrimer may be a “small molecule,” and it is believed that all dendrimers currently used in the field of OLEDs are small molecules.

As used herein, “top” means furthest away from the substrate, while “bottom” means closest to the substrate. Where a first layer is described as “disposed over” a second layer, the first layer is disposed further away from substrate. There may be other layers between the first and second layer, unless it is specified that the first layer is “in contact with” the second layer. For example, a cathode may be described as “disposed over” an anode, even though there are various organic layers in between.

As used herein, “solution processable” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

As used herein, and as would be generally understood by one skilled in the art, a first “Highest Occupied Molecular Orbital” (HOMO) or “Lowest Unoccupied Molecular Orbital” (LUMO) energy level is “greater than” or “higher than” a second HOMO or LUMO energy level if the first energy level is closer to the vacuum energy level. Since ionization potentials (IP) are measured as a negative energy relative to a vacuum level, a higher HOMO energy level corresponds to an IP having a smaller absolute value (an IP that is less negative). Similarly, a higher LUMO energy level corresponds to an electron affinity (EA) having a smaller absolute value (an EA that is less negative). On a conventional energy level diagram, with the vacuum level at the top, the LUMO energy level of a material is higher than the HOMO energy level of the same material. A “higher” HOMO or LUMO energy level appears closer to the top of such a diagram than a “lower” HOMO or LUMO energy level.

As used herein, and as would be generally understood by one skilled in the art, a first work function is “greater than” or “higher than” a second work function if the first work function has a higher absolute value. Because work functions are generally measured as negative numbers relative to vacuum level, this means that a “higher” work function is more negative. On a conventional energy level diagram, with the vacuum level at the top, a “higher” work function is illustrated as further away from the vacuum level in the downward direction. Thus, the definitions of HOMO and LUMO energy levels follow a different convention than work functions.

The terms “halo,” “halogen,” and “halide” are used interchangeably and refer to fluorine, chlorine, bromine, and iodine.

The term “acyl” refers to a substituted carbonyl radical (C(O)—Rₛ).

The term “ester” refers to a substituted oxycarbonyl (—O—C(O)—Rₛ or —C(O)—O—Rₛ) radical.

The term “ether” refers to an —ORₛ radical.

The terms “sulfanyl” or “thio-ether” are used interchangeably and refer to a —SRₛ radical.

The term “selenyl” refers to a —SeRₛ radical.

The term “sulfinyl” refers to a —S(O)—Rₛ radical.

The term “sulfonyl” refers to a —SO₂—Rₛ radical.

The term “phosphino” refers to a —P(Rₛ)₃ radical, wherein each Rₛ can be same or different.

The term “silyl” refers to a —Si(Rₛ)₃ radical, wherein each Rₛ can be same or different.

The term “germyl” refers to a —Ge(Rₛ)₃ radical, wherein each Rₛ can be same or different.

The term “boryl” refers to a —B(Rₛ)₂ radical or its Lewis adduct —B(Rₛ)₃ radical, wherein Rₛ can be same or different.

In each of the above, Rₛ can be hydrogen or a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, and combination thereof. Preferred Rₛ is selected from the group consisting of alkyl, cycloalkyl, aryl, heteroaryl, and combination thereof.

The term “alkyl” refers to and includes both straight and branched chain alkyl radicals. Preferred alkyl groups are those containing from one to fifteen carbon atoms and includes methyl, ethyl, propyl, 1-methylethyl, butyl, 1-methylpropyl, 2-methylpropyl, pentyl, 1-methylbutyl, 2-methylbutyl, 3-methylbutyl, 1,1-dimethylpropyl, 1,2-dimethylpropyl, 2,2-dimethylpropyl, and the like. Additionally, the alkyl group may be optionally substituted.

The term “cycloalkyl” refers to and includes monocyclic, polycyclic, and spiro alkyl radicals. Preferred cycloalkyl groups are those containing 3 to 12 ring carbon atoms and includes cyclopropyl, cyclopentyl, cyclohexyl, bicyclo[3.1.1]heptyl, spiro[4.5]decyl, spiro[5.5]undecyl, adamantyl, and the like. Additionally, the cycloalkyl group may be optionally substituted.

The terms “heteroalkyl” or “heterocycloalkyl” refer to an alkyl or a cycloalkyl radical, respectively, having at least one carbon atom replaced by a heteroatom. Optionally the at least one heteroatom is selected from O, S, N, P, B, Si and Se, preferably, O, S or N. Additionally, the heteroalkyl or heterocycloalkyl group may be optionally substituted.

The term “alkenyl” refers to and includes both straight and branched chain alkene radicals. Alkenyl groups are essentially alkyl groups that include at least one carbon-carbon double bond in the alkyl chain. Cycloalkenyl groups are essentially cycloalkyl groups that include at least one carbon-carbon double bond in the cycloalkyl ring. The term “heteroalkenyl” as used herein refers to an alkenyl radical having at least one carbon atom replaced by a heteroatom. Optionally the at least one heteroatom is selected from O, S, N, P, B, Si, and Se, preferably, O, S, or N. Preferred alkenyl, cycloalkenyl, or heteroalkenyl groups are those containing two to fifteen carbon atoms. Additionally, the alkenyl, cycloalkenyl, or heteroalkenyl group may be optionally substituted.

The term “alkynyl” refers to and includes both straight and branched chain alkyne radicals. Alkynyl groups are essentially alkyl groups that include at least one carbon-carbon triple bond in the alkyl chain. Preferred alkynyl groups are those containing two to fifteen carbon atoms. Additionally, the alkynyl group may be optionally substituted.

The terms “aralkyl” or “arylalkyl” are used interchangeably and refer to an alkyl group that is substituted with an aryl group. Additionally, the aralkyl group may be optionally substituted.

The term “heterocyclic group” refers to and includes aromatic and non-aromatic cyclic radicals containing at least one heteroatom. Optionally the at least one heteroatom is selected from O, S, N, P, B, Si, and Se, preferably, O, S, or N. Hetero-aromatic cyclic radicals may be used interchangeably with heteroaryl. Preferred hetero-non-aromatic cyclic groups are those containing 3 to 7 ring atoms which includes at least one hetero atom, and includes cyclic amines such as morpholino, piperidino, pyrrolidino, and the like, and cyclic ethers/thio-ethers, such as tetrahydrofuran, tetrahydropyran, tetrahydrothiophene, and the like. Additionally, the heterocyclic group may be optionally substituted.

The term “aryl” refers to and includes both single-ring aromatic hydrocarbyl groups and polycyclic aromatic ring systems. The polycyclic rings may have two or more rings in which two carbons are common to two adjoining rings (the rings are “fused”) wherein at least one of the rings is an aromatic hydrocarbyl group, e.g., the other rings can be cycloalkyls, cycloalkenyls, aryl, heterocycles, and/or heteroaryls. Preferred aryl groups are those containing six to thirty carbon atoms, preferably six to twenty carbon atoms, more preferably six to twelve carbon atoms. Especially preferred is an aryl group having six carbons, ten carbons or twelve carbons. Suitable aryl groups include phenyl, biphenyl, triphenyl, triphenylene, tetraphenylene, naphthalene, anthracene, phenalene, phenanthrene, fluorene, pyrene, chrysene, perylene, and azulene, preferably phenyl, biphenyl, triphenyl, triphenylene, fluorene, and naphthalene. Additionally, the aryl group may be optionally substituted.

The term “heteroaryl” refers to and includes both single-ring aromatic groups and polycyclic aromatic ring systems that include at least one heteroatom. The heteroatoms include, but are not limited to O, S, N, P, B, Si, and Se. In many instances, O, S, or N are the preferred heteroatoms. Hetero-single ring aromatic systems are preferably single rings with 5 or 6 ring atoms, and the ring can have from one to six heteroatoms. The hetero-polycyclic ring systems can have two or more rings in which two atoms are common to two adjoining rings (the rings are “fused”) wherein at least one of the rings is a heteroaryl, e.g., the other rings can be cycloalkyls, cycloalkenyls, aryl, heterocycles, and/or heteroaryls. The hetero-polycyclic aromatic ring systems can have from one to six heteroatoms per ring of the polycyclic aromatic ring system. Preferred heteroaryl groups are those containing three to thirty carbon atoms, preferably three to twenty carbon atoms, more preferably three to twelve carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Of the aryl and heteroaryl groups listed above, the groups of triphenylene, naphthalene, anthracene, dibenzothiophene, dibenzofuran, dibenzoselenophene, carbazole, indolocarbazole, imidazole, pyridine, pyrazine, pyrimidine, triazine, and benzimidazole, and the respective aza-analogs of each thereof are of particular interest.

The terms alkyl, cycloalkyl, heteroalkyl, heterocycloalkyl, alkenyl, cycloalkenyl, heteroalkenyl, alkynyl, aralkyl, heterocyclic group, aryl, and heteroaryl, as used herein, are independently unsubstituted, or independently substituted, with one or more General Substituents.

In many instances, the General Substituents are selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, selenyl, sulfinyl, sulfonyl, phosphino, and combinations thereof.

In some instances, the Preferred General Substituents are selected from the group consisting of deuterium, fluorine, alkyl, cycloalkyl, heteroalkyl, alkoxy, aryloxy, amino, silyl, germyl, boryl, alkenyl, cycloalkenyl, heteroalkenyl, aryl, heteroaryl, nitrile, isonitrile, sulfanyl, and combinations thereof.

In some instances, the More Preferred General Substituents are selected from the group consisting of deuterium, fluorine, alkyl, cycloalkyl, alkoxy, aryloxy, amino, silyl, aryl, heteroaryl, sulfanyl, and combinations thereof.

In yet other instances, the Most Preferred General Substituents are selected from the group consisting of deuterium, fluorine, alkyl, cycloalkyl, aryl, heteroaryl, and combinations thereof.

The terms “substituted” and “substitution” refer to a substituent other than H that is bonded to the relevant position, e.g., a carbon or nitrogen. For example, when R¹ represents mono-substitution, then one R¹ must be other than H (i.e., a substitution). Similarly, when R¹ represents di-substitution, then two of R¹ must be other than H. Similarly, when R¹ represents zero or no substitution, R¹, for example, can be a hydrogen for available valencies of ring atoms, as in carbon atoms for benzene and the nitrogen atom in pyrrole, or simply represents nothing for ring atoms with fully filled valencies, e.g., the nitrogen atom in pyridine. The maximum number of substitutions possible in a ring structure will depend on the total number of available valencies in the ring atoms.

As used herein, “combinations thereof” indicates that one or more members of the applicable list are combined to form a known or chemically stable arrangement that one of ordinary skill in the art can envision from the applicable list. For example, an alkyl and deuterium can be combined to form a partial or fully deuterated alkyl group; a halogen and alkyl can be combined to form a halogenated alkyl substituent; and a halogen, alkyl, and aryl can be combined to form a halogenated arylalkyl. In one instance, the term substitution includes a combination of two to four of the listed groups. In another instance, the term substitution includes a combination of two to three groups. In yet another instance, the term substitution includes a combination of two groups. Preferred combinations of substituent groups are those that contain up to fifty atoms that are not hydrogen or deuterium, or those which include up to forty atoms that are not hydrogen or deuterium, or those that include up to thirty atoms that are not hydrogen or deuterium. In many instances, a preferred combination of substituent groups will include up to twenty atoms that are not hydrogen or deuterium.

The “aza” designation in the fragments described herein, i.e. aza-dibenzofuran, aza-dibenzothiophene, etc. means that one or more of the C—H groups in the respective aromatic ring can be replaced by a nitrogen atom, for example, and without any limitation, azatriphenylene encompasses both dibenzo[f,h]quinoxaline and dibenzo[f,h]quinoline. One of ordinary skill in the art can readily envision other nitrogen analogs of the aza-derivatives described above, and all such analogs are intended to be encompassed by the terms as set forth herein.

As used herein, “deuterium” refers to an isotope of hydrogen. Deuterated compounds can be readily prepared using methods known in the art. For example, U.S. Pat. No. 8,557,400, Patent Pub. No. WO 2006/095951, and U.S. Pat. Application Pub. No. US 2011/0037057, which are hereby incorporated by reference in their entireties, describe the making of deuterium-substituted organometallic complexes. Further reference is made to Ming Yan, et al., Tetrahedron 2015, 71, 1425-30 and Atzrodt et al., Angew. Chem. Int. Ed. (Reviews) 2007, 46, 7744-65, which are incorporated by reference in their entireties, describe the deuteration of the methylene hydrogens in benzyl amines and efficient pathways to replace aromatic ring hydrogens with deuterium, respectively.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or attached fragment are considered to be equivalent.

In some instance, a pair of adjacent substituents can be optionally joined or fused into a ring. The preferred ring is a five, six, or seven-membered carbocyclic or heterocyclic ring, includes both instances where the portion of the ring formed by the pair of substituents is saturated and where the portion of the ring formed by the pair of substituents is unsaturated. As used herein, “adjacent” means that the two substituents involved can be on the same ring next to each other, or on two neighboring rings having the two closest available substitutable positions, such as 2, 2′ positions in a biphenyl, or 1, 8 position in a naphthalene, as long as they can form a stable fused ring system.

### B. The Compounds of the Present Disclosure

In one aspect, the present disclosure provides a compound consisting of a first group and a second group; wherein:
each of the first group and the second group independently comprises at least one continuous moiety where each of the at least one continuous moiety independently comprising at least three rings;
atoms of the first group do not overlap with atoms of the second group and no fused ring system is part of both the first group and the second group;
the compound has a HOMO and a LUMO;
the second group contains at least 70% of the electron densities of the HOMO and the LUMO; and
the first group is at least 30% deuterated.

As used herein, the first group and the second group refer to discrete groups of atoms that form the subject compound. Because the compound consists of the first group and the second group, all atoms of the compound belong to either the first group or the second group. As used herein, “atoms of the first group do not overlap with the second group” means that no atom is in both the first group and the second group. “No fused ring system is part of both groups” means a fused ring system cannot be divided, and it cannot straddle into the first group and the second group at the same time. In other words, a fused ring system can only be in one group.

In some embodiments, each non-cyclic moiety is contained entirely within one of the first group and the second group.

Each of the at least one continuous moiety comprising at least three rings can be at least three rings that are bonded together directly or they can be bonded together via a linking group (e.g., Si(Ph)₃) or the continuous moiety can be three rings fused together (e.g., carbazole moiety). For instance, considering only the definitions of the first group and the second group (not electron densities, percent deuteration, etc.), the following compound can be grouped several ways:

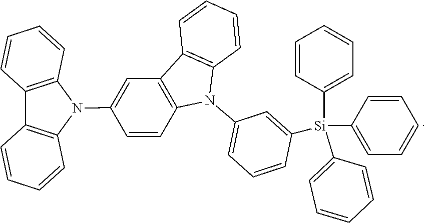 [SMILES: c1ccc([Si](c2ccccc2)(c2ccccc2)c2cccc(-n3c4ccccc4c4cc(-n5c6ccccc6c6ccccc65)ccc43)c2)cc1]

For instance, in a first grouping, the first group can be the Si(Ph)₃ moiety and the second group can be the two carbazoles and the phenyl linker. In a second grouping, the first group can be the Si(Ph)₃ moiety and the phenyl linker, while the second group can be the two carbazoles. In a third grouping, the first group can be the Si(Ph)₃ moiety, the phenyl linker, and the carbazole attached thereto, while the second group can be the terminal carbazole. However, fused ring systems must be part of the same moiety and, therefore, the same group.

In some embodiments, the compound consists of a first group and a second group, where each group consists of at least one moiety independently comprising at least three rings that are continuous. Although not explicitly stated, this requires that neither the first group nor the second group includes any atoms or rings that are not part of a continuous moiety comprising at least three rings.

In some embodiments, the first group can be continuous. In some embodiments, the second group can be continuous. In some embodiments, the first group and the second group both can be continuous.

In some embodiments, the first group can be non-continuous. For example, the first group can include two continuous moieties comprising at least three rings as in the Si(Ph)₃ moieties in the following compound:

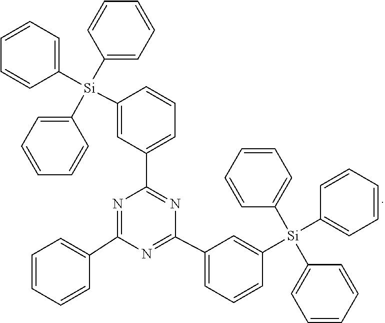 [SMILES: c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1]

The central triazine ring with pendant phenyl groups would be the second group.

In some embodiments, the second group can be non-continuous (e.g., more than one continuous moiety comprising at least three rings. In some embodiments, the first group and the second group can be both non-continuous. In some embodiments, the first group can be continuous and the second group can be non-continuous or vice versa.

In some embodiments, the first group can have a minimum percentage of deuteration of 30% (i.e., at least 30% deuterated), 50%, 70%, 90%, 99%, and 100%. For example, the first group is at least 30% deuterated, or at least 50% deuterated, or at least 70% deuterated, or at least 90% deuterated, or at least 99% deuterated, or at least 100% deuterated.

In some embodiments, the second group can comprise a minimum percentage of the electron densities of both the HOMO and the LUMO of the compound of 70% (i.e., at least 70% of electron densities), 75%, 80%, 85%, 90%, 95%, and 100%. For example, the second group comprises at least 70% of the electron densities of both the HOMO and the LUMO, or at least 75% of the electron densities of both the HOMO and the LUMO, or at least 80% of the electron densities of both the HOMO and the LUMO, or at least 85% of the electron densities of both the HOMO and the LUMO, or at least 90% of the electron densities of both the HOMO and the LUMO, or at least 95% of the electron densities of both the HOMO and the LUMO, or at least 100% of the electron densities of both the HOMO and the LUMO.

In some embodiments, the first group can have a lowest triplet energy T₁ of at least 3.00 eV. In some embodiments, the first group can have a lowest triplet energy T₁ of at least 3.05 eV. In some embodiments, the first group can have a lowest triplet energy T₁ of at least 3.10 eV. In some embodiments, the first group can have a lowest triplet energy T₁ of at least 3.15 eV. In some embodiments, the first group can have a lowest triplet energy T₁ of at least 3.20 eV.

In some embodiments, the second group is not deuterated.

In some embodiments, the second group can have a maximum percentage of deuteration of 20% (i.e., up to 20% deuterated), 10%, 5%, and 0%. For example, the second group can have a maximum percentage of deuteration of 20%, or a maximum percentage of deuteration of 10%, or a maximum percentage of deuteration of 5%, or a maximum percentage of deuteration of 0%.

In another aspect, the present disclosure provides a compound consisting of a first group and a second group; wherein:
each of the first group and the second group independently comprises at least one continuous moiety comprising at least three rings;
atoms of the first group do not overlap with atoms of the second group, and no fused ring system is part of both groups;
the first group comprises at least one first moiety selected from the group consisting of X—[Ar]ₙ, triphenylene, tetraphenylene, terphenyl, xanthene, and 9,10-dihydroanthracene; where X is selected from the group consisting of sp³ carbon atom, sp³ silicon atom, and sp³ germanium atom; n is an integer from 1 to 4;
when n is an integer from 2 to 4, each Ar is partially or fully deuterated and can be same or different;
each Ar is not bonded or fused to another Ar;
wherein each Ar is independently a 5-membered or 6-membered carbocyclic or heterocyclic aromatic ring or a fused ring system comprising at least two rings that are each independently a 5-membered and/or 6-membered carbocyclic or heterocyclic ring;
the second group comprises at least one second moiety selected from the group consisting of carbazole, indolocarbazole, dibenzothiophene, indolodibenzothiophene, dibenzofuran, indolodibenzofuran, dibenzoselenophene, indolodibenzoselenophene, 5,9-dioxa-13b-boranaphtho[3,2,1-de]anthracene, imidazole, pyridine, pyrimidine, pyrazine, pyridazine, triazine, benzo[d]benzo[4,5]imidazo[1,2-a]imidazole; and aza-variants thereof; and
wherein each benzene ring of the triphenylene, tetraphenylene, terphenyl, xanthene, or 9,10-dihydroanthracene of the first moiety comprises at least one D atom.

In some embodiments, each Ar is independently a 5-membered or 6-membered aryl or heteroaryl ring or a fused ring system comprising at least two rings that are each independently a 5-membered and/or 6-membered aryl or heteroaryl ring.

In some embodiments, the at least one first moiety comprises triphenylene and each benzene ring comprises at least one D atom. In some embodiments, the at least one first moiety comprises tetraphenylene and each benzene ring comprises at least one D atom. In some embodiments, the at least one first moiety comprises terphenyl and each benzene ring comprises at least one D atom. In some embodiments, the at least one first moiety comprises xanthene and each benzene ring comprises at least one D atom. In some embodiments, the at least one first moiety comprises 9,10-dihydroanthracene and each benzene ring comprises at least one D atom.

In some embodiments, the at least one first moiety is X—[Ar]ₙ and each Ar comprises at least one D atom.

In some embodiments, each Ar comprises at least two D atoms or each benzene ring of the triphenylene, tetraphenylene, terphenyl, xanthene, or 9,10-dihydroanthracene of the first moiety comprises at least two D atoms.

In some embodiments, each Ar or each benzene ring of the triphenylene, tetraphenylene, terphenyl, xanthene, or 9,10-dihydroanthracene of the first moiety is fully deuterated.

In some embodiments, X is an sp³ carbon atom.

In some embodiments, X is an sp³ silicon atom.

In some embodiments, X is an sp³ germanium atom.

In some embodiments, the first group of the compound comprises at least two X—[Ar]ₙ groups, which can be the same or different. In some embodiments, the first group of the compound comprises at least two X—[Ar]ₙ groups, which can be continuous or non-continuous.

In some of the above embodiments, n is an integer of 3 or 4.

In some embodiments n is 3 and X is attached to an additional ring Ar^{H}, wherein Ar^{H} may be further fused or substituted and wherein Ar^{H} is not deuterated. In some embodiments, Ar^{H} is not deuterated and other rings in the molecule are fully or partially deuterated. In some embodiments, Ar^{H} is not deuterated and each Ar group is fully deuterated. In some embodiments, the second moiety comprises Ar^{H}. In some embodiments, the second moiety is connected through one or more bond to Ar^{H}. In some embodiments, at least 20% of the HOMO density is localized on Ar^{H}. In some embodiments, at least 20% of the LUMO density is localized on Ar^{H}. In some embodiments Ar^{H} is a 6 membered aromatic ring. In some embodiments Ar^{H} is selected from the group consisting of benzene, pyridine, pyrimidine, pyrazine, and triazine, and which may be further substituted or unsubstituted.

In some embodiments n is 2 and X is attached to at least one moiety, A^{N}, selected from the group consisting of alkyl, heteroalkyl, cycloalkyl, heterocycloalkyl silyl, germyl, and combinations thereof. In some embodiments A^{N} is fully or partially deuterated.

In some of the above embodiments, each Ar can be phenyl.

In some of the above embodiments, when Ar is present, each Ar has a minimum percentage of the deuteration of 30% (e.g., at least 30% deuterated), 50%, 70%, 90%, 99%, or 100%.

In some of the above embodiments, when tetraphenylene is present, each benzene ring, including any substituents that are part of the first group as the benzene ring, of the tetraphenylene has a minimum percentage of deuteration selected from the group consisting of 30%, 50%, 70%, 90%, 99%, and 100%.

In some of the above embodiments, the second group is not deuterated.

In some of the embodiments, the first group has a minimum percentage of deuteration of 30% (e.g., at least 30% deuterated), 50%, 70%, 90%, 99%, or 100%.

In some of the embodiments, the second group consists of a minimum percentage of the electron densities of the HOMO and the LUMO of the compound selected from the group consisting of 70%, 75%, 80%, 85%, 90%, 95%, and 100%.

In some embodiments, the compound has a LUMO less than −2.4 eV. In some embodiments, the compound has a LUMO less than −2.5 eV. In some embodiments, the compound has a LUMO less than −2.6 eV. In some embodiments, the compound has a LUMO less than −2.7 eV.

In some of the above embodiments, the first group may have a lowest triplet energy T₁ of at least 3.00 eV. In some of the above embodiments, the first group may have a lowest triplet energy T₁ of at least 3.05 eV. In some of the above embodiments, the first group may have a lowest triplet energy T₁ of at least 3.10 eV. In some of the above embodiments, the first group may have a lowest triplet energy T₁ of at least 3.15 eV. In some of the above embodiments, the first group may have a lowest triplet energy T₁ of at least 3.20 eV.

In some of the above embodiments, the second group has a maximum percentage of deuteration of 20% (e.g., up to 30% deuterated), 10%, 5%, or 0%.

In some embodiments, the second group comprises at least one structure selected from the group consisting of:

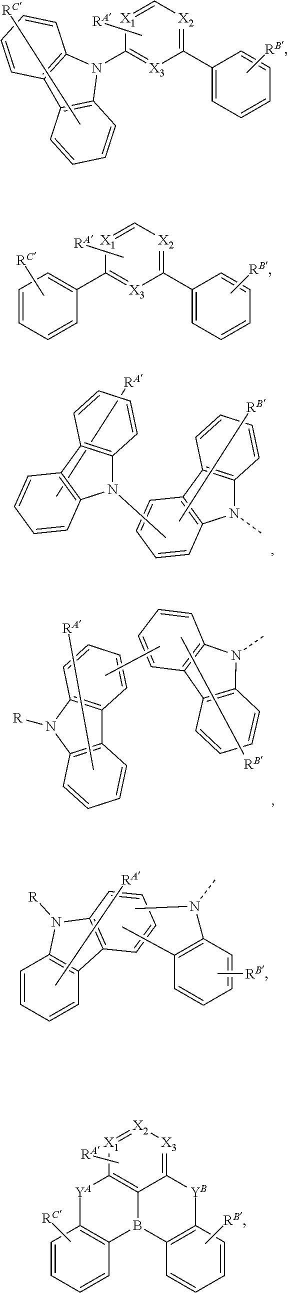

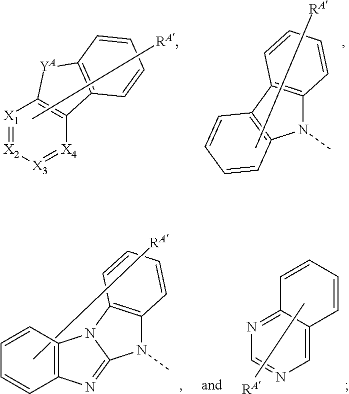

wherein:
   each of X₁ to X₄ is independently selected from the group consisting of C and N;
   each of Y^{A} and Y^{B} is independently selected from the group consisting of BR, NR, PR, O, S, Se, C═O, S═O, SO₂, CRR′, SiRR′, and GeRR′;
   each of R^{A′}, R^{B′}, and R^{C′} is independently represent from mono to the maximum possible number of substitutions, or no substitution;
   each R, R′, R^{A′}, R^{B′}, and R^{C′} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, selenyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
   wherein any two R, R′, R^{A′}, R^{B′}, and R^{C′} can be fused or joined to form a ring.

In some embodiments, the second group comprises at least one

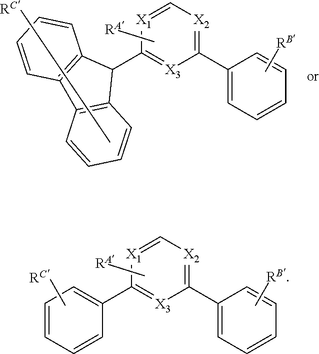

In some such embodiments, at least one X₁ to X₃ is N. In some such embodiments, at least two of X₁ to X₃ are N. In some such embodiments, each of X₁ to X₃ is N.

In some embodiments, the second group comprises at least one

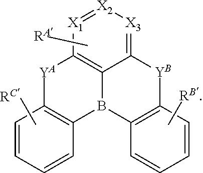

In some such embodiments, Y^{A} and Y^{B} are both O. In some such embodiments, at least one of X₁ to X₃ is N. In some such embodiments, at least two of X₁ to X₃ are N.

In some embodiments, the second group comprises at least one

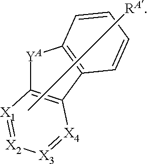

In some such embodiments, at least one of X₁ to X₄ is N. In some such embodiments, at least two of X₁ to X₄ are N.

In some embodiments, the first group comprises at least one structure selected from the group consisting of:

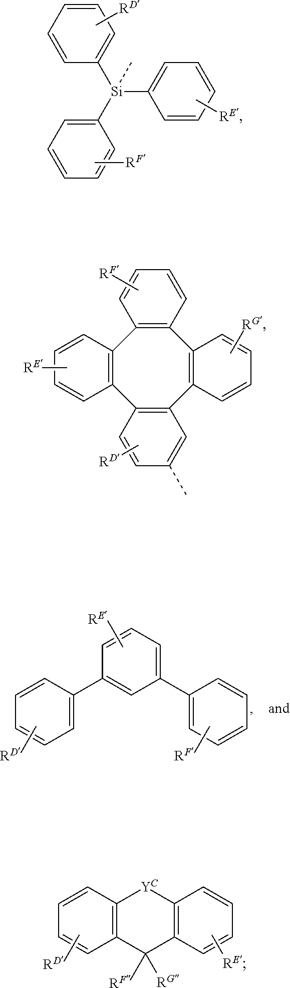

wherein:
Y^{C} is selected from the group consisting of BR, NR, PR, O, S, Se, C═O, S═O, SO₂, CRR′, SiRR′, and GeRR′;
each of R^{D′}, R^{E′}, R^{F′}, and R^{G′} independently represents from mono to the maximum possible number of substitutions, or no substitutions;
wherein each R, R′, R^{D′}, R^{E′}, R^{F′}, R^{F″}, R^{G′}, and R^{G″} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, selenyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein R, R′, R^{D′}, R^{E′}, R^{F′}, and R^{G′} are at least 30% deuterated.

In some of the embodiments, each of R^{D′}, R^{E′}, R^{F′}, and R^{G′} is independently selected from the group consisting of deuterium, alkyl, cycloalkyl, aryl, heteroaryl, and combinations thereof.

In the structures for the first group and second group shown above, where no dashed line is provided, the structure can be bonded to adjacent structures at any point in the structure.

In some embodiments, each R, R′, R^{D′}, R^{E′}, R^{F′}, R^{F″}, R^{G′}, and R^{G″} is independently selected from the group consisting of deuterium, alkyl, cycloalkyl, aryl, heteroaryl, and combinations thereof.

In some embodiments, at least one R, R′, R^{D′}, R^{E′}, R^{F′}, R^{F″}, R^{G′}, or R^{G″} comprises aryl or heteroaryl.

In some of the above embodiments, the compound can be selected from the group consisting of the structures of the following LIST 1:

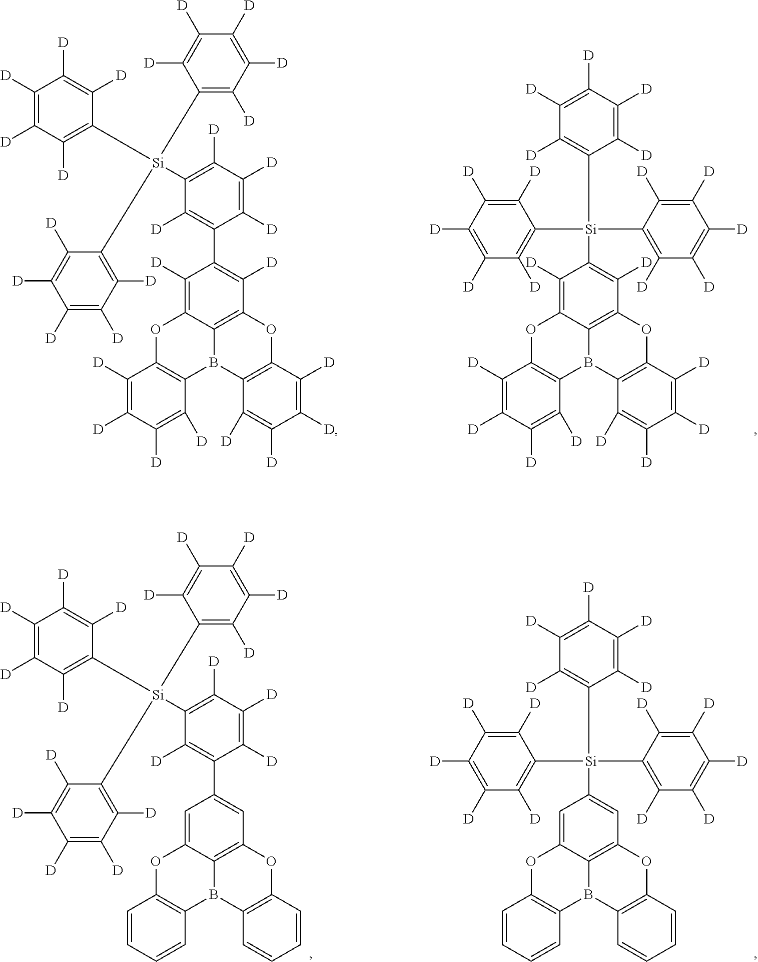

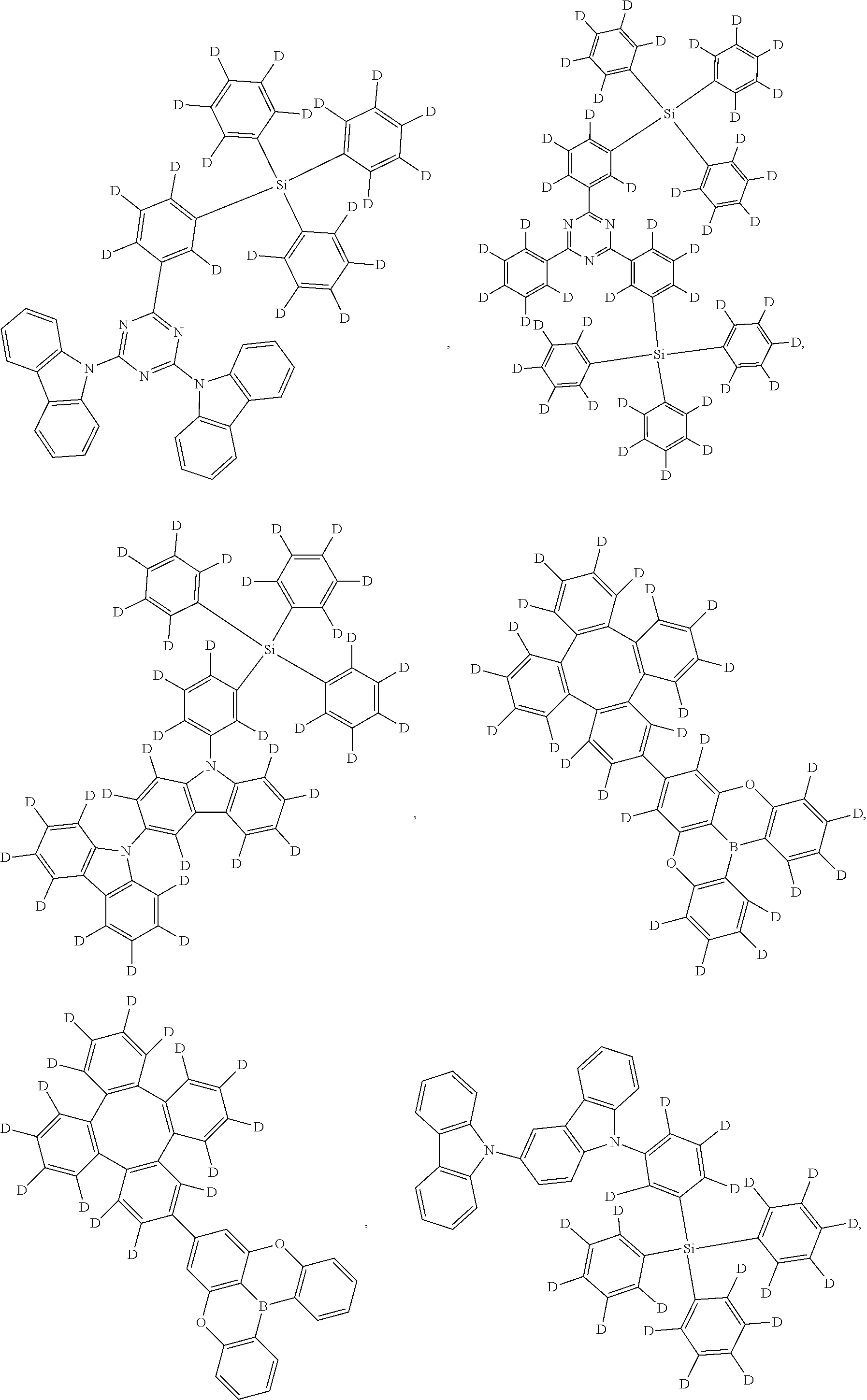

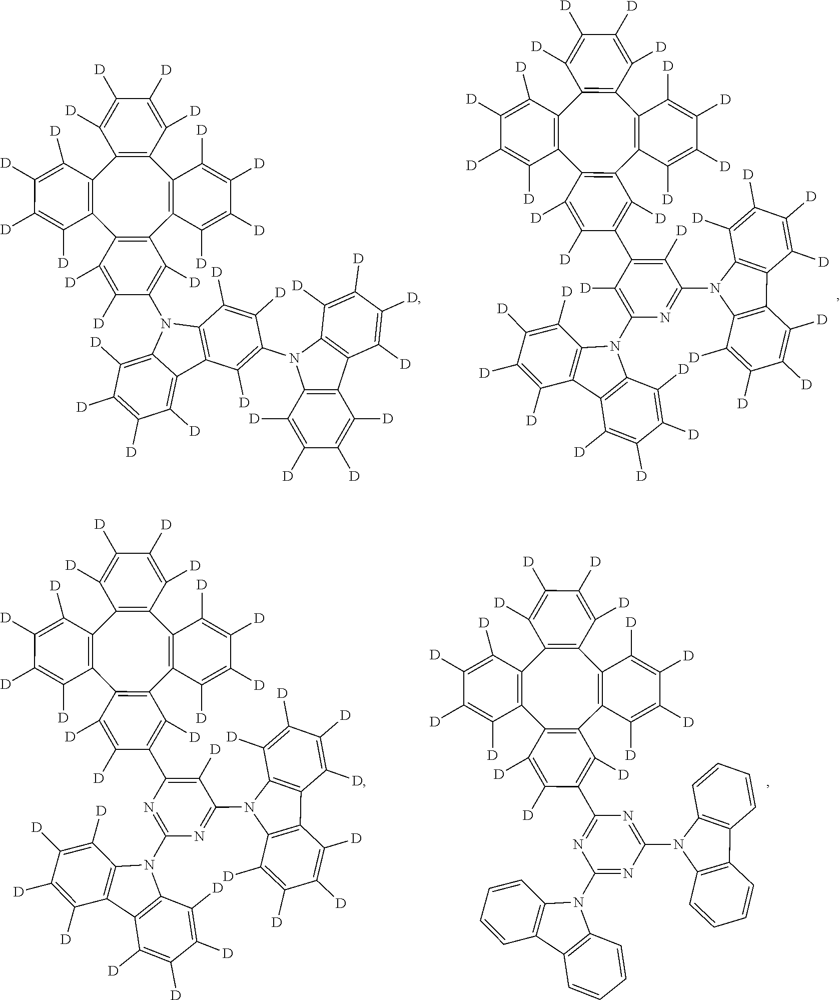

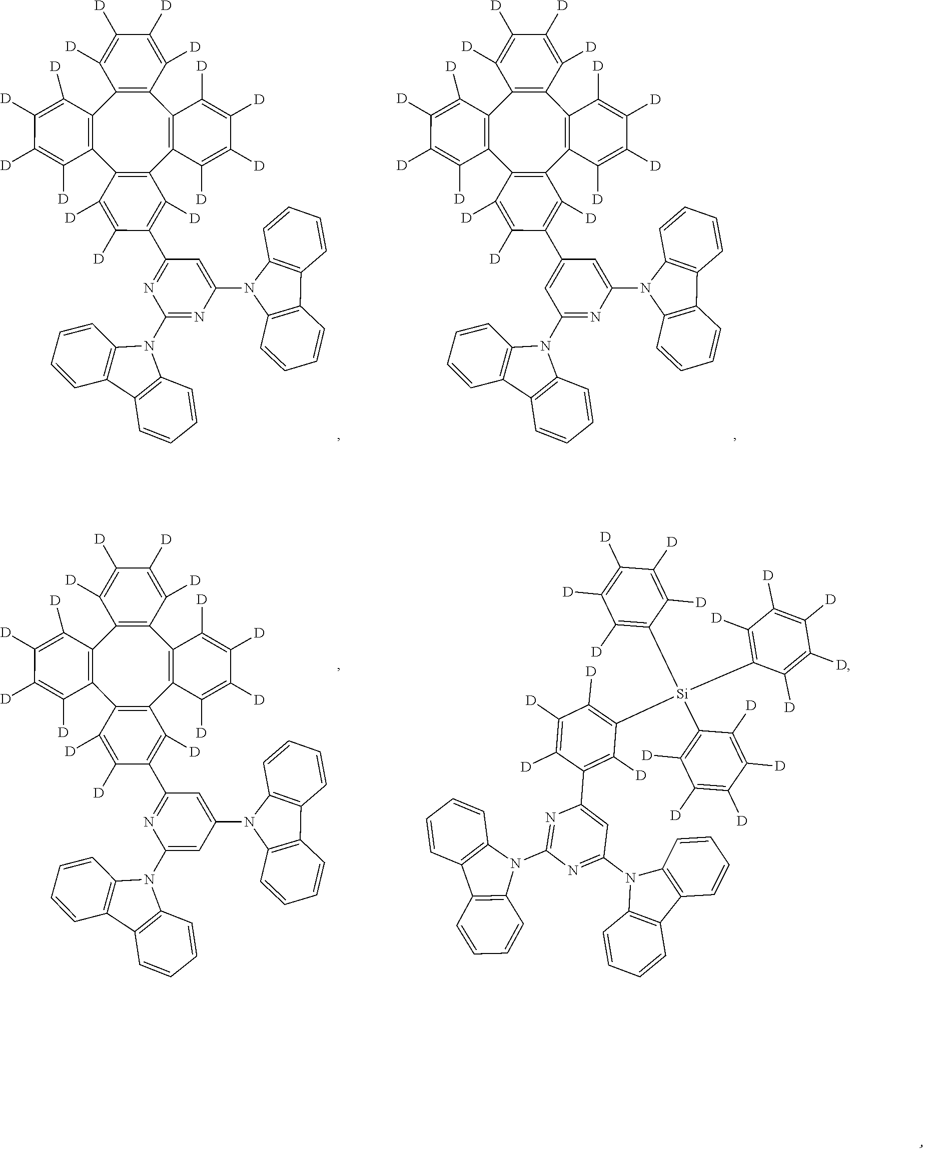

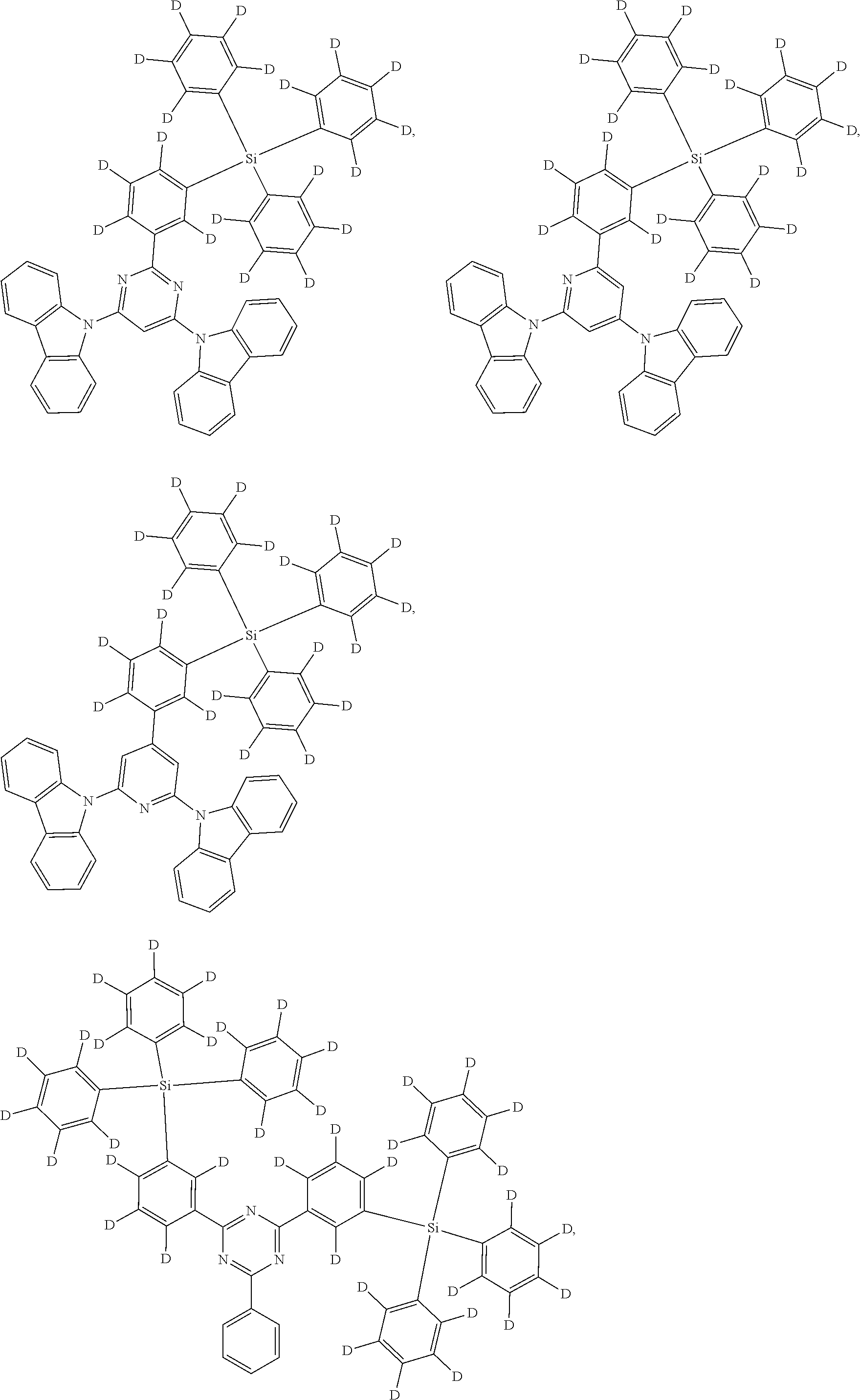

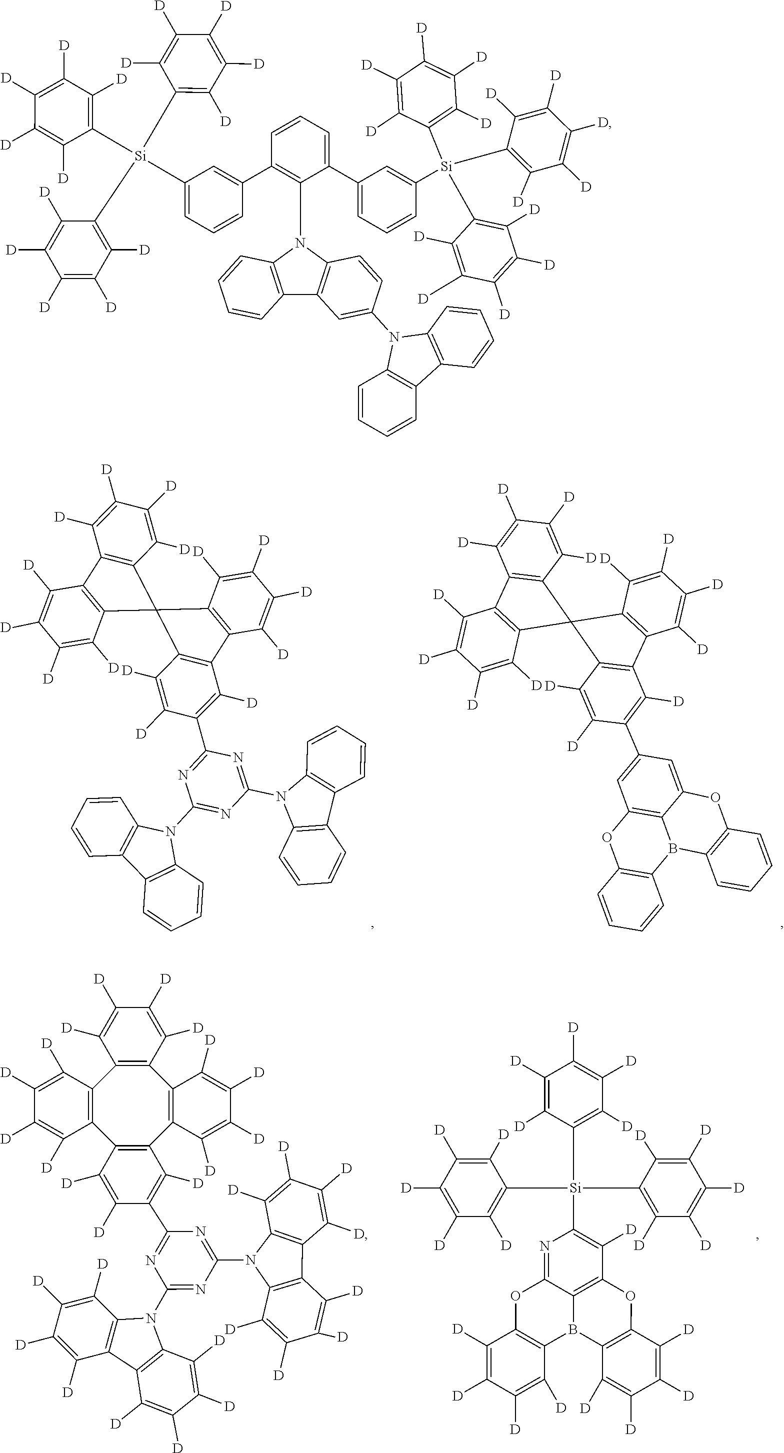

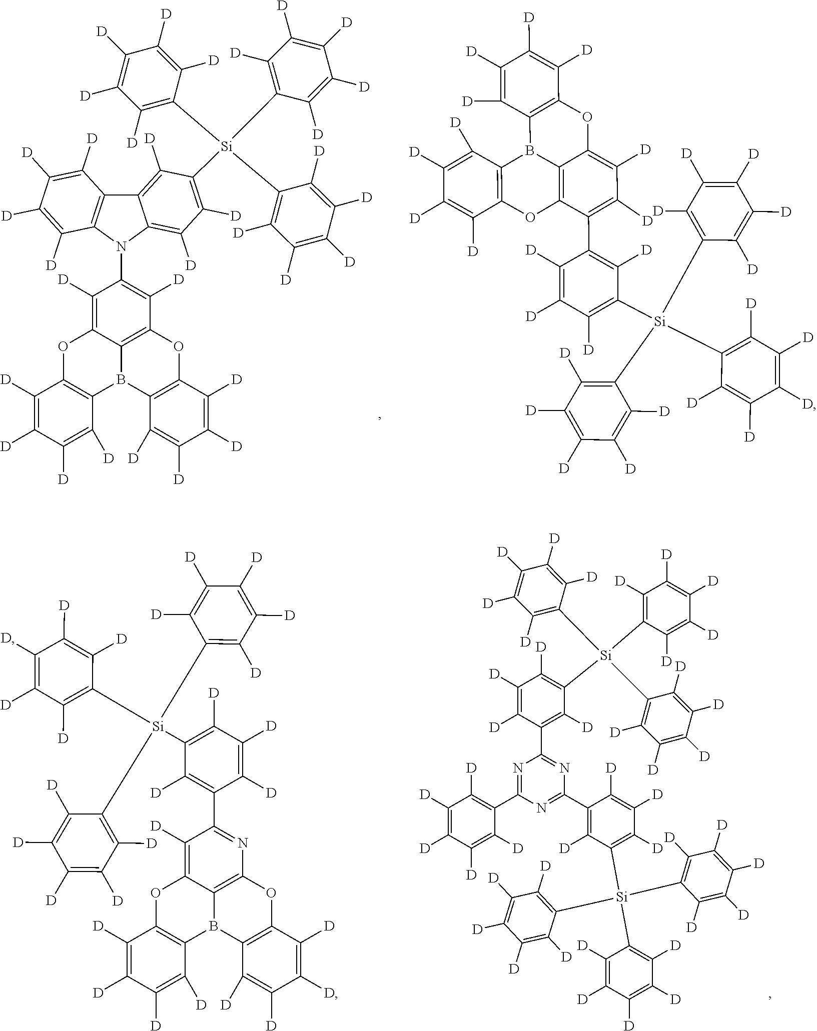

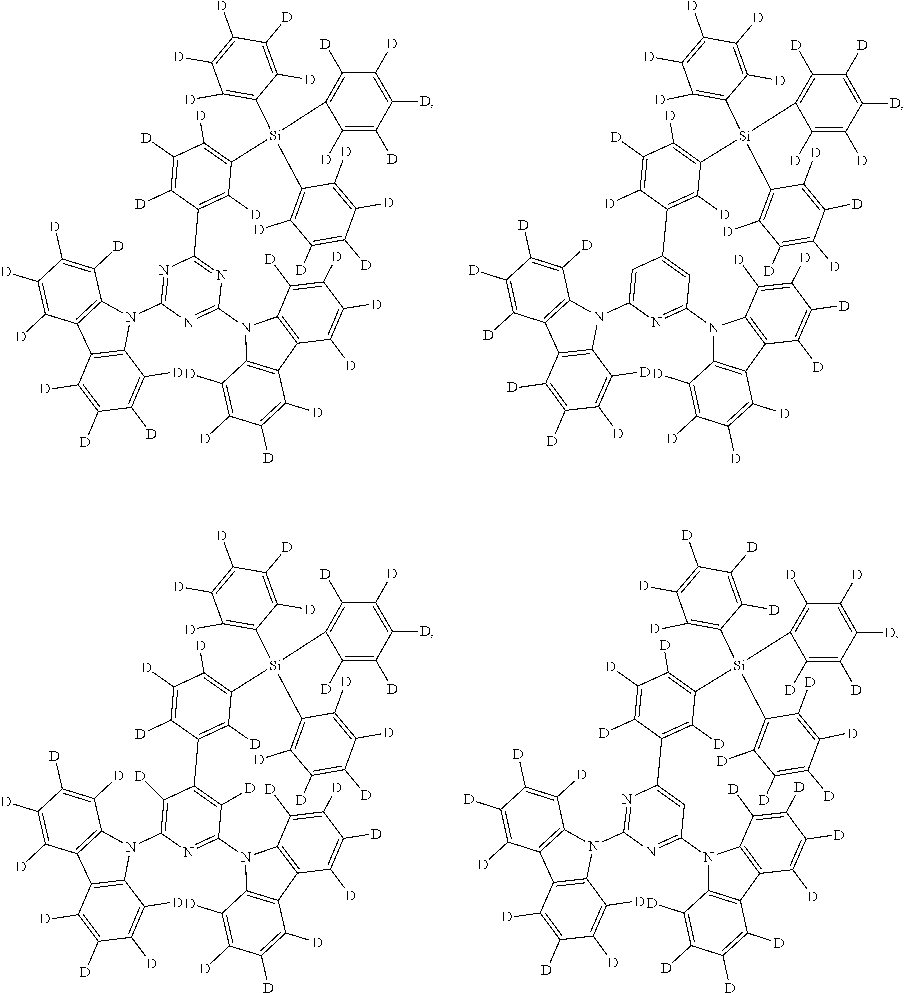

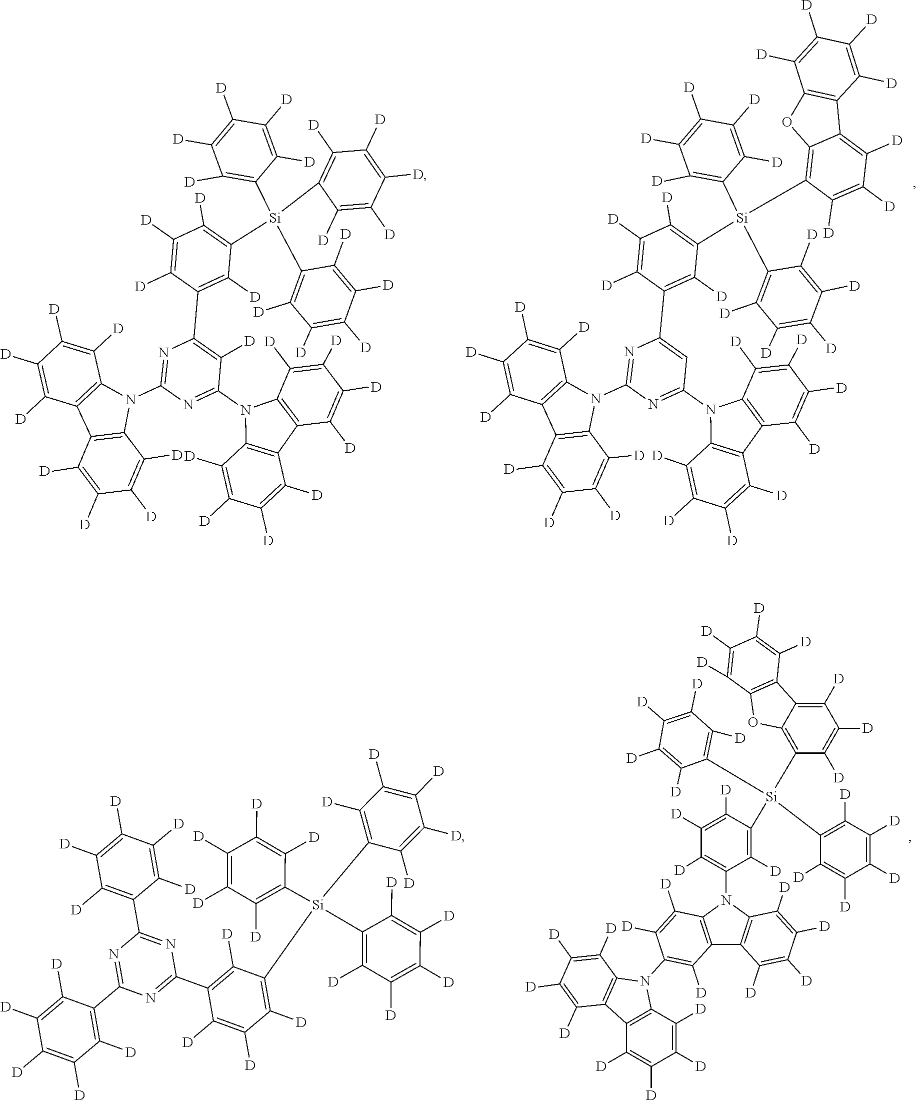

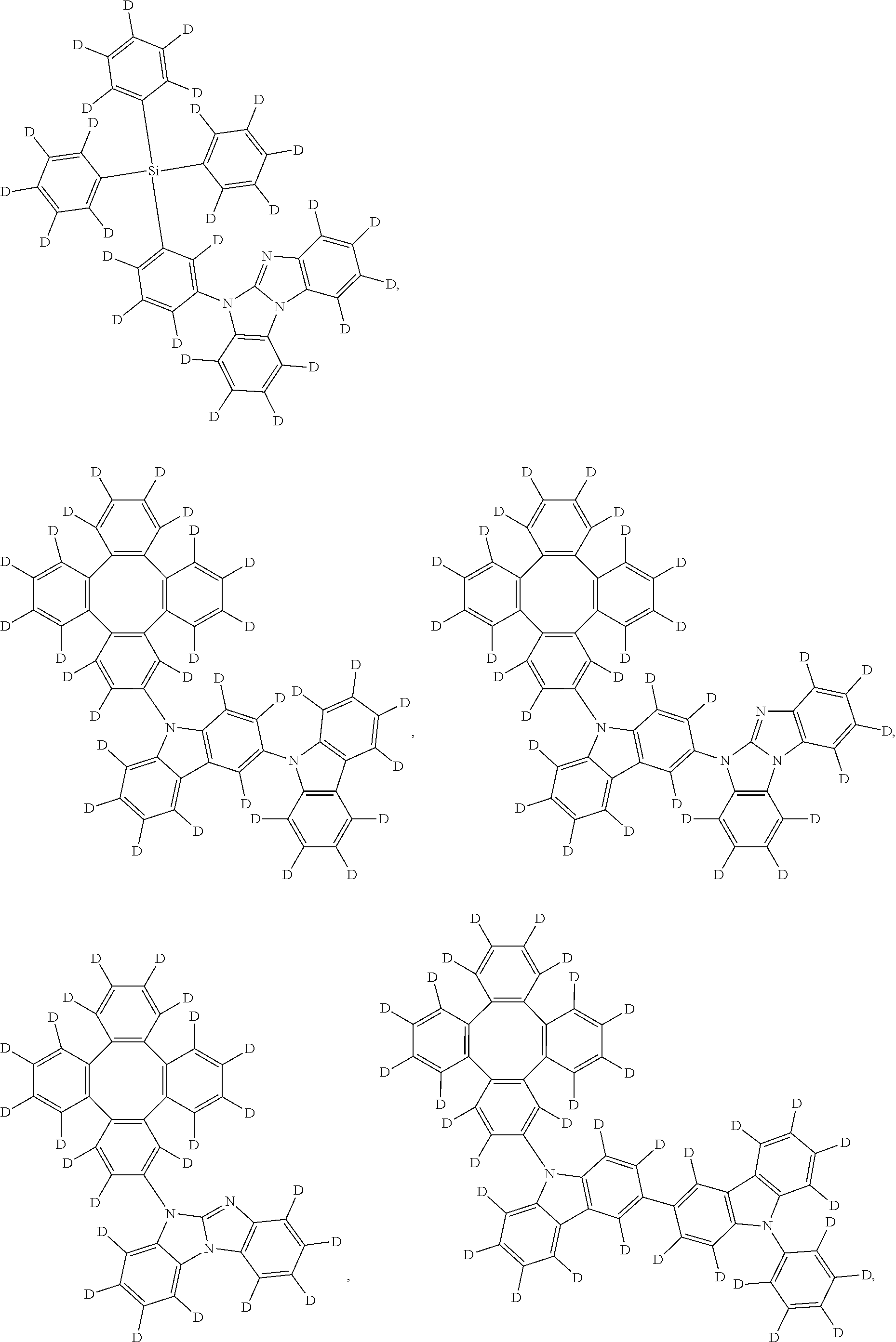

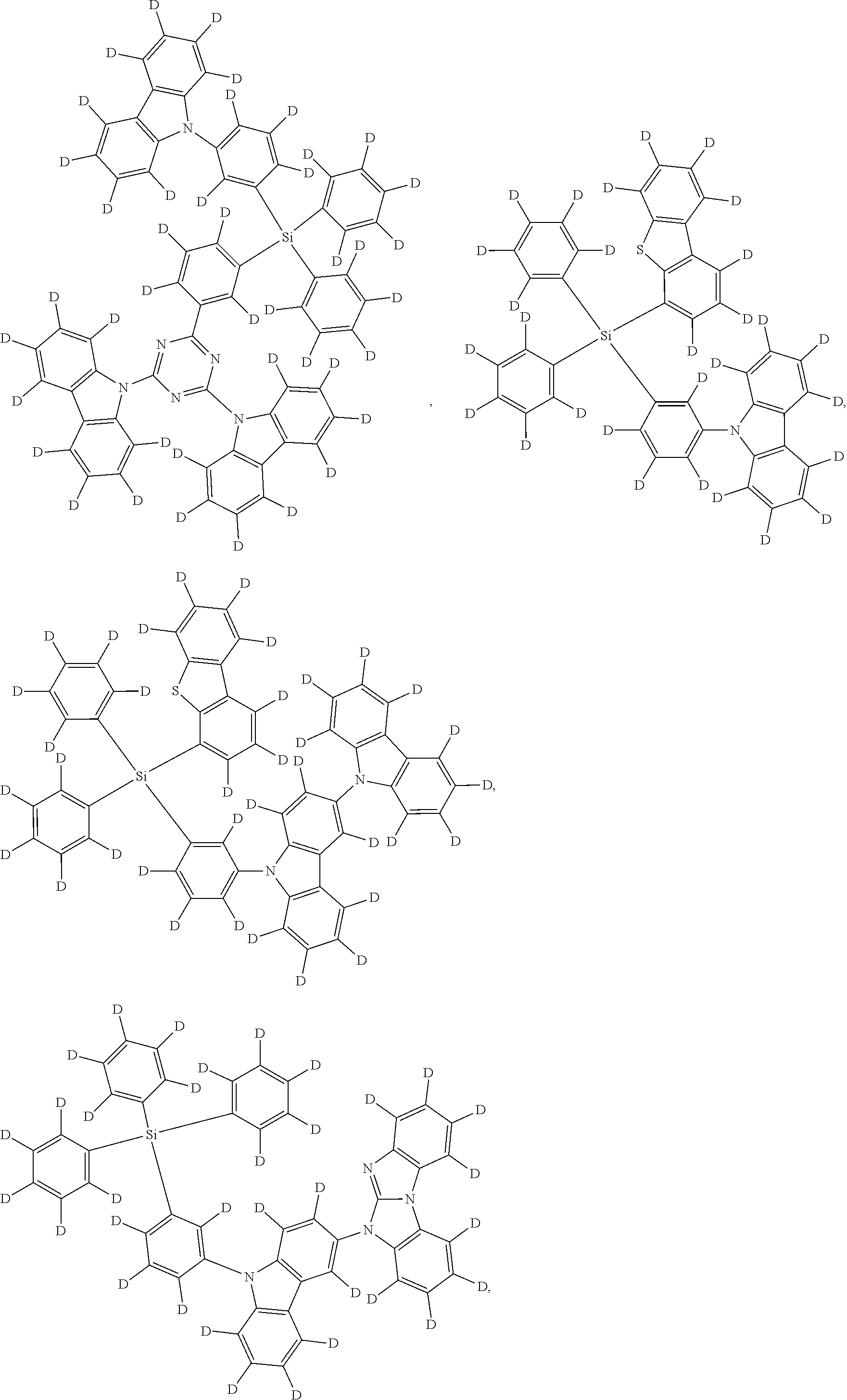

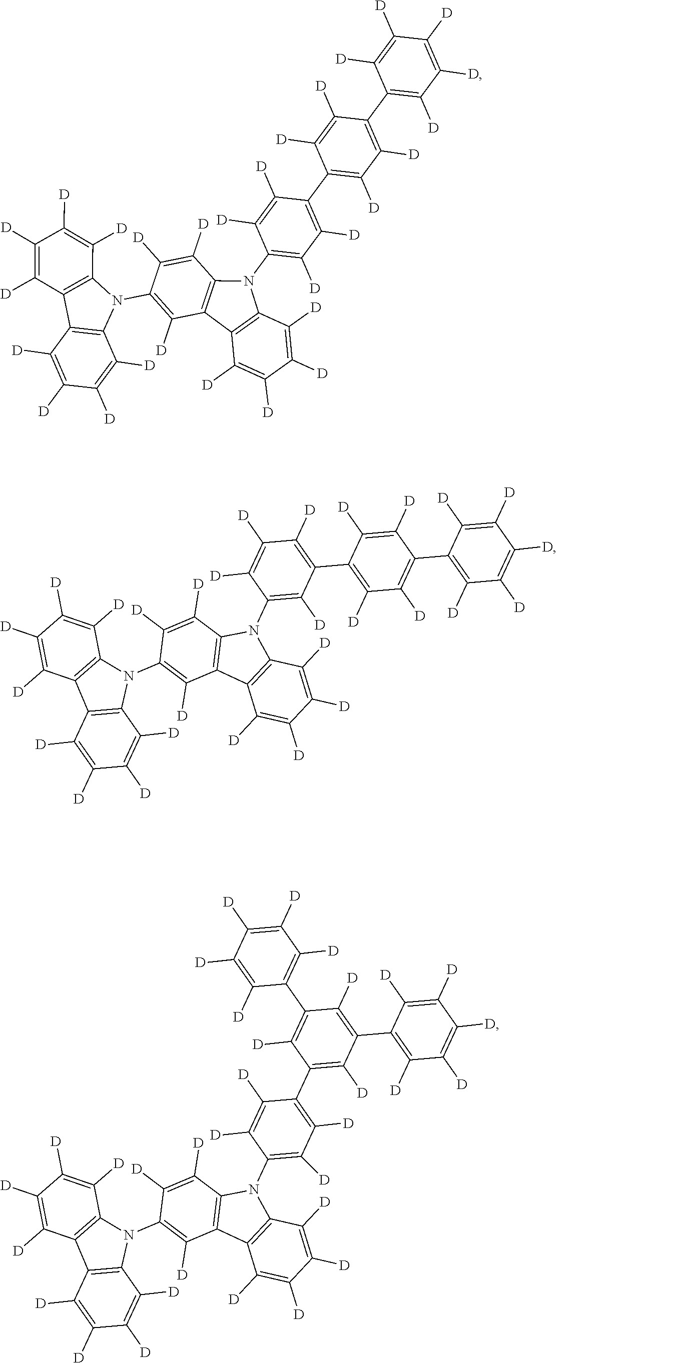

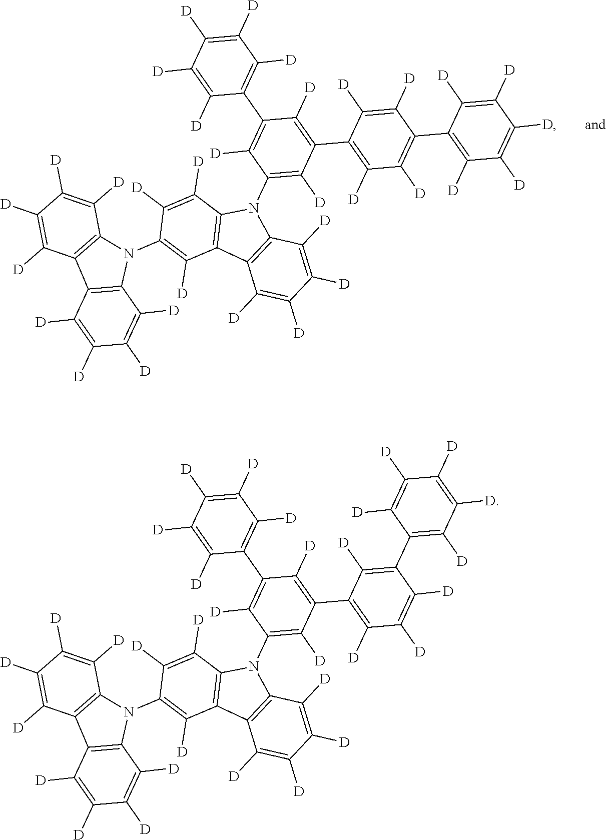

It should be understood that all the compounds including LIST 1 described herein can be appropriately applied to other embodiments or aspects of the present disclosure.

It should also be understood that the first group of the compound as described herein can be used for all the embodiments or aspects of the present disclosure, and likewise, the second group of the compound as described herein can also be used for all the embodiments or aspects of the present disclosure.

In yet another aspect, the present disclosure further provides an inventive compound consisting of a first group and a second group;

wherein:
each of the first group and the second group independently comprises at least one continuous moiety comprising at least three rings;
atoms of the first group do not overlap with atoms of the second group, and no fused ring system is part of both groups;
wherein the device lifetime LT1 of an OLED with the inventive compound used as a host is at least 30% higher than the device lifetime LT2 of a first test OLED with a first comparative compound used as a host;
wherein the first comparative compound has the same chemical structure as the inventive compound (i.e., the first comparative compound also consists of a first group and a second group) except that the first group of the first comparative compound is not deuterated; and
wherein the OLED and the first test OLED are different only in the deuteration levels of the inventive compound and the first comparative compound.

In some embodiments, the inventive compound and the first comparative compound are in the emissive layer of their respective OLEDs and the only difference between the OLED and the first test OLED is the deuteration levels of the inventive compound and the first comparative compound in the emissive layer.

In some of the above embodiments, the device lifetime LT1 is at least 35% higher than the device lifetime LT2. In some of the above embodiments, the device lifetime LT1 is at least 40% higher than the device lifetime LT2. In some of the above embodiments, the device lifetime LT1 is at least 45% higher than the device lifetime LT2. In some of the above embodiments, the device lifetime LT1 is at least 50% higher than the device lifetime LT2. In some of the above embodiments, the device lifetime LT1 is at least 55% higher than the device lifetime LT2. In some of the above embodiments, the device lifetime LT1 is at least 60% higher than the device lifetime LT2. In some of the above embodiments, the device lifetime LT1 is at least 65% higher than the device lifetime LT2. In some of the above embodiments, the device lifetime LT1 is at least 70% higher than the device lifetime LT2. In some of the above embodiments, the device lifetime LT1 is at least 80% higher than the device lifetime LT2. In some of the above embodiments, the device lifetime LT1 is at least 90% higher than the device lifetime LT2. In some of the above embodiments, the device lifetime LT1 is at least 100% higher than the device lifetime LT2. In some of the above embodiments, the device lifetime LT1 is at least 125% higher than the device lifetime LT2. In some of the above embodiments, the device lifetime LT1 is at least 150% higher than the device lifetime LT2. In some of the above embodiments, the device lifetime LT1 is at least 175% higher than the device lifetime LT2. In some of the above embodiments, the device lifetime LT1 is at least 200% higher than the device lifetime LT2.

In some of the above embodiments, the device lifetime LT1 of an OLED with the inventive compound used as a host is no more than 20% higher or lower than the device lifetime LT3 of a second test OLED with a second comparative compound used as a host;
wherein the second comparative compound has the same chemical structure as the inventive compound (i.e., the second comparative compound also consists of a first group and a second group) except that the second group of the second comparative compound is fully deuterated; and wherein the OLED and the second test OLED are different only in the deuteration levels of the inventive compound and the second comparative compound.

In some embodiments, the inventive compound is in the emissive layer and the only difference between the OLED and the second test OLED is the deuteration levels of the inventive compound and the second comparative compound in the emissive layer.

In some of the above embodiments, the device lifetime LT1 is no more than 15% higher or lower than the device lifetime LT3. In some of the above embodiments, the device lifetime LT1 is no more than 10% higher or lower than the device lifetime LT3. In some of the above embodiments, the device lifetime LT1 is no more than 5% higher or lower than the device lifetime LT3.

In some of the embodiments, the device lifetime LT1 of an OLED with the inventive compound used as a host is no more than 20% higher or lower than a device lifetime LT4 of a third test OLED with a third comparative compound used as a host; wherein the third comparative compound has the same chemical structure as the inventive compound (i.e., the third comparative compound also consists of a first group and a second group) except that the second group of the third comparative compound is not deuterated; and wherein the OLED and the third test OLED are different only in the deuteration levels of the inventive compound and the third comparative compound.

In some embodiments, the inventive compound is in the emissive layer and the only difference between the OLED and the third test OLED is the deuteration levels of the inventive compound and the third comparative compound in the emissive layer.

In some of the above embodiments, the device lifetime LT1 is no more than 15% higher or lower than the device lifetime LT4. In some of the above embodiments, the device lifetime LT1 is no more than 10% higher or lower than the device lifetime LT4. In some of the above embodiments, the device lifetime LT1 is no more than 5% higher or lower than the device lifetime LT4.

In some of the above embodiments, the second group is not deuterated.

In some of the above embodiments, the first group has a minimum percentage of deuteration of 30%, 50%, 70%, 90%, 99%, or 100%.

In some of the above embodiments, the second group consists of a minimum percentage of the electron densities of the HOMO and the LUMO of the compound of 70%, 75%, 80%, 85%, 90%, 95%, or 100%.

In some of the above embodiments, the first group may have a lowest triplet energy T₁ of at least 3.00 eV. In some of the above embodiments, the first group may have a lowest triplet energy T₁ of at least 3.05 eV. In some of the above embodiments, the first group may have a lowest triplet energy T₁ of at least 3.10 eV. In some of the above embodiments, the first group may have a lowest triplet energy T₁ of at least 3.15 eV. In some of the above embodiments, the first group may have a lowest triplet energy T₁ of at least 3.20 eV.

In some embodiments, the second group is not deuterated.

In some embodiments, the second group has a maximum percentage of deuteration of 20%, 10%, 5%, or 0%.

As used herein, percent deuteration has its ordinary meaning and includes the percent of possible hydrogen atoms (e.g., positions that are hydrogen, deuterium) that are replaced by deuterium atoms. Basically, it is the total number of deuterium divided by the total number of deuterium plus hydrogen.

It should be understood that the electron densities of the HOMO and the LUMO can be obtained through DFT calculations. DFT calculations were performed using the B3LYP functional with a 6-31G* basis set. Geometry optimizations were performed in vacuum. Excitation energies were obtained at these optimized geometries using time-dependent density functional theory (TDDFT). A continuum solvent model was applied in the TDDFT calculation to simulate tetrahydrofuran solvent. All calculations were carried out using the program Gaussian.

The calculations obtained with the above-identified DFT functional set and basis set are theoretical.

Computational composite protocols, such as Gaussian with the 6-31G* basis set used herein, rely on the assumption that electronic effects are additive and, therefore, larger basis sets can be used to extrapolate to the complete basis set (CBS) limit. However, when the goal of a study is to understand variations in HOMO, LUMO, S₁, T₁, excited-state localization, etc. over a series of structurally related compounds, the additive effects are expected to be similar. Accordingly, while absolute errors from using the B3LYP may be significant compared to other computational methods, the relative differences between the HOMO, LUMO, S₁, and T₁ values calculated with B3LYP protocol are expected to reproduce experiment quite well. See, e.g., Hong et al., Chem. Mater. 2016, 28, 5791-98, 5792-93 and Supplemental Information (discussing the reliability of DFT calculations in the context of OLED materials).

Analysis of Excited-State Localization: For a determination of the extent of HOMO and LUMO localization for a given molecule, the respective molecular orbitals from B3LYP may be subjected to a Löwdin population analysis as described by Löwdin, P.-O. J Chem Phys 1950, 18, 365 and Löwdin, P.-O. Adv Quantum Chem 1970, 5, 185. This is accomplished by partitioning the HOMO and LUMO into disjoint, atom-centered contributions that collectively compose a molecule (these sets of atoms are hereafter referred to as moieties or groups). These contributions are then collected according to the moieties or groups (e.g., the first group and the second group) defined earlier.

### C. The OLEDs and the Devices of the Present Disclosure

In another aspect, the present disclosure also provides an OLED device comprising a first organic layer that contains an inventive compound of the present disclosure.

In some embodiments, such OLED comprises an anode, a cathode, and a first organic layer disposed between the anode and the cathode. The first organic layer can comprise a compound of the present disclosure.

In some embodiments, the organic layer may be an emissive layer and the compound as described herein can be an emissive dopant or a non-emissive dopant.

In some embodiments, the compound is a host, and the organic layer is an emissive layer that comprises a phosphorescent material.

In some embodiments, the OLED may comprise a compound selected from the group consisting of a delayed fluorescence material, a phosphorescent material, and combination thereof.

In some embodiments, the phosphorescent material is an emitter which emits light within the OLED. In some embodiments, the phosphorescent material does not emit light within the OLED. In some embodiments, the phosphorescent material energy transfers its excited state to another material within the OLED. In some embodiments, the phosphorescent material participates in charge transport within the OLED. In some embodiments, the phosphorescent material is a sensitizer, and the OLED further comprises an acceptor.

In some embodiments, the delayed fluorescence material is an emitter which emits light within the OLED. In some embodiments, the delayed fluorescence material does not emit light within the OLED. In some embodiments, the delayed fluorescence material energy transfers its excited state to another material within the OLED. In some embodiments, the delayed fluorescence material participates in charge transport within the OLED. In some embodiments, the delayed fluorescence material is a sensitizer, and the OLED further comprises an acceptor.

In some embodiments, the compound may be an acceptor, and the OLED may further comprise a sensitizer selected from the group consisting of a delayed fluorescence material, a phosphorescent material, and combination thereof.

In some embodiments, the compound may be a fluorescent emitter, a delayed fluorescence material, or a component of an exciplex that is a fluorescent emitter or a delayed fluorescence material.

In some embodiments, the compound is a host and the OLED comprises an acceptor that is an emitter and a sensitizer selected from the group consisting of a delayed fluorescence material, a phosphorescent material, and combination thereof; wherein the sensitizer transfers energy to the acceptor.

In some embodiments, where the compound is a host, the compound can be an electron transporting host. In some of these embodiments, the compound has a LUMO less than −2.4 eV. In some of these embodiments, the compound has a LUMO less than −2.5 eV. In some of these embodiments, the compound has a LUMO less than −2.6 eV. In some of these embodiments, the compound has a LUMO less than −2.7 eV.

In some embodiments, the phosphorescent material can be a metal coordination complex having a metal-carbon bond, a metal-nitrogen bond, or a metal-oxygen bond. In some embodiments, the metal is selected from the group consisting of Ir, Rh, Re, Ru, Os, Pt, Pd, Au, and Cu. In some embodiments, the metal is Ir. In some embodiments, the metal is Pt. In some embodiments, the sensitizer compound has the formula of M(L¹)ₓ(L²)_{y}(L³)_{z};
wherein L¹, L², and L³ can be the same or different;
wherein x is 1, 2, or 3;
wherein y is 0, 1, or 2;
wherein z is 0, 1, or 2;
wherein x+y+z is the oxidation state of the metal M;
wherein L¹ is selected from the group consisting of the structures of LIGAND LIST:

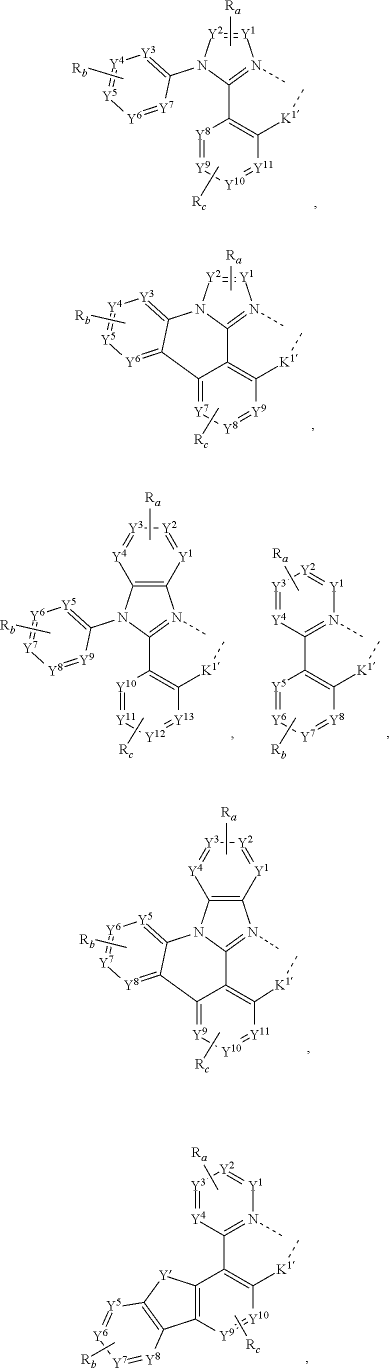

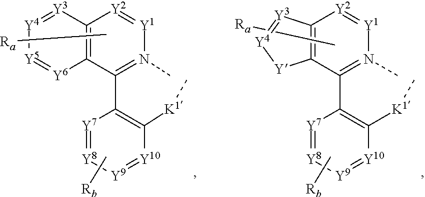

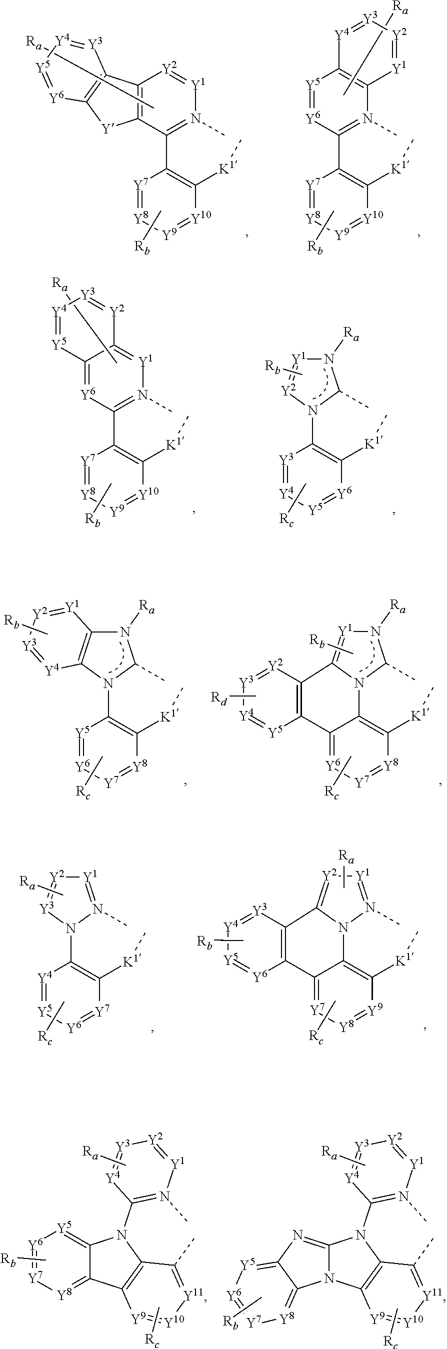

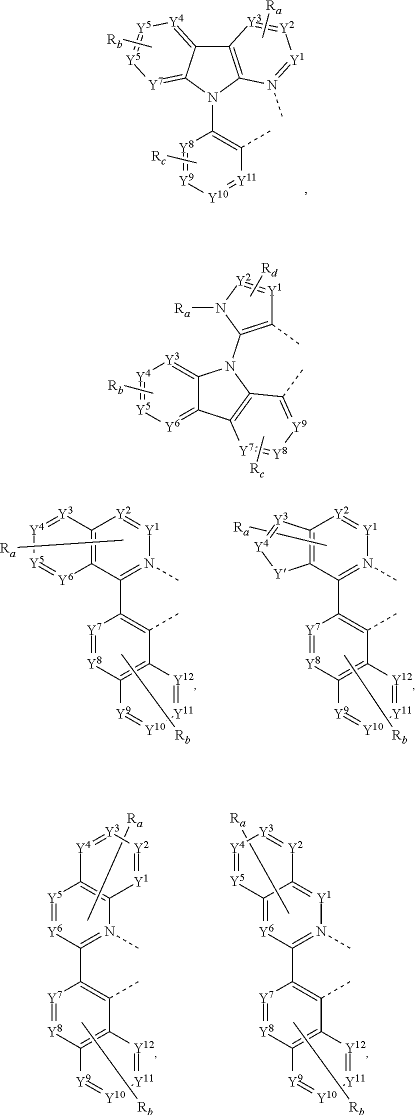

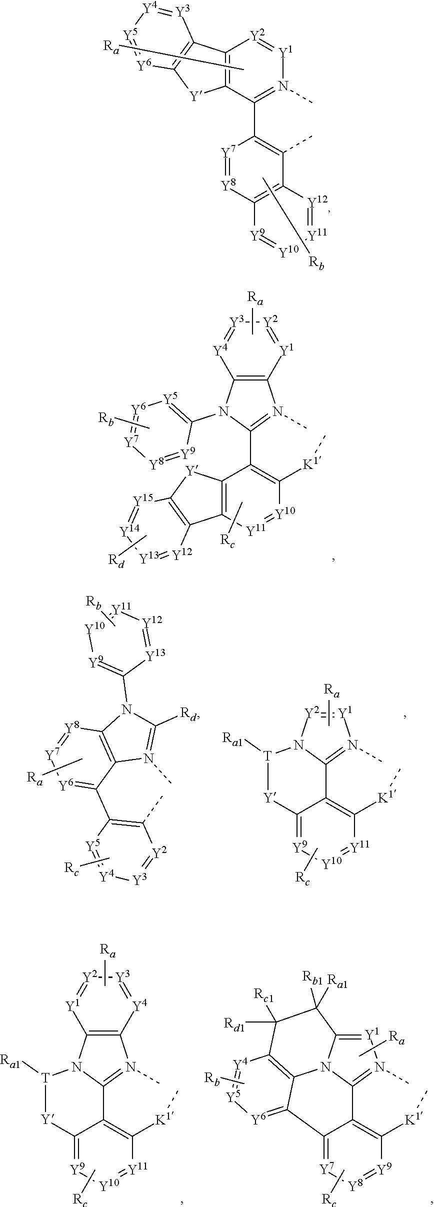

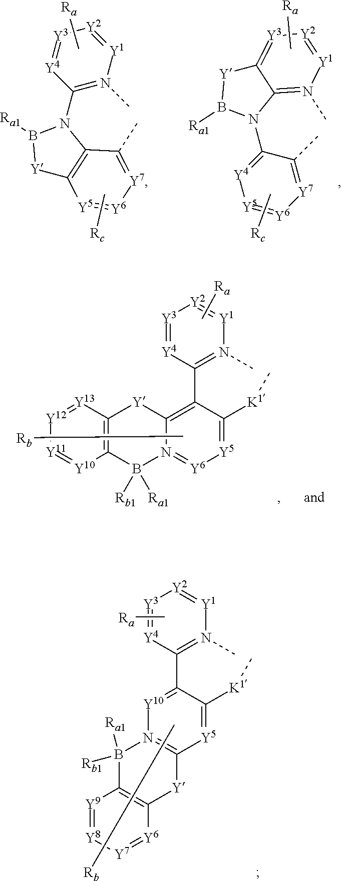

wherein L² and L³ are independently selected from the group consisting of

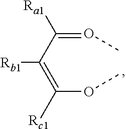

and the structures of LIGAND LIST; wherein:
T is selected from the group consisting of B, Al, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of BRₑ, NRₑ, PRₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of the General Substituents as defined herein; and
   wherein any two of Rₐ₁, R_{b1}, R_{c1}, R_{d1}, Rₐ, R_{b}, R_{c}, and R_{d} can be fused or joined to form a ring or form a multidentate ligand.

In some embodiments, the metal in formula M(L¹)ₓ(L²)_{y}(L³)_{z} is selected from the group consisting of Cu, Ag, or Au.

In some embodiments of the OLED, the phosphorescent material has a formula selected from the group consisting of Ir(L_{A})₃, Ir(L_{A})(L_{B})₂, Ir(L_{A})₂(L_{B}), Ir(L_{A})₂(L_{C}), Ir(L_{A})(L_{B})(L_{C}), and Pt(L_{A})(L_{B});
wherein L_{A}, L_{B}, and L_{C} are different from each other in the Ir compounds;
wherein L_{A} and L_{B} can be the same or different in the Pt compounds; and
wherein L_{A} and L_{B} can be connected to form a tetradentate ligand in the Pt compounds.

In some embodiments of the OLED, the phosphorescent material is selected from the group consisting of the following compounds:

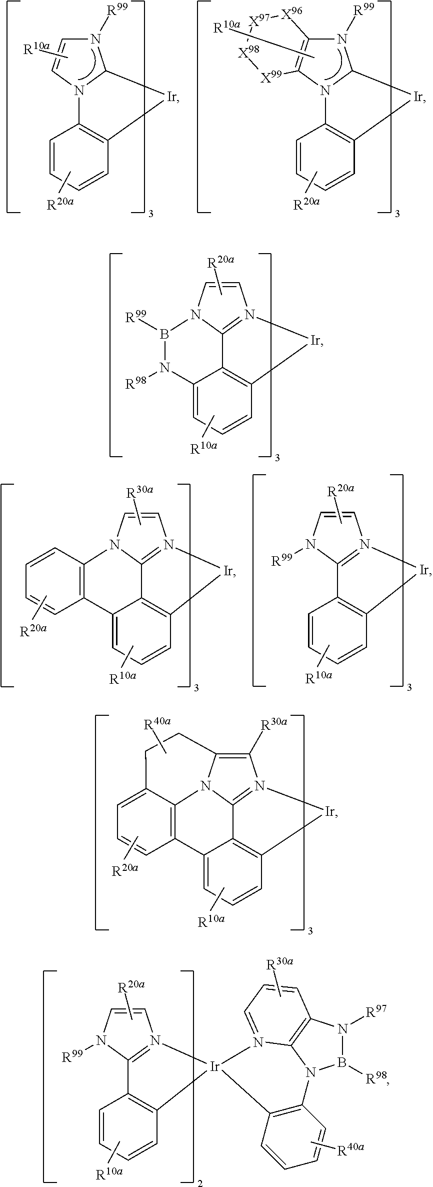

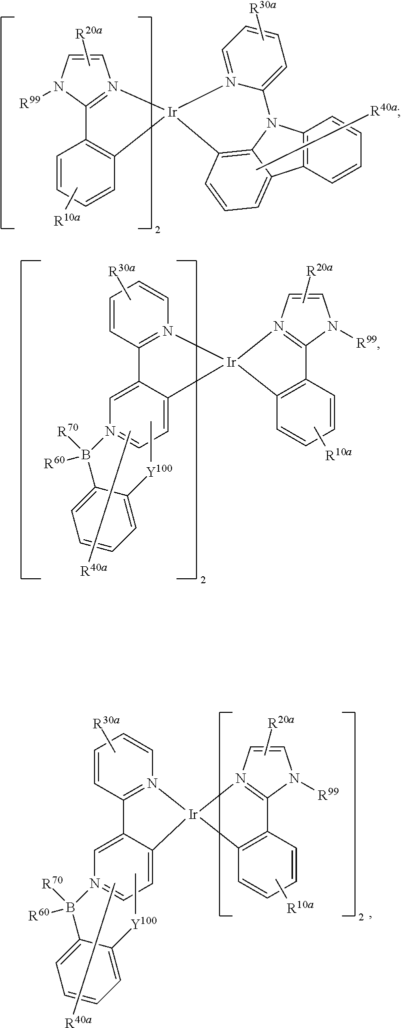

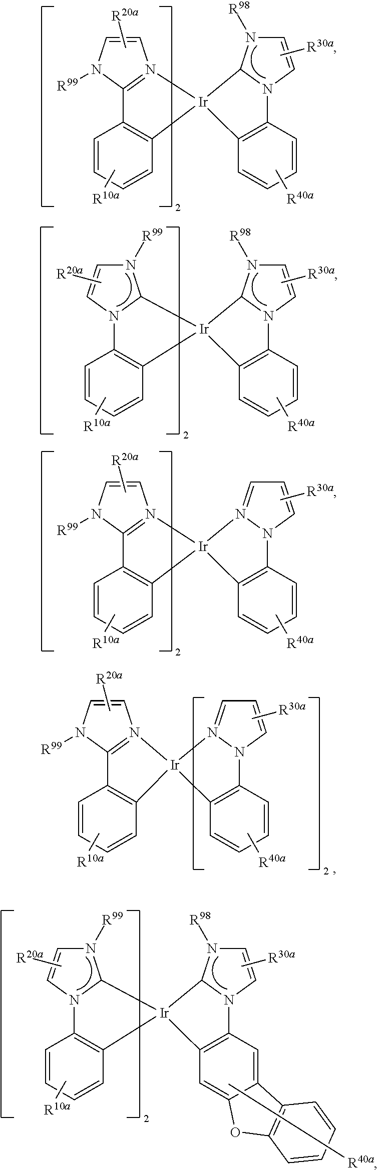

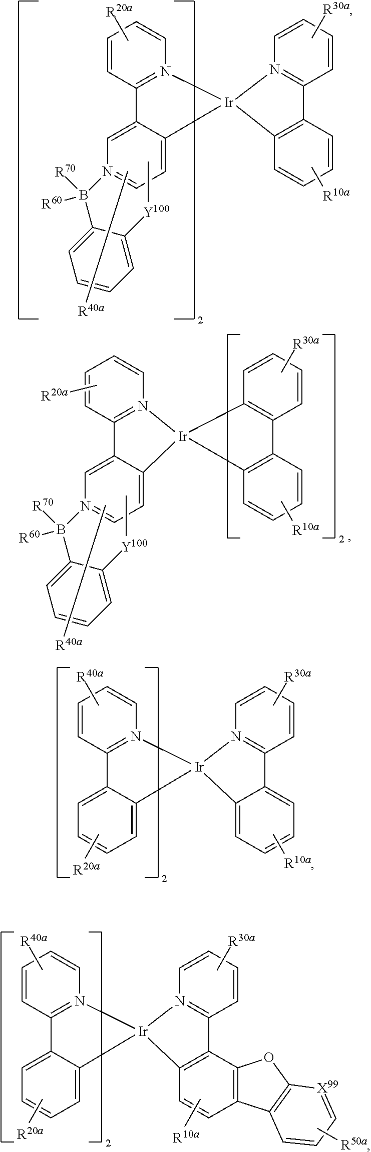

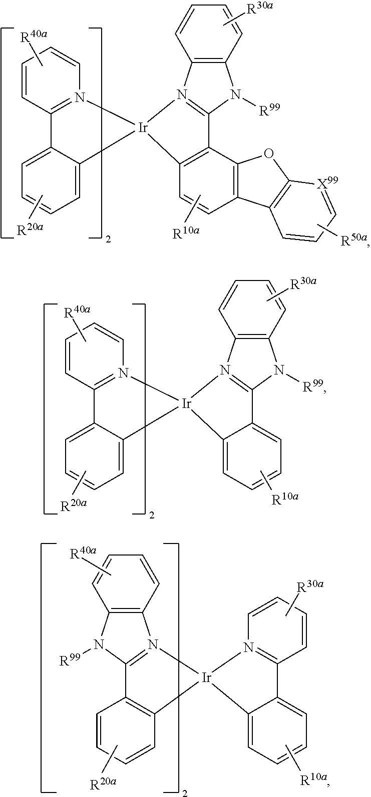

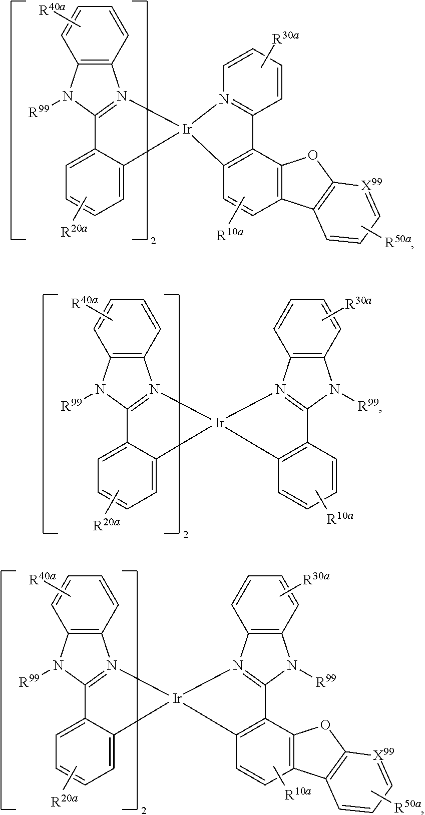

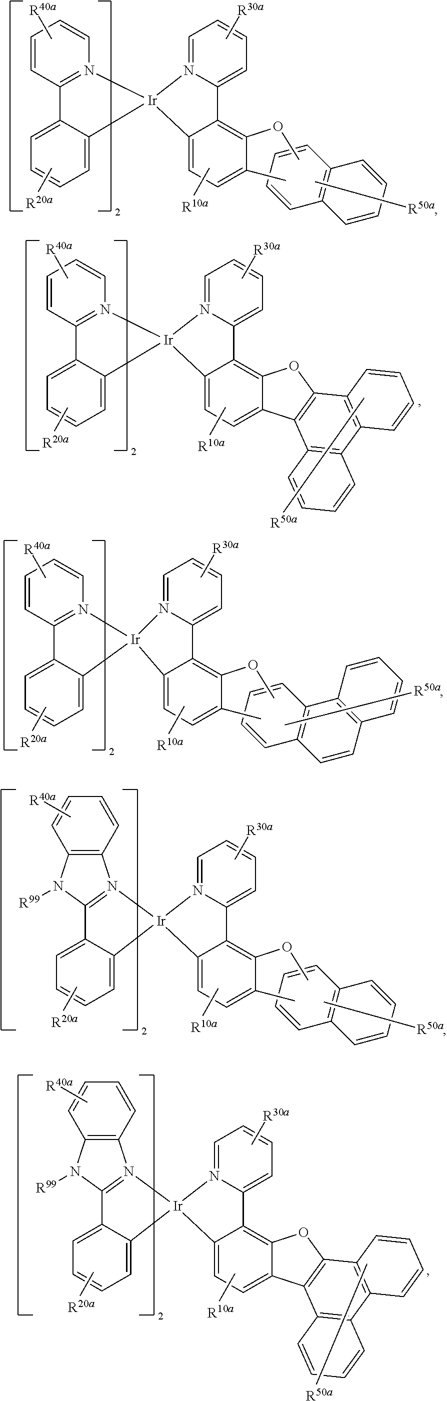

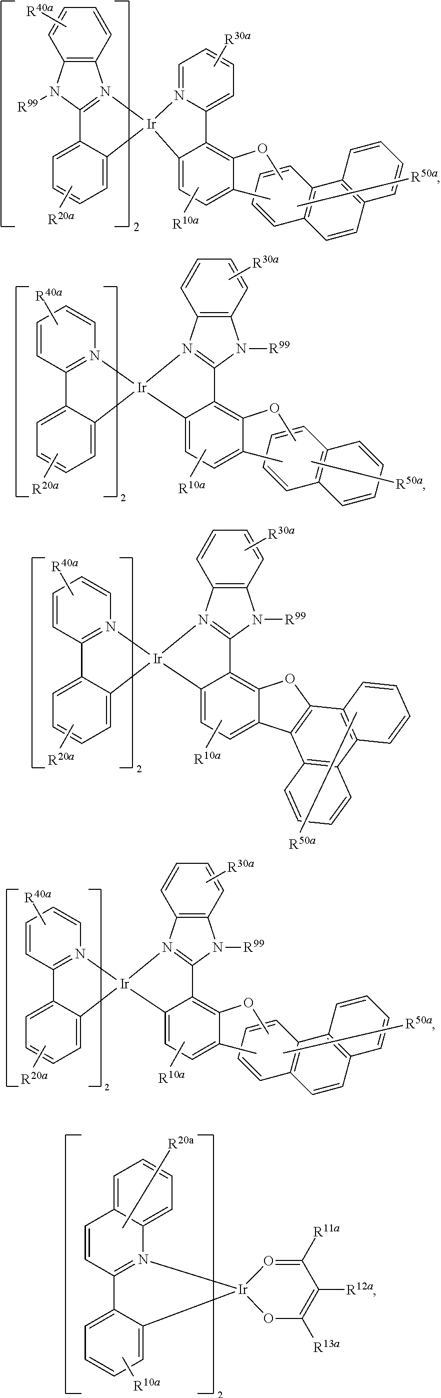

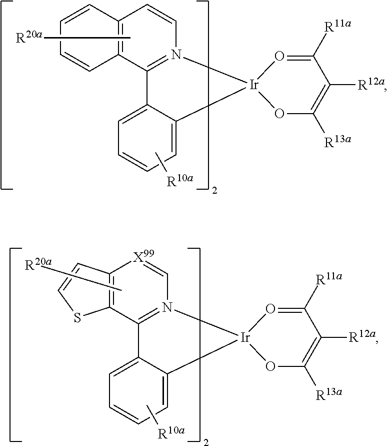

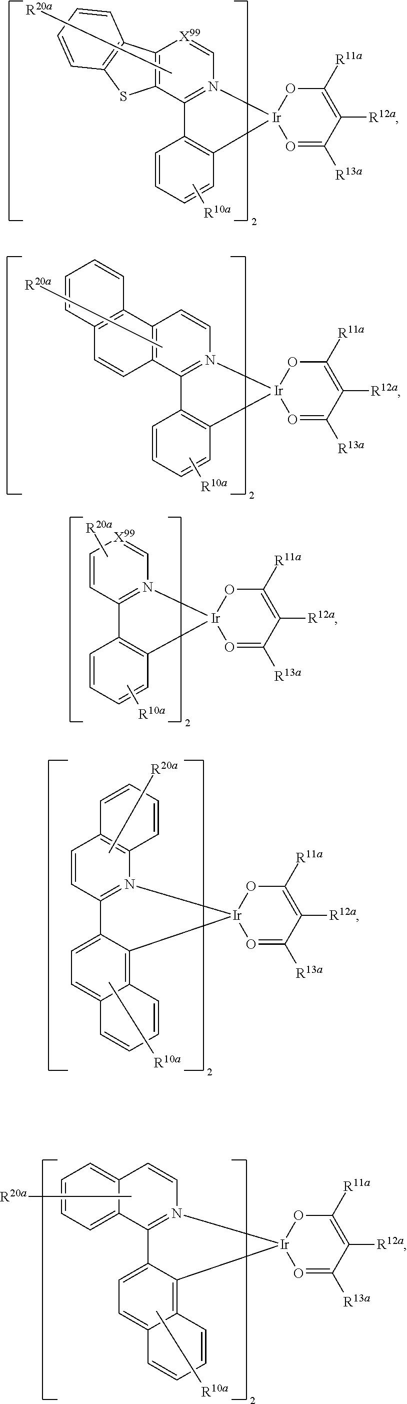

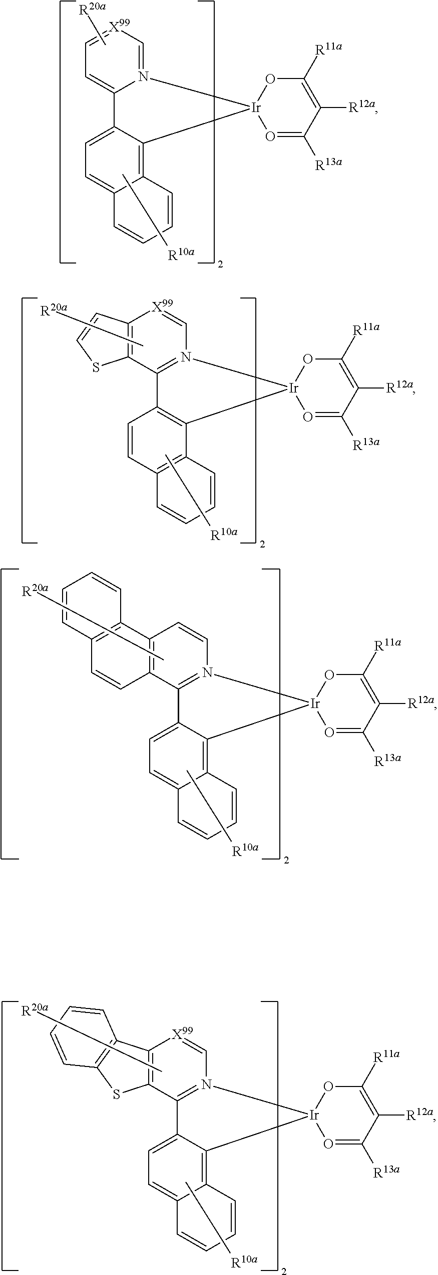

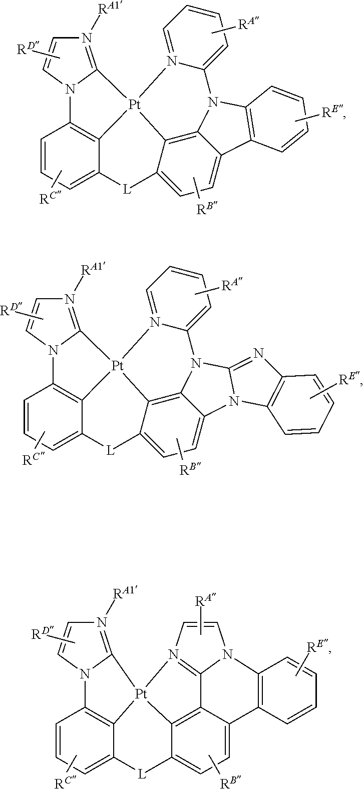

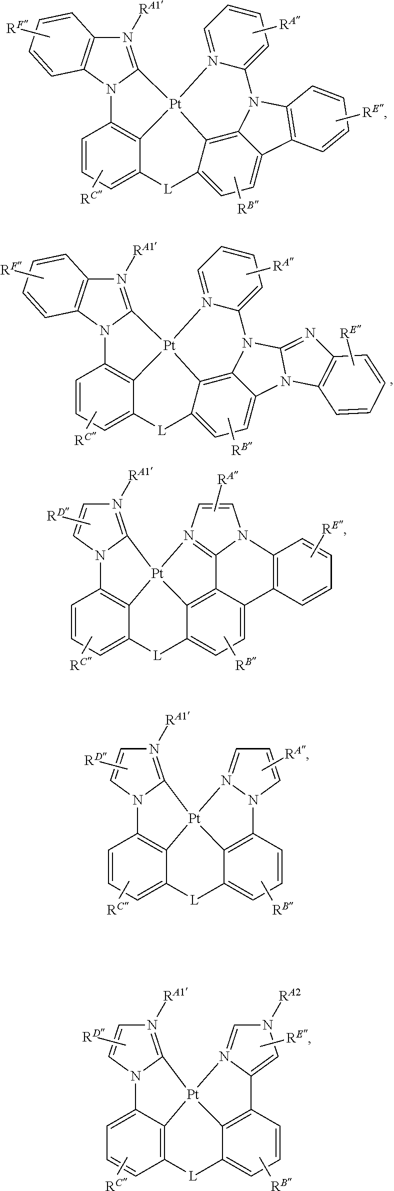

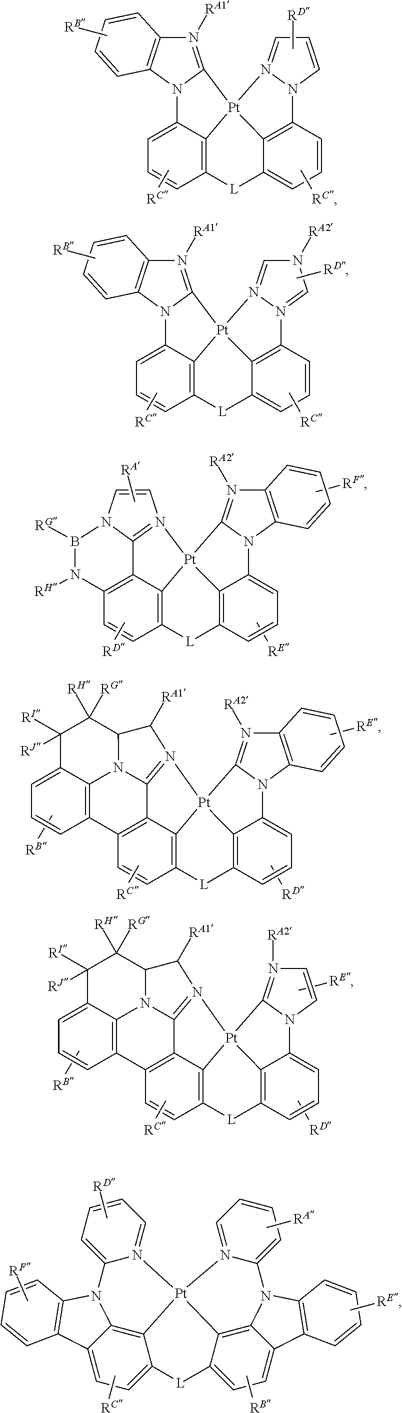

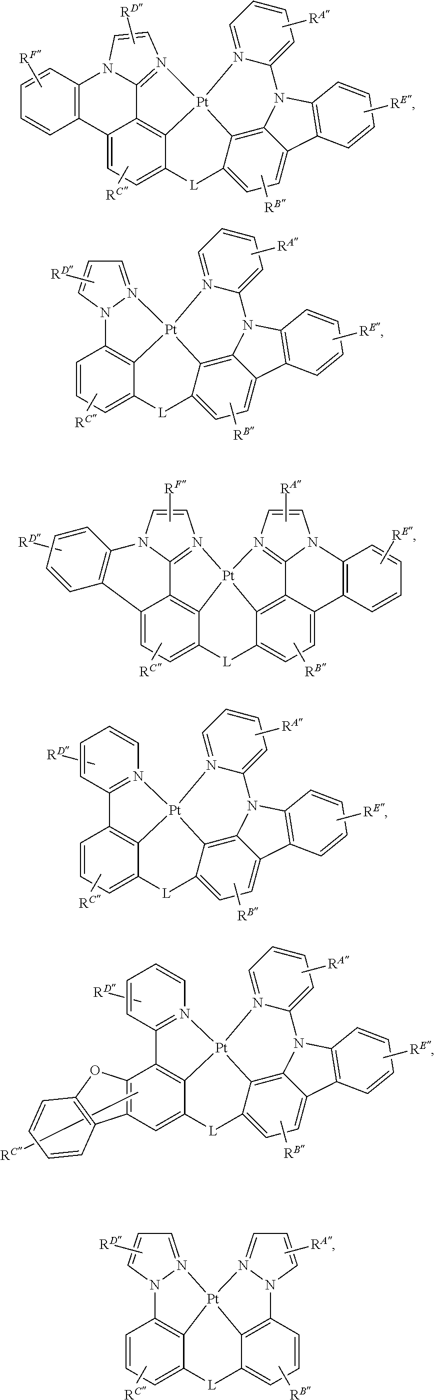

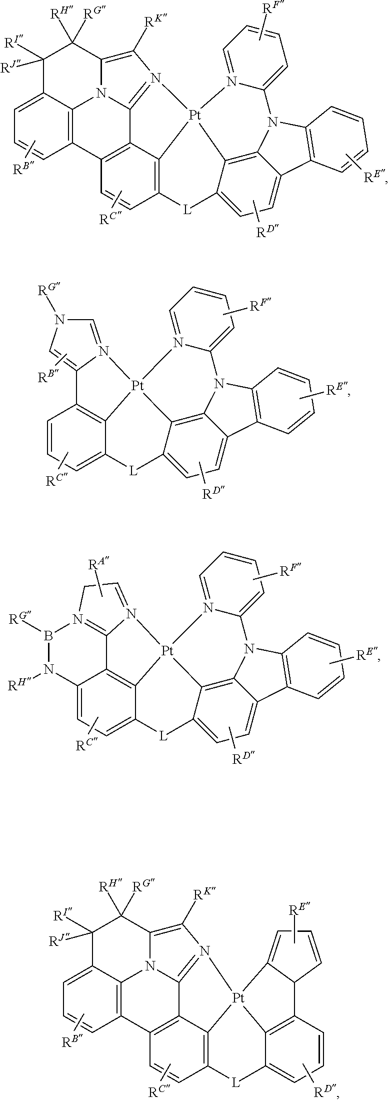

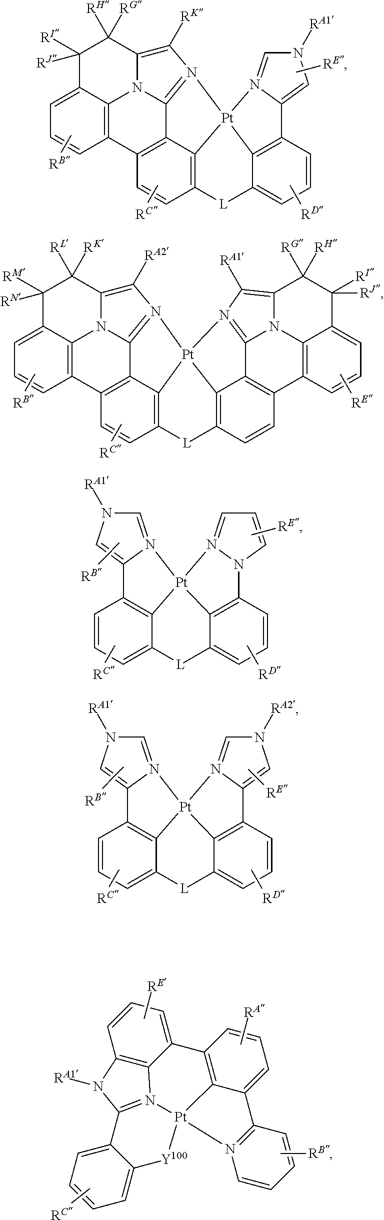

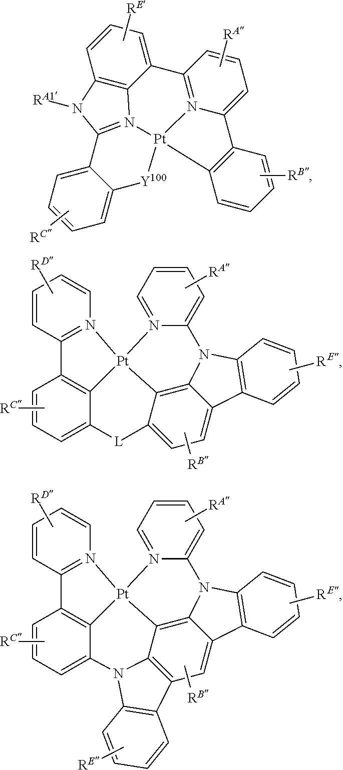

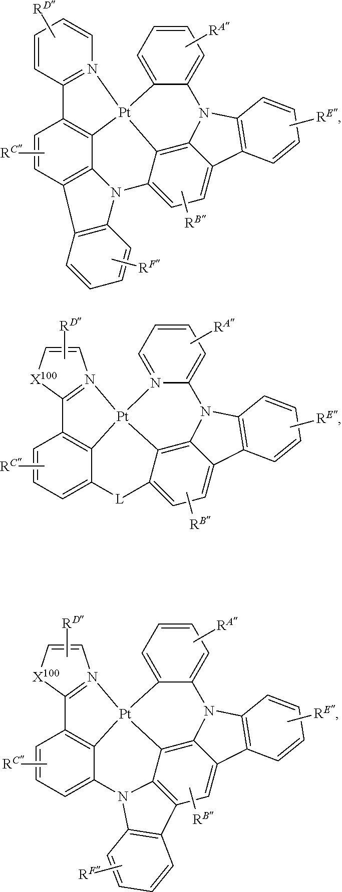

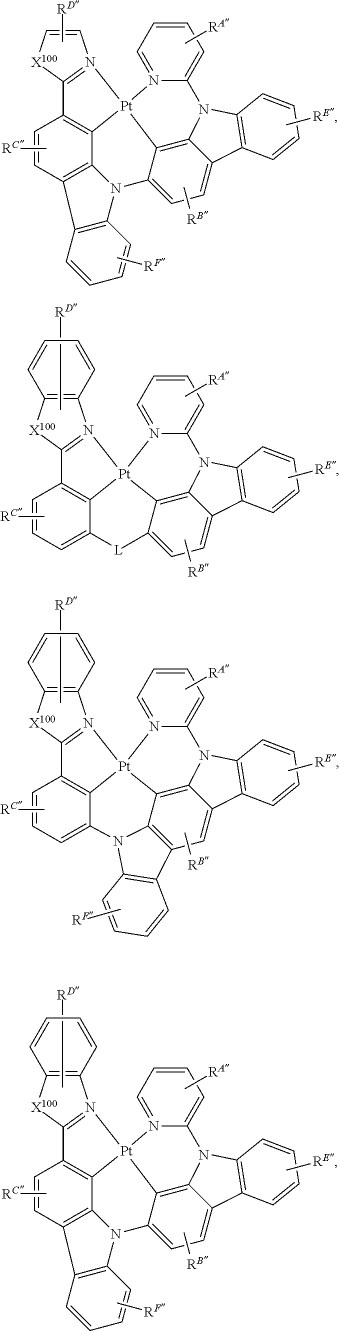

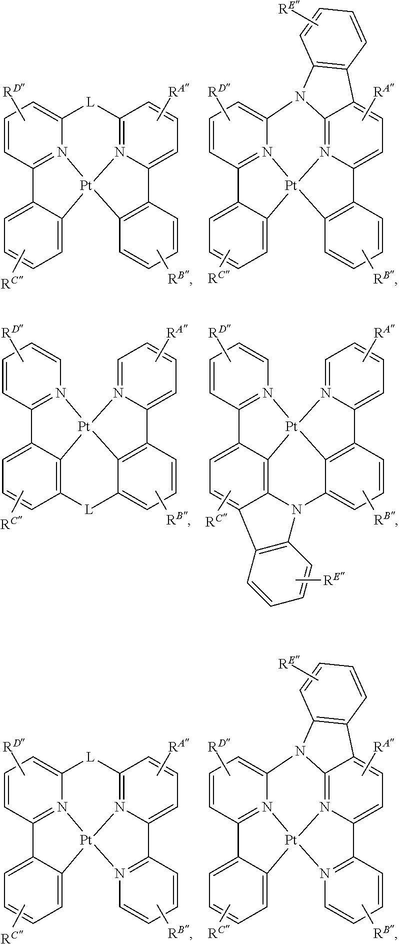

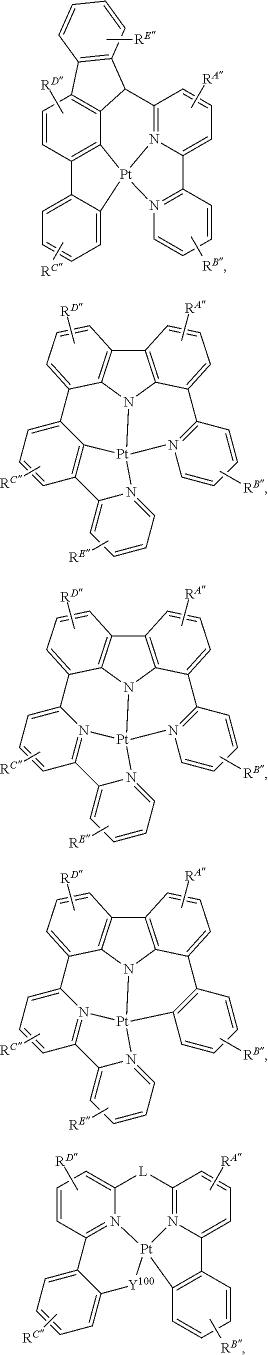

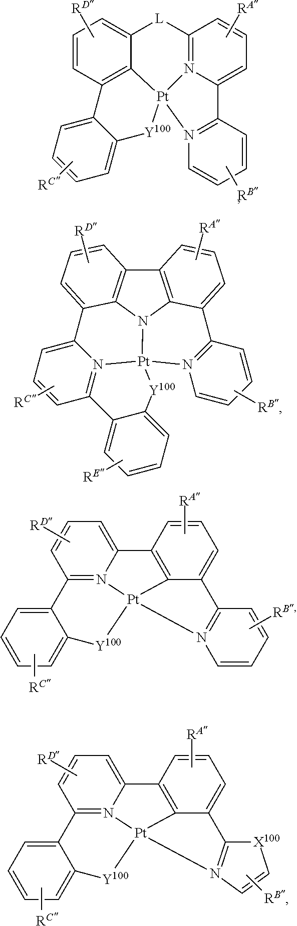

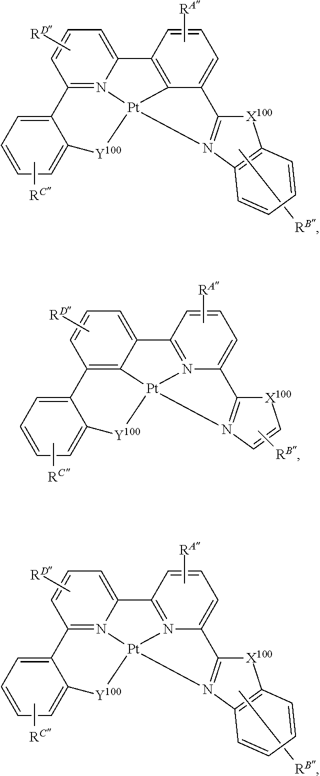

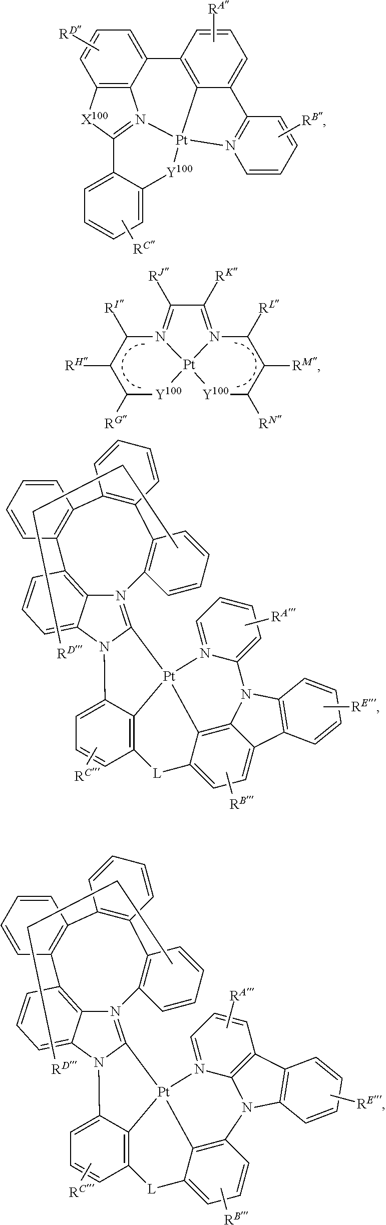

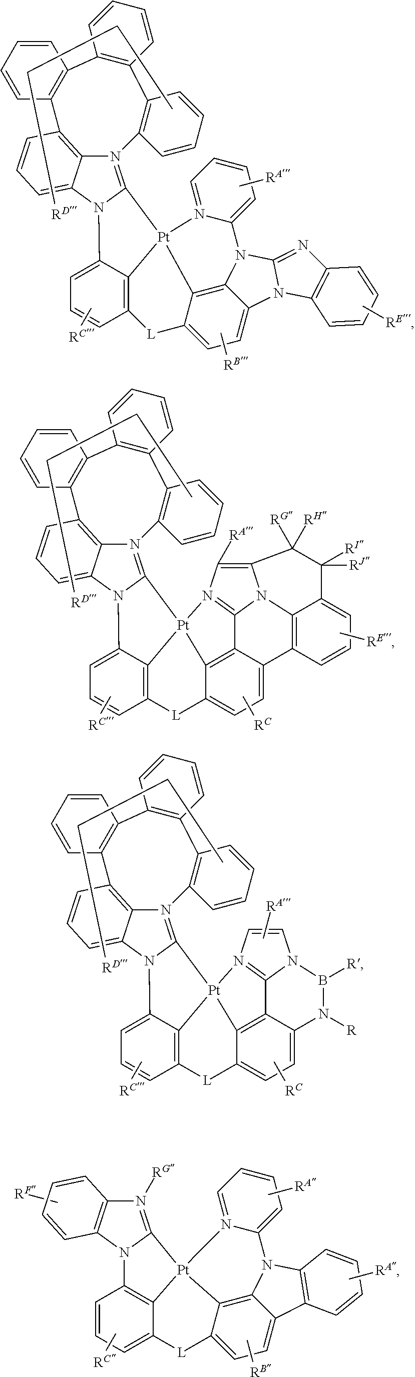

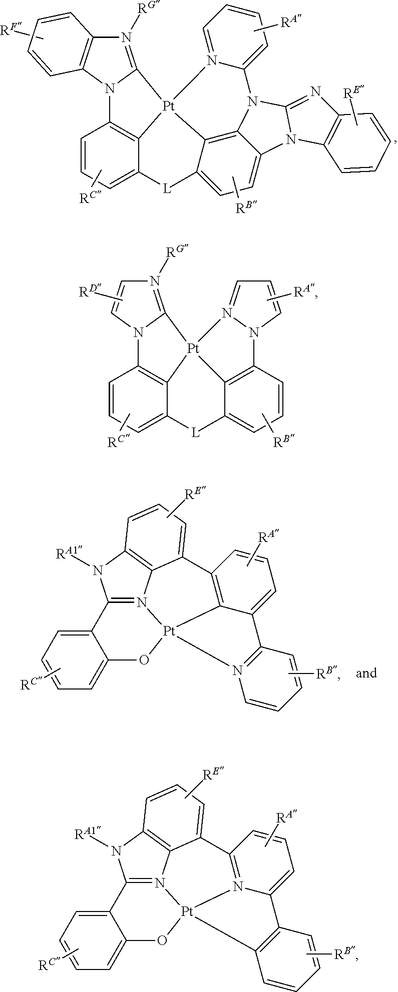

wherein:
each of X⁹⁶ to X⁹⁹ is independently C or N;
each Y¹⁰⁰ is independently selected from the group consisting of a NR″, O, S, and Se;
L is independently selected from the group consisting of a direct bond, BR″, BR″R′″, NR″, PR″, O, S, Se, C═O, C═S, C═Se, C═NR″, C═CR″R′″, S═O, SO₂, CR″, CR″R′″, SiR″R′″, GeR″R′″, alkyl, cycloalkyl, aryl, heteroaryl, and combinations thereof;
X¹⁰⁰ for each occurrence is selected from the group consisting of O, S, Se, NR″, and CR″R′″;
each R^{10a}, R^{20a}, R^{30a}, R^{40a}, and R^{50a}, R^{A″}, R^{B″}, R^{C″}, R^{D″}, R^{E″}, and R^{F″} independently represents mono-, up to the maximum substitutions, or no substitutions;
each of R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N″} is independently a hydrogen or a substituent selected from the group consisting of deuterium, halide, alkyl, cycloalkyl, heteroalkyl, arylalkyl, alkoxy, aryloxy, amino, silyl, germyl, boryl, selenyl, alkenyl, cycloalkenyl, heteroalkenyl, alkynyl, aryl, heteroaryl, acyl, carbonyl, carboxylic acid, ester, nitrile, isonitrile, sulfanyl, sulfinyl, sulfonyl, phosphino, combinations thereof.

In some embodiments of the OLED where the phosphorescent material is selected from the group consisting of the structures in the SENSITIZER LIST, one or more of R, R′, R″, R′″, R^{10a}, R^{11a}, R^{12a}, R^{13a}, R^{20a}, R^{30a}, R^{40a}, R^{50a}, R⁶⁰, R⁷⁰, R⁹⁷, R⁹⁸, R⁹⁹, R^{A1′}, R^{A2′}, R^{A″}, R^{B″}, R^{C″}, R^{D″}, R^{E″}, R^{F″}, R^{G″}, R^{H″}, R^{I″}, R^{J″}, R^{K″}, R^{L″}, R^{M″}, and R^{N″} comprises a moiety selected from the group consisting of fully or partially deuterated aryl, fully or partially deuterated alkyl, boryl, silyl, germyl, 2,6-terphenyl, 2-biphenyl, 2-(tert-butyl)phenyl, tetraphenylene, tetrahydronaphthalene, and combinations thereof.

In some embodiments, the emissive dopant or emitter can be a phosphorescent or fluorescent emitter. Phosphorescence generally refers to emission of a photon with a change in electron spin, i.e., the initial and final states of the emission have different multiplicity, such as from T₁ to S₀ state. Ir and Pt complexes currently widely used in the OLED belong to phosphorescent material. In some embodiments, if an exciplex formation involves a triplet emitter, such exciplex can also emit phosphorescent light. On the other hand, fluorescent emitters generally refer to emission of a photon without a change in electron spin, such as from S₁ to S₀ state. Fluorescent emitters can be delayed fluorescent or non-delayed fluorescent emitters. Depending on the spin state, fluorescent emitter can be a singlet emitter or a doublet emitter, or other multiplet emitter. It is believed that the internal quantum efficiency (IQE) of fluorescent OLEDs can exceed the 25% spin statistics limit through delayed fluorescence. There are two types of delayed fluorescence, i.e. P-type and E-type delayed fluorescence. P-type delayed fluorescence is generated from triplet-triplet annihilation (TTA). On the other hand, E-type delayed fluorescence does not rely on the collision of two triplets, but rather on the thermal population between the triplet states and the singlet excited states. Thermal energy can activate the transition from the triplet state back to the singlet state. This type of delayed fluorescence is also known as thermally activated delayed fluorescence (TADF). E-type delayed fluorescence characteristics can be found in an exciplex system or in a single compound. Without being bound by theory, it is believed that TADF requires a compound or an exciplex having a small singlet-triplet energy gap (ΔE_{S-T}) less than or equal to 300, 250, 200, 150, 100, or 50 meV. There are two major types of TADF emitters, one is called donor-acceptor type TADF, the other one is called multiple resonance (MR) TADF. Often, donor-acceptor single compounds are constructed by connecting an electron donor moiety such as amino- or carbazole-derivatives and an electron acceptor moiety such as N-containing six-membered aromatic ring. Donor-acceptor exciplex can be formed between a hole transporting compound and an electron transporting compound. The examples for MR-TADF include a highly conjugated boron-containing compounds. In some embodiments, the reverse intersystem crossing time from T1 to S1 of the delayed fluorescent emission at 293K is less than or equal to 10 microseconds. In some embodiments, such time can be greater than 10 microseconds and less than 100 microseconds.

In some embodiments of the OLED, the TADF emitter comprises at least one donor group and at least one acceptor group. In some embodiments, the TADF emitter is a metal complex. In some embodiments, the TADF emitter is a non-metal complex. In some embodiments, the TADF emitter is a Cu, Ag, or Au complex.

In some embodiments of the OLED, the TADF emitter has the formula of M(L⁵)(L⁶), wherein M is Cu, Ag, or Au, L⁵ and L⁶ are different, and L⁵ and L⁶ are independently selected from the group consisting of:

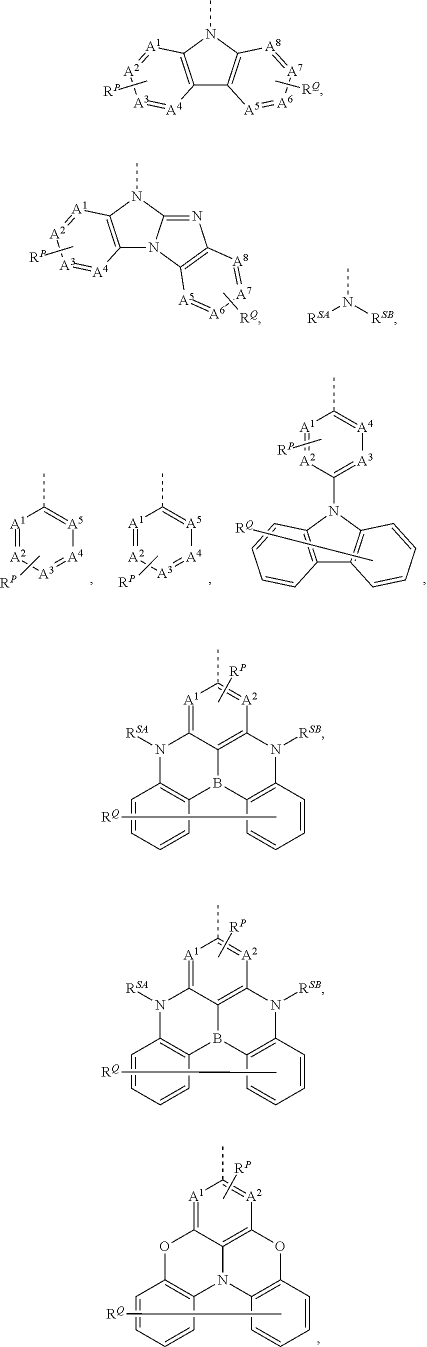

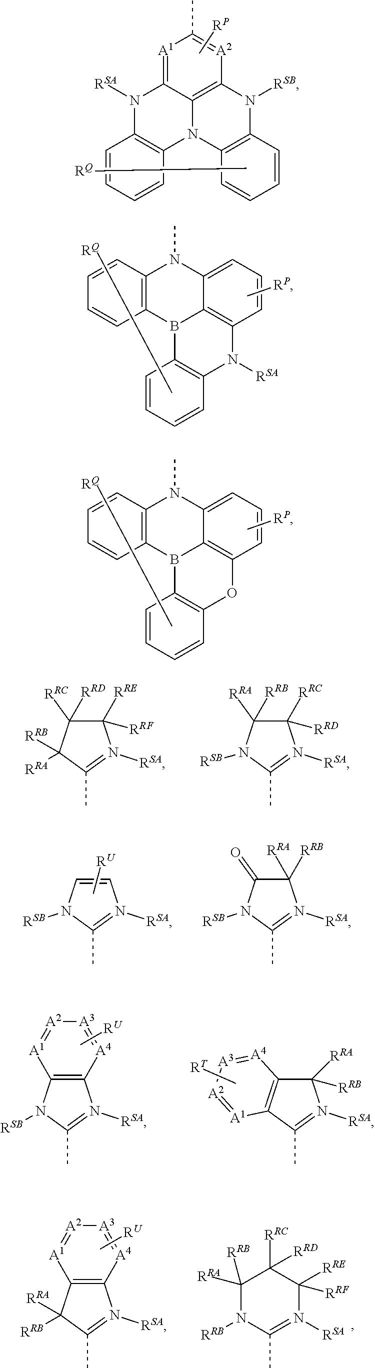

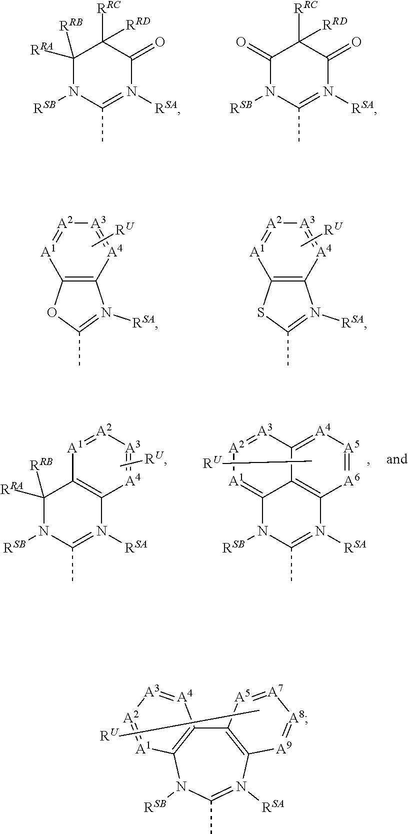

wherein A¹-A⁹ are each independently selected from C or N;
wherein each R^{P}, R^{P}, R^{U}, R^{SA}, R^{SB}, R^{RA}, R^{RB}, R^{RC}, R^{RD}, R^{RE}, and R^{RF} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In some embodiments of the OLED, the TADF emitter is selected from the group consisting of the structures in the following TADF LIST:

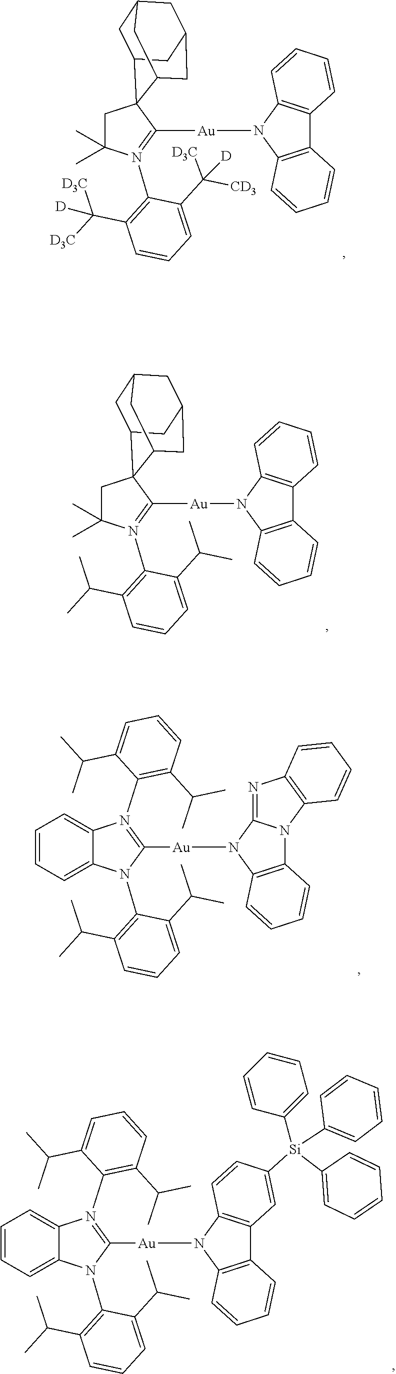

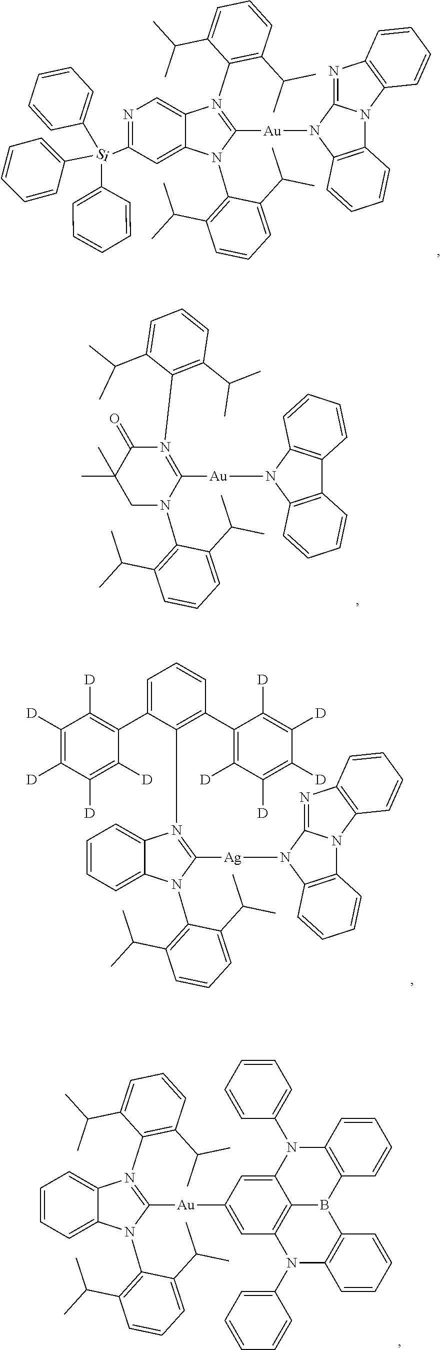

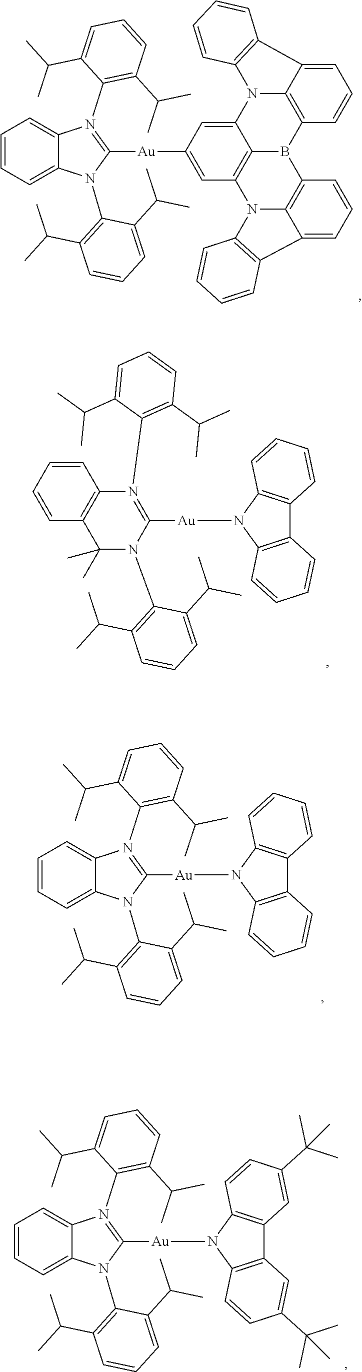

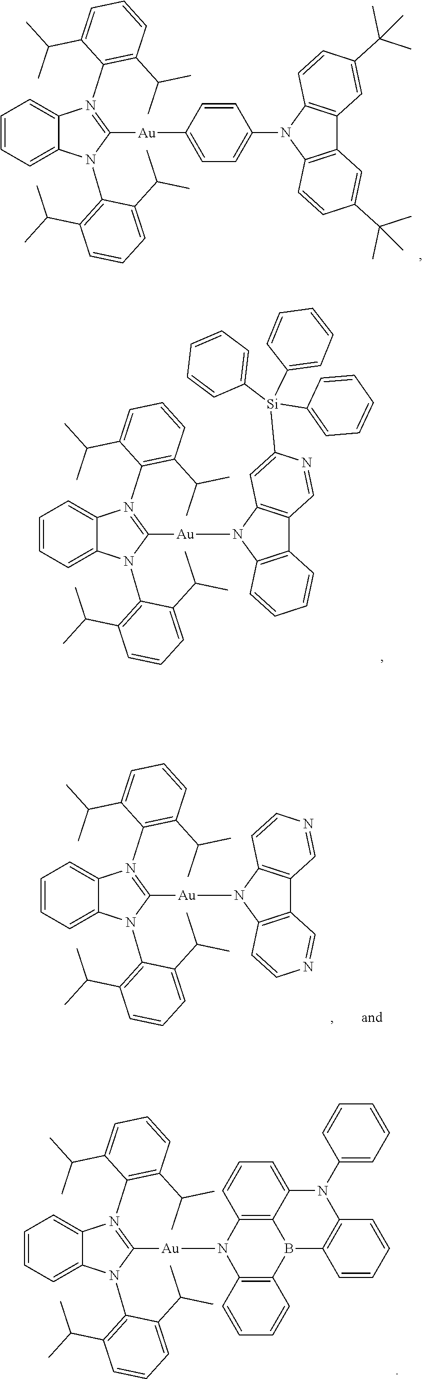

In some embodiments of the OLED, the TADF emitter comprises at least one of the chemical moieties selected from the group consisting of:

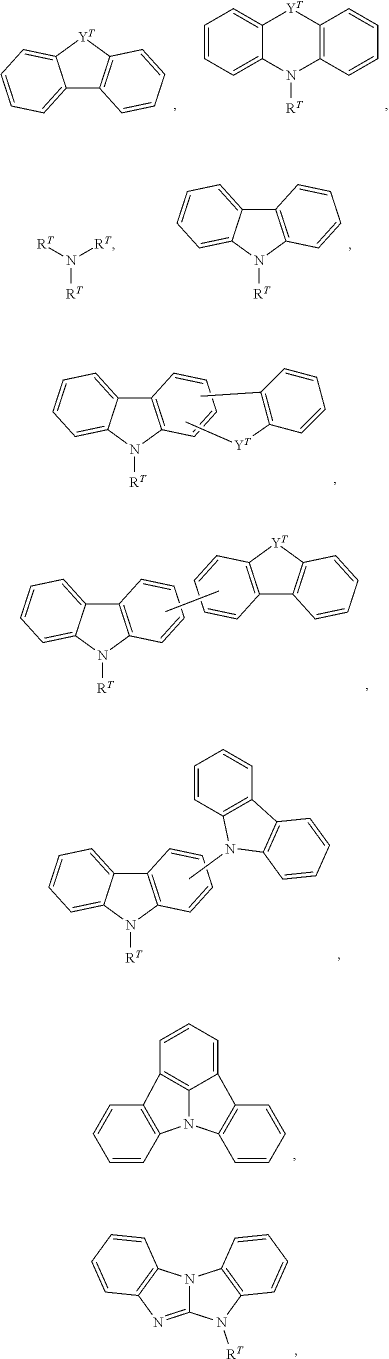

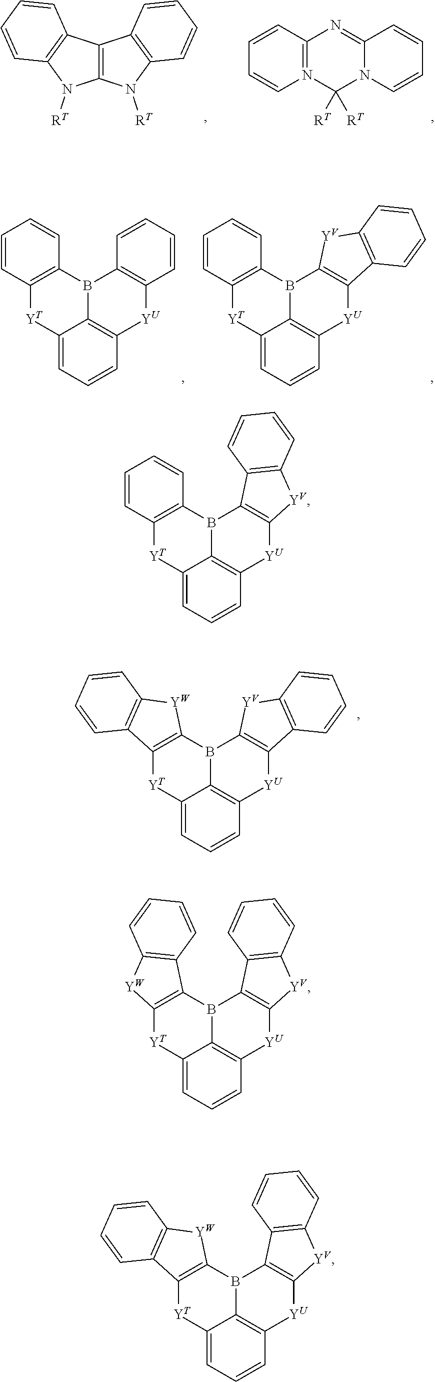

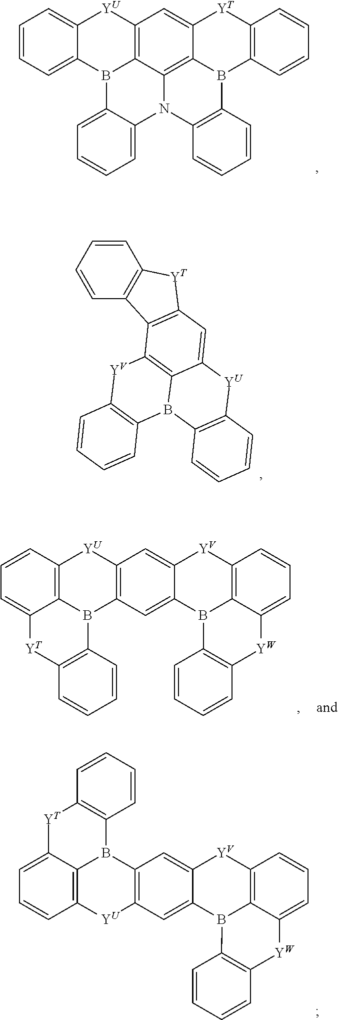

wherein Y^{T}, Y^{U}, Y^{V}, and Y^{W} are each independently selected from the group consisting of BR, NR, PR, O, S, Se, C═O, S═O, SO₂, BRR′, CRR′, SiRR′, and GeRR′;
wherein each R^{T} can be the same or different and each R^{T} is independently a donor, an acceptor group, an organic linker bonded to a donor, an organic linker bonded to an acceptor group, or a terminal group selected from the group consisting of alkyl, cycloalkyl, heteroalkyl, heterocycloalkyl, arylalkyl, aryl, heteroaryl, and combinations thereof; and
R, and R′ are each independently a hydrogen or a substituent selected from the group consisting of deuterium, halogen, alkyl, cycloalkyl, heteroalkyl, heterocycloalkyl, boryl, arylalkyl, alkoxy, aryloxy, amino, silyl, germyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, selenyl, and combinations thereof.

In some of the above embodiments, any carbon ring atoms up to maximum of a total number of three, together with their substituents, in each phenyl ring of any of above structures can be replaced with N.

In some embodiments, the TADF emitter comprises at least one of the chemical moieties selected from the group consisting of nitrile, isonitrile, borane, fluoride, pyridine, pyrimidine, pyrazine, triazine, aza-carbazole, aza-dibenzothiophene, aza-dibenzofuran, aza-dibenzoselenophene, aza-triphenylene, imidazole, pyrazole, oxazole, thiazole, isoxazole, isothiazole, triazole, thiadiazole, and oxadiazole.

In some embodiments, the fluorescent compound comprises at least one of the chemical moieties selected from the group consisting of:

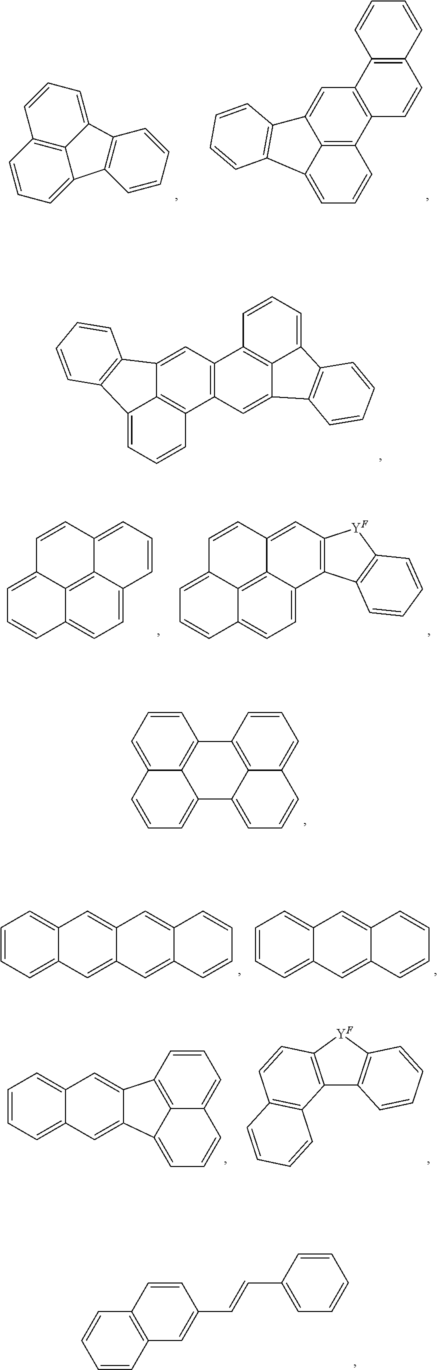

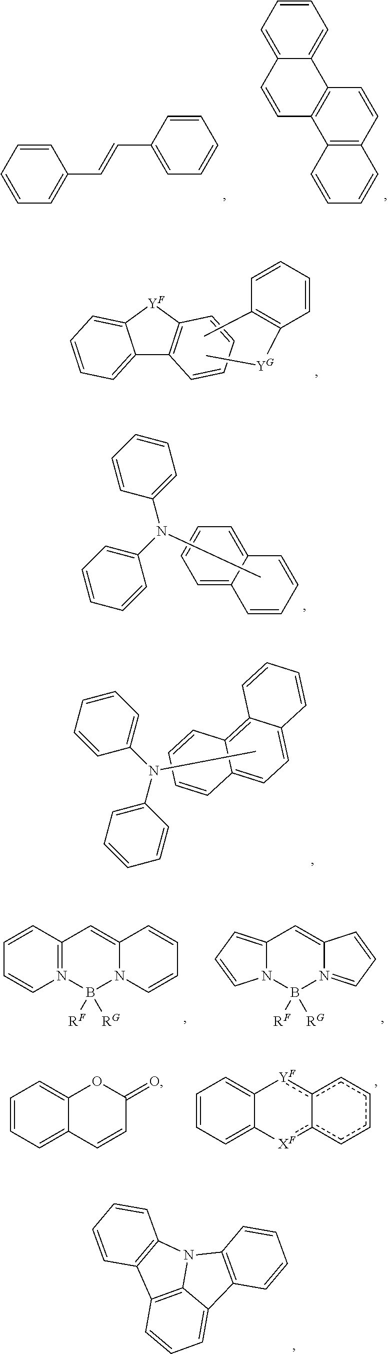

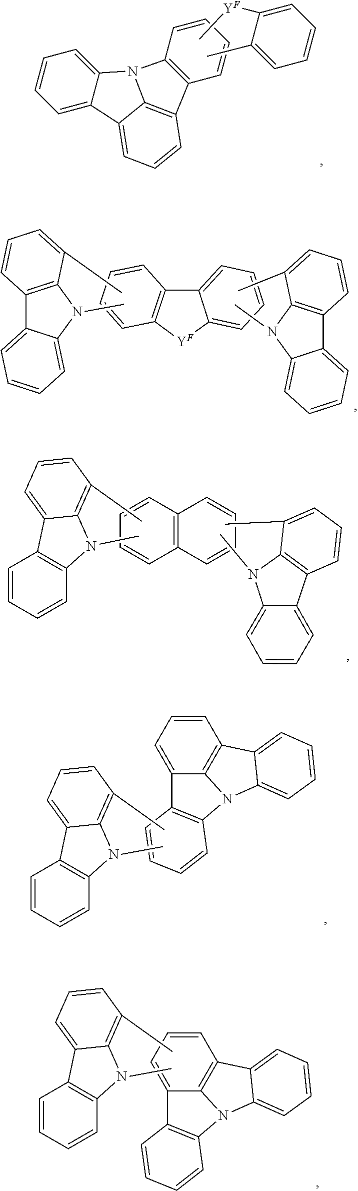

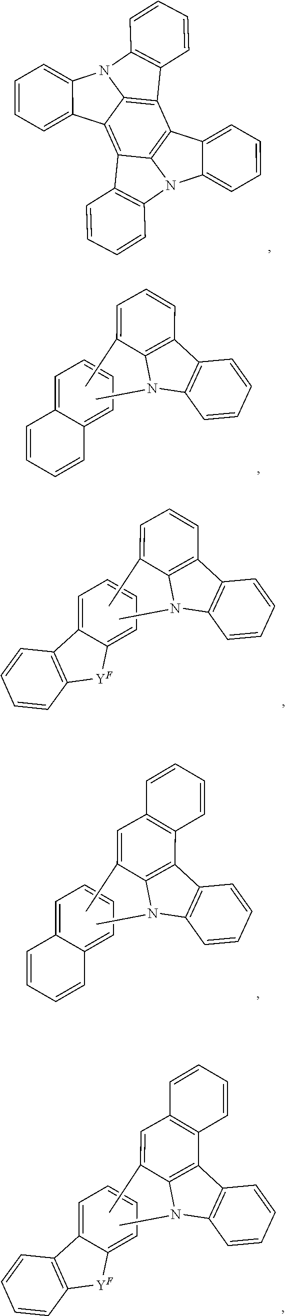

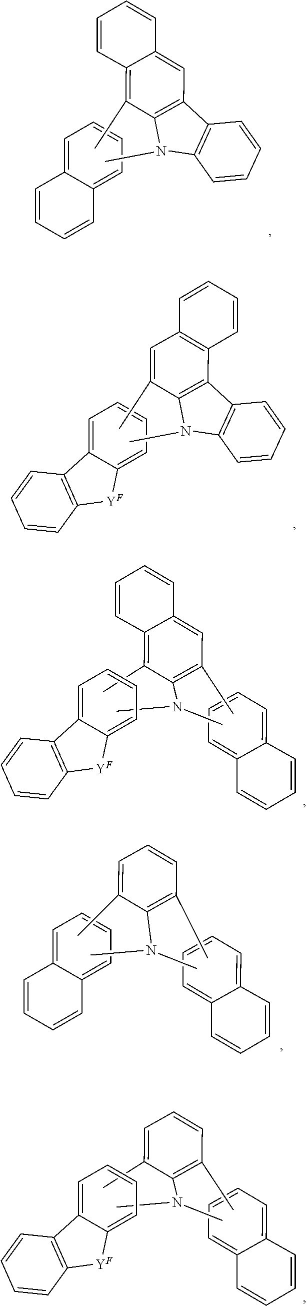

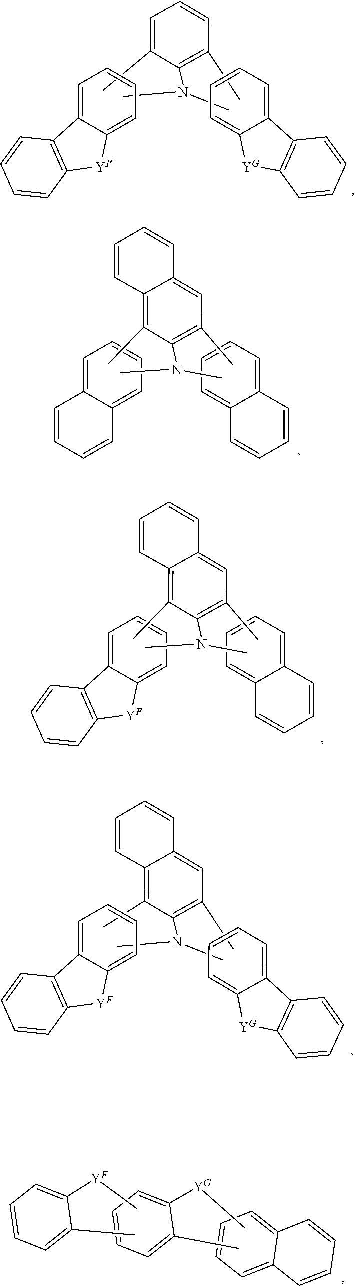

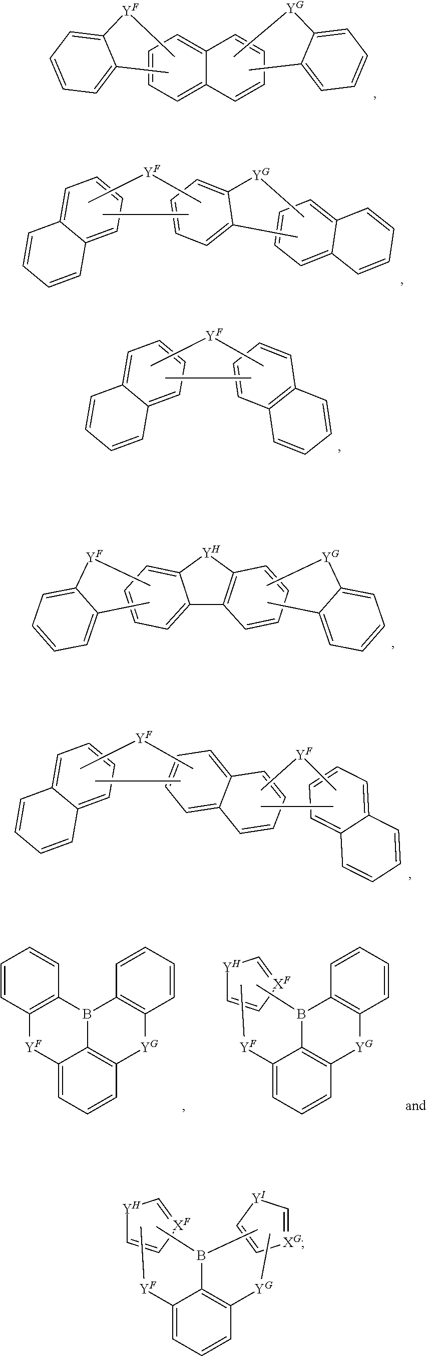

wherein Y^{F}, Y^{G}, Y^{H}, and Y^{I} are each independently selected from the group consisting of BR, NR, PR, O, S, Se, C═O, S═O, SO₂, BRR′, CRR′, SiRR′, and GeRR′;
wherein X^{F} and Y^{G} are each independently selected from the group consisting of C and N; and
wherein R^{F}, R^{G}, R, and R′ are each independently a hydrogen or a substituent selected from the group consisting of the General Substituents as defined herein.

In some of the above embodiments, any carbon ring atoms up to maximum of a total number of three, together with their substituents, in each phenyl ring of any of above structures can be replaced with N.

In some embodiments of the OLED, the fluorescent compound is selected from the group consisting of:

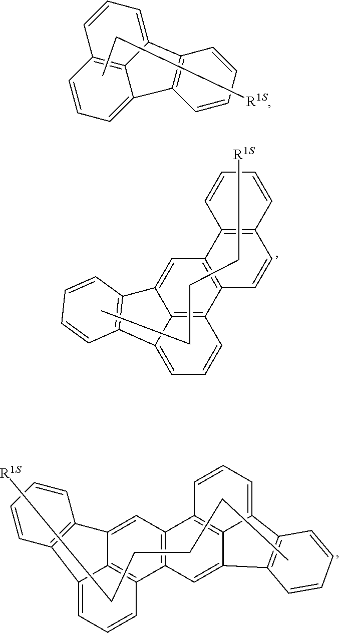

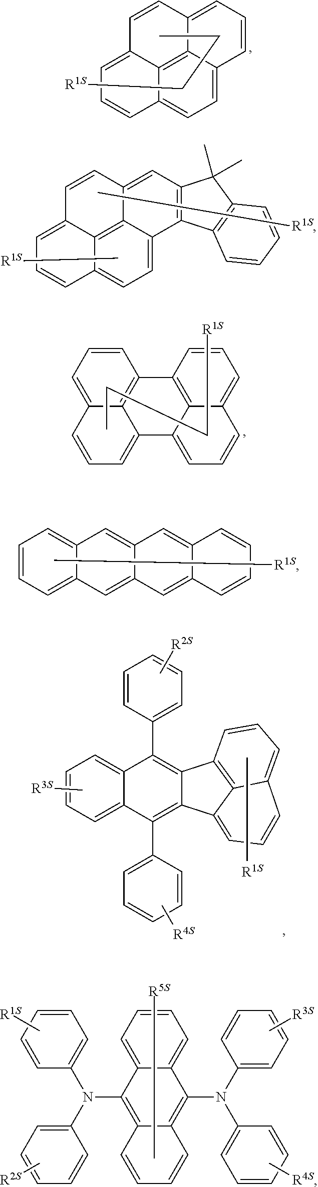

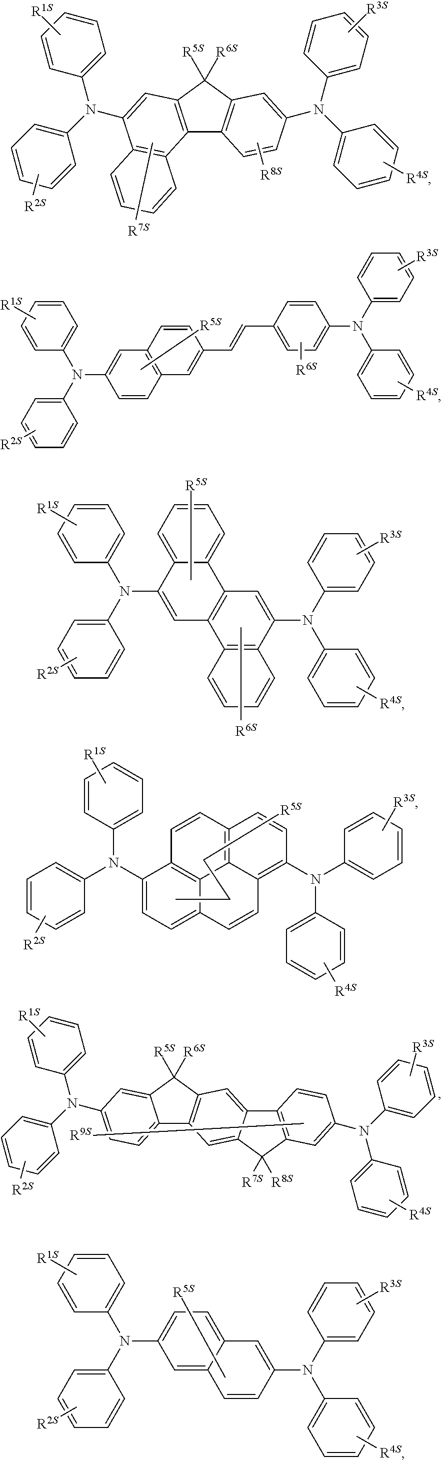

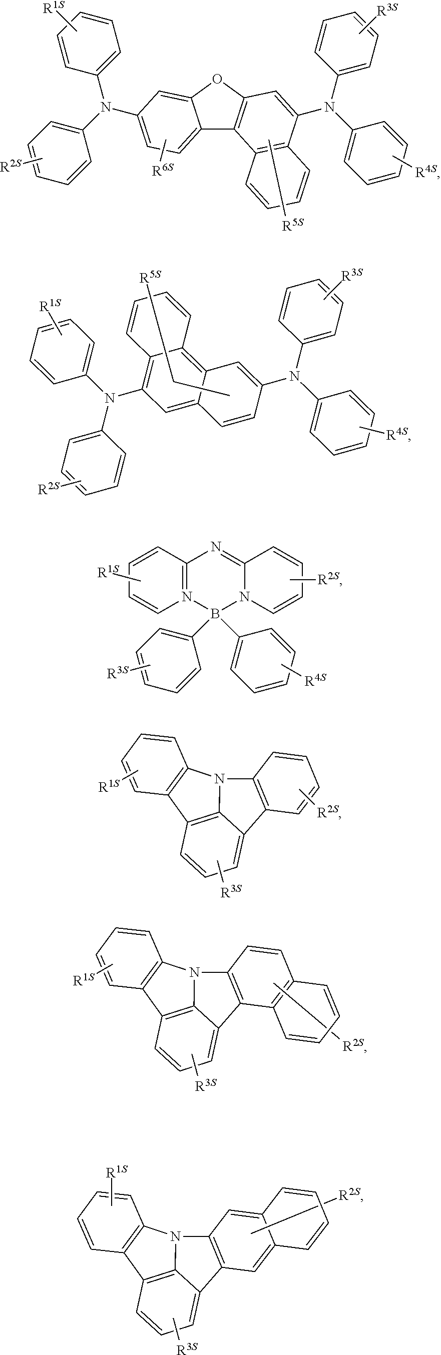

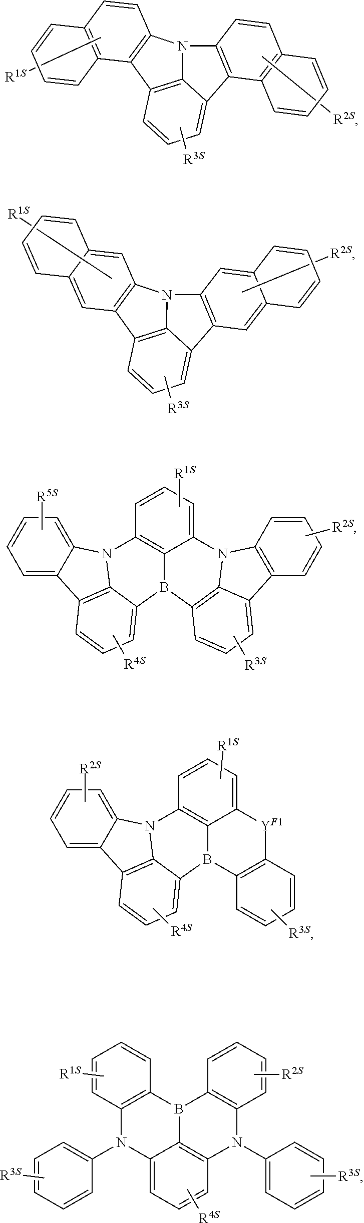

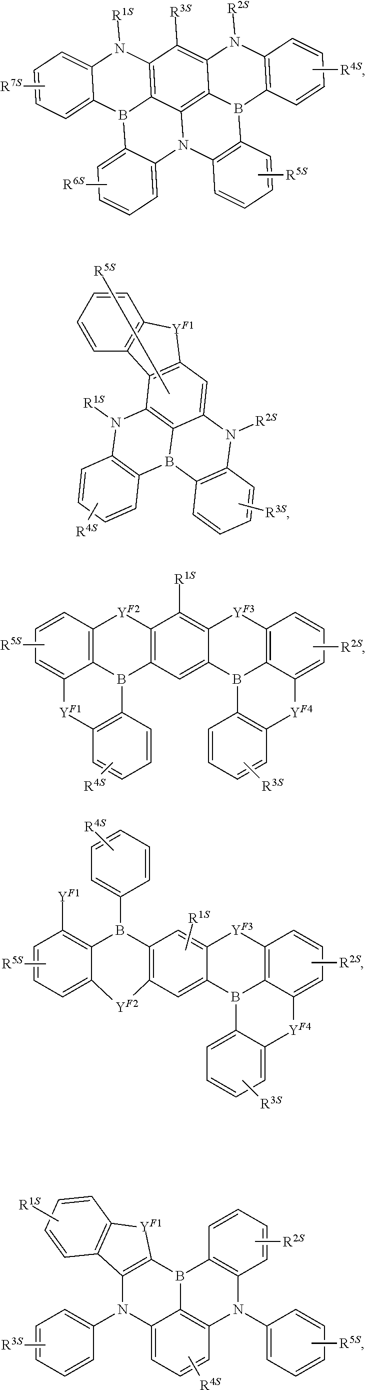

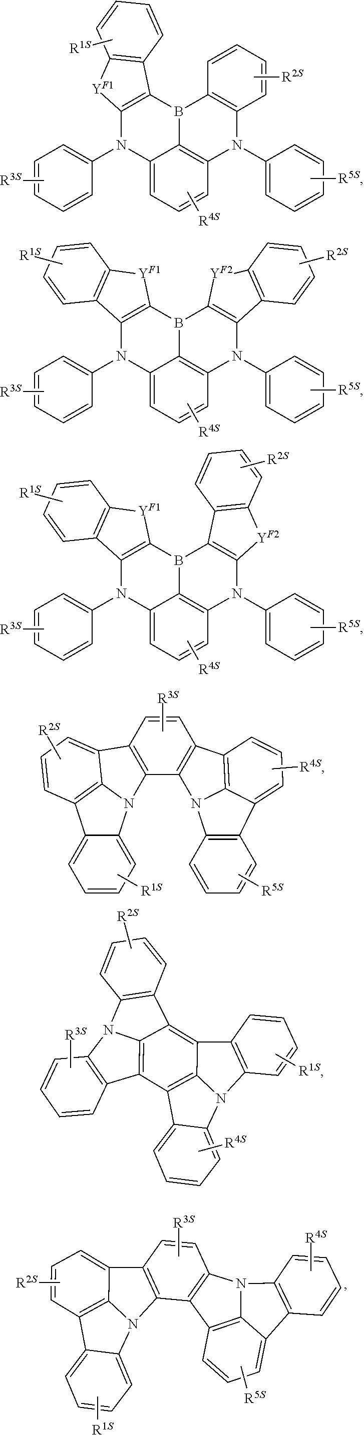

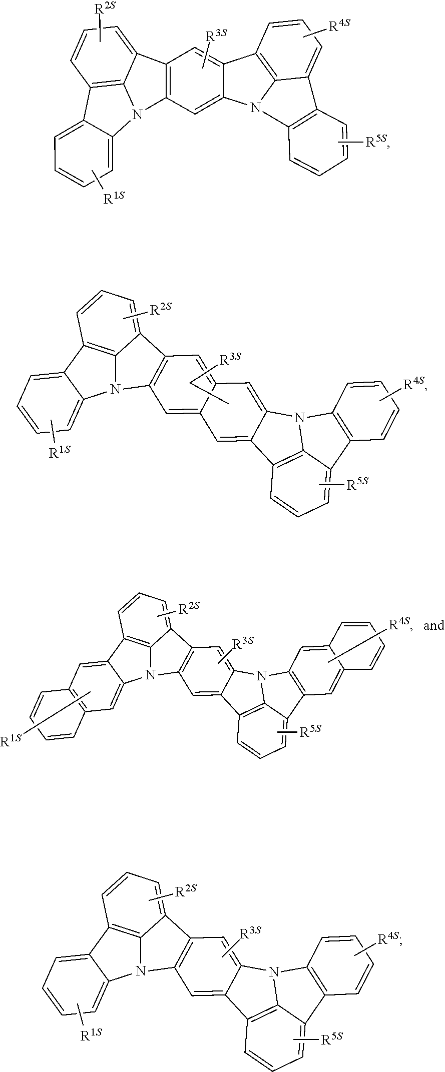

wherein Y^{F1} to Y^{F4} are each independently selected from O, S, and NR^{F1};
wherein R^{F1} and R^{1S} to R^{9S} each independently represents from mono to maximum possible number of substitutions, or no substitution; and
wherein R^{F1} and R^{1S} to R^{9S} are each independently a hydrogen or a substituent selected from the group consisting of the general substituents as defined herein.

In some embodiments, the emitter is selected from the group consisting of the following structures:

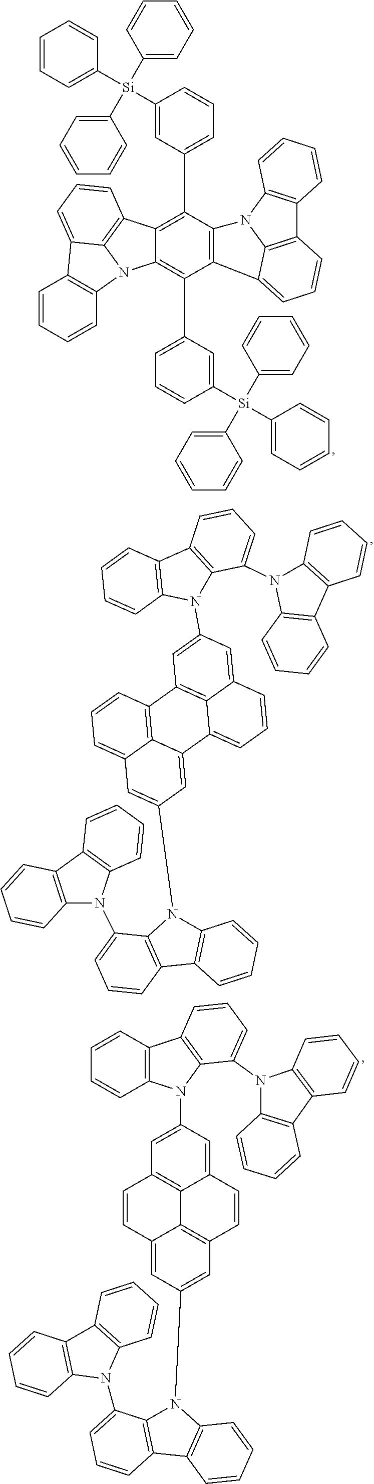

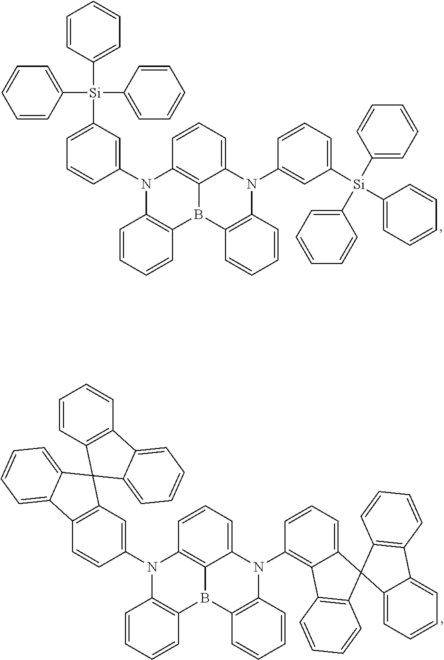 [SMILES: c1ccc([Si](c2ccccc2)(c2ccccc2)c2cccc(N3c4ccccc4B4c5ccccc5N(c5cccc([Si](c6ccccc6)(c6ccccc6)c6ccccc6)c5)c5cccc3c54)c2)cc1 ; c1ccc2c(c1)B1c3ccccc3N(c3cccc4c3-c3ccccc3C43c4ccccc4-c4ccccc43)c3cccc(c31)N2c1ccc2c(c1)C1(c3ccccc3-c3ccccc31)c1ccccc1-2]

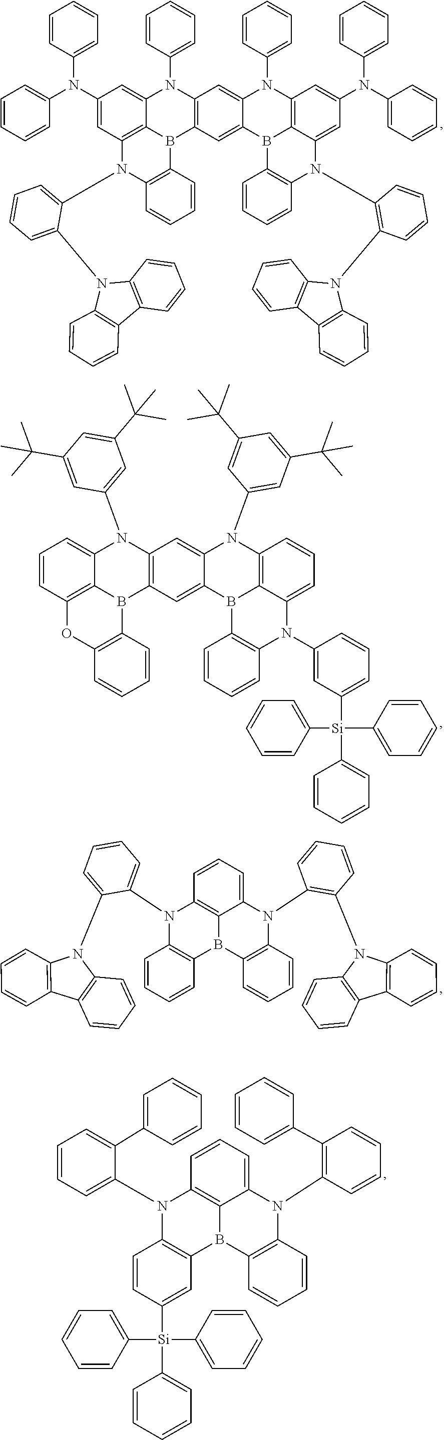

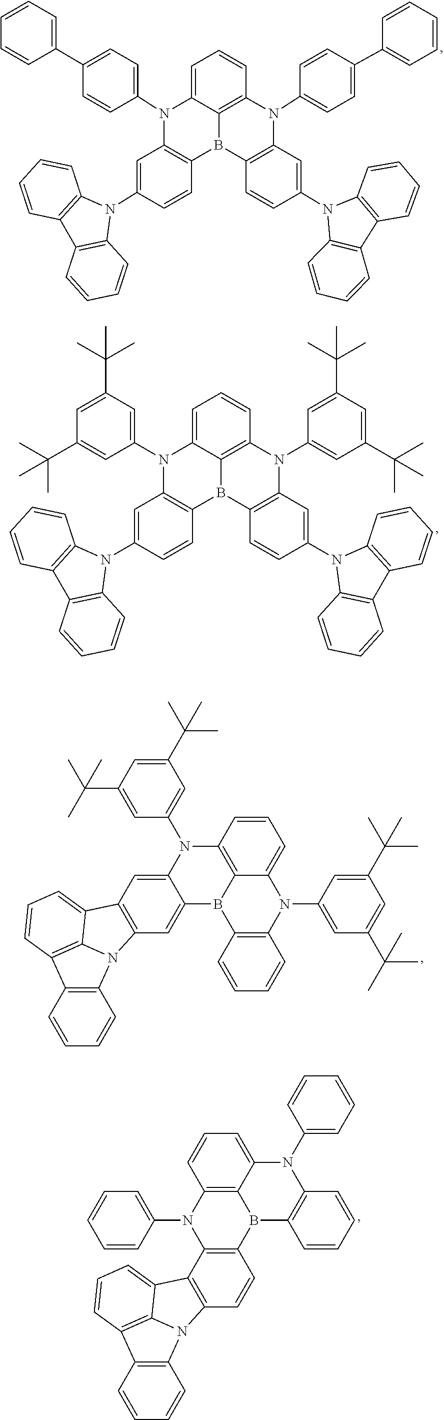

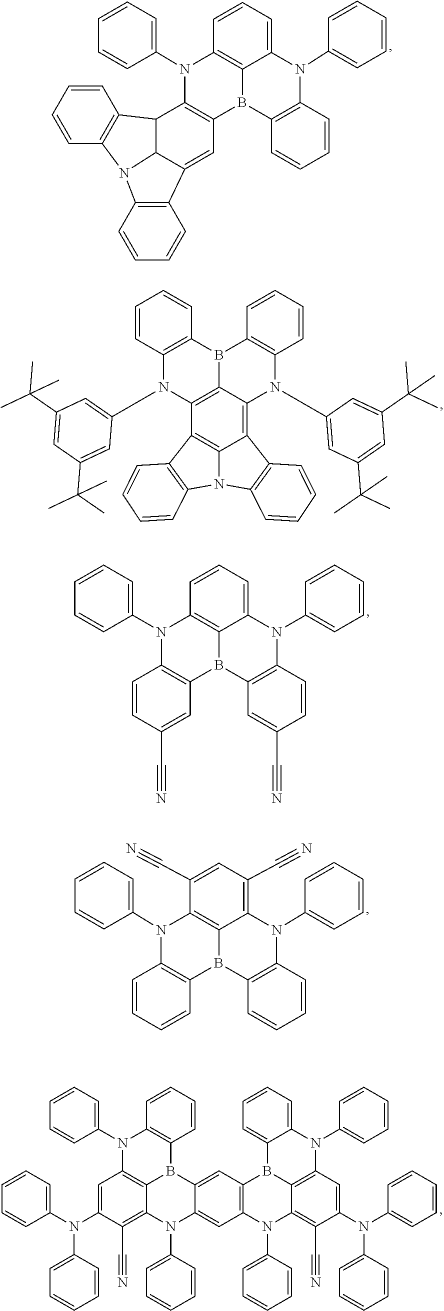

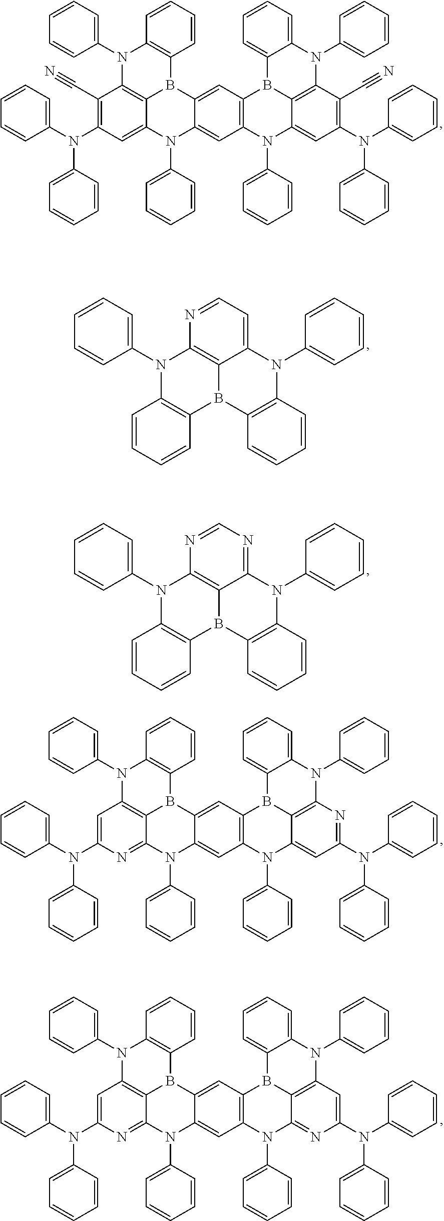

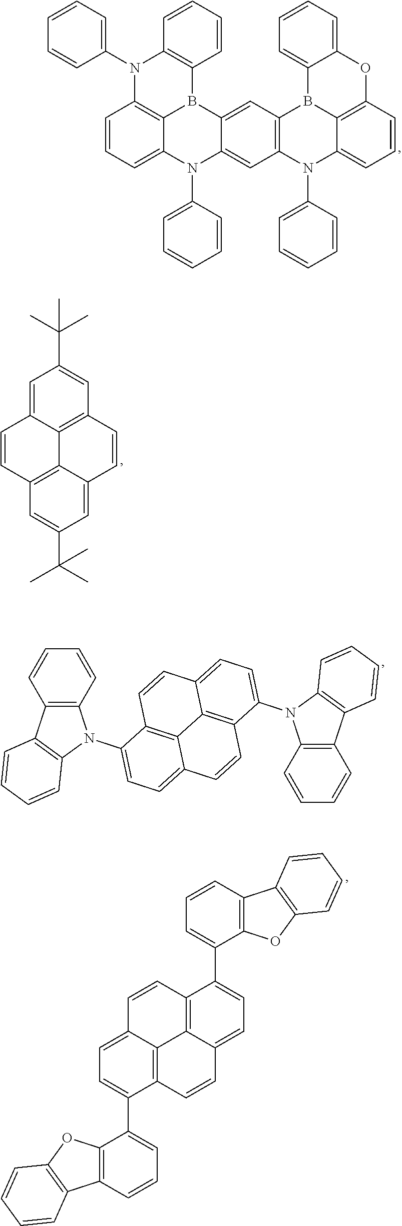

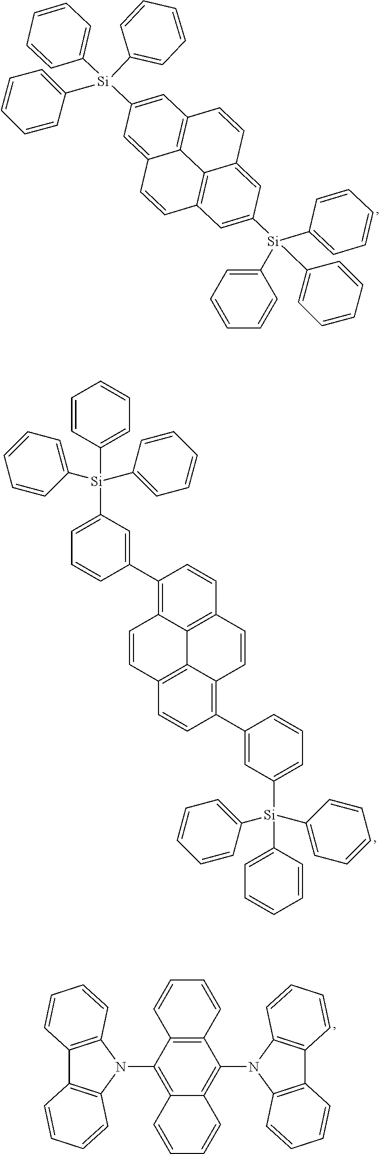 [SMILES: c1ccc([Si](c2ccccc2)(c2ccccc2)c2cc3ccc4cc([Si](c5ccccc5)(c5ccccc5)c5ccccc5)cc5ccc(c2)c3c45)cc1 ; c1ccc([Si](c2ccccc2)(c2ccccc2)c2cccc(-c3ccc4ccc5c(-c6cccc([Si](c7ccccc7)(c7ccccc7)c7ccccc7)c6)ccc6ccc3c4c65)c2)cc1 ; c1ccc2c(-n3c4ccccc4c4ccccc43)c3ccccc3c(-n3c4ccccc4c4ccccc43)c2c1]

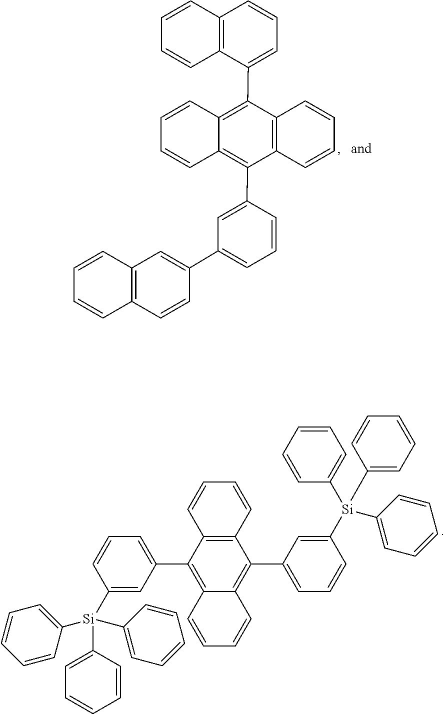

In some of the above embodiments, any carbon ring atoms up to maximum of a total number of three, together with their substituents, in each phenyl ring of any of above structures can be replaced with N. In some embodiments, the compound may be an acceptor, and the OLED may further comprise a sensitizer selected from the group consisting of a delayed fluorescence material, a phosphorescent material, and combination thereof.

In some embodiments, the compound may be a fluorescent emitter, a delayed fluorescence material, or a component of an exciplex that is a fluorescent emitter or a delayed fluorescence material.

In yet another aspect, the OLED of the present disclosure may also comprise an emissive region containing a compound as disclosed in the above compounds section of the present disclosure. In some embodiments, the emissive layer further comprises an additional host, wherein the additional host comprises a triphenylene containing benzo-fused thiophene or benzo-fused furan;
wherein any substituent in the host is an unfused substituent independently selected from the group consisting of CₙH₂ₙ₊₁, OCₙH₂ₙ₊₁, OAr₁, N(CₙH₂ₙ₊₁)₂, N(Ar₁)(Ar₂), CH═CH—CₙH₂ₙ₊₁, C≡CCₙH₂ₙ₊₁, Ar₁, Ar₁-Ar₂, CₙH₂ₙ—Ar₁, or no substitution; wherein n is an integer from 1 to 10; and wherein Ar₁ and Ar₂ are independently selected from the group consisting of benzene, biphenyl, naphthalene, triphenylene, carbazole, and heteroaromatic analogs thereof.

In some embodiments, the emissive layer further comprises an additional host, wherein the additional host comprises at least one chemical moiety selected from the group consisting of triphenylene, carbazole, indolocarbazole, dibenzothiophene, dibenzofuran, dibenzoselenophene, 5λ²-benzo[d]benzo[4,5]imidazo[3,2-a]imidazole, 5,9-dioxa-13b-boranaphtho[3,2,1-de]anthracene, triazine, boryl, silyl, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

In some embodiments, the additional host can be selected from the group consisting of:

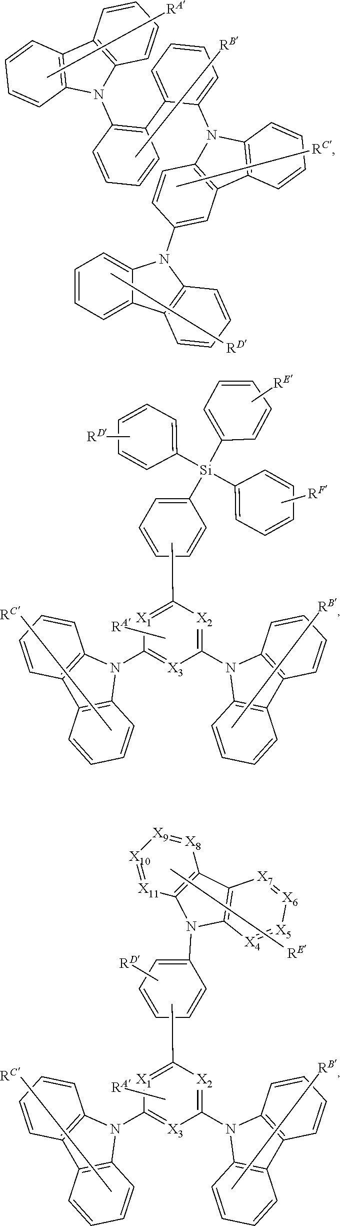

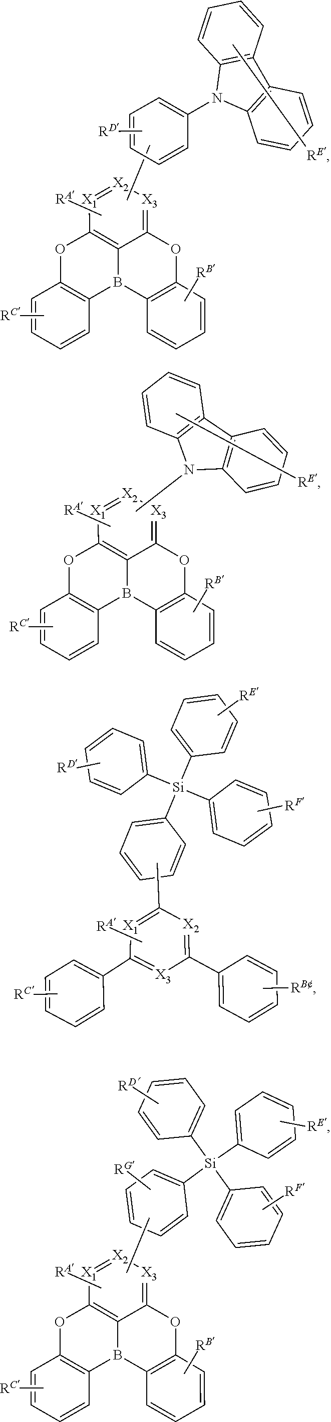

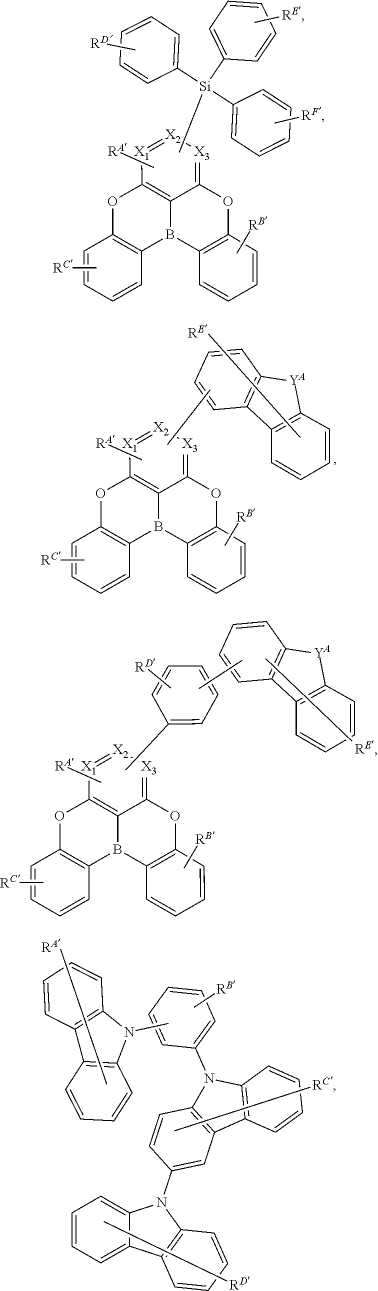

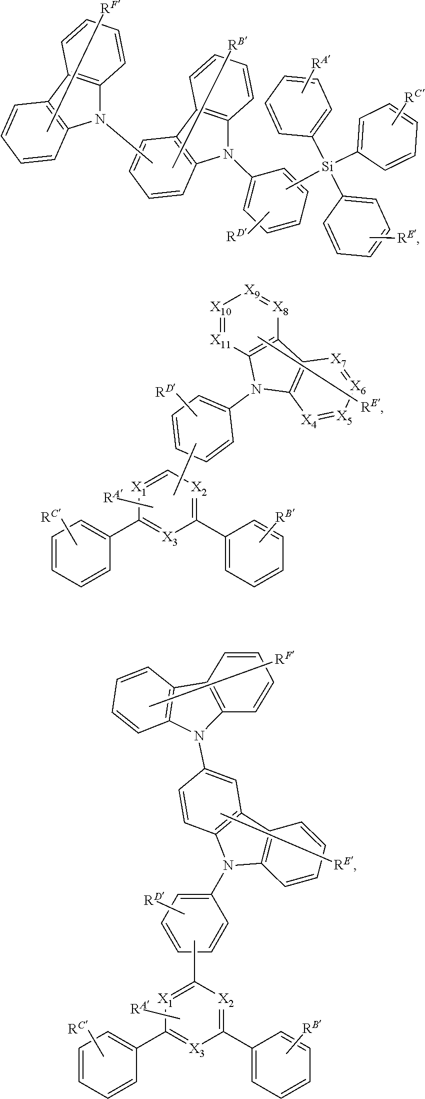

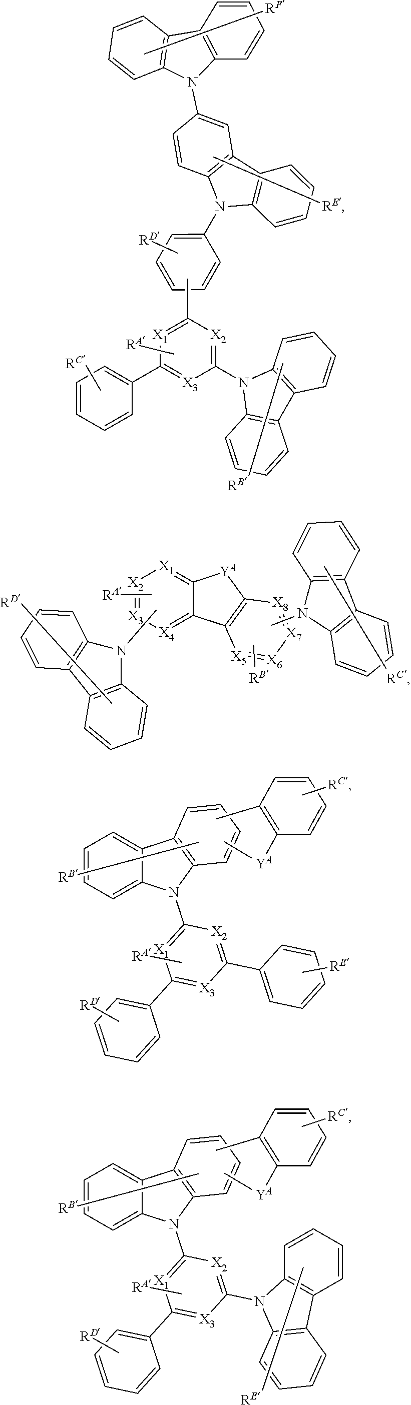

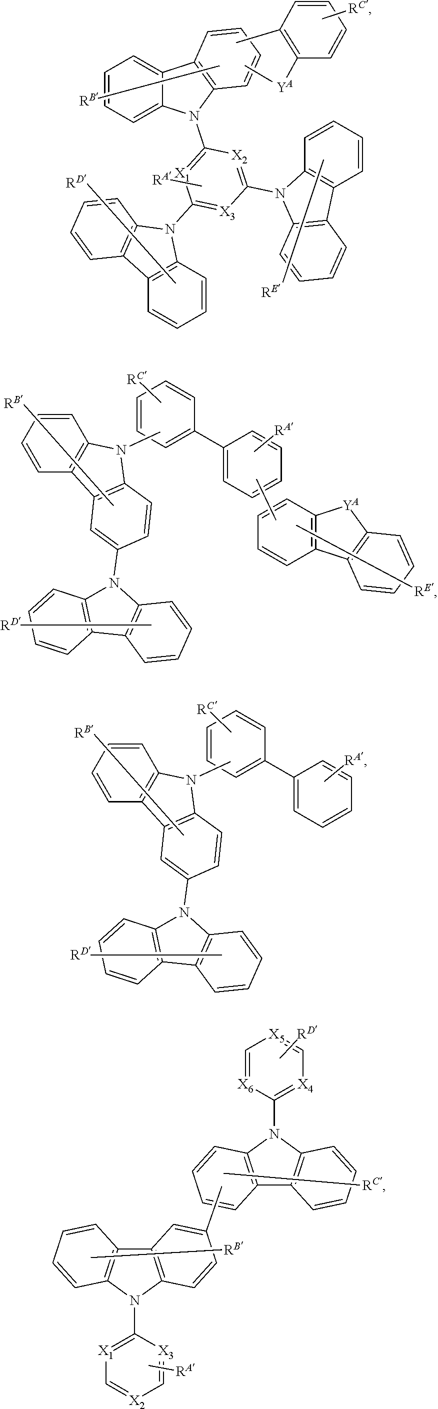

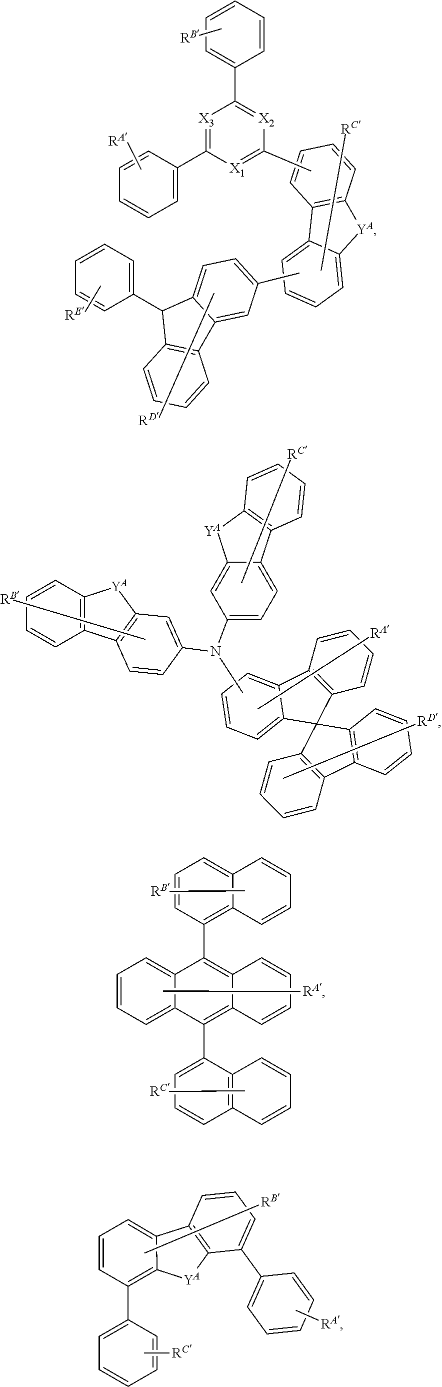

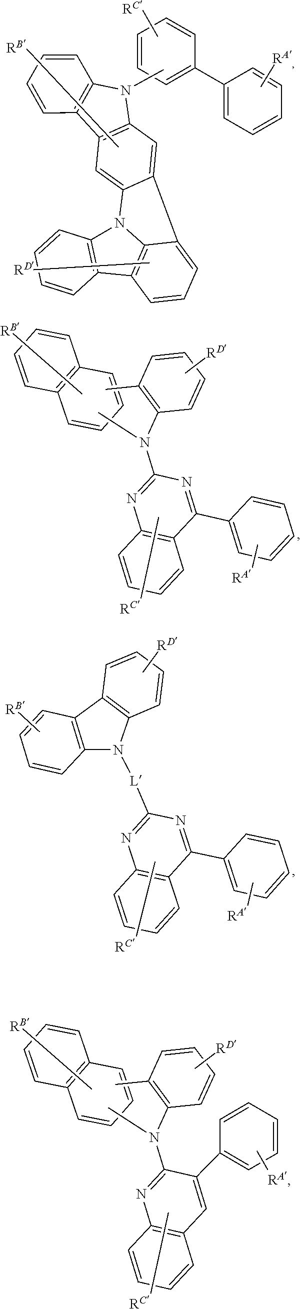

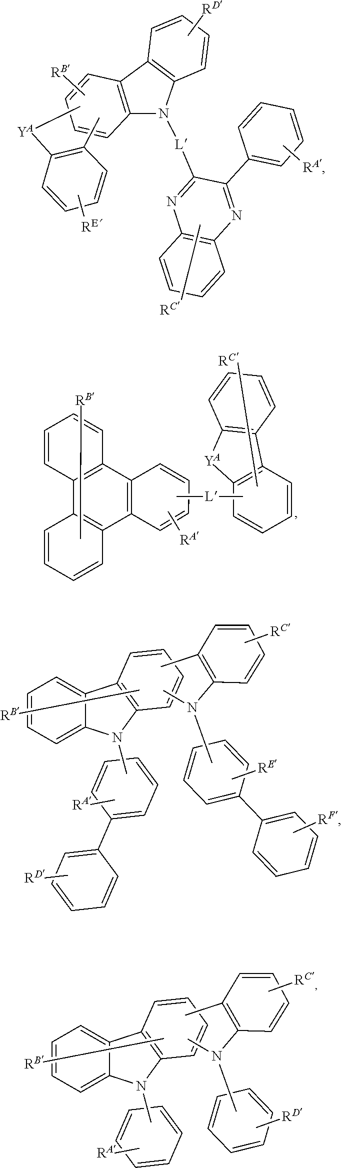

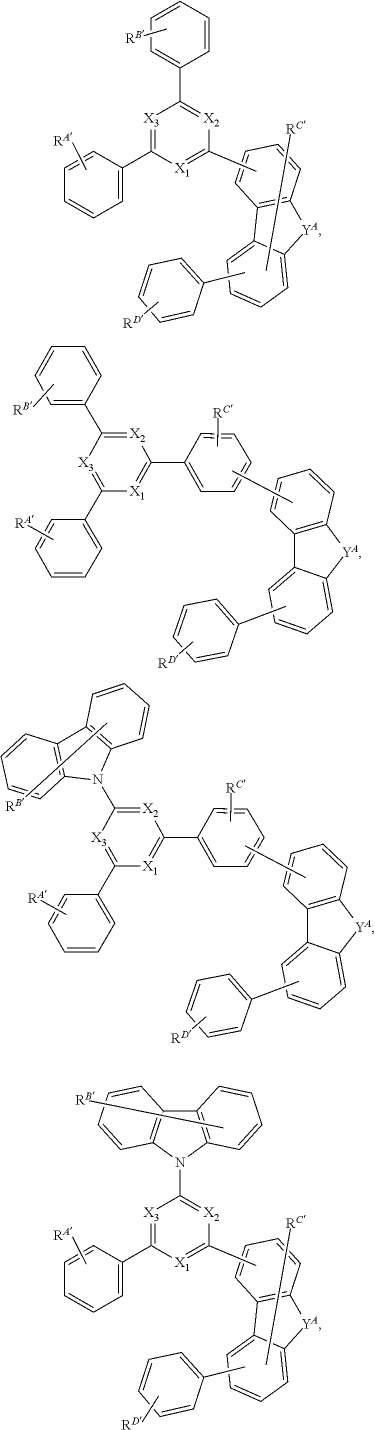

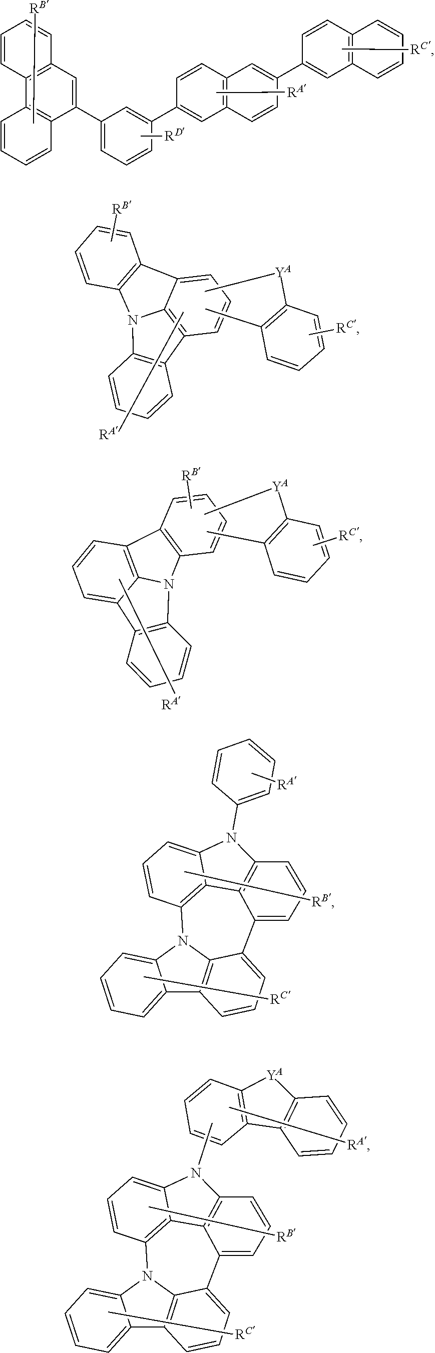

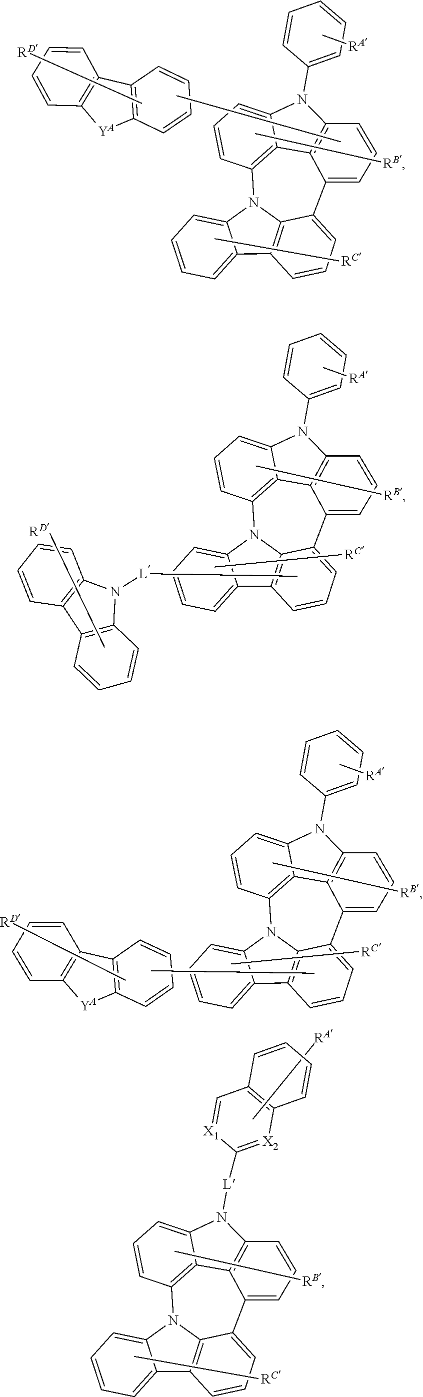

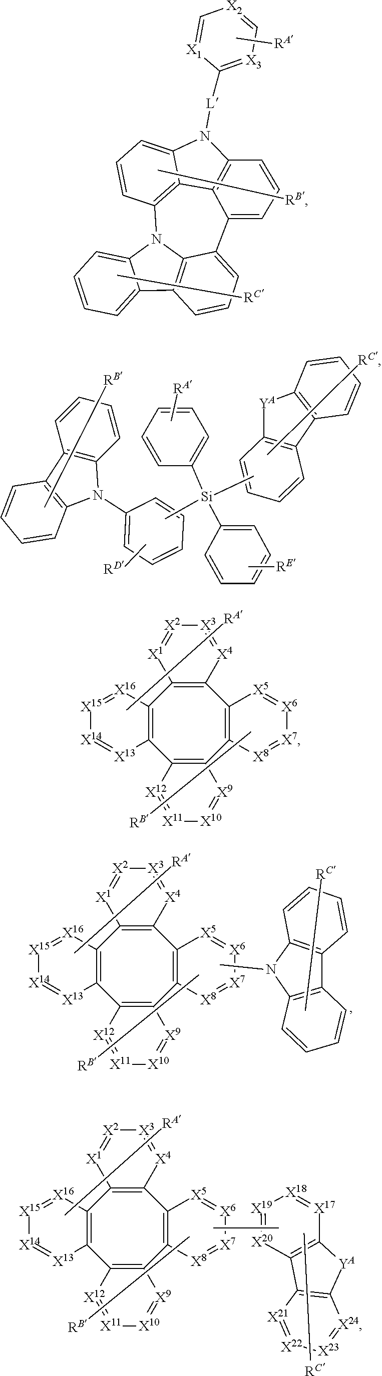

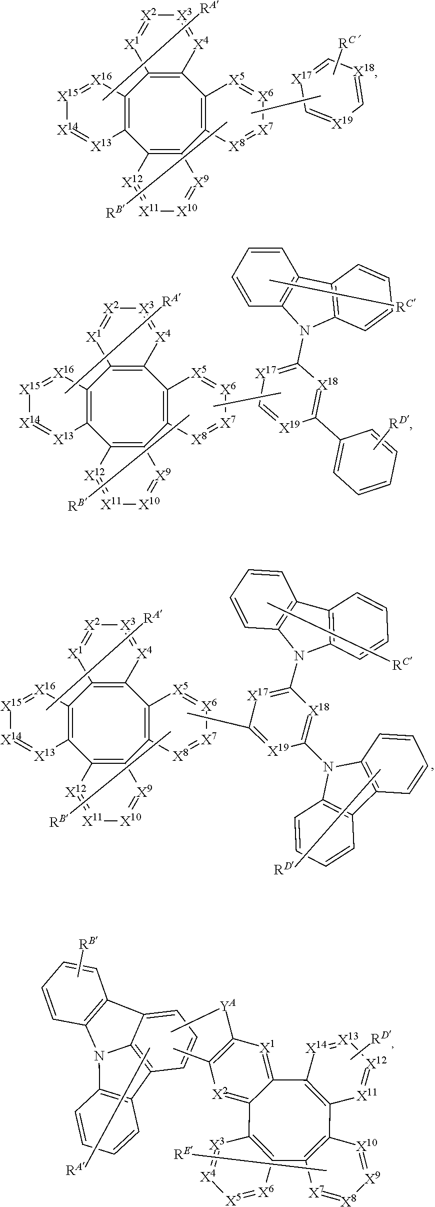

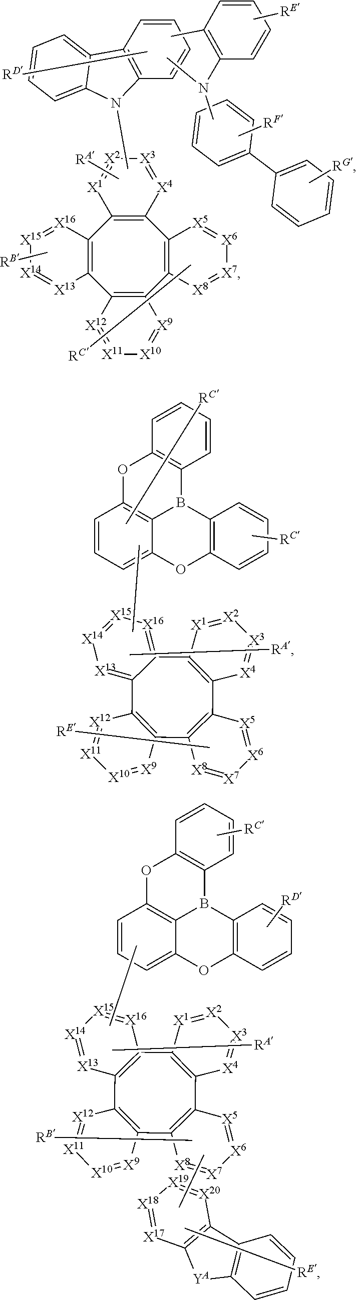

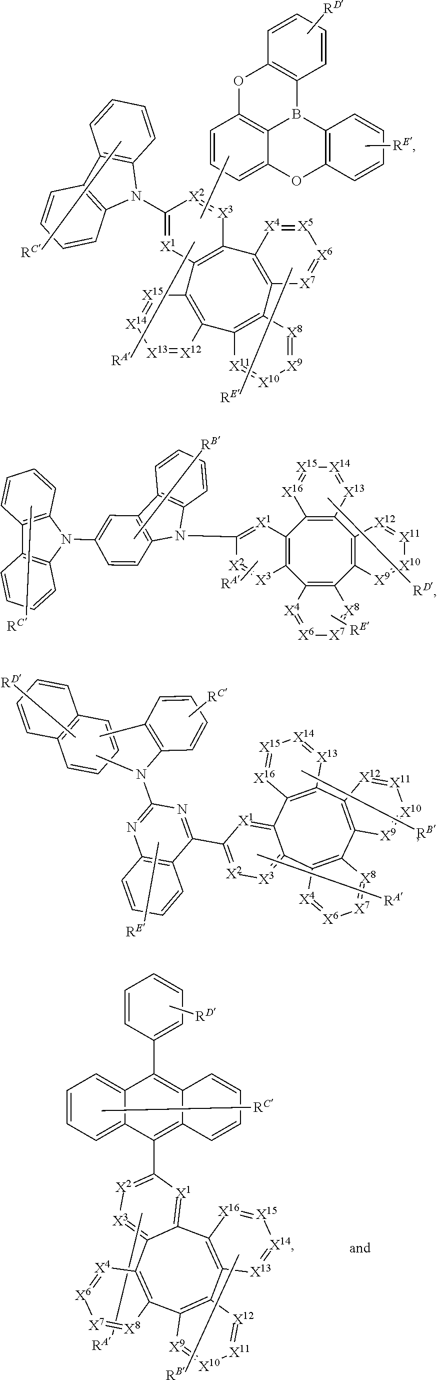

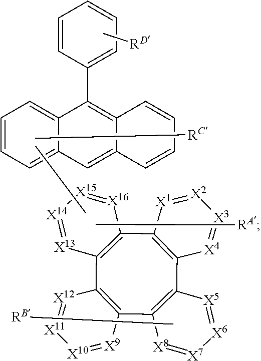

wherein:
each of X¹ to X²⁴ is independently C or N;
L′ is a direct bond or an organic linker;
each Y^{A} is independently selected from the group consisting of absent a bond, O, S, Se, CRR′, SiRR′, GeRR′, NR, BR, BRR′;
each of R^{A′}, R^{B′}, R^{C′}, R^{D′}, R^{E′}, R^{F′}, and R^{G′} independently represents mono, up to the maximum substitutions, or no substitutions;
each R, R′, R^{A′}, R^{B′}, R^{C′}, R^{D′}, R^{E′}, R^{F′} and R^{G′} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, selenyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, boryl, and combinations thereof;
two adjacent of R^{A′}, R^{B′}, R^{C′}, R^{D′}, R^{E′}, R^{F′}, and R^{G′} are optionally joined or fused to form a ring.

In some embodiments, the additional host can be selected from the group consisting of:

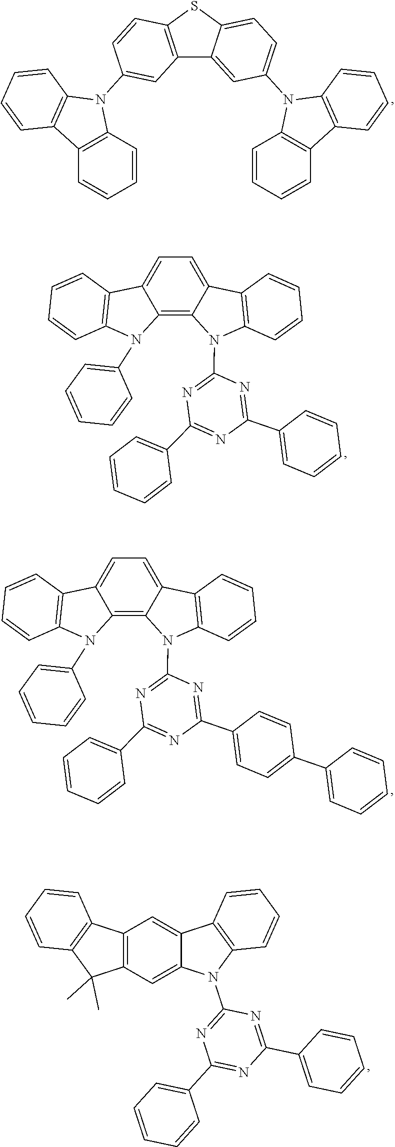 [SMILES: c1ccc2c(c1)c1ccccc1n2-c1ccc2sc3ccc(-n4c5ccccc5c5ccccc54)cc3c2c1 ; c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; CC1(C)c2ccccc2-c2cc3c4ccccc4n(-c4nc(-c5ccccc5)nc(-c5ccccc5)n4)c3cc21]

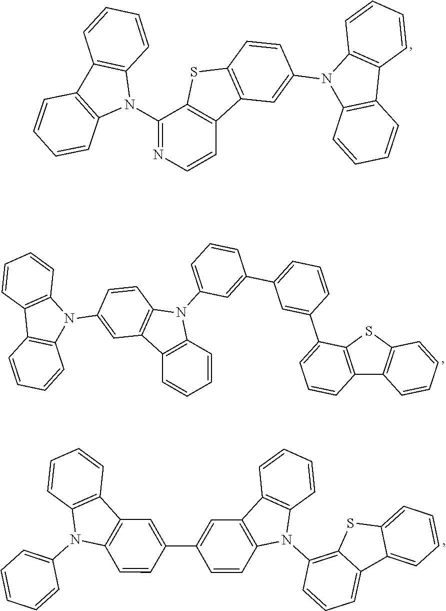 [SMILES: c1ccc2c(c1)c1ccccc1n2-c1ccc2sc3c(-n4c5ccccc5c5ccccc54)nccc3c2c1 ; c1cc(-c2cccc(-n3c4ccccc4c4cc(-n5c6ccccc6c6ccccc65)ccc43)c2)cc(-c2cccc3c2sc2ccccc23)c1 ; c1ccc(-n2c3ccccc3c3cc(-c4ccc5c(c4)c4ccccc4n5-c4cccc5c4sc4ccccc45)ccc32)cc1]

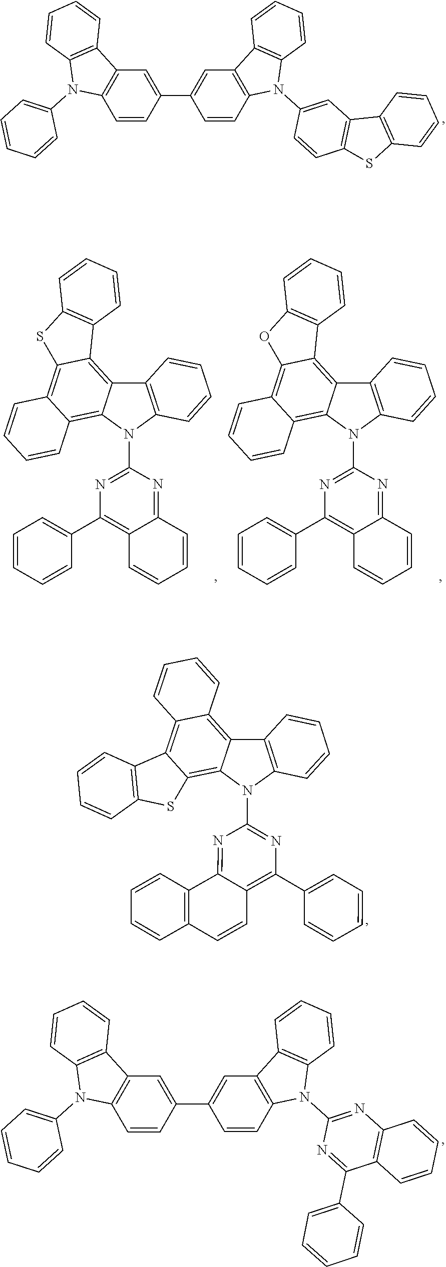

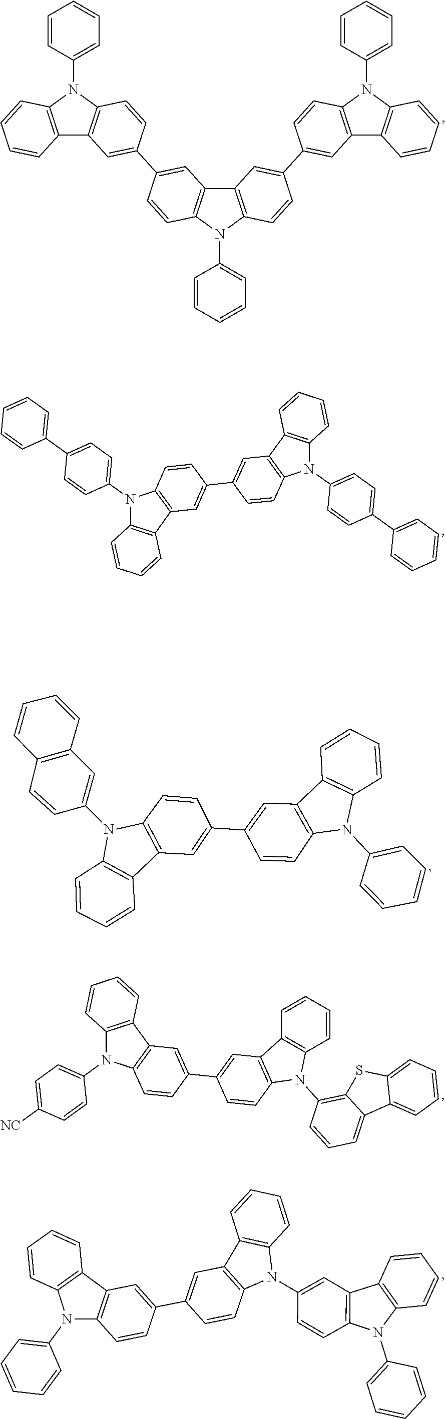 [SMILES: c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-n2c3ccccc3c3cc(-c4ccc5c(c4)c4ccccc4n5-c4ccc5ccccc5c4)ccc32)cc1 ; N#Cc1ccc(-n2c3ccccc3c3cc(-c4ccc5c(c4)c4ccccc4n5-c4cccc5c4sc4ccccc45)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

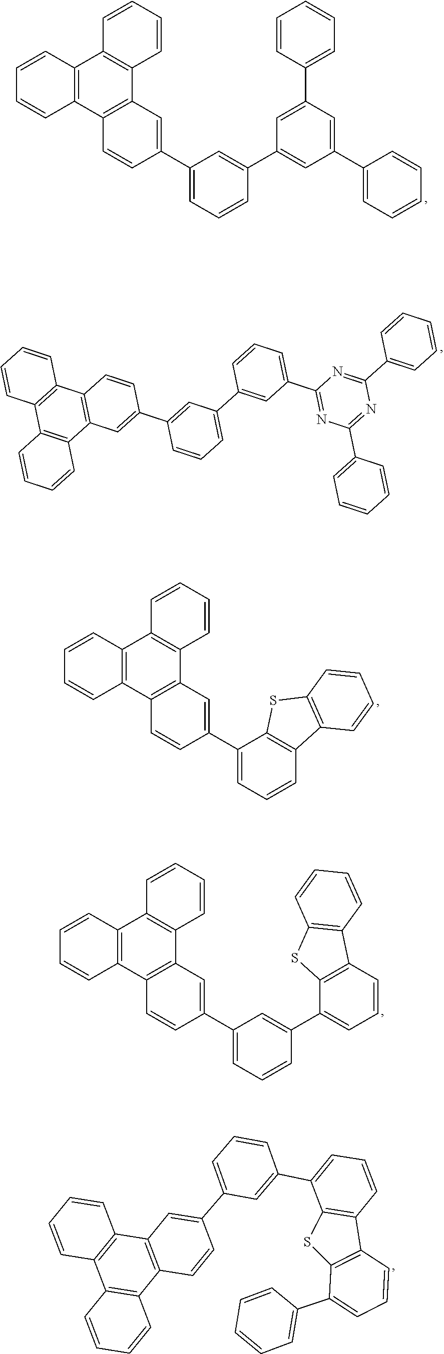

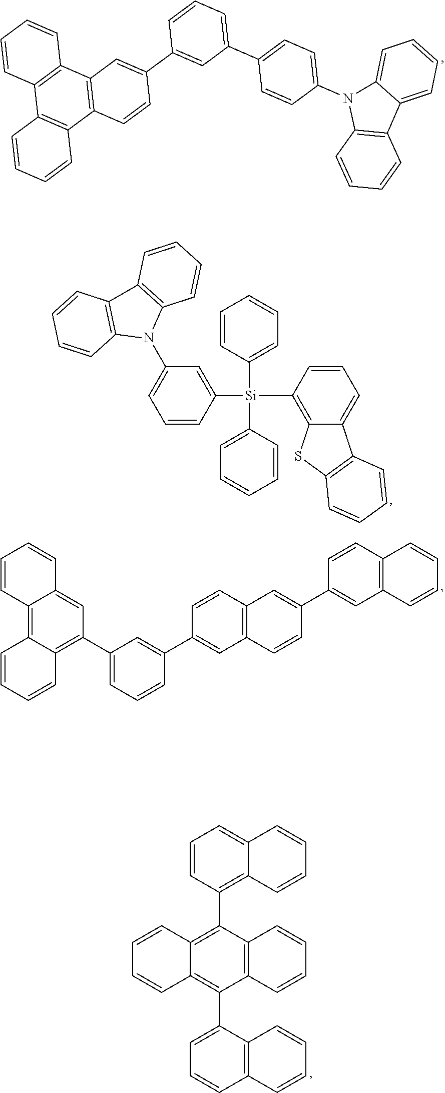

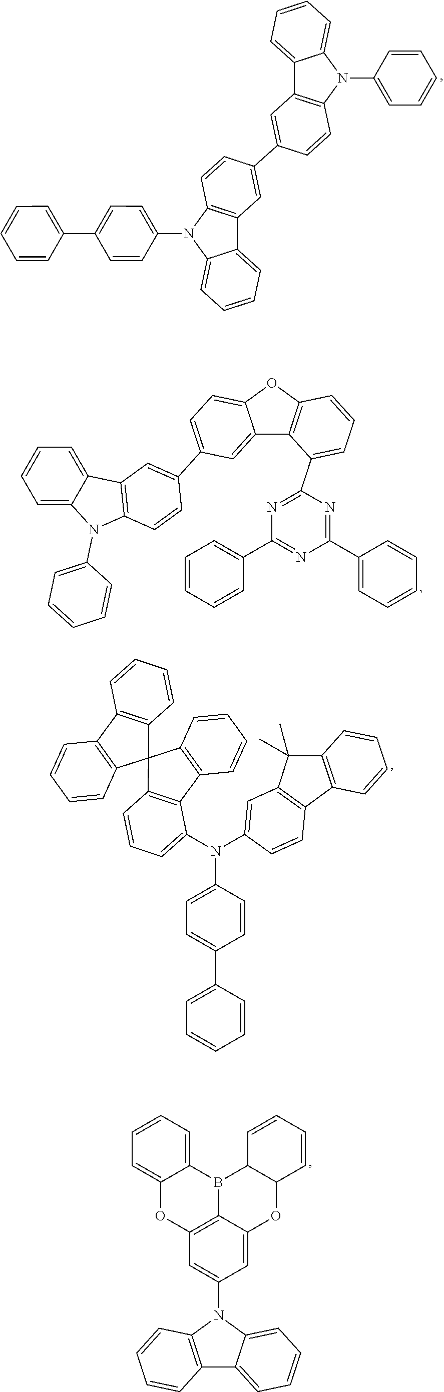 [SMILES: c1ccc(-c2ccc(-n3c4ccccc4c4cc(-c5ccc6c(c5)c5ccccc5n6-c5ccccc5)ccc43)cc2)cc1 ; c1ccc(-c2nc(-c3ccccc3)nc(-c3cccc4oc5ccc(-c6ccc7c(c6)c6ccccc6n7-c6ccccc6)cc5c34)n2)cc1 ; CC1(C)c2ccccc2-c2ccc(N(c3ccc(-c4ccccc4)cc3)c3cccc4c3-c3ccccc3C43c4ccccc4-c4ccccc43)cc21 ; C1=CC2Oc3cc(-n4c5ccccc5c5ccccc54)cc4c3B(c3ccccc3O4)C2C=C1]

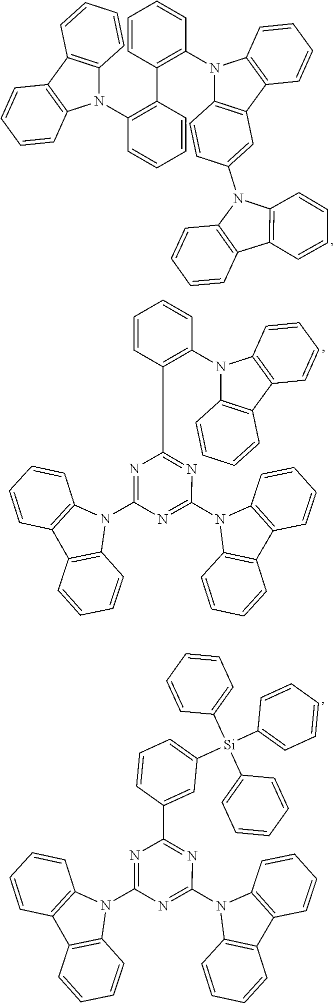

and combinations thereof.

In some embodiments, the emissive region can comprise a compound as described herein.

In some embodiments, at least one of the anode, the cathode, or a new layer disposed over the organic emissive layer functions as an enhancement layer. The enhancement layer comprises a plasmonic material exhibiting surface plasmon resonance that non-radiatively couples to the emitter material and transfers excited state energy from the emitter material to non-radiative mode of surface plasmon polariton. The enhancement layer is provided no more than a threshold distance away from the organic emissive layer, wherein the emitter material has a total non-radiative decay rate constant and a total radiative decay rate constant due to the presence of the enhancement layer and the threshold distance is where the total non-radiative decay rate constant is equal to the total radiative decay rate constant. In some embodiments, the OLED further comprises an outcoupling layer. In some embodiments, the outcoupling layer is disposed over the enhancement layer on the opposite side of the organic emissive layer. In some embodiments, the outcoupling layer is disposed on opposite side of the emissive layer from the enhancement layer but still outcouples energy from the surface plasmon mode of the enhancement layer. The outcoupling layer scatters the energy from the surface plasmon polaritons. In some embodiments this energy is scattered as photons to free space. In other embodiments, the energy is scattered from the surface plasmon mode into other modes of the device such as but not limited to the organic waveguide mode, the substrate mode, or another waveguiding mode. If energy is scattered to the non-free space mode of the OLED other outcoupling schemes could be incorporated to extract that energy to free space. In some embodiments, one or more intervening layer can be disposed between the enhancement layer and the outcoupling layer. The examples for intervening layer(s) can be dielectric materials, including organic, inorganic, perovskites, oxides, and may include stacks and/or mixtures of these materials.

The enhancement layer modifies the effective properties of the medium in which the emitter material resides resulting in any or all of the following: a decreased rate of emission, a modification of emission line-shape, a change in emission intensity with angle, a change in the stability of the emitter material, a change in the efficiency of the OLED, and reduced efficiency roll-off of the OLED device. Placement of the enhancement layer on the cathode side, anode side, or on both sides results in OLED devices which take advantage of any of the above-mentioned effects. In addition to the specific functional layers mentioned herein and illustrated in the various OLED examples shown in the figures, the OLEDs according to the present disclosure may include any of the other functional layers often found in OLEDs.

The enhancement layer can be comprised of plasmonic materials, optically active metamaterials, or hyperbolic metamaterials. As used herein, a plasmonic material is a material in which the real part of the dielectric constant crosses zero in the visible or ultraviolet region of the electromagnetic spectrum. In some embodiments, the plasmonic material includes at least one metal. In such embodiments the metal may include at least one of Ag, Al, Au, Ir, Pt, Ni, Cu, W, Ta, Fe, Cr, Mg, Ga, Rh, Ti, Ru, Pd, In, Bi, Ca alloys or mixtures of these materials, and stacks of these materials. In general, a metamaterial is a medium composed of different materials where the medium as a whole acts differently than the sum of its material parts. In particular, we define optically active metamaterials as materials which have both negative permittivity and negative permeability. Hyperbolic metamaterials, on the other hand, are anisotropic media in which the permittivity or permeability are of different sign for different spatial directions. Optically active metamaterials and hyperbolic metamaterials are strictly distinguished from many other photonic structures such as Distributed Bragg Reflectors (“DBRs”) in that the medium should appear uniform in the direction of propagation on the length scale of the wavelength of light. Using terminology that one skilled in the art can understand: the dielectric constant of the metamaterials in the direction of propagation can be described with the effective medium approximation. Plasmonic materials and metamaterials provide methods for controlling the propagation of light that can enhance OLED performance in a number of ways.

In some embodiments, the enhancement layer is provided as a planar layer. In other embodiments, the enhancement layer has wavelength-sized features that are arranged periodically, quasi-periodically, or randomly, or sub-wavelength-sized features that are arranged periodically, quasi-periodically, or randomly. In some embodiments, the wavelength-sized features and the sub-wavelength-sized features have sharp edges.

In some embodiments, the outcoupling layer has wavelength-sized features that are arranged periodically, quasi-periodically, or randomly, or sub-wavelength-sized features that are arranged periodically, quasi-periodically, or randomly. In some embodiments, the outcoupling layer may be composed of a plurality of nanoparticles and in other embodiments the outcoupling layer is composed of a plurality of nanoparticles disposed over a material. In these embodiments the outcoupling may be tunable by at least one of varying a size of the plurality of nanoparticles, varying a shape of the plurality of nanoparticles, changing a material of the plurality of nanoparticles, adjusting a thickness of the material, changing the refractive index of the material or an additional layer disposed on the plurality of nanoparticles, varying a thickness of the enhancement layer, and/or varying the material of the enhancement layer. The plurality of nanoparticles of the device may be formed from at least one of metal, dielectric material, semiconductor materials, an alloy of metal, a mixture of dielectric materials, a stack or layering of one or more materials, and/or a core of one type of material and that is coated with a shell of a different type of material. In some embodiments, the outcoupling layer is composed of at least metal nanoparticles wherein the metal is selected from the group consisting of Ag, Al, Au, Ir, Pt, Ni, Cu, W, Ta, Fe, Cr, Mg, Ga, Rh, Ti, Ru, Pd, In, Bi, Ca, alloys or mixtures of these materials, and stacks of these materials. The plurality of nanoparticles may have additional layer disposed over them. In some embodiments, the polarization of the emission can be tuned using the outcoupling layer. Varying the dimensionality and periodicity of the outcoupling layer can select a type of polarization that is preferentially outcoupled to air. In some embodiments the outcoupling layer also acts as an electrode of the device.

In yet another aspect, the present disclosure also provides a consumer product comprising an organic light-emitting device (OLED) having an anode; a cathode; and an organic layer disposed between the anode and the cathode, wherein the organic layer may comprise a compound as disclosed in the above compounds section of the present disclosure.

In some embodiments, the consumer product comprises an OLED having an anode; a cathode; and an organic layer disposed between the anode and the cathode, wherein the organic layer may comprise the compound of the present disclosure.

In some embodiments, the consumer product can be one of a flat panel display, a computer monitor, a medical monitor, a television, a billboard, a light for interior or exterior illumination and/or signaling, a heads-up display, a fully or partially transparent display, a flexible display, a laser printer, a telephone, a cell phone, tablet, a phablet, a personal digital assistant (PDA), a wearable device, a laptop computer, a digital camera, a camcorder, a viewfinder, a micro-display that is less than 2 inches diagonal, a 3-D display, a virtual reality or augmented reality display, a vehicle, a video wall comprising multiple displays tiled together, a theater or stadium screen, a light therapy device, and a sign.

Generally, an OLED comprises at least one organic layer disposed between and electrically connected to an anode and a cathode. When a current is applied, the anode injects holes and the cathode injects electrons into the organic layer(s). The injected holes and electrons each migrate toward the oppositely charged electrode. When an electron and hole localize on the same molecule, an “exciton,” which is a localized electron-hole pair having an excited energy state, is formed. Light is emitted when the exciton relaxes via a photoemissive mechanism. In some cases, the exciton may be localized on an excimer or an exciplex. Non-radiative mechanisms, such as thermal relaxation, may also occur, but are generally considered undesirable.

Several OLED materials and configurations are described in U.S. Pat. Nos. 5,844,363, 6,303,238, and 5,707,745, which are incorporated herein by reference in their entirety.

The initial OLEDs used emissive molecules that emitted light from their singlet states (“fluorescence”) as disclosed, for example, in U.S. Pat. No. 4,769,292, which is incorporated by reference in its entirety. Fluorescent emission generally occurs in a time frame of less than 10 nanoseconds.

More recently, OLEDs having emissive materials that emit light from triplet states (“phosphorescence”) have been demonstrated. Baldo et al., “Highly Efficient Phosphorescent Emission from Organic Electroluminescent Devices,” Nature, vol. 395, 151-154, 1998; (“Baldo-I”) and Baldo et al., “Very high-efficiency green organic light-emitting devices based on electrophosphorescence,” Appl. Phys. Lett., vol. 75, No. 3, 4-6 (1999) (“Baldo-II”), are incorporated by reference in their entireties. Phosphorescence is described in more detail in U.S. Pat. No. 7,279,704 at cols. 5-6, which are incorporated by reference.

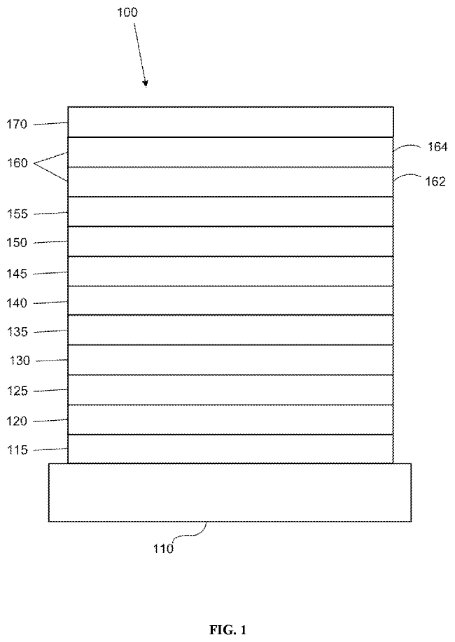

FIG. 1 shows an organic light emitting device 100. The figures are not necessarily drawn to scale. Device 100 may include a substrate 110, an anode 115, a hole injection layer 120, a hole transport layer 125, an electron blocking layer 130, an emissive layer 135, a hole blocking layer 140, an electron transport layer 145, an electron injection layer 150, a protective layer 155, a cathode 160, and a barrier layer 170. Cathode 160 is a compound cathode having a first conductive layer 162 and a second conductive layer 164. Device 100 may be fabricated by depositing the layers described, in order. The properties and functions of these various layers, as well as example materials, are described in more detail in U.S. Pat. No. 7,279,704 at cols. 6-10, which are incorporated by reference.

More examples for each of these layers are available. For example, a flexible and transparent substrate-anode combination is disclosed in U.S. Pat. No. 5,844,363, which is incorporated by reference in its entirety. An example of a p-doped hole transport layer is m-MTDATA doped with F₄-TCNQ at a molar ratio of 50:1, as disclosed in U.S. Patent Application Publication No. 2003/0230980, which is incorporated by reference in its entirety. Examples of emissive and host materials are disclosed in U.S. Pat. No. 6,303,238 to Thompson et al., which is incorporated by reference in its entirety. An example of an n-doped electron transport layer is BPhen doped with Li at a molar ratio of 1:1, as disclosed in U.S. Patent Application Publication No. 2003/0230980, which is incorporated by reference in its entirety. U.S. Pat. Nos. 5,703,436 and 5,707,745, which are incorporated by reference in their entireties, disclose examples of cathodes including compound cathodes having a thin layer of metal such as Mg:Ag with an overlying transparent, electrically-conductive, sputter-deposited ITO layer. The theory and use of blocking layers is described in more detail in U.S. Pat. No. 6,097,147 and U.S. Patent Application Publication No. 2003/0230980, which are incorporated by reference in their entireties. Examples of injection layers are provided in U.S. Patent Application Publication No. 2004/0174116, which is incorporated by reference in its entirety. A description of protective layers may be found in U.S. Patent Application Publication No. 2004/0174116, which is incorporated by reference in its entirety.

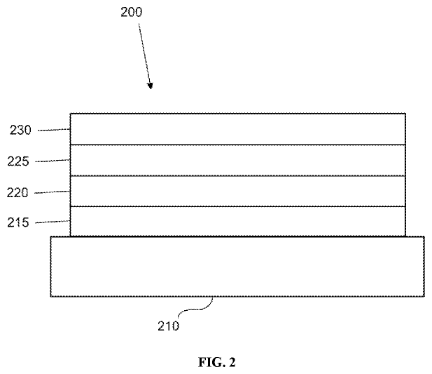

FIG. 2 shows an inverted OLED 200. The device includes a substrate 210, a cathode 215, an emissive layer 220, a hole transport layer 225, and an anode 230. Device 200 may be fabricated by depositing the layers described, in order. Because the most common OLED configuration has a cathode disposed over the anode, and device 200 has cathode 215 disposed under anode 230, device 200 may be referred to as an “inverted” OLED. Materials similar to those described with respect to device 100 may be used in the corresponding layers of device 200. FIG. 2 provides one example of how some layers may be omitted from the structure of device 100.

The simple layered structure illustrated in FIGS. 1 and 2 is provided by way of non-limiting example, and it is understood that embodiments of the present disclosure may be used in connection with a wide variety of other structures. The specific materials and structures described are exemplary in nature, and other materials and structures may be used. Functional OLEDs may be achieved by combining the various layers described in different ways, or layers may be omitted entirely, based on design, performance, and cost factors. Other layers not specifically described may also be included. Materials other than those specifically described may be used. Although many of the examples provided herein describe various layers as comprising a single material, it is understood that combinations of materials, such as a mixture of host and dopant, or more generally a mixture, may be used. Also, the layers may have various sublayers. The names given to the various layers herein are not intended to be strictly limiting. For example, in device 200, hole transport layer 225 transports holes and injects holes into emissive layer 220, and may be described as a hole transport layer or a hole injection layer. In one embodiment, an OLED may be described as having an “organic layer” disposed between a cathode and an anode. This organic layer may comprise a single layer, or may further comprise multiple layers of different organic materials as described, for example, with respect to FIGS. 1 and 2.

Structures and materials not specifically described may also be used, such as OLEDs comprised of polymeric materials (PLEDs) such as disclosed in U.S. Pat. No. 5,247,190 to Friend et al., which is incorporated by reference in its entirety. By way of further example, OLEDs having a single organic layer may be used. OLEDs may be stacked, for example as described in U.S. Pat. No. 5,707,745 to Forrest et al, which is incorporated by reference in its entirety. The OLED structure may deviate from the simple layered structure illustrated in FIGS. 1 and 2. For example, the substrate may include an angled reflective surface to improve outcoupling, such as a mesa structure as described in U.S. Pat. No. 6,091,195 to Forrest et al., and/or a pit structure as described in U.S. Pat. No. 5,834,893 to Bulovic et al., which are incorporated by reference in their entireties.

Unless otherwise specified, any of the layers of the various embodiments may be deposited by any suitable method. For the organic layers, preferred methods include thermal evaporation, ink-jet, such as described in U.S. Pat. Nos. 6,013,982 and 6,087,196, which are incorporated by reference in their entireties, organic vapor phase deposition (OVPD, also referred to as organic vapor jet deposition (OVJD)), such as described in U.S. Pat. No. 6,337,102 to Forrest et al., which is incorporated by reference in its entirety, and deposition by organic vapor jet printing (OVJP), such as described in U.S. Pat. No. 7,431,968, which is incorporated by reference in its entirety. Other suitable deposition methods include spin coating and other solution based processes. Solution based processes are preferably carried out in nitrogen or an inert atmosphere. For the other layers, preferred methods include thermal evaporation. Preferred patterning methods include deposition through a mask, cold welding such as described in U.S. Pat. Nos. 6,294,398 and 6,468,819, which are incorporated by reference in their entireties, and patterning associated with some of the deposition methods such as ink-jet and organic vapor jet printing (OVJP, also referred to as organic vapor jet deposition (OVJD)). Other methods may also be used. The materials to be deposited may be modified to make them compatible with a particular deposition method. For example, substituents such as alkyl and aryl groups, branched or unbranched, and preferably containing at least 3 carbons, may be used in small molecules to enhance their ability to undergo solution processing. Substituents having 20 carbons or more may be used, and 3-20 carbons are a preferred range. Materials with asymmetric structures may have better solution processability than those having symmetric structures, because asymmetric materials may have a lower tendency to recrystallize. Dendrimer substituents may be used to enhance the ability of small molecules to undergo solution processing.

Devices fabricated in accordance with embodiments of the present disclosure may further optionally comprise a barrier layer. One purpose of the barrier layer is to protect the electrodes and organic layers from damaging exposure to harmful species in the environment including moisture, vapor and/or gases, etc. The barrier layer may be deposited over, under or next to a substrate, an electrode, or over any other parts of a device including an edge. The barrier layer may comprise a single layer, or multiple layers. The barrier layer may be formed by various known chemical vapor deposition techniques and may include compositions having a single phase as well as compositions having multiple phases. Any suitable material or combination of materials may be used for the barrier layer. The barrier layer may incorporate an inorganic or an organic compound or both. The preferred barrier layer comprises a mixture of a polymeric material and a non-polymeric material as described in U.S. Pat. No. 7,968,146, PCT Pat. Application Nos. PCT/US2007/023098 and PCT/US2009/042829, which are herein incorporated by reference in their entireties. To be considered a “mixture”, the aforesaid polymeric and non-polymeric materials comprising the barrier layer should be deposited under the same reaction conditions and/or at the same time. The weight ratio of polymeric to non-polymeric material may be in the range of 95:5 to 5:95. The polymeric material and the non-polymeric material may be created from the same precursor material. In one example, the mixture of a polymeric material and a non-polymeric material consists essentially of polymeric silicon and inorganic silicon.

Devices fabricated in accordance with embodiments of the present disclosure can be incorporated into a wide variety of electronic component modules (or units) that can be incorporated into a variety of electronic products or intermediate components. Examples of such electronic products or intermediate components include display screens, lighting devices such as discrete light source devices or lighting panels, etc. that can be utilized by the end-user product manufacturers. Such electronic component modules can optionally include the driving electronics and/or power source(s). Devices fabricated in accordance with embodiments of the present disclosure can be incorporated into a wide variety of consumer products that have one or more of the electronic component modules (or units) incorporated therein. A consumer product comprising an OLED that includes the compound of the present disclosure in the organic layer in the OLED is disclosed. Such consumer products would include any kind of products that include one or more light source(s) and/or one or more of some type of visual displays. Some examples of such consumer products include flat panel displays, curved displays, computer monitors, medical monitors, televisions, billboards, lights for interior or exterior illumination and/or signaling, heads-up displays, fully or partially transparent displays, flexible displays, rollable displays, foldable displays, stretchable displays, laser printers, telephones, mobile phones, tablets, phablets, personal digital assistants (PDAs), wearable devices, laptop computers, digital cameras, camcorders, viewfinders, micro-displays (displays that are less than 2 inches diagonal), 3-D displays, virtual reality or augmented reality displays, vehicles, video walls comprising multiple displays tiled together, theater or stadium screen, a light therapy device, and a sign. Various control mechanisms may be used to control devices fabricated in accordance with the present disclosure, including passive matrix and active matrix. Many of the devices are intended for use in a temperature range comfortable to humans, such as 18 degrees C. to 30 degrees C., and more preferably at room temperature (20-25° C.), but could be used outside this temperature range, for example, from −40 degree C. to +80° C.

More details on OLEDs, and the definitions described above, can be found in U.S. Pat. No. 7,279,704, which is incorporated herein by reference in its entirety.

The materials and structures described herein may have applications in devices other than OLEDs. For example, other optoelectronic devices such as organic solar cells and organic photodetectors may employ the materials and structures. More generally, organic devices, such as organic transistors, may employ the materials and structures.

In some embodiments, the OLED has one or more characteristics selected from the group consisting of being flexible, being rollable, being foldable, being stretchable, and being curved. In some embodiments, the OLED is transparent or semi-transparent. In some embodiments, the OLED further comprises a layer comprising carbon nanotubes.

In some embodiments, the OLED further comprises a layer comprising a delayed fluorescent material. In some embodiments, the OLED comprises a RGB pixel arrangement or white plus color filter pixel arrangement. In some embodiments, the OLED is a mobile device, a hand held device, or a wearable device. In some embodiments, the OLED is a display panel having less than 10 inch diagonal or 50 square inch area. In some embodiments, the OLED is a display panel having at least 10 inch diagonal or 50 square inch area. In some embodiments, the OLED is a lighting panel.

In some embodiments, the compound can be an emissive dopant. In some embodiments, the compound can produce emissions via phosphorescence, fluorescence, thermally activated delayed fluorescence, i.e., TADF (also referred to as E-type delayed fluorescence; see, e.g., U.S. application Ser. No. 15/700,352, which is hereby incorporated by reference in its entirety), triplet-triplet annihilation, or combinations of these processes. In some embodiments, the emissive dopant can be a racemic mixture, or can be enriched in one enantiomer. In some embodiments, the compound can be homoleptic (each ligand is the same). In some embodiments, the compound can be heteroleptic (at least one ligand is different from others). When there are more than one ligand coordinated to a metal, the ligands can all be the same in some embodiments. In some other embodiments, at least one ligand is different from the other ligands. In some embodiments, every ligand can be different from each other. This is also true in embodiments where a ligand being coordinated to a metal can be linked with other ligands being coordinated to that metal to form a tridentate, tetradentate, pentadentate, or hexadentate ligands. Thus, where the coordinating ligands are being linked together, all of the ligands can be the same in some embodiments, and at least one of the ligands being linked can be different from the other ligand(s) in some other embodiments.

In some embodiments, the compound can be used as a phosphorescent sensitizer in an OLED where one or multiple layers in the OLED contains an acceptor in the form of one or more fluorescent and/or delayed fluorescence emitters. In some embodiments, the compound can be used as one component of an exciplex to be used as a sensitizer. As a phosphorescent sensitizer, the compound must be capable of energy transfer to the acceptor and the acceptor will emit the energy or further transfer energy to a final emitter. The acceptor concentrations can range from 0.001% to 100%. The acceptor could be in either the same layer as the phosphorescent sensitizer or in one or more different layers. In some embodiments, the acceptor is a TADF emitter. In some embodiments, the acceptor is a fluorescent emitter. In some embodiments, the emission can arise from any or all of the sensitizer, acceptor, and final emitter.

According to another aspect, a formulation comprising the compound described herein is also disclosed.

The OLED disclosed herein can be incorporated into one or more of a consumer product, an electronic component module, and a lighting panel. The organic layer can be an emissive layer and the compound can be an emissive dopant in some embodiments, while the compound can be a non-emissive dopant in other embodiments.

In yet another aspect of the present disclosure, a formulation that comprises the novel compound disclosed herein is described. The formulation can include one or more components selected from the group consisting of a solvent, a host, a hole injection material, hole transport material, electron blocking material, hole blocking material, and an electron transport material, disclosed herein.

The present disclosure encompasses any chemical structure comprising the novel compound of the present disclosure, or a monovalent or polyvalent variant thereof. In other words, the inventive compound, or a monovalent or polyvalent variant thereof, can be a part of a larger chemical structure. Such chemical structure can be selected from the group consisting of a monomer, a polymer, a macromolecule, and a supramolecule (also known as supermolecule). As used herein, a “monovalent variant of a compound” refers to a moiety that is identical to the compound except that one hydrogen has been removed and replaced with a bond to the rest of the chemical structure. As used herein, a “polyvalent variant of a compound” refers to a moiety that is identical to the compound except that more than one hydrogen has been removed and replaced with a bond or bonds to the rest of the chemical structure. In the instance of a supramolecule, the inventive compound can also be incorporated into the supramolecule complex without covalent bonds.

### D. Combination of the Compounds of the Present Disclosure with Other Materials

The materials described herein as useful for a particular layer in an organic light emitting device may be used in combination with a wide variety of other materials present in the device. For example, emissive dopants disclosed herein may be used in conjunction with a wide variety of hosts, transport layers, blocking layers, injection layers, electrodes and other layers that may be present. The materials described or referred to below are non-limiting examples of materials that may be useful in combination with the compounds disclosed herein, and one of skill in the art can readily consult the literature to identify other materials that may be useful in combination.

a) Conductivity Dopants:

A charge transport layer can be doped with conductivity dopants to substantially alter its density of charge carriers, which will in turn alter its conductivity. The conductivity is increased by generating charge carriers in the matrix material, and depending on the type of dopant, a change in the Fermi level of the semiconductor may also be achieved. Hole-transporting layer can be doped by p-type conductivity dopants and n-type conductivity dopants are used in the electron-transporting layer.

Non-limiting examples of the conductivity dopants that may be used in an OLED in combination with materials disclosed herein are exemplified below together with references that disclose those materials: EP01617493, EP01968131, EP2020694, EP2684932, US20050139810, US20070160905, US20090167167, US2010288362, WO06081780, WO2009003455, WO2009008277, WO2009011327, WO2014009310, US2007252140, US2015060804, US20150123047, and US2012146012.

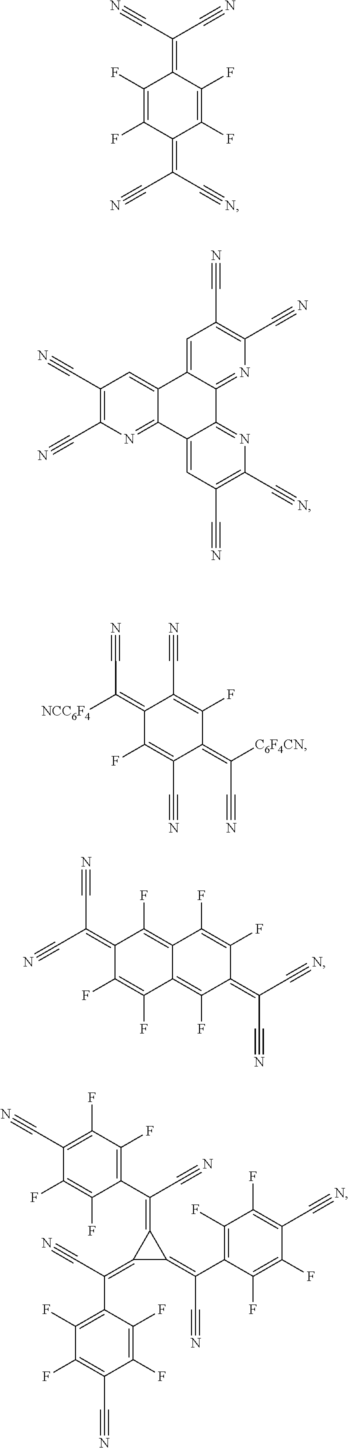

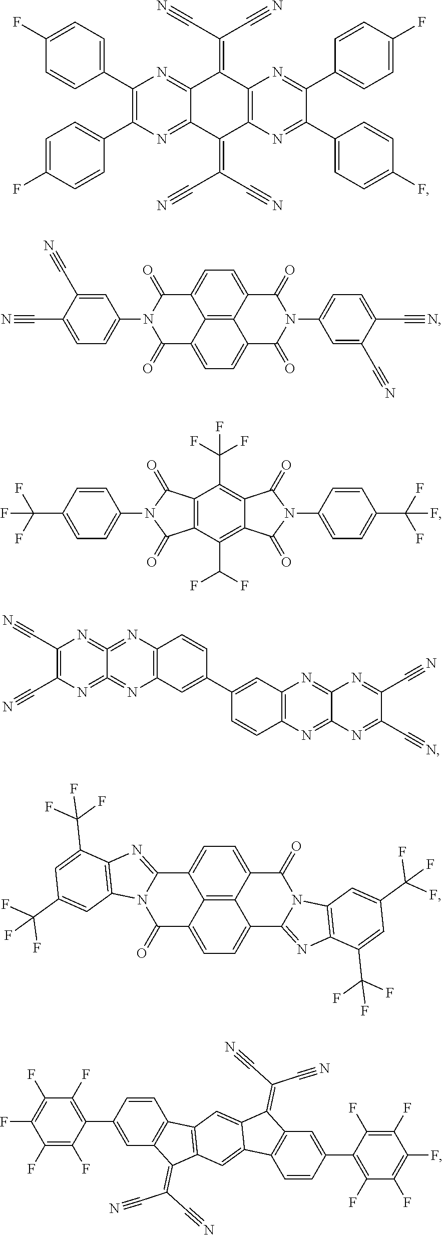

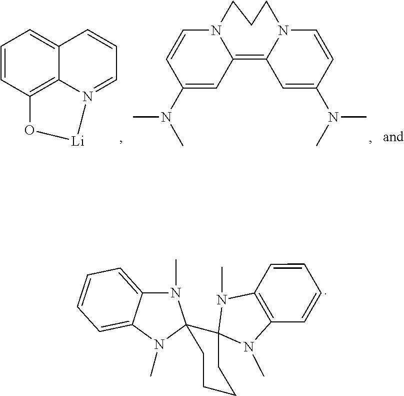

b) HIL/HTL:

A hole injecting/transporting material to be used in the present disclosure is not particularly limited, and any compound may be used as long as the compound is typically used as a hole injecting/transporting material. Examples of the material include, but are not limited to: a phthalocyanine or porphyrin derivative; an aromatic amine derivative; an indolocarbazole derivative; a polymer containing fluorohydrocarbon; a polymer with conductivity dopants; a conducting polymer, such as PEDOT/PSS; a self-assembly monomer derived from compounds such as phosphonic acid and silane derivatives; a metal oxide derivative, such as MoOₓ; a p-type semiconducting organic compound, such as 1,4,5,8,9,12-Hexaazatriphenylenehexacarbonitrile; a metal complex, and a cross-linkable compounds.

Examples of aromatic amine derivatives used in HIL or HTL include, but not limit to the following general structures:

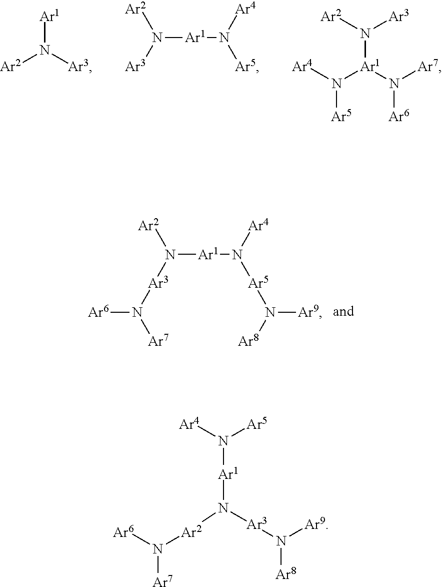

Each of Ar¹ to Ar⁹ is selected from the group consisting of aromatic hydrocarbon cyclic compounds such as benzene, biphenyl, triphenyl, triphenylene, naphthalene, anthracene, phenalene, phenanthrene, fluorene, pyrene, chrysene, perylene, and azulene; the group consisting of aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and the group consisting of 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Each Ar may be unsubstituted or may be substituted by a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acids, ether, ester, nitrile, isonitrile, sulfanyl, sulfinyl, sulfonyl, phosphino, and combinations thereof.

In one aspect, Ar¹ to Ar⁹ is independently selected from the group consisting of:

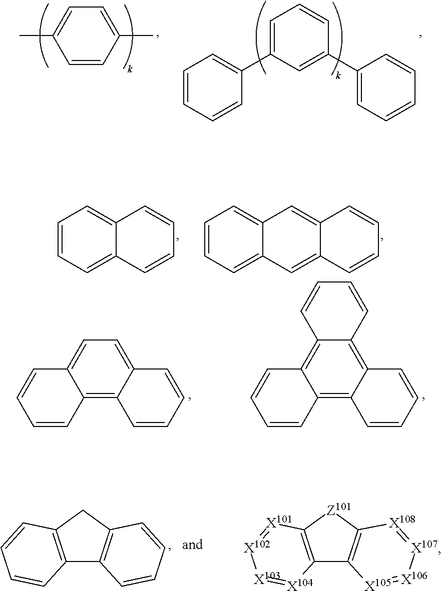

wherein k is an integer from 1 to 20; X¹⁰¹ to X¹⁰⁸ is C (including CH) or N; Z¹⁰¹ is NAr¹, O, or S; Ar¹ has the same group defined above.

Examples of metal complexes used in HIL or HTL include, but are not limited to the following general formula:

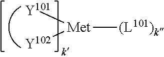

wherein Met is a metal, which can have an atomic weight greater than 40; (Y¹⁰¹-Y¹⁰²) is a bidentate ligand, Y¹⁰¹ and Y¹⁰² are independently selected from C, N, O, P, and S; L¹⁰¹ is an ancillary ligand; k′ is an integer value from 1 to the maximum number of ligands that may be attached to the metal; and k′+k″ is the maximum number of ligands that may be attached to the metal.

In one aspect, (Y¹⁰¹-Y¹⁰²) is a 2-phenylpyridine derivative. In another aspect, (Y¹⁰¹-Y¹⁰²) is a carbene ligand. In another aspect, Met is selected from Ir, Pt, Os, and Zn. In a further aspect, the metal complex has a smallest oxidation potential in solution vs. Fc⁺/Fc couple less than about 0.6 V.

Non-limiting examples of the HIL and HTL materials that may be used in an OLED in combination with materials disclosed herein are exemplified below together with references that disclose those materials: CN102702075, DE102012005215, EP01624500, EP01698613, EP01806334, EP01930964, EP01972613, EP01997799, EP02011790, EP02055700, EP02055701, EP1725079, EP2085382, EP2660300, EP650955, JP07-073529, JP2005112765, JP2007091719, JP2008021687, JP2014-009196, KR20110088898, KR20130077473, TW201139402, U.S. Ser. No. 06/517,957, US20020158242, US20030162053, US20050123751, US20060182993, US20060240279, US20070145888, US20070181874, US20070278938, US20080014464, US20080091025, US20080106190, US20080124572, US20080145707, US20080220265, US20080233434, US20080303417, US2008107919, US20090115320, US20090167161, US2009066235, US2011007385, US20110163302, US2011240968, US2011278551, US2012205642, US2013241401, US20140117329, US2014183517, U.S. Pat. Nos. 5,061,569, 5,639,914, WO05075451, WO07125714, WO08023550, WO08023759, WO2009145016, WO2010061824, WO2011075644, WO2012177006, WO2013018530, WO2013039073, WO2013087142, WO2013118812, WO2013120577, WO2013157367, WO2013175747, WO2014002873, WO2014015935, WO2014015937, WO2014030872, WO2014030921, WO2014034791, WO2014104514, WO2014157018.

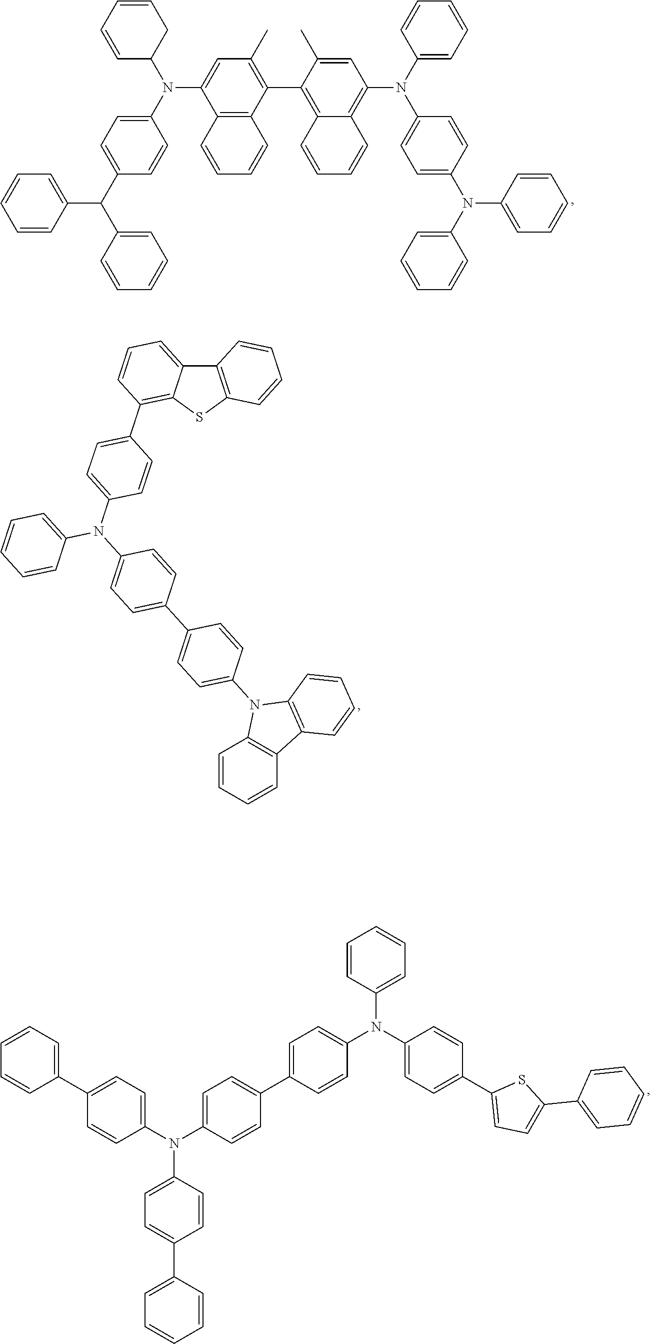 [SMILES: Cc1cc(N(c2ccccc2)c2ccc(N(c3ccccc3)c3ccccc3)cc2)c2ccccc2c1-c1c(C)cc(N(c2ccc(C(c3ccccc3)c3ccccc3)cc2)C2C=CC=CC2)c2ccccc12 ; c1ccc(N(c2ccc(-c3ccc(-n4c5ccccc5c5ccccc54)cc3)cc2)c2ccc(-c3cccc4c3sc3ccccc34)cc2)cc1 ; c1ccc(-c2ccc(N(c3ccc(-c4ccccc4)cc3)c3ccc(-c4ccc(N(c5ccccc5)c5ccc(-c6ccc(-c7ccccc7)s6)cc5)cc4)cc3)cc2)cc1]

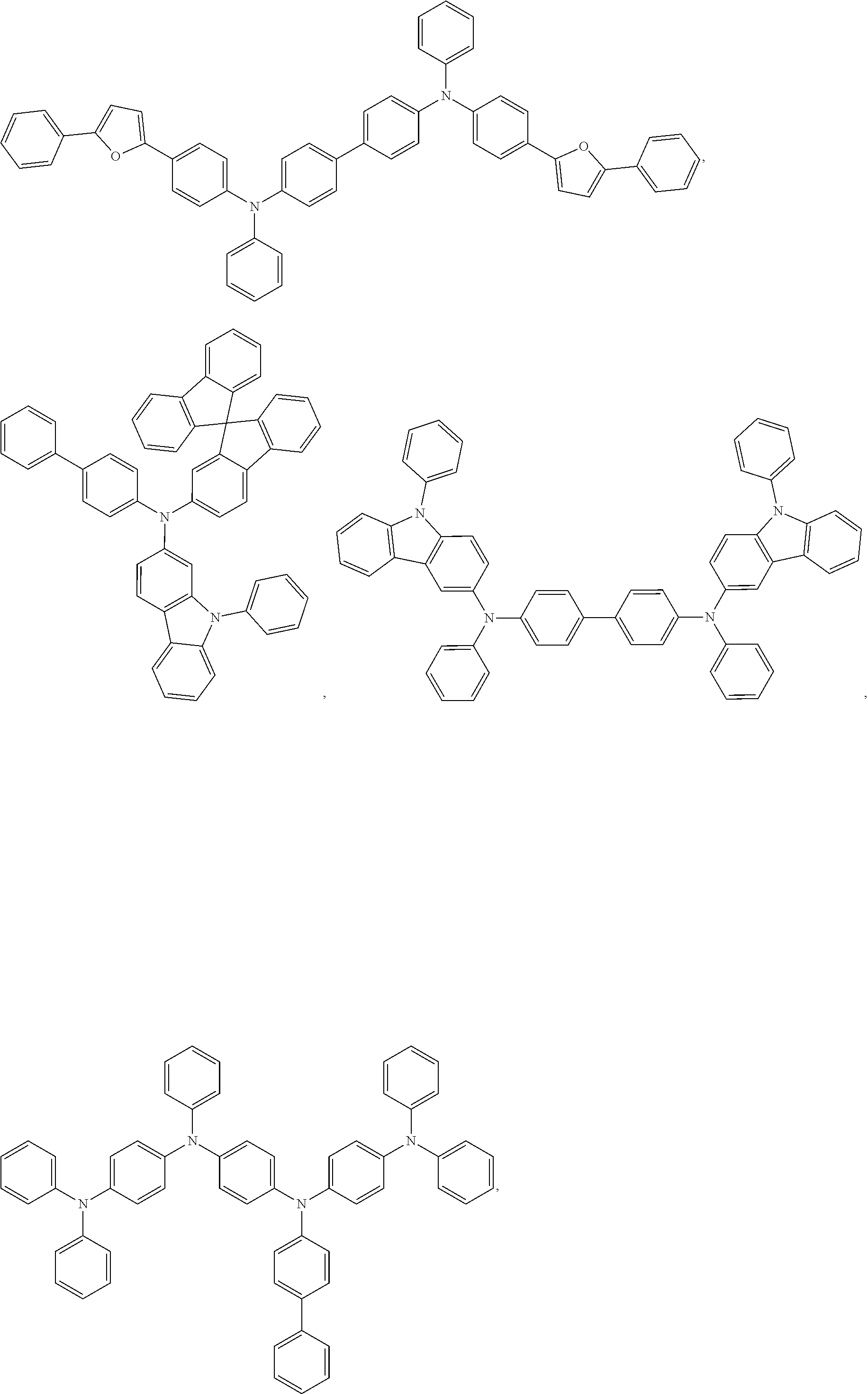

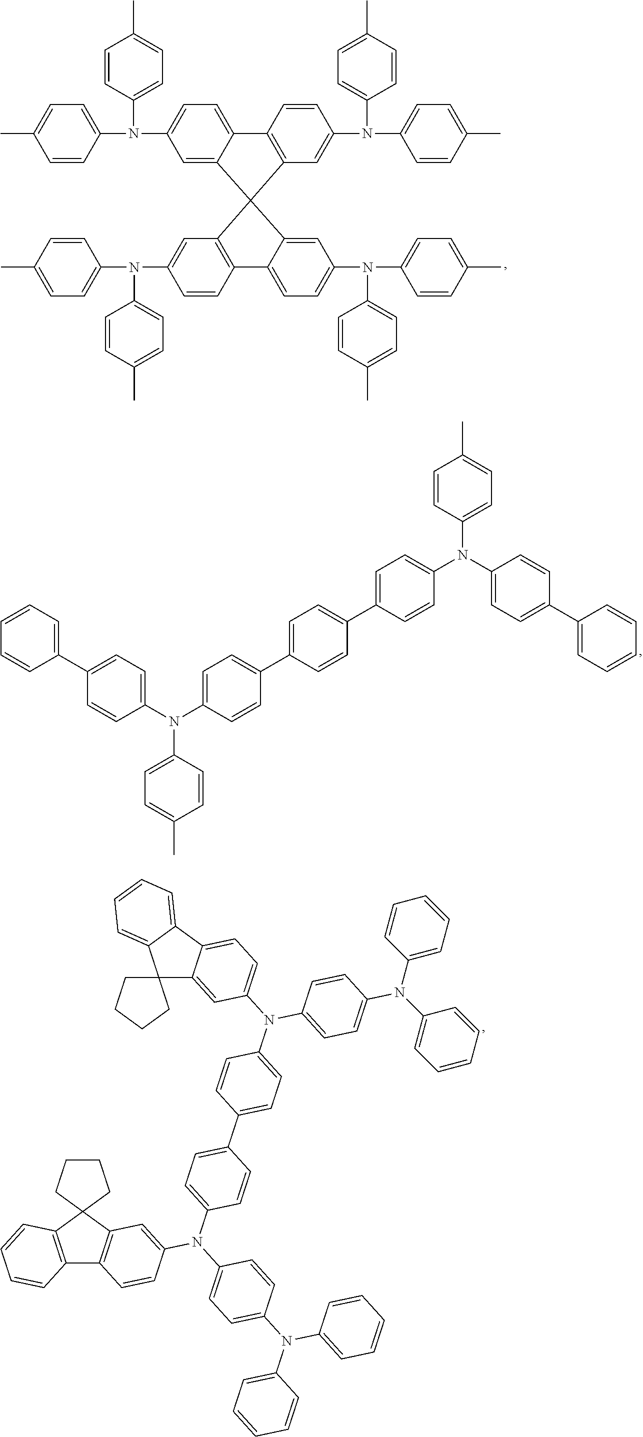

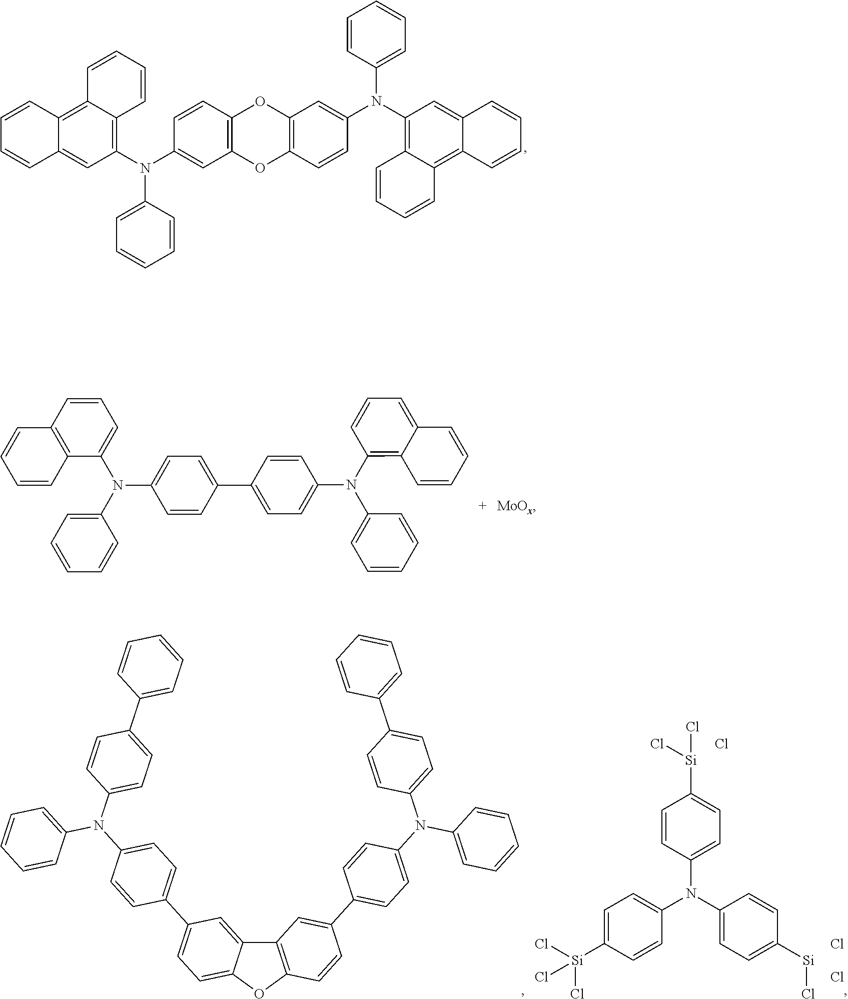

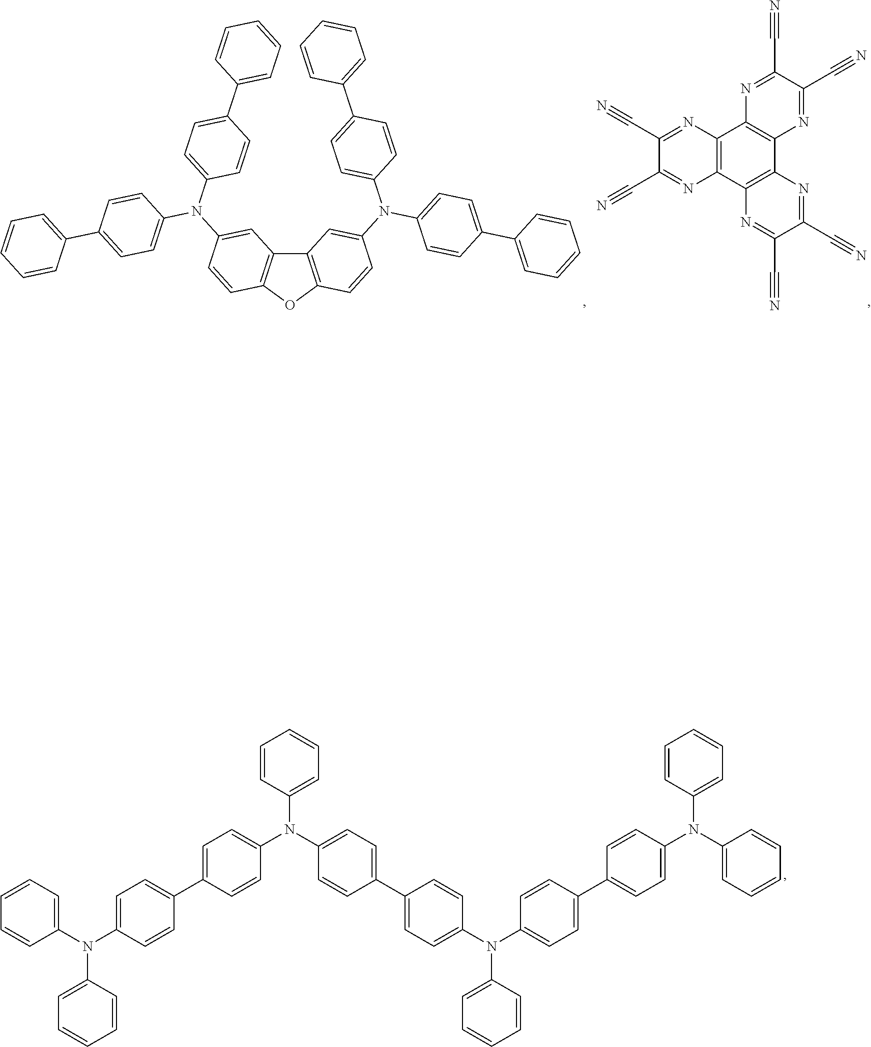

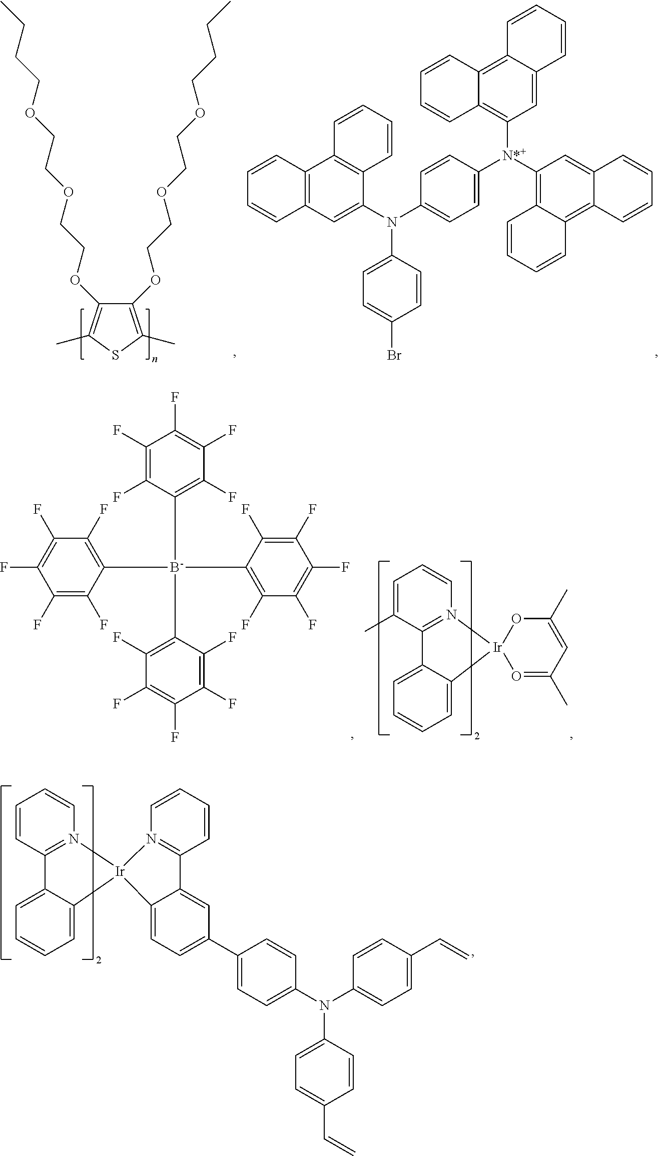

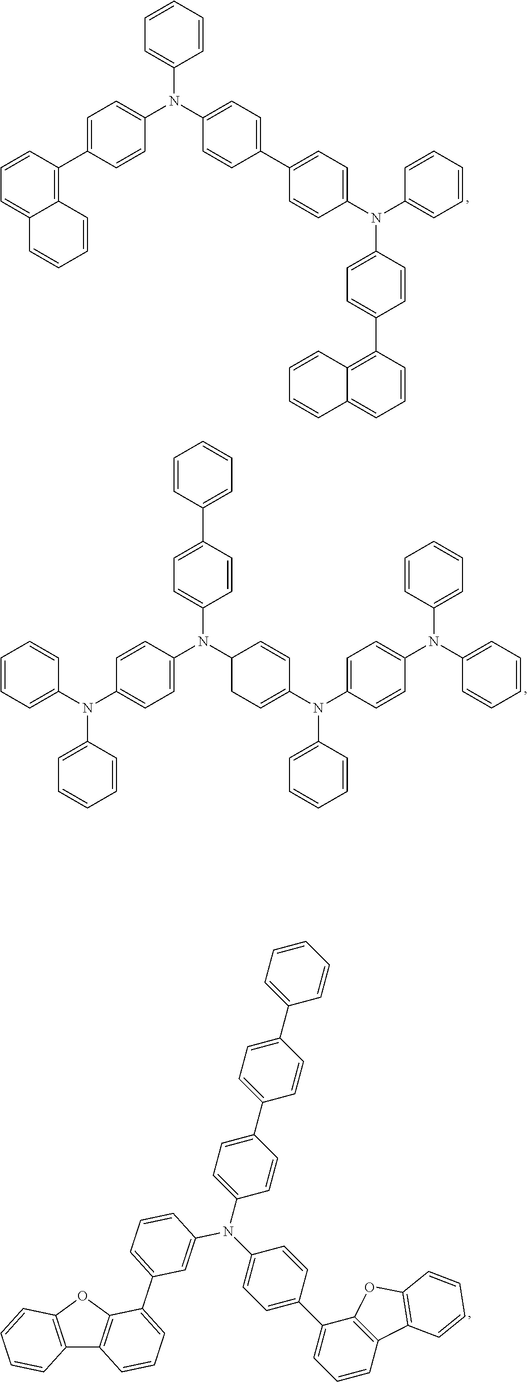 [SMILES: c1ccc(N(c2ccc(-c3ccc(N(c4ccccc4)c4ccc(-c5cccc6ccccc56)cc4)cc3)cc2)c2ccc(-c3cccc4ccccc34)cc2)cc1 ; C1=CC(N(c2ccc(-c3ccccc3)cc2)c2ccc(N(c3ccccc3)c3ccccc3)cc2)CC=C1N(c1ccccc1)c1ccc(N(c2ccccc2)c2ccccc2)cc1 ; c1ccc(-c2ccc(-c3ccc(N(c4ccc(-c5cccc6c5oc5ccccc56)cc4)c4cccc(-c5cccc6c5oc5ccccc56)c4)cc3)cc2)cc1]

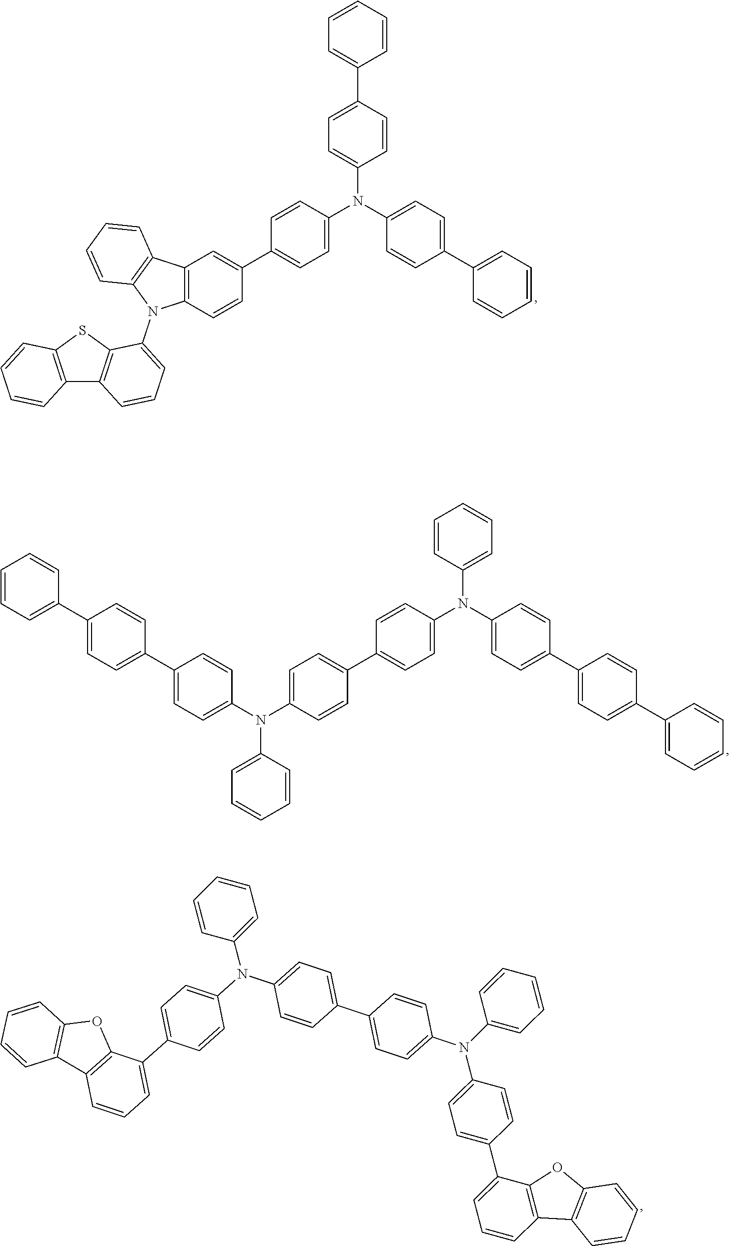 [SMILES: c1ccc(-c2ccc(N(c3ccc(-c4ccccc4)cc3)c3ccc(-c4ccc5c(c4)c4ccccc4n5-c4cccc5c4sc4ccccc45)cc3)cc2)cc1 ; c1ccc(-c2ccc(-c3ccc(N(c4ccccc4)c4ccc(-c5ccc(N(c6ccccc6)c6ccc(-c7ccc(-c8ccccc8)cc7)cc6)cc5)cc4)cc3)cc2)cc1 ; c1ccc(N(c2ccc(-c3ccc(N(c4ccccc4)c4ccc(-c5cccc6c5oc5ccccc56)cc4)cc3)cc2)c2ccc(-c3cccc4c3oc3ccccc34)cc2)cc1]

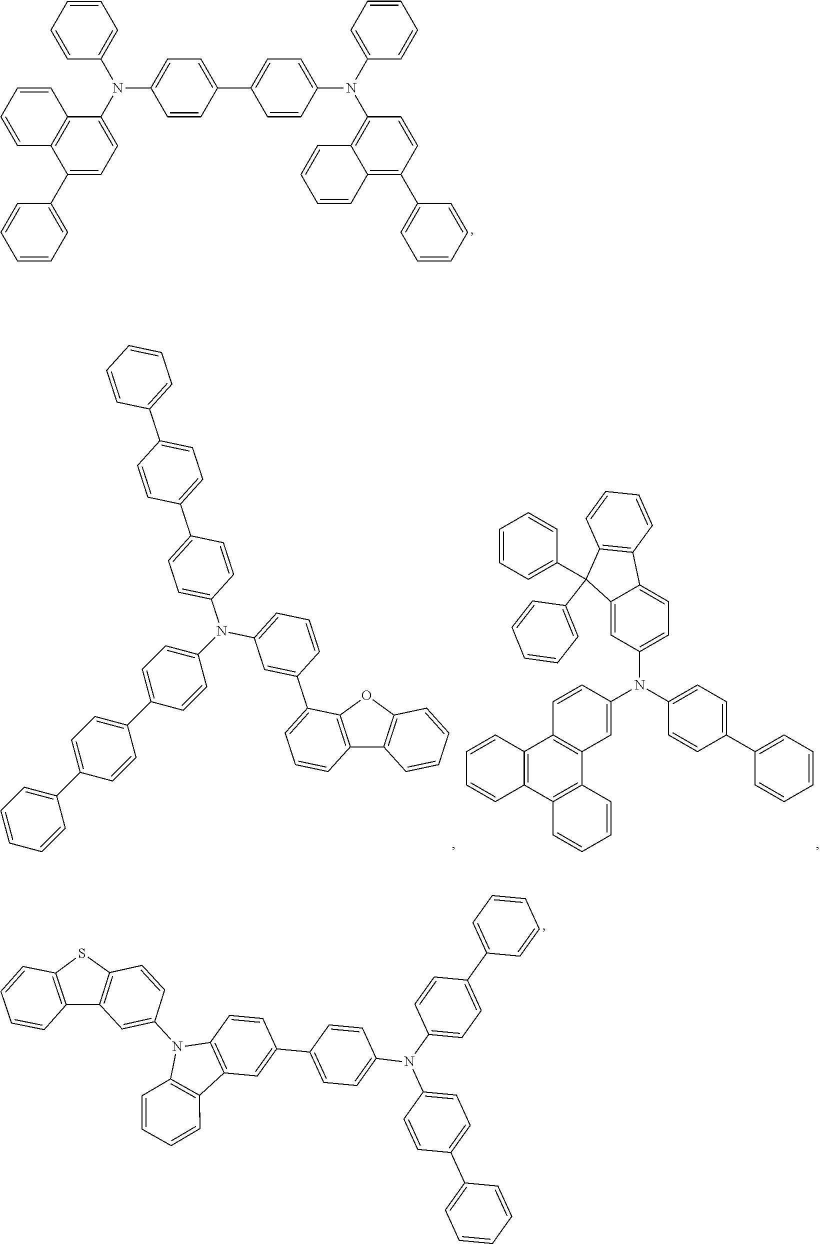

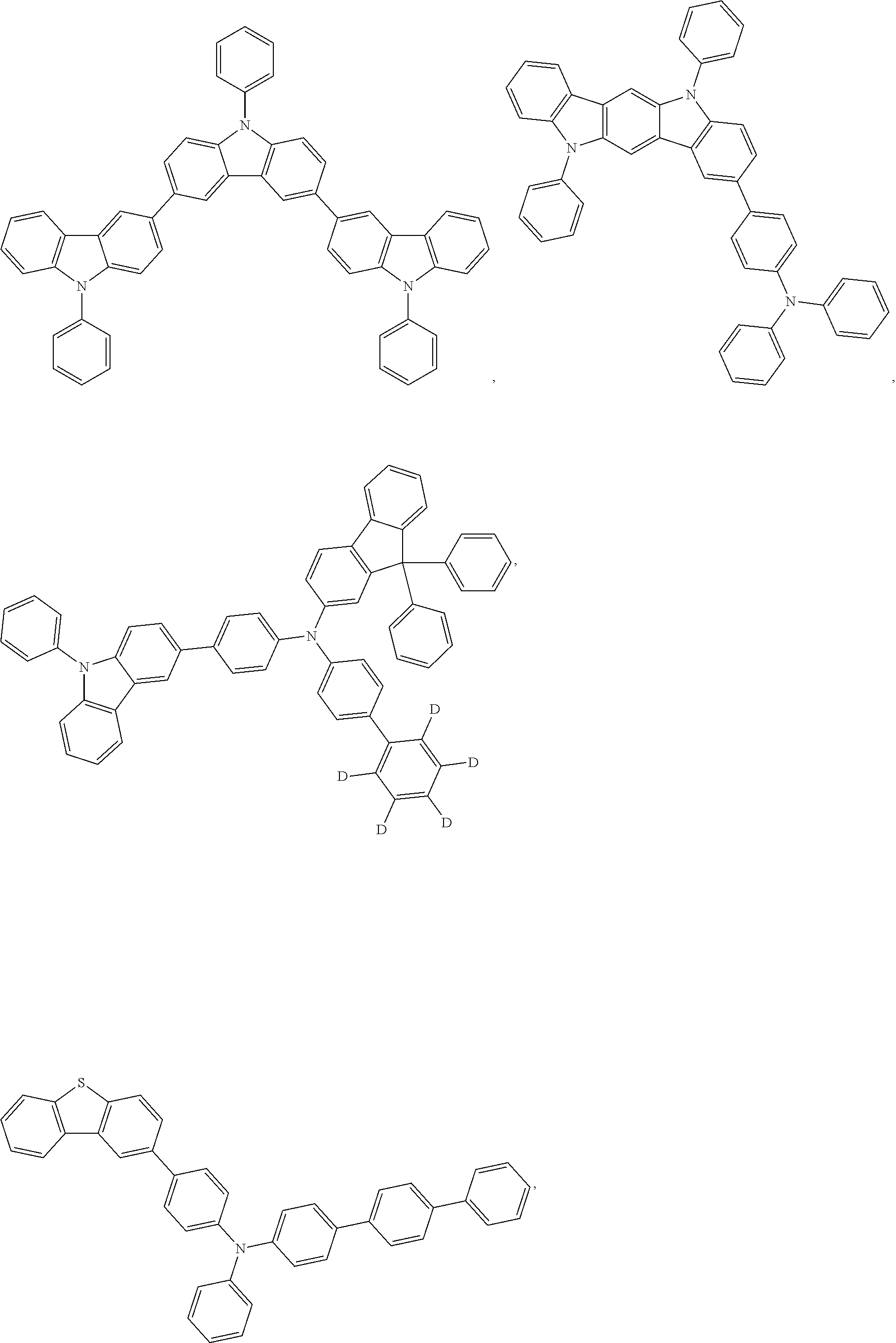

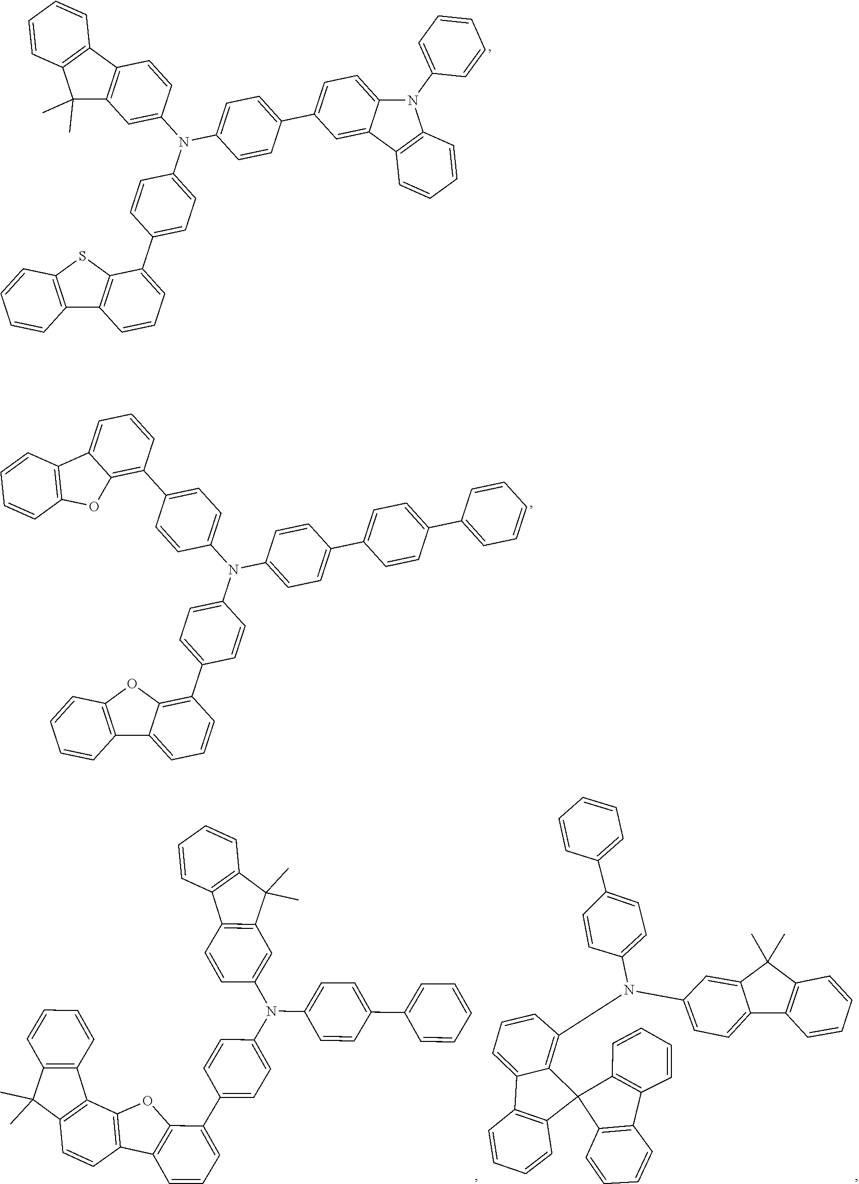

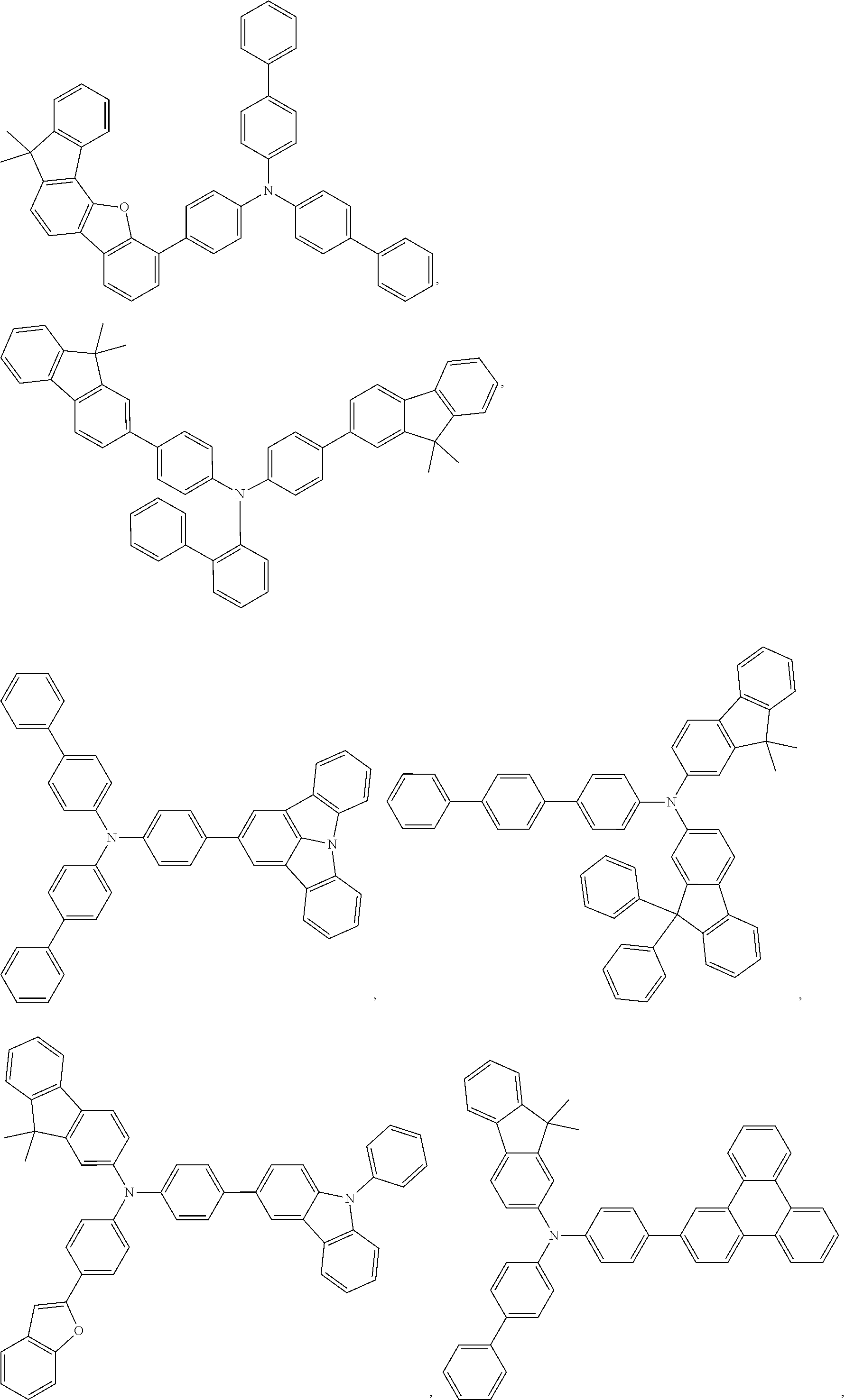

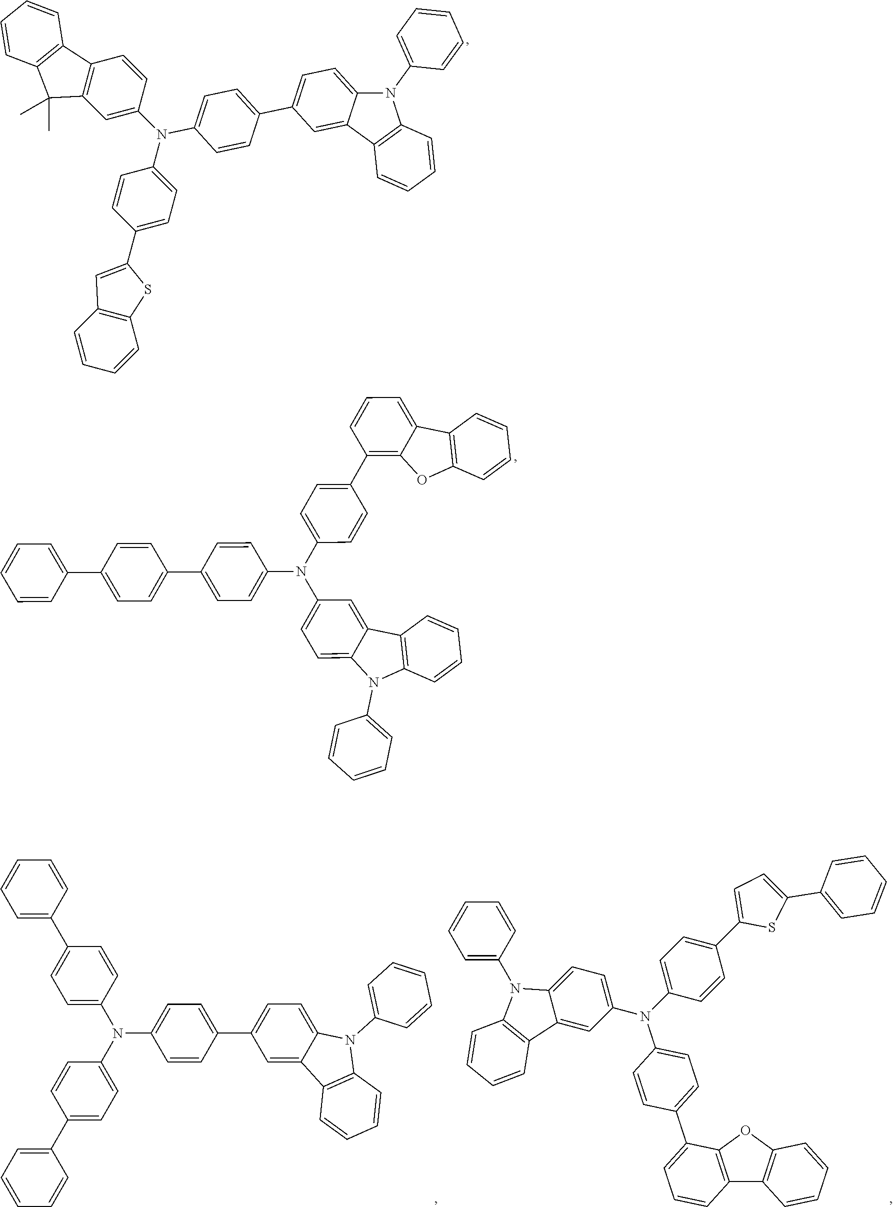

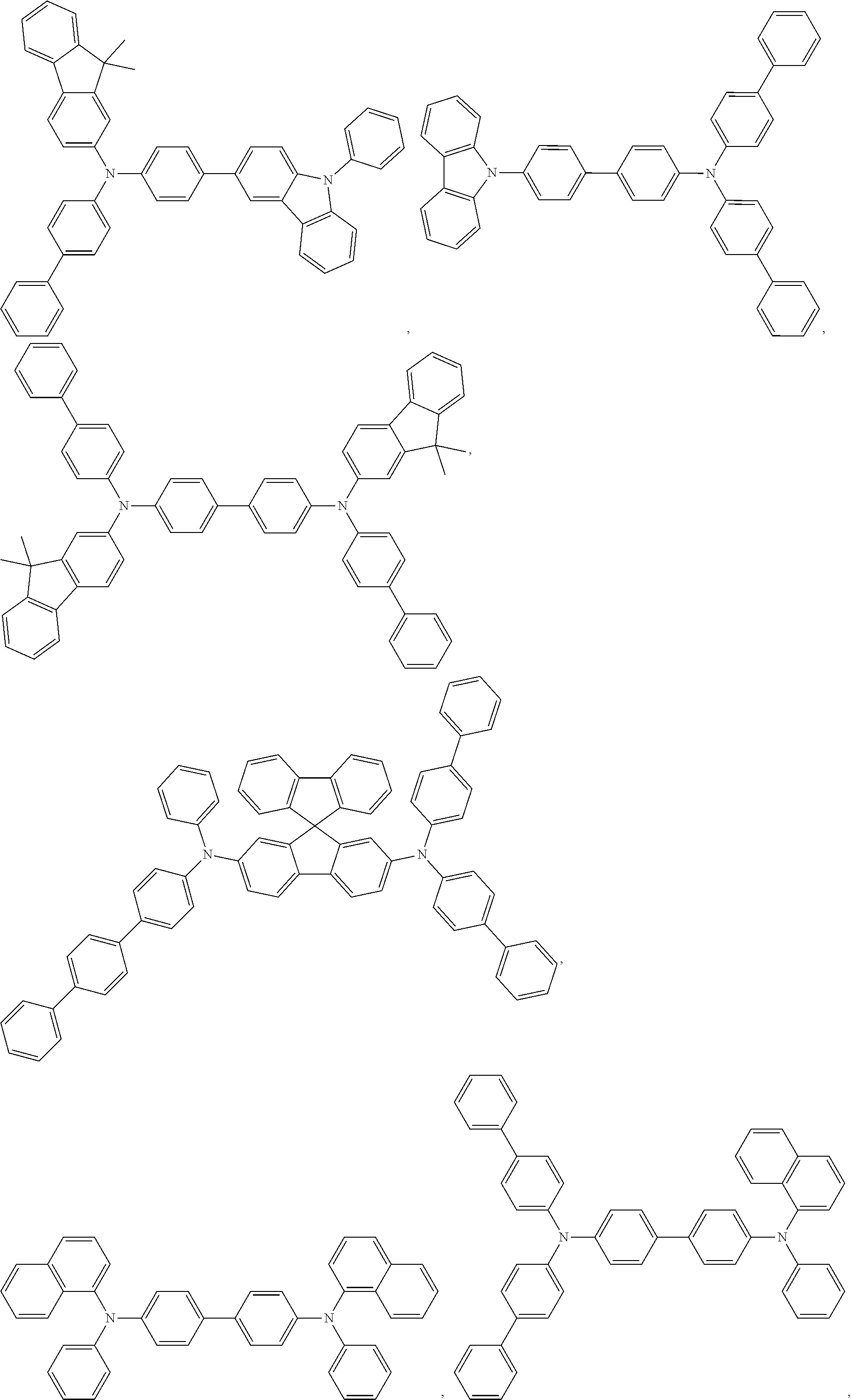

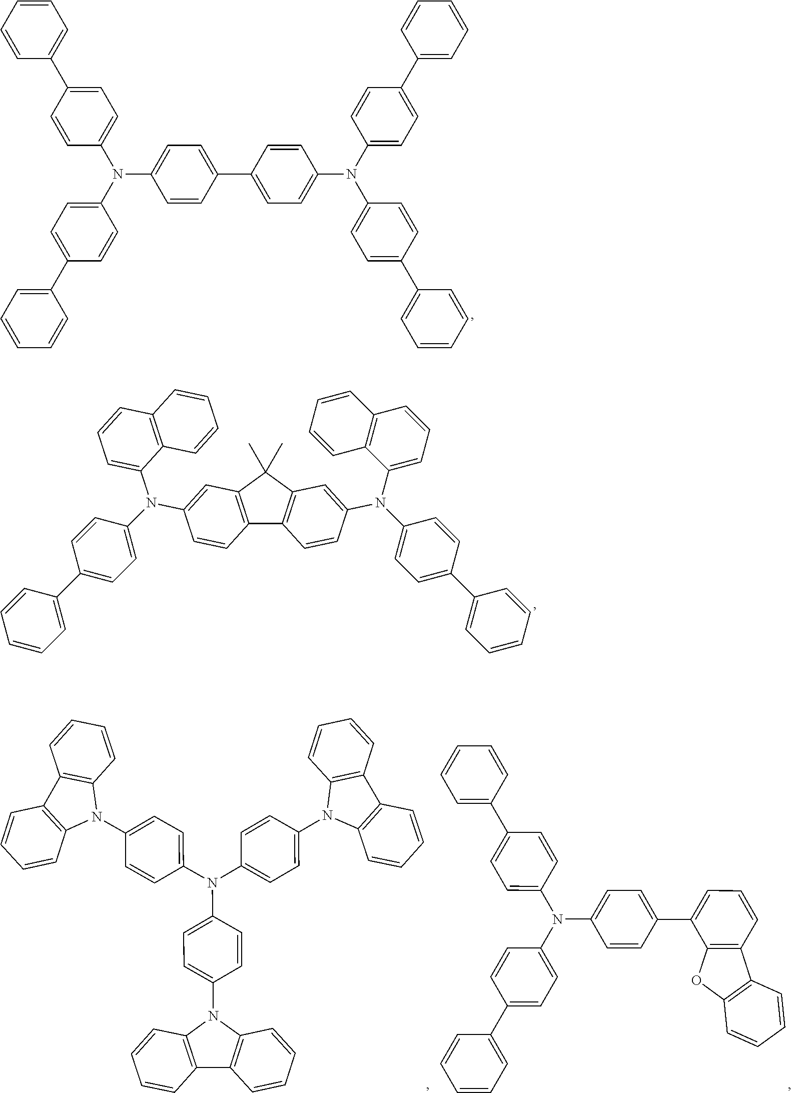

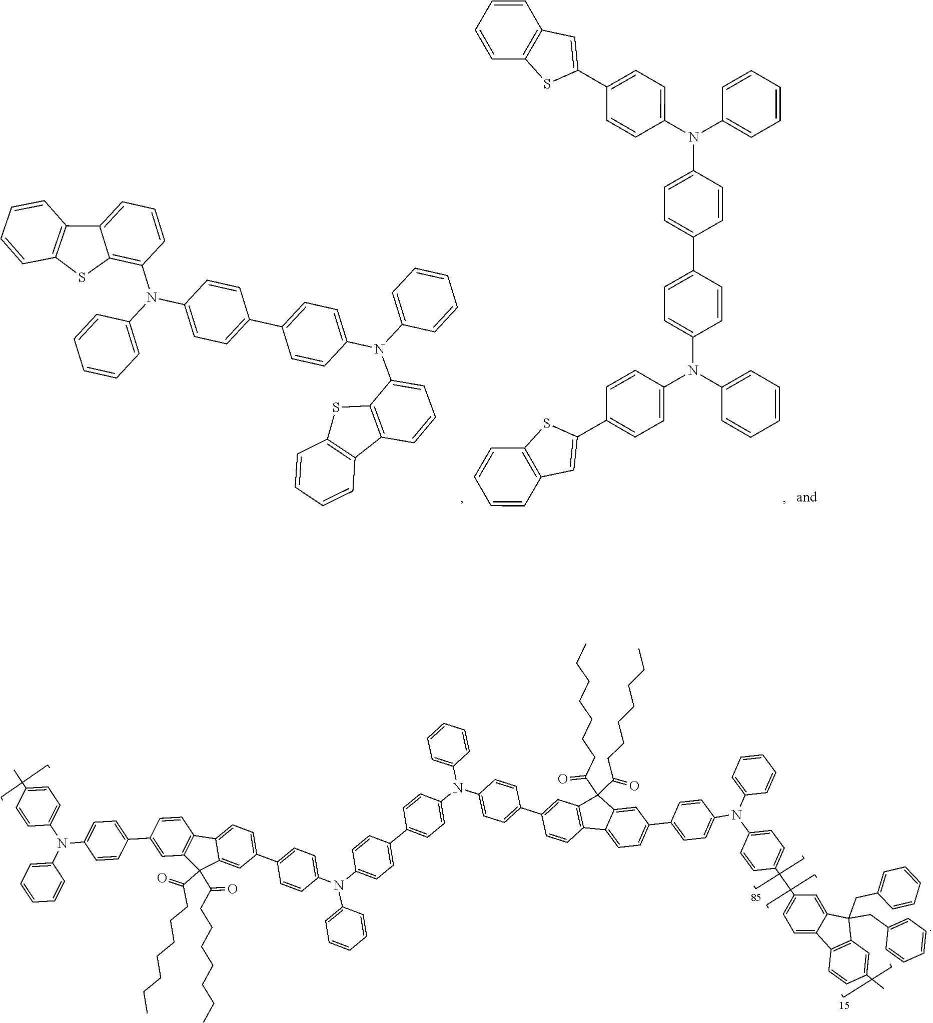

c) EBL:

An electron blocking layer (EBL) may be used to reduce the number of electrons and/or excitons that leave the emissive layer. The presence of such a blocking layer in a device may result in substantially higher efficiencies, and/or longer lifetime, as compared to a similar device lacking a blocking layer. Also, a blocking layer may be used to confine emission to a desired region of an OLED. In some embodiments, the EBL material has a higher LUMO (closer to the vacuum level) and/or higher triplet energy than the emitter closest to the EBL interface. In some embodiments, the EBL material has a higher LUMO (closer to the vacuum level) and/or higher triplet energy than one or more of the hosts closest to the EBL interface. In one aspect, the compound used in EBL contains the same molecule or the same functional groups used as one of the hosts described below.

d) Hosts:

The light emitting layer of the organic EL device of the present disclosure preferably contains at least a metal complex as light emitting material, and may contain a host material using the metal complex as a dopant material. Examples of the host material are not particularly limited, and any metal complexes or organic compounds may be used as long as the triplet energy of the host is larger than that of the dopant. Any host material may be used with any dopant so long as the triplet criteria is satisfied.

Examples of metal complexes used as host are preferred to have the following general formula:

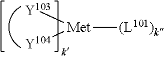

wherein Met is a metal; (Y¹⁰³-Y¹⁰⁴) is a bidentate ligand, Y¹⁰³ and Y¹⁰⁴ are independently selected from C, N, O, P, and S; L¹⁰¹ is an another ligand; k′ is an integer value from 1 to the maximum number of ligands that may be attached to the metal; and k′+k″ is the maximum number of ligands that may be attached to the metal.

In one aspect, the metal complexes are:

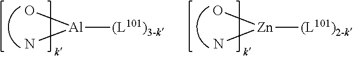

wherein (O—N) is a bidentate ligand, having metal coordinated to atoms O and N.

In another aspect, Met is selected from Ir and Pt. In a further aspect, (Y¹⁰³-Y¹⁰⁴) is a carbene ligand.

In one aspect, the host compound contains at least one of the following groups selected from the group consisting of aromatic hydrocarbon cyclic compounds such as benzene, biphenyl, triphenyl, triphenylene, tetraphenylene, naphthalene, anthracene, phenalene, phenanthrene, fluorene, pyrene, chrysene, perylene, and azulene; the group consisting of aromatic heterocyclic compounds such as dibenzothiophene, dibenzofuran, dibenzoselenophene, furan, thiophene, benzofuran, benzothiophene, benzoselenophene, carbazole, indolocarbazole, pyridylindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, phenoxazine, benzofuropyridine, furodipyridine, benzothienopyridine, thienodipyridine, benzoselenophenopyridine, and selenophenodipyridine; and the group consisting of 2 to 10 cyclic structural units which are groups of the same type or different types selected from the aromatic hydrocarbon cyclic group and the aromatic heterocyclic group and are bonded to each other directly or via at least one of oxygen atom, nitrogen atom, sulfur atom, silicon atom, phosphorus atom, boron atom, chain structural unit and the aliphatic cyclic group. Each option within each group may be unsubstituted or may be substituted by a substituent selected from the group consisting of deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acids, ether, ester, nitrile, isonitrile, sulfanyl, sulfinyl, sulfonyl, phosphino, and combinations thereof.

In one aspect, the host compound contains at least one of the following groups in the molecule:

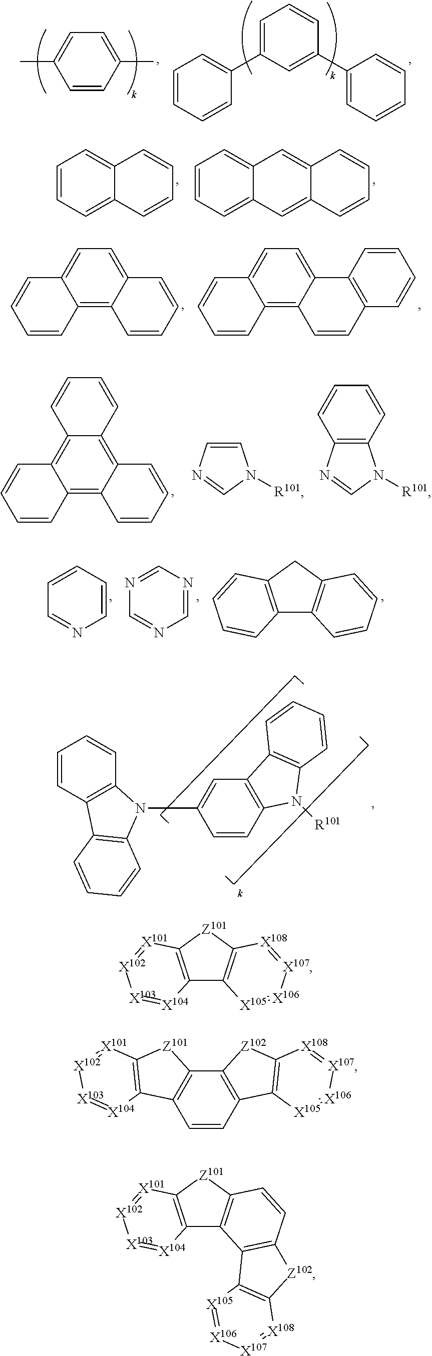

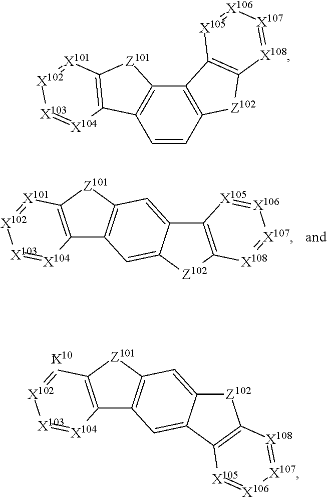

wherein R¹⁰¹ is selected from the group consisting of hydrogen, deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acids, ether, ester, nitrile, isonitrile, sulfanyl, sulfinyl, sulfonyl, phosphino, and combinations thereof, and when it is aryl or heteroaryl, it has the similar definition as Ar's mentioned above. k is an integer from 0 to 20 or 1 to 20. X¹⁰¹ to X¹⁰⁸ are independently selected from C (including CH) or N. Z¹⁰¹ and Z¹⁰² are independently selected from NR¹⁰¹, O, or S.

Non-limiting examples of the host materials that may be used in an OLED in combination with materials disclosed herein are exemplified below together with references that disclose those materials: EP2034538, EP2034538A, EP2757608, JP2007254297, KR20100079458, KR20120088644, KR20120129733, KR20130115564, TW201329200, US20030175553, US20050238919, US20060280965, US20090017330, US20090030202, US20090167162, US20090302743, US20090309488, US20100012931, US20100084966, US20100187984, US2010187984, US2012075273, US2012126221, US2013009543, US2013105787, US2013175519, US2014001446, US20140183503, US20140225088, US2014034914, U.S. Pat. No. 7,154,114, WO2001039234, WO2004093207, WO2005014551, WO2005089025, WO2006072002, WO2006114966, WO2007063754, WO2008056746, WO2009003898, WO2009021126, WO2009063833, WO2009066778, WO2009066779, WO2009086028, WO2010056066, WO2010107244, WO2011081423, WO2011081431, WO2011086863, WO2012128298, WO2012133644, WO2012133649, WO2013024872, WO2013035275, WO2013081315, WO2013191404, WO2014142472, US20170263869, US20160163995, U.S. Pat. No. 9,466,803,

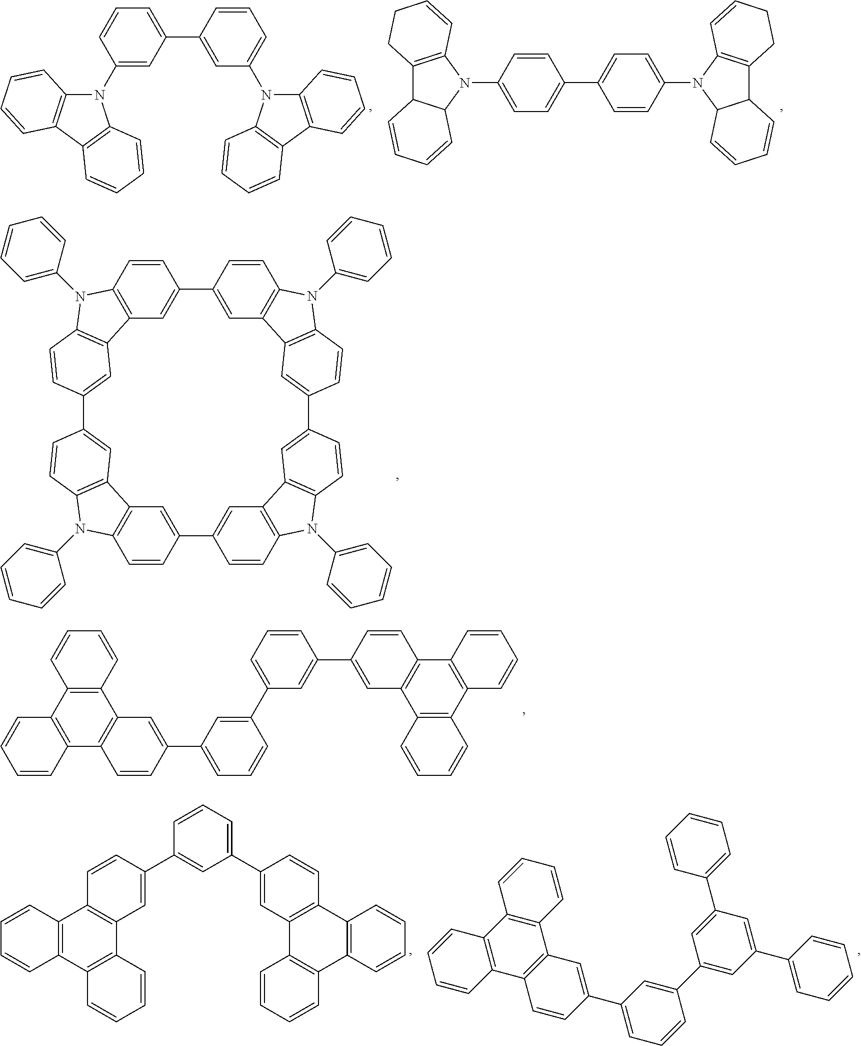

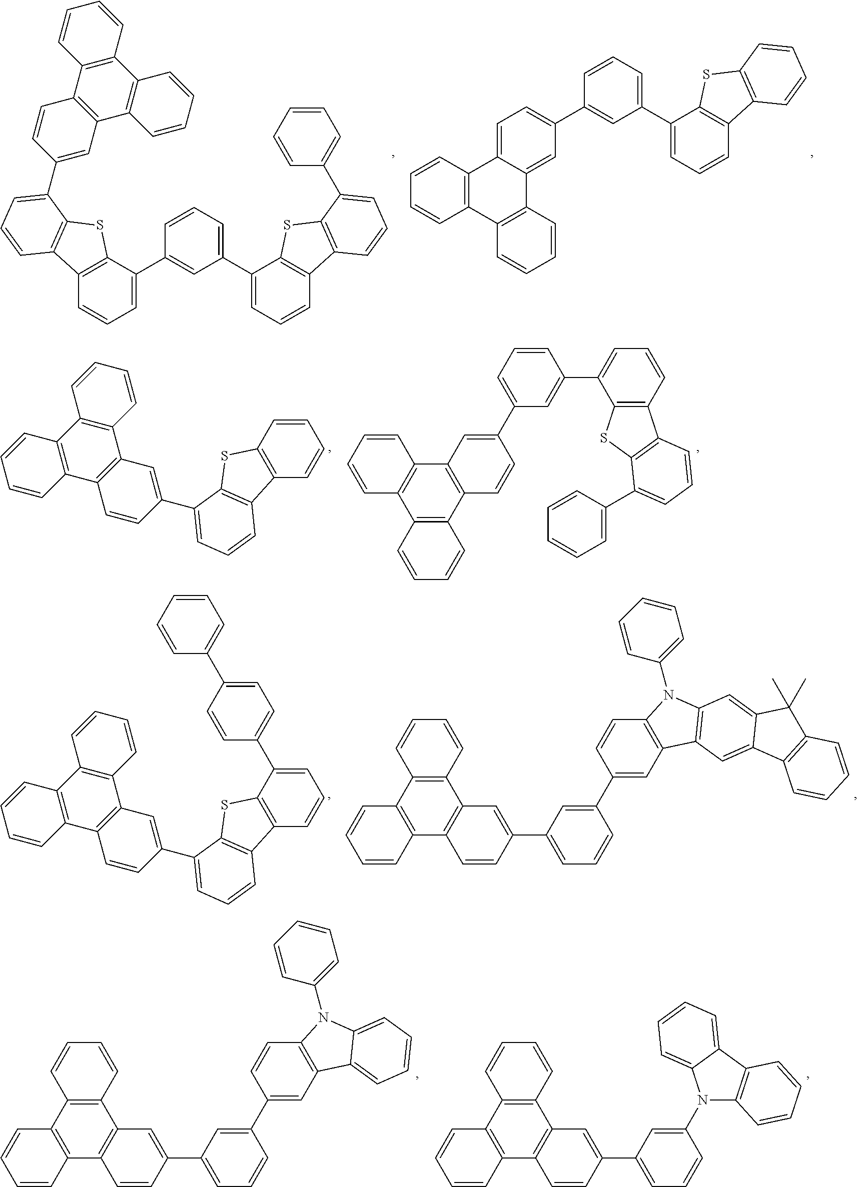

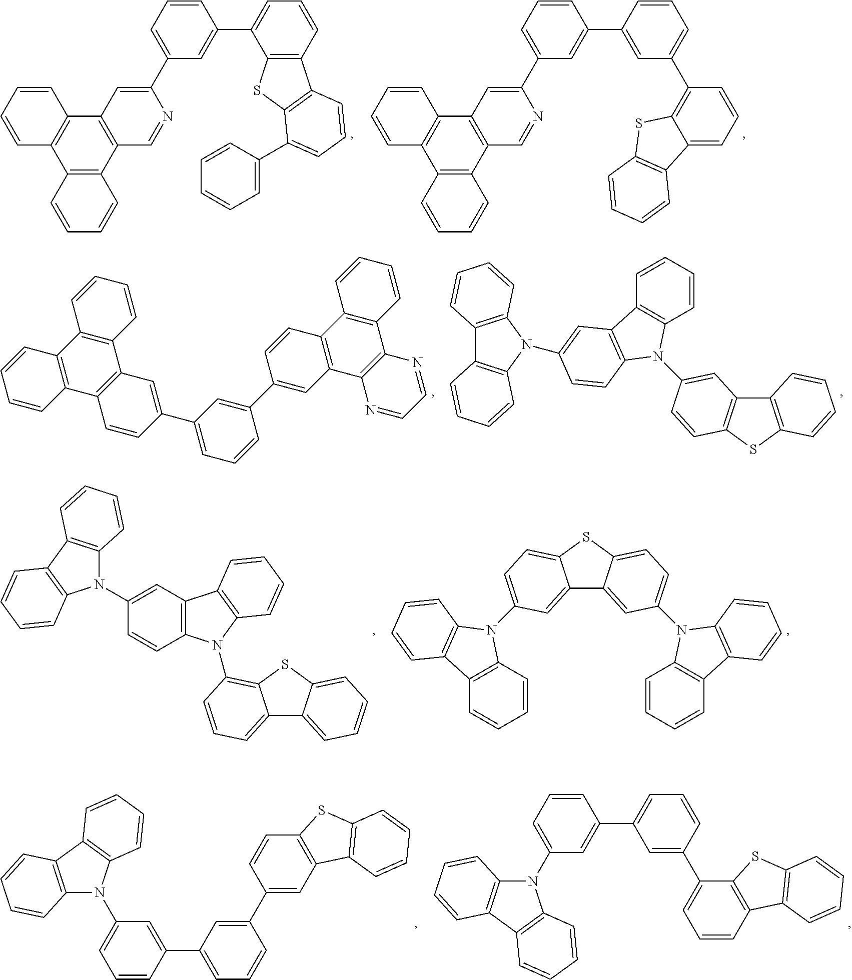

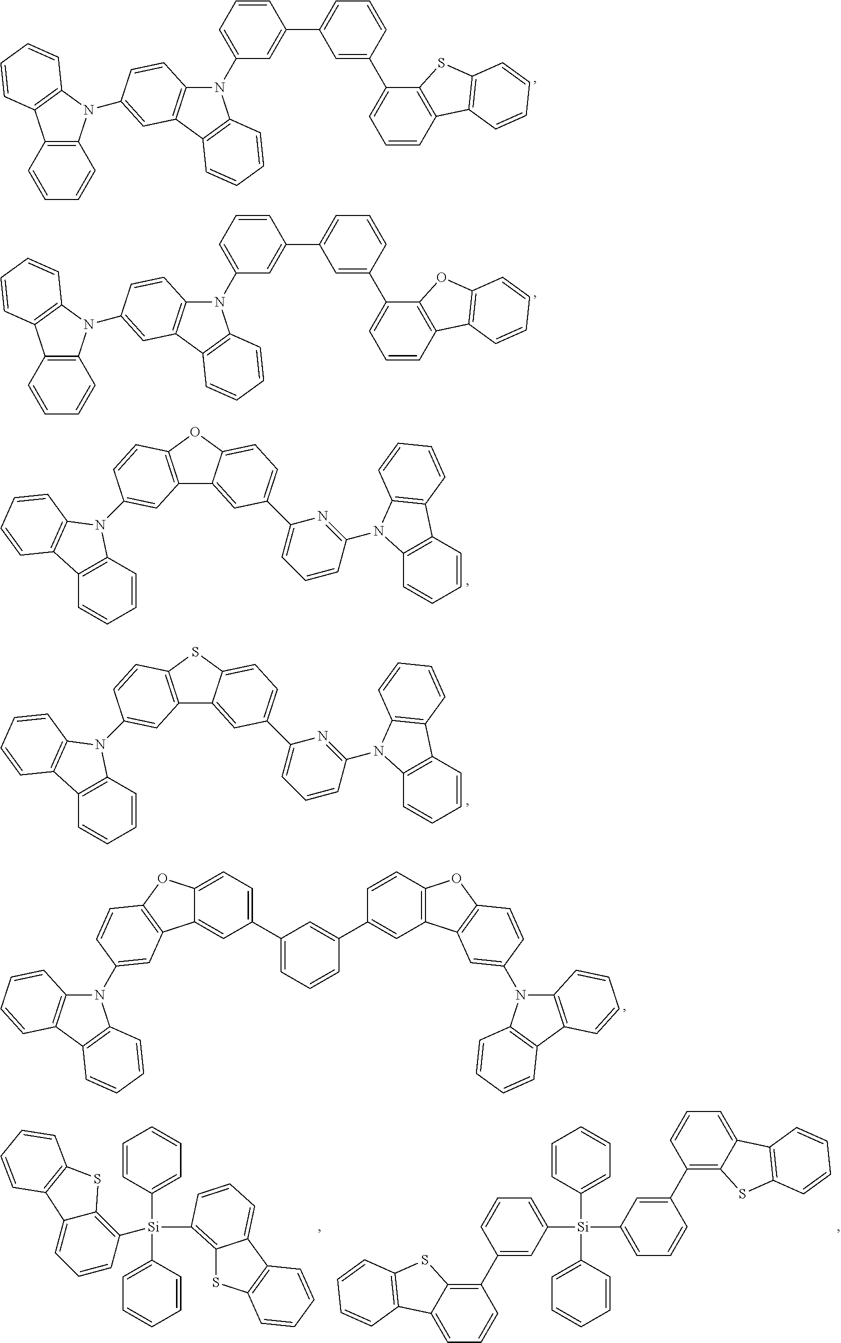

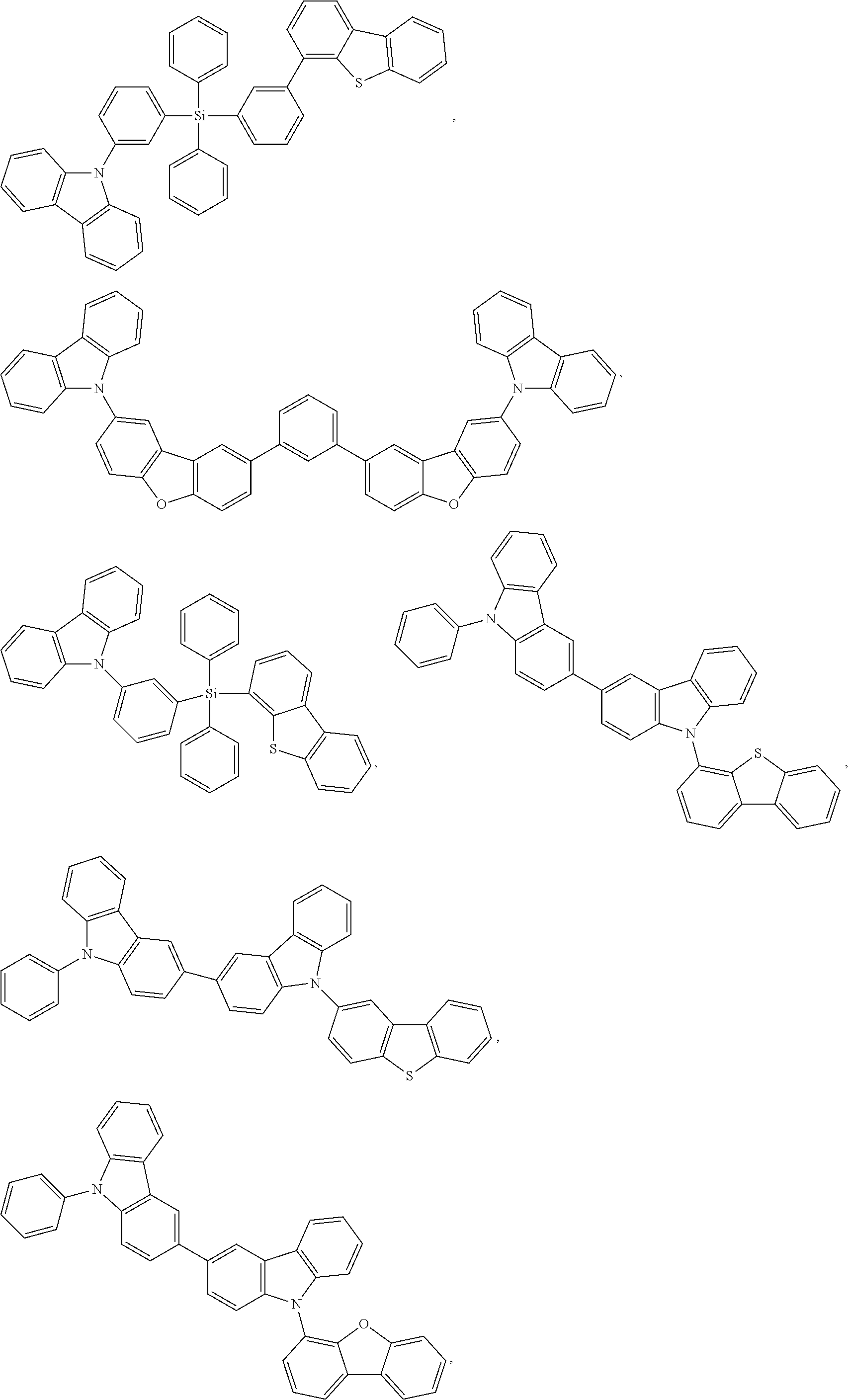

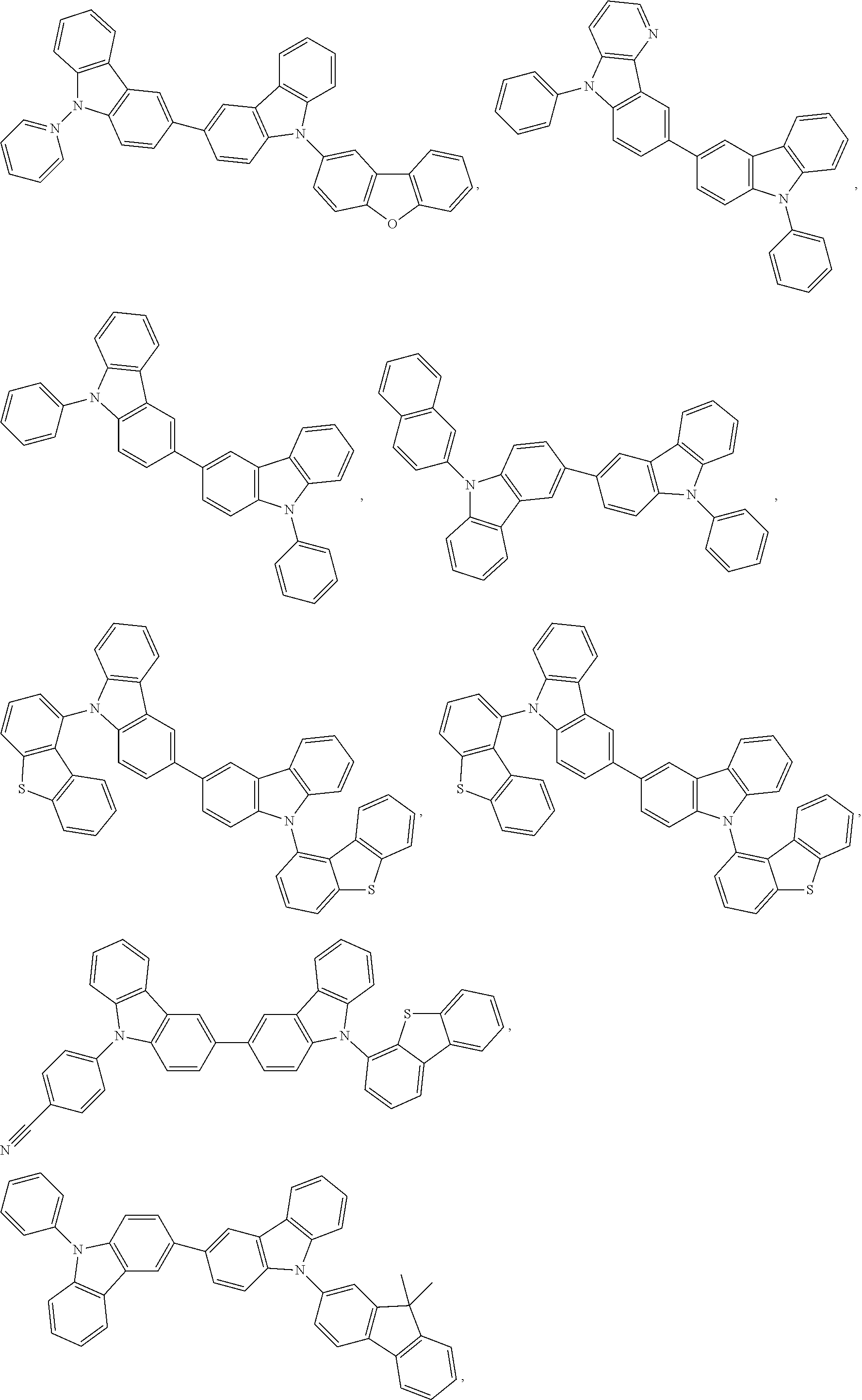

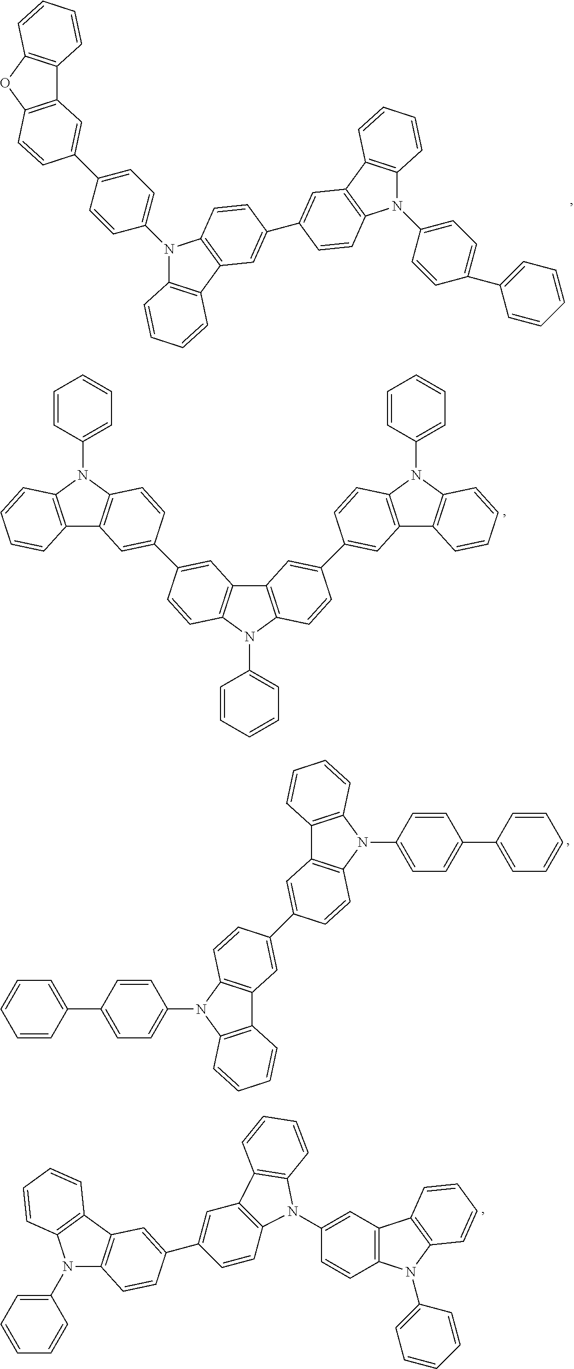

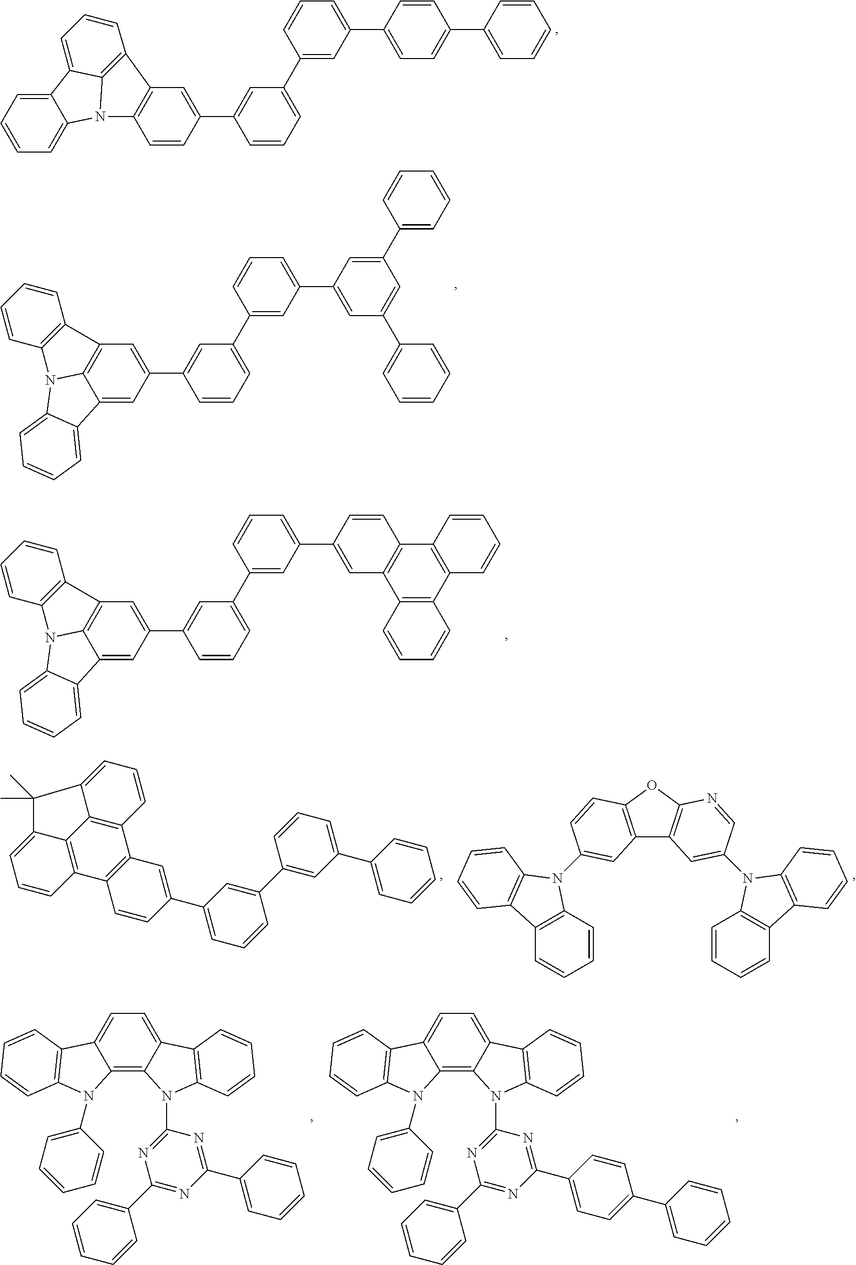

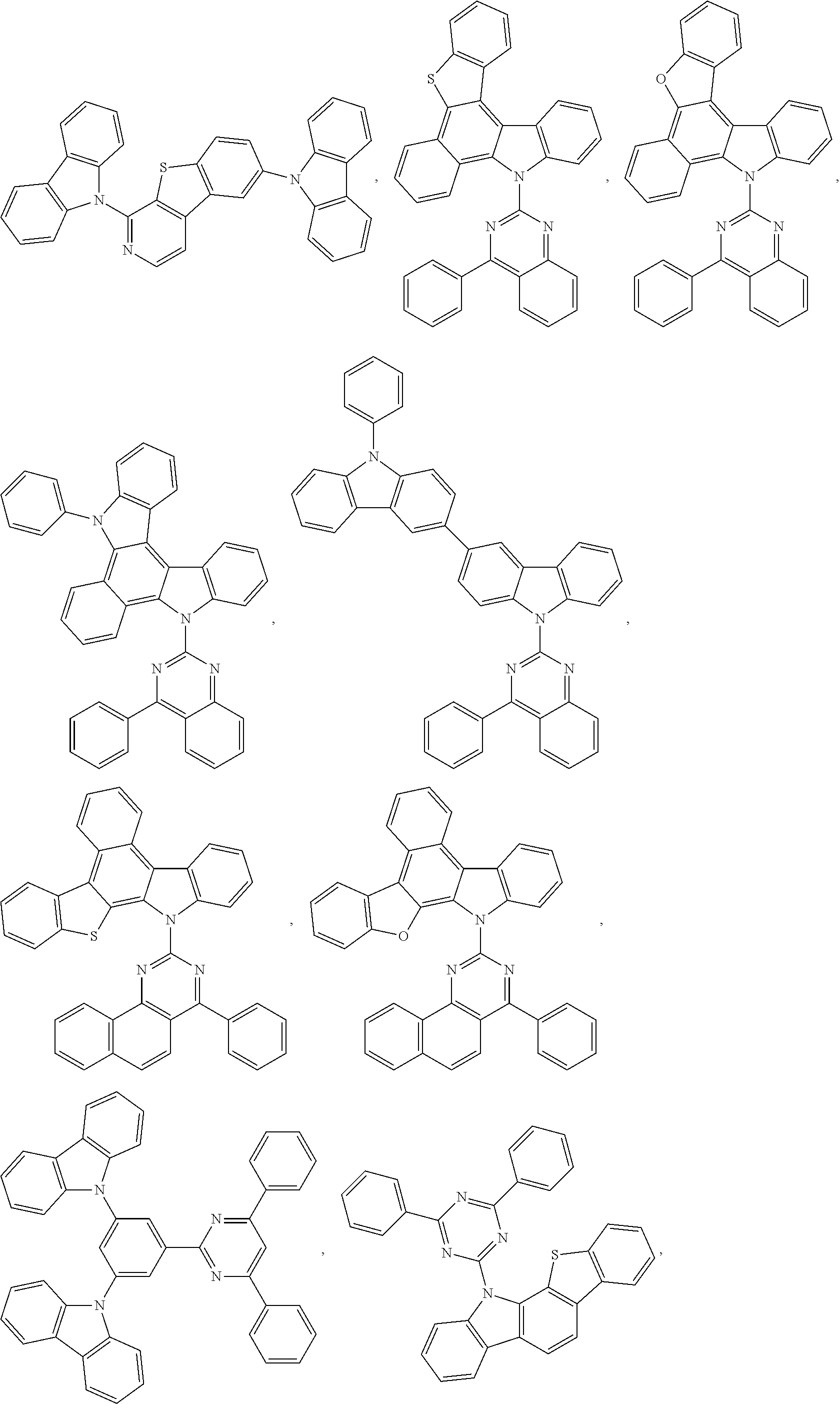

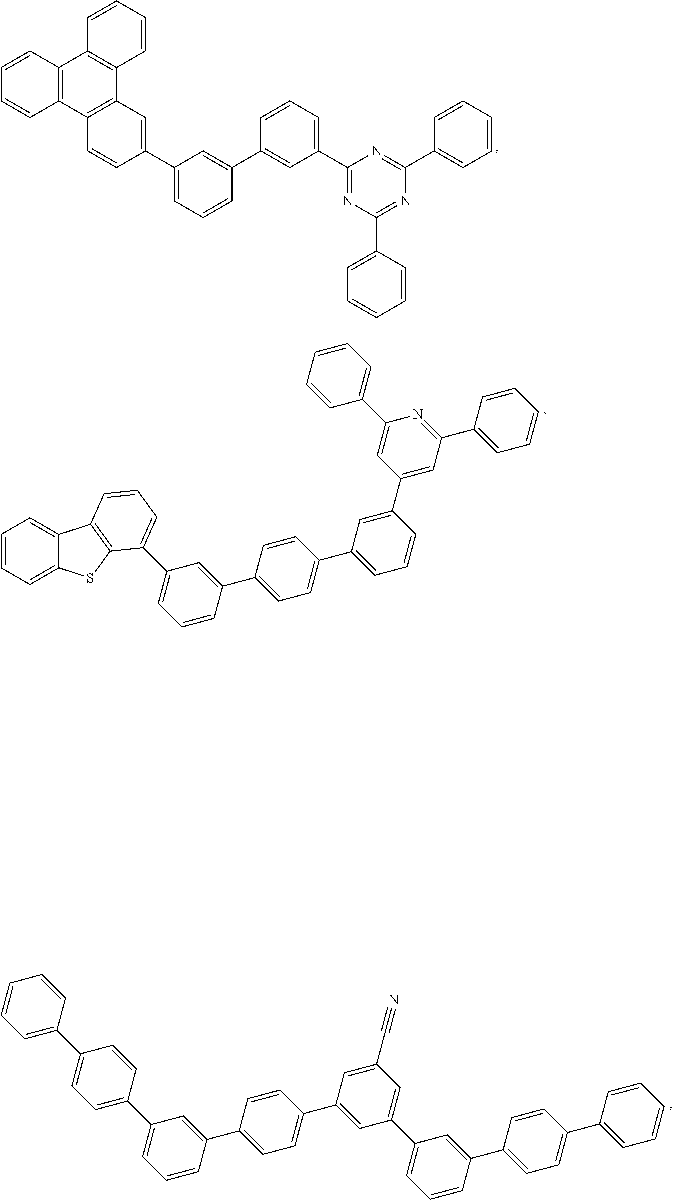

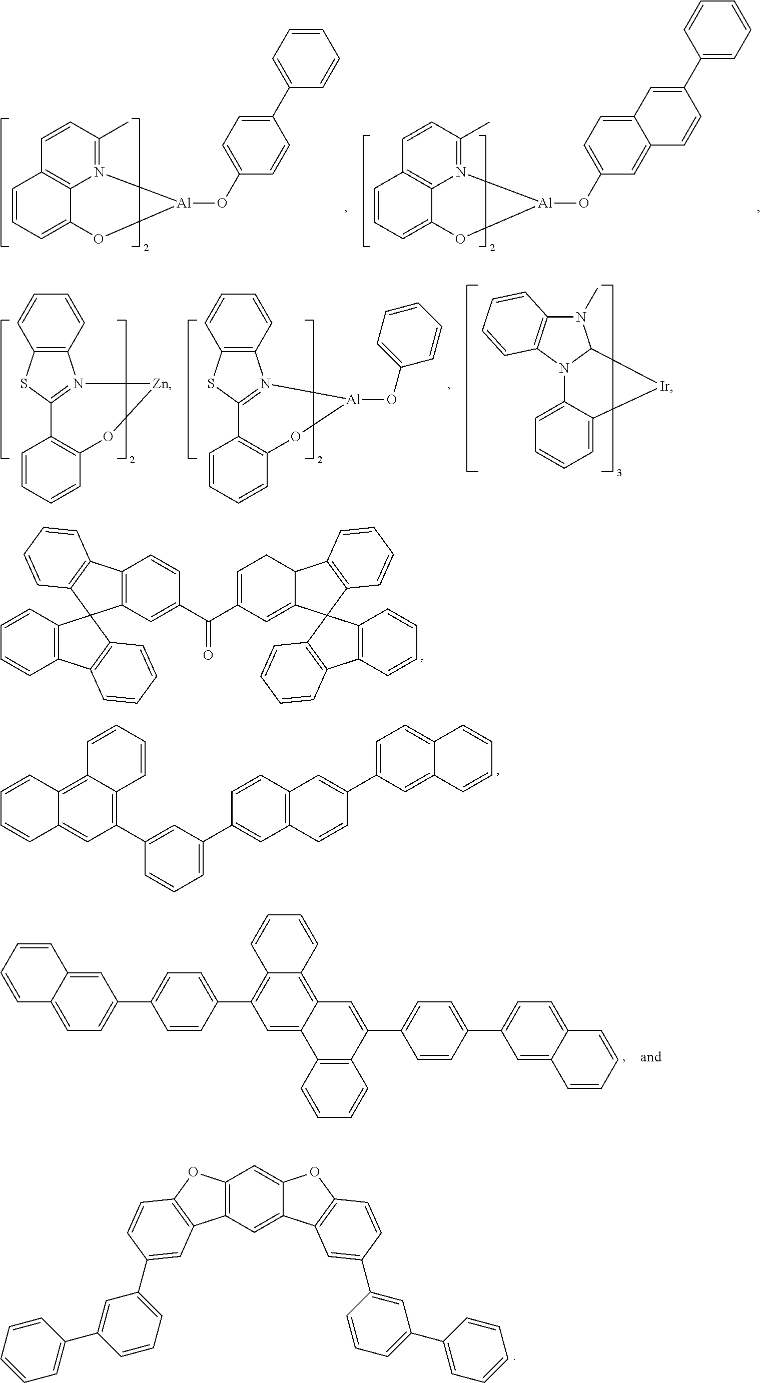

e) Additional Emitters:

One or more additional emitter dopants may be used in conjunction with the compound of the present disclosure. Examples of the additional emitter dopants are not particularly limited, and any compounds may be used as long as the compounds are typically used as emitter materials. Examples of suitable emitter materials include, but are not limited to, compounds which can produce emissions via phosphorescence, fluorescence, thermally activated delayed fluorescence, i.e., TADF (also referred to as E-type delayed fluorescence), triplet-triplet annihilation, or combinations of these processes.

Non-limiting examples of the emitter materials that may be used in an OLED in combination with materials disclosed herein are exemplified below together with references that disclose those materials: CN103694277, CN1696137, EB01238981, EP01239526, EP01961743, EP1239526, EP1244155, EP1642951, EP1647554, EP1841834, EP1841834B, EP2062907, EP2730583, JP2012074444, JP2013110263, JP4478555, KR1020090133652, KR20120032054, KR20130043460, TW201332980, U.S. Ser. No. 06/699,599, U.S. Ser. No. 06/916,554, US20010019782, US20020034656, US20030068526, US20030072964, US20030138657, US20050123788, US20050244673, US2005123791, US2005260449, US20060008670, US20060065890, US20060127696, US20060134459, US20060134462, US20060202194, US20060251923, US20070034863, US20070087321, US20070103060, US20070111026, US20070190359, US20070231600, US2007034863, US2007104979, US2007104980, US2007138437, US2007224450, US2007278936, US20080020237, US20080233410, US20080261076, US20080297033, US200805851, US2008161567, US2008210930, US20090039776, US20090108737, US20090115322, US20090179555, US2009085476, US2009104472, US20100090591, US20100148663, US20100244004, US20100295032, US2010102716, US2010105902, US2010244004, US2010270916, US20110057559, US20110108822, US20110204333, US2011215710, US2011227049, US2011285275, US2012292601, US20130146848, US2013033172, US2013165653, US2013181190, US2013334521, US20140246656, US2014103305, U.S. Pat. Nos. 6,303,238, 6,413,656, 6,653,654, 6,670,645, 6,687,266, 6,835,469, 6,921,915, 7,279,704, 7,332,232, 7,378,162, 7,534,505, 7,675,228, 7,728,137, 7,740,957, 7,759,489, 7,951,947, 8,067,099, 8,592,586, 8,871,361, WO06081973, WO06121811, WO07018067, WO07108362, WO07115970, WO07115981, WO08035571, WO2002015645, WO2003040257, WO2005019373, WO2006056418, WO2008054584, WO2008078800, WO2008096609, WO2008101842, WO2009000673, WO2009050281, WO2009100991, WO2010028151, WO2010054731, WO2010086089, WO2010118029, WO2011044988, WO2011051404, WO2011107491, WO2012020327, WO2012163471, WO2013094620, WO2013107487, WO2013174471, WO2014007565, WO2014008982, WO2014023377, WO2014024131, WO2014031977, WO2014038456, WO2014112450.

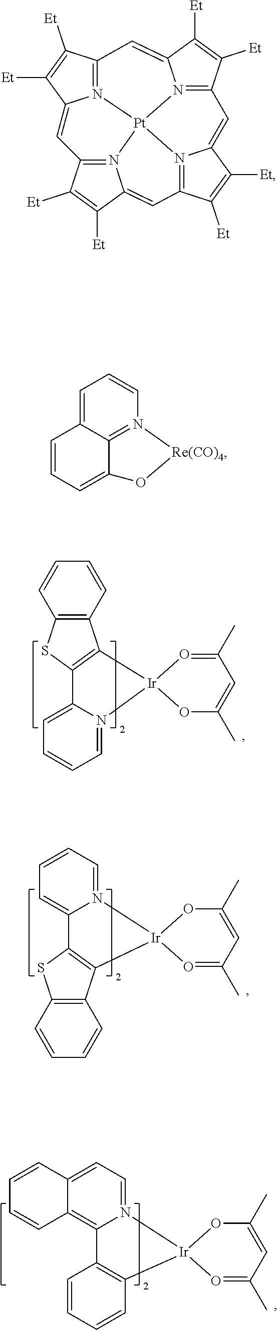

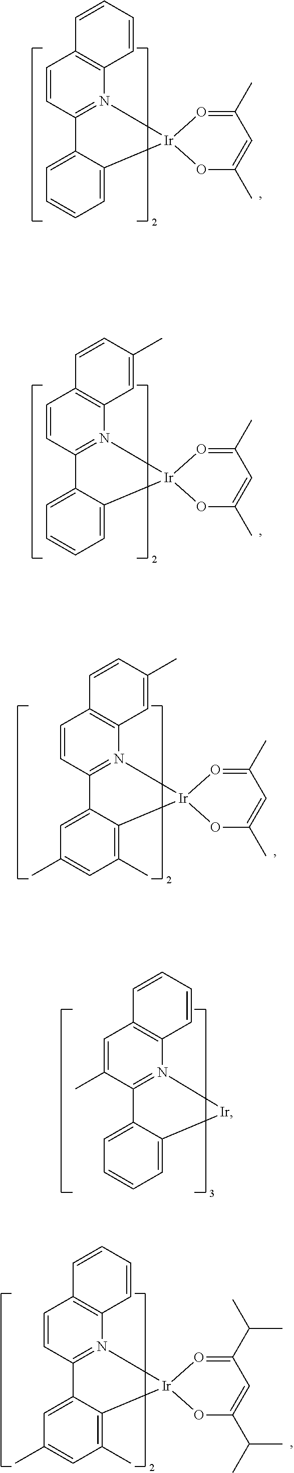

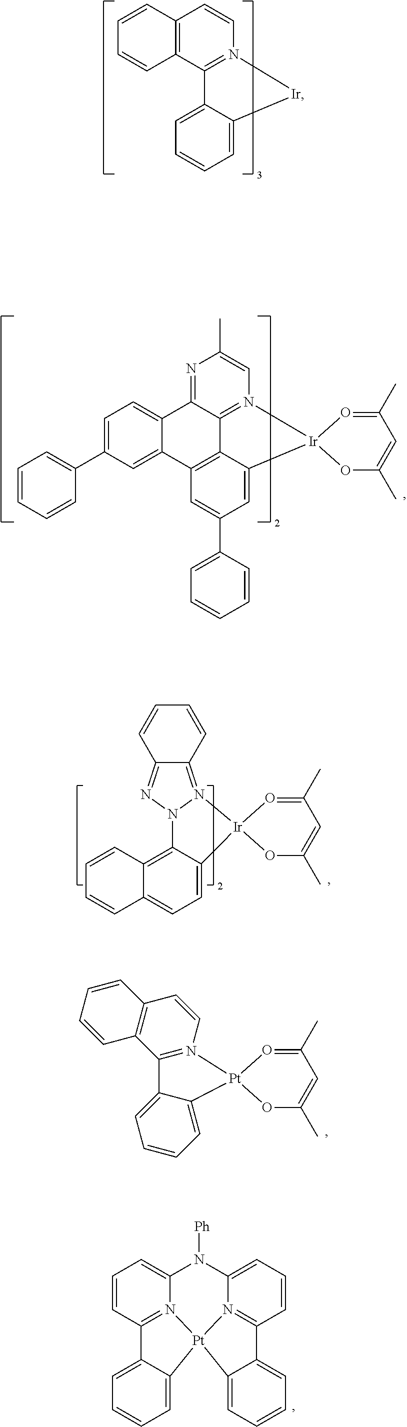

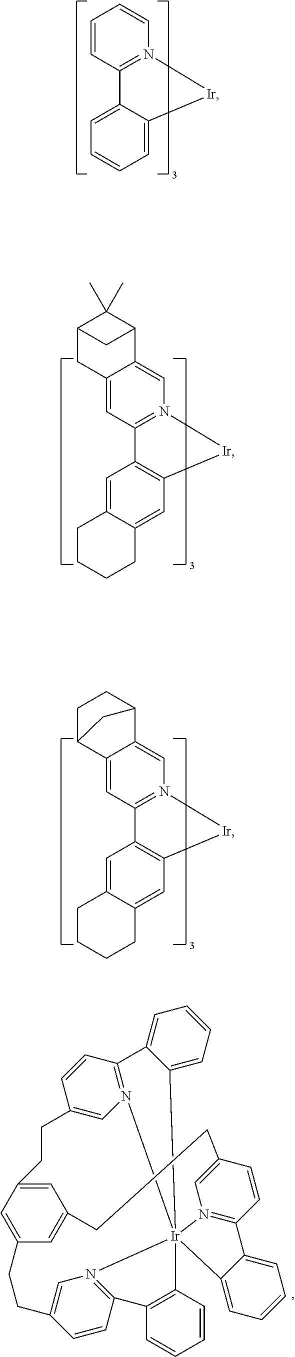

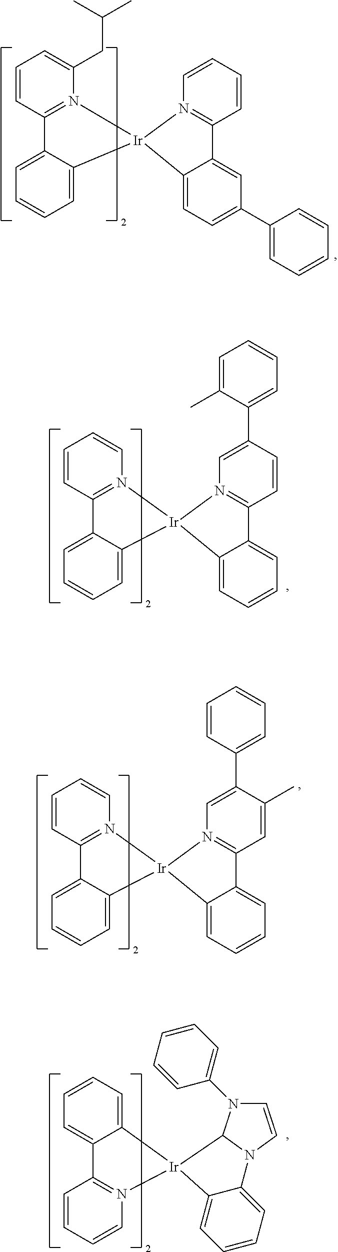

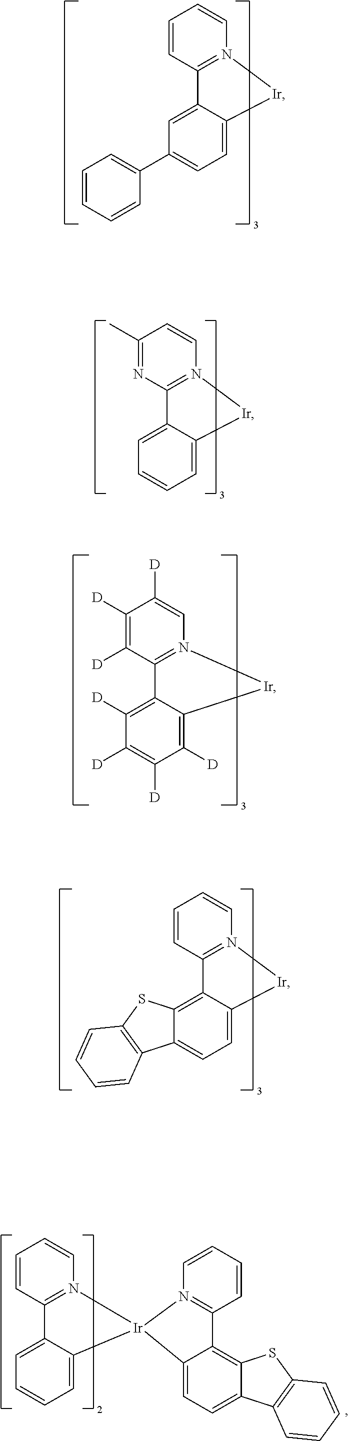

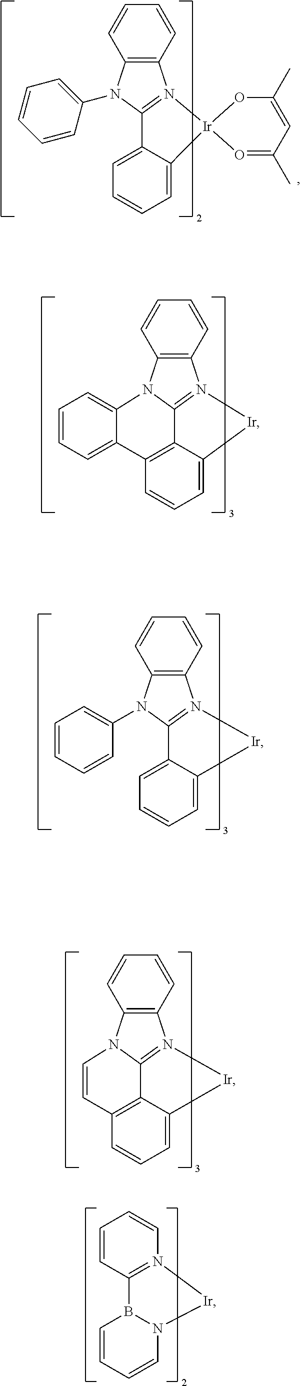

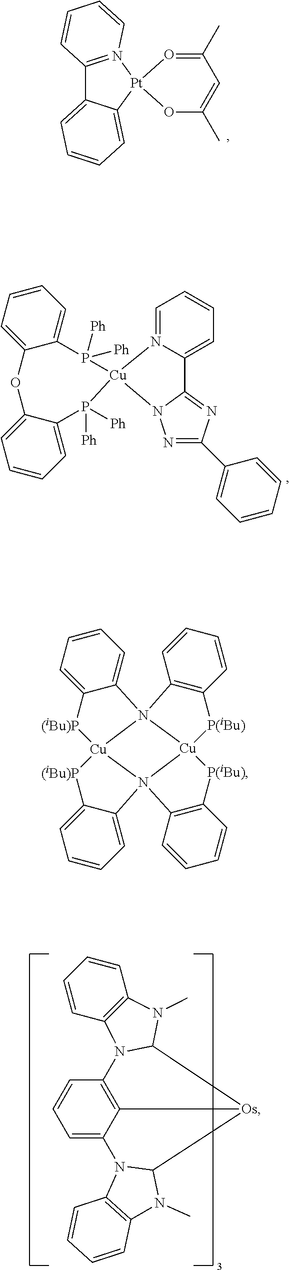

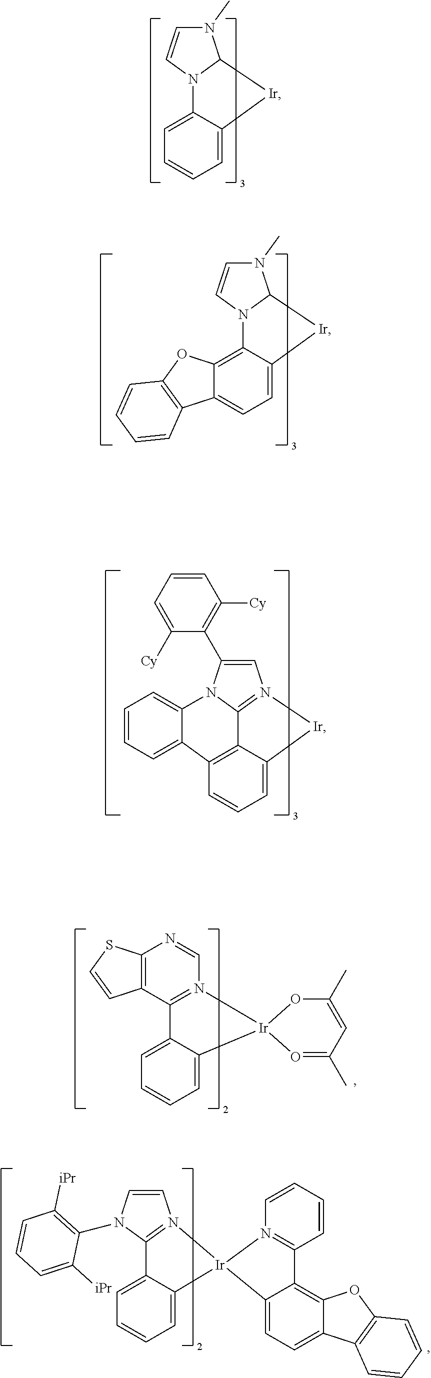

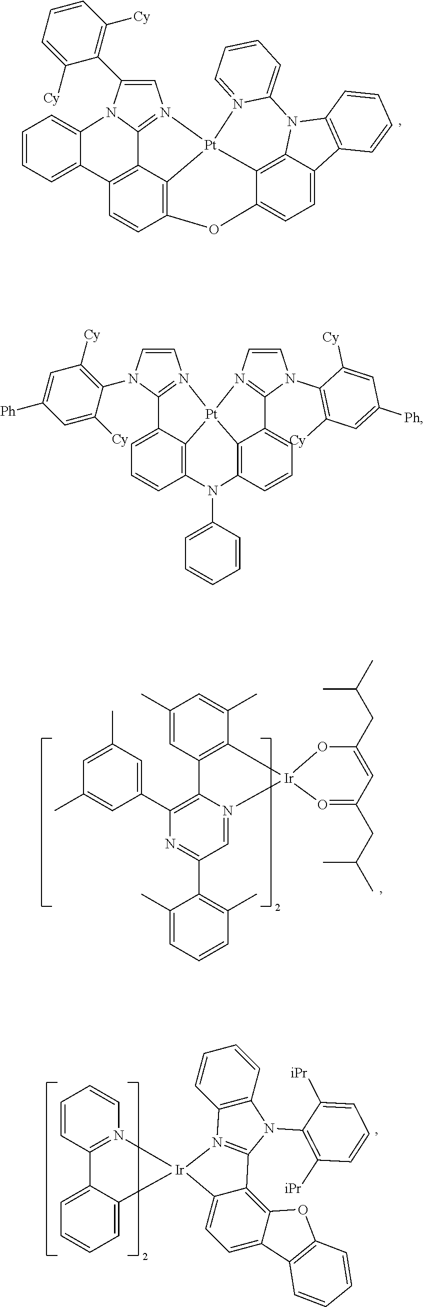

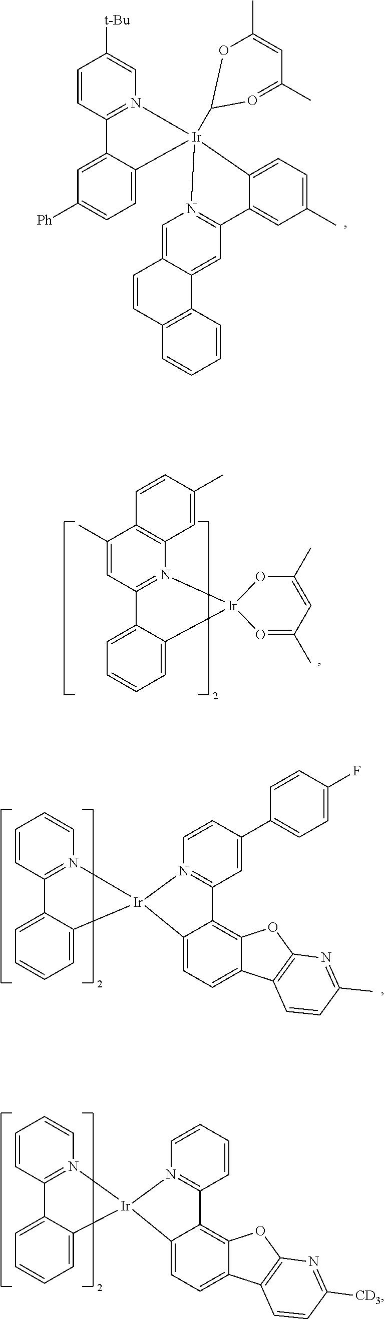

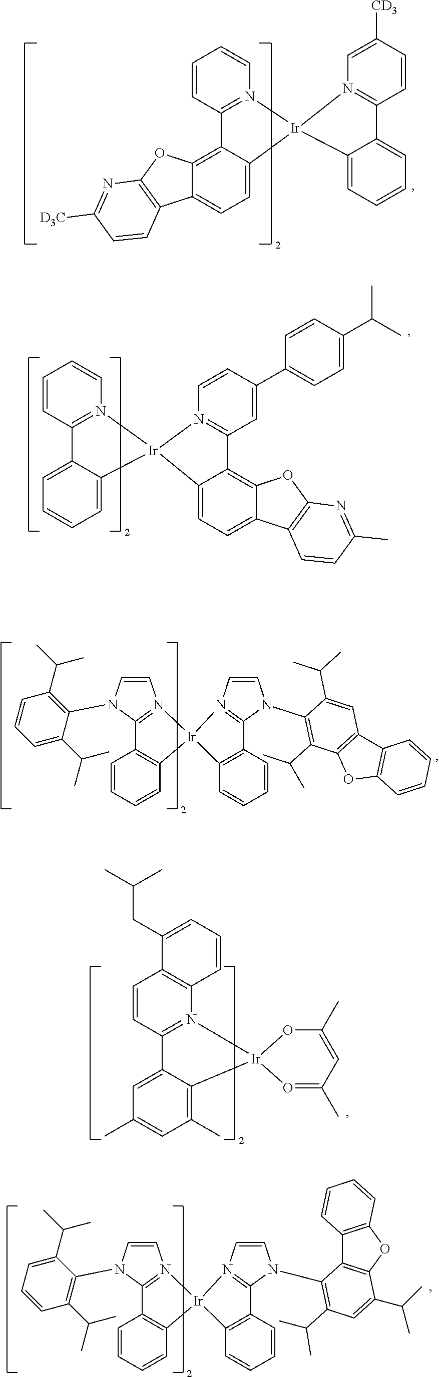

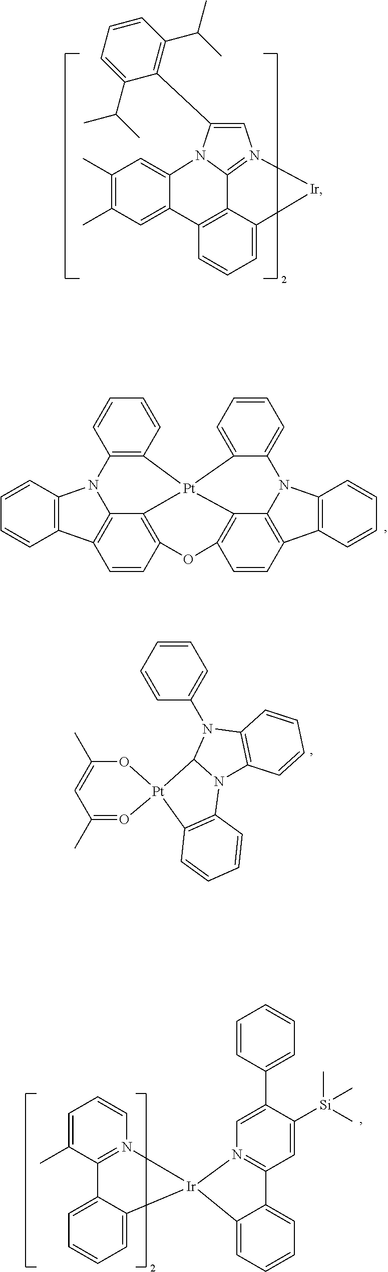

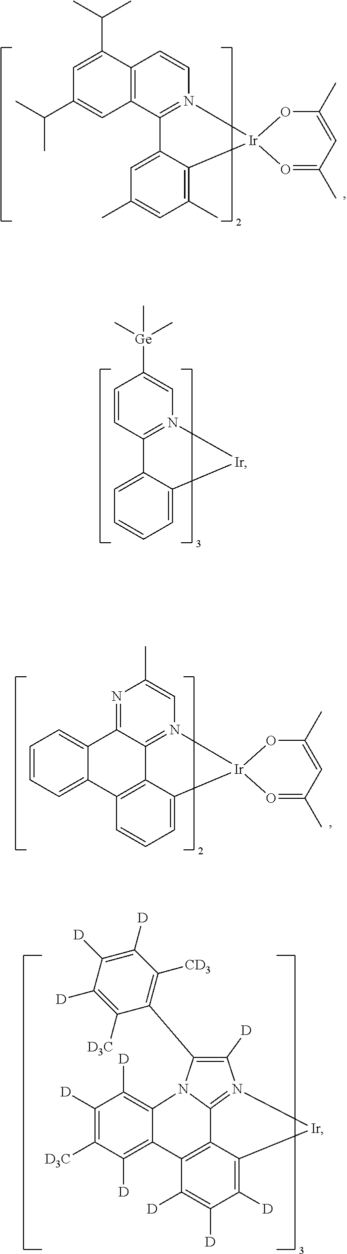

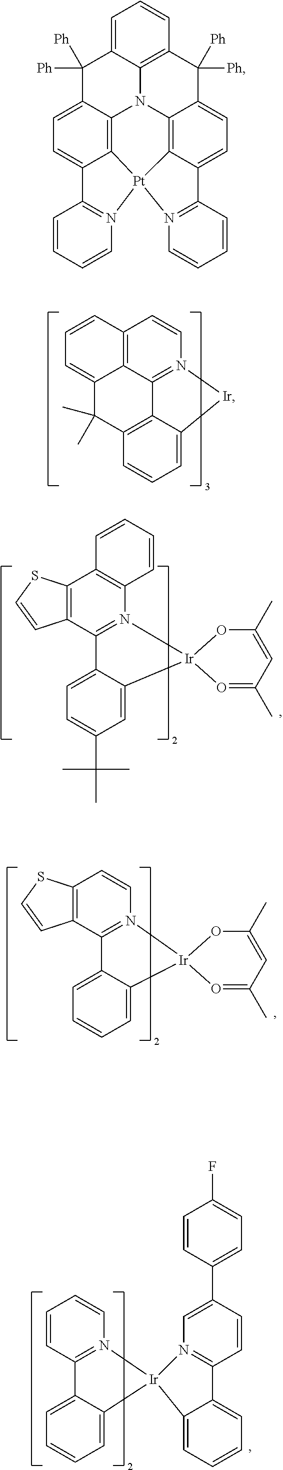

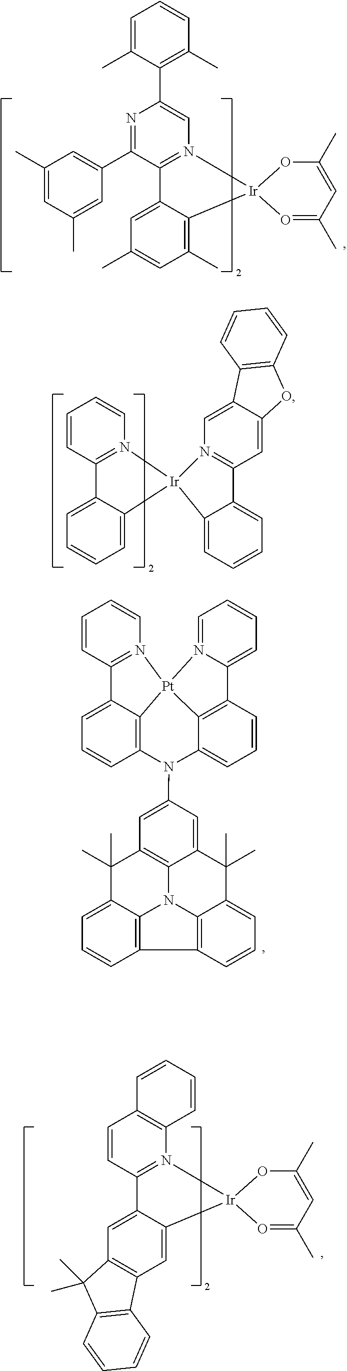

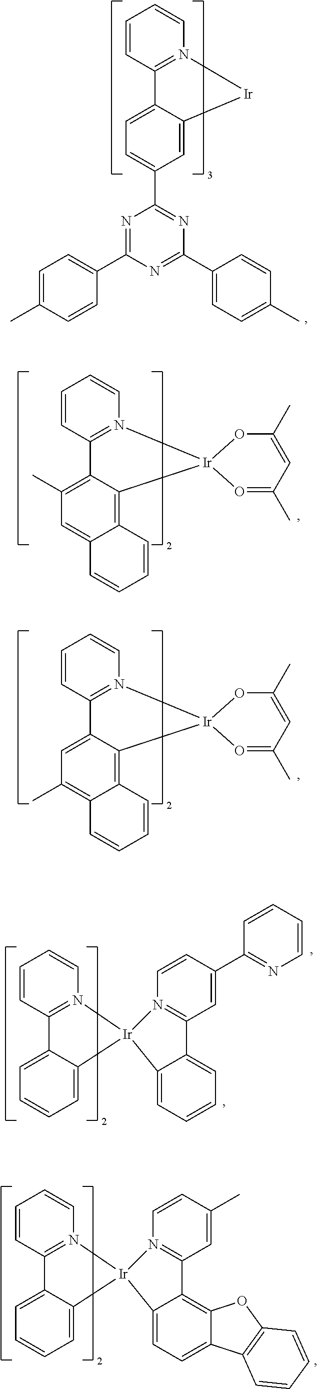

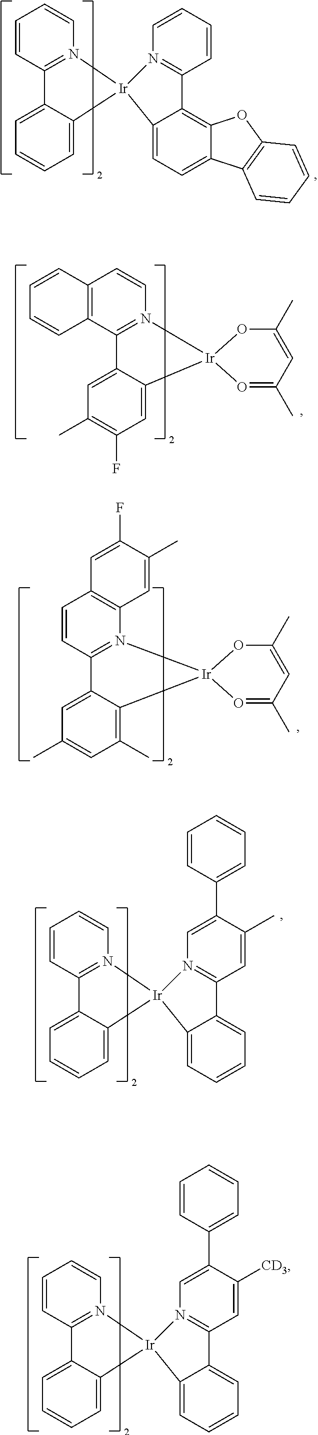

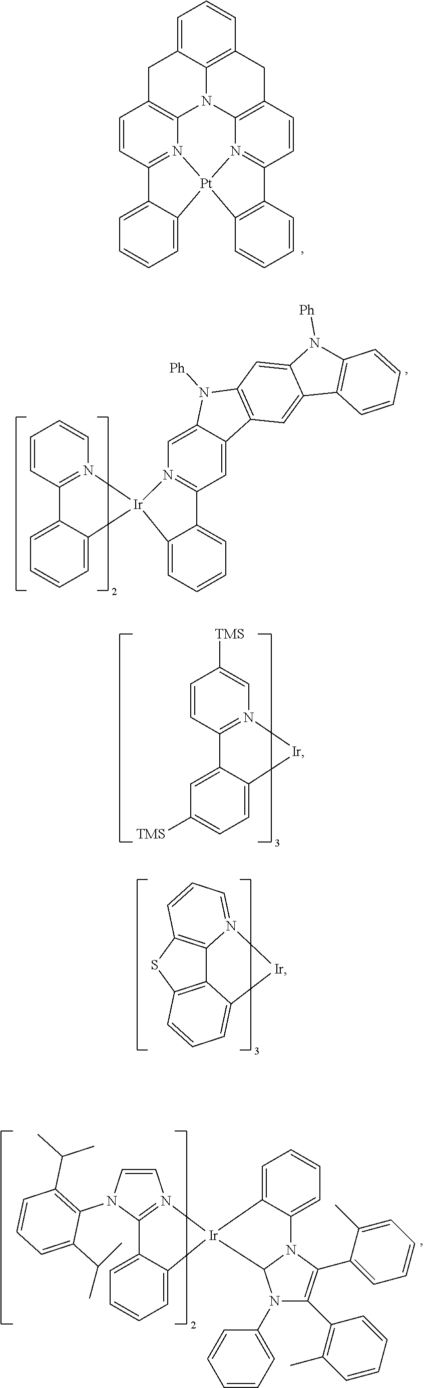

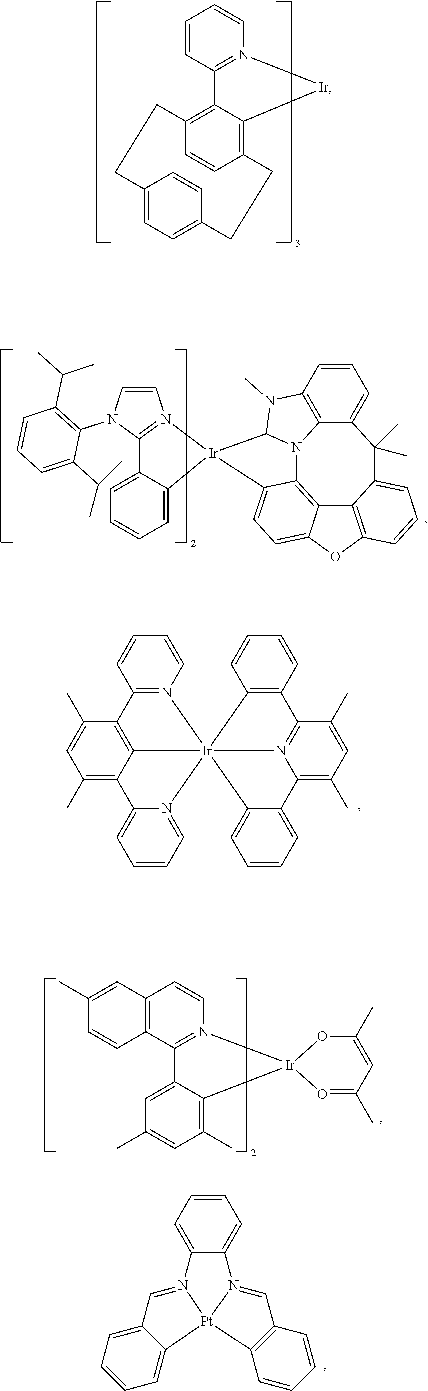

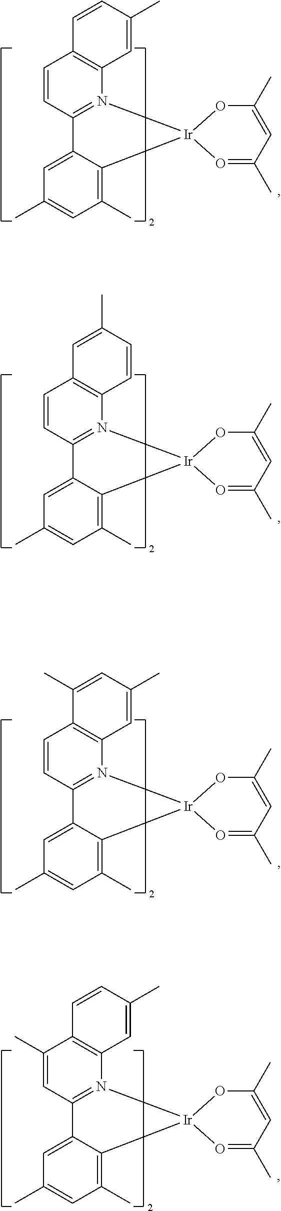

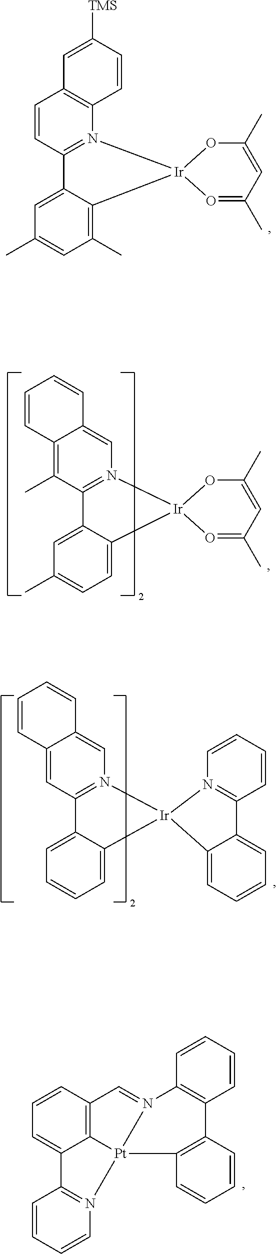

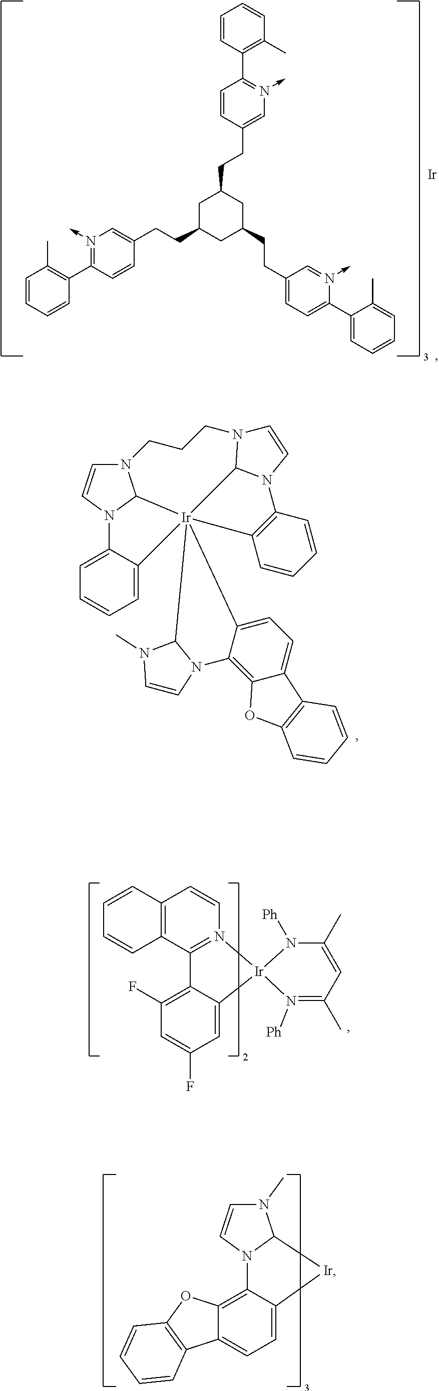

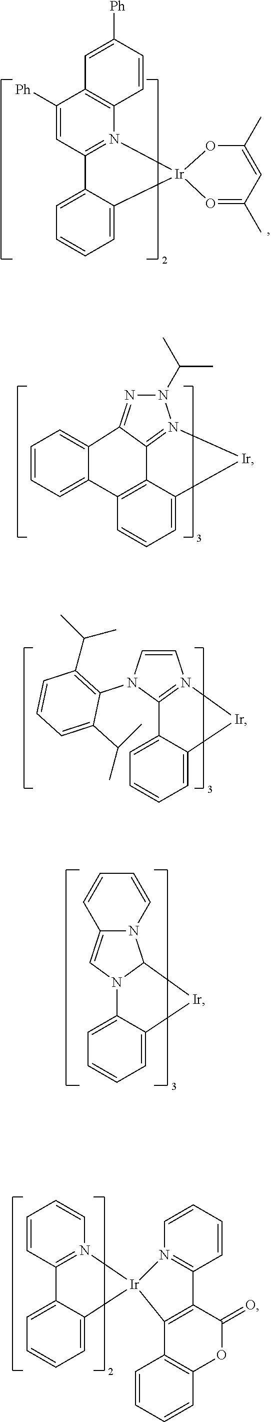

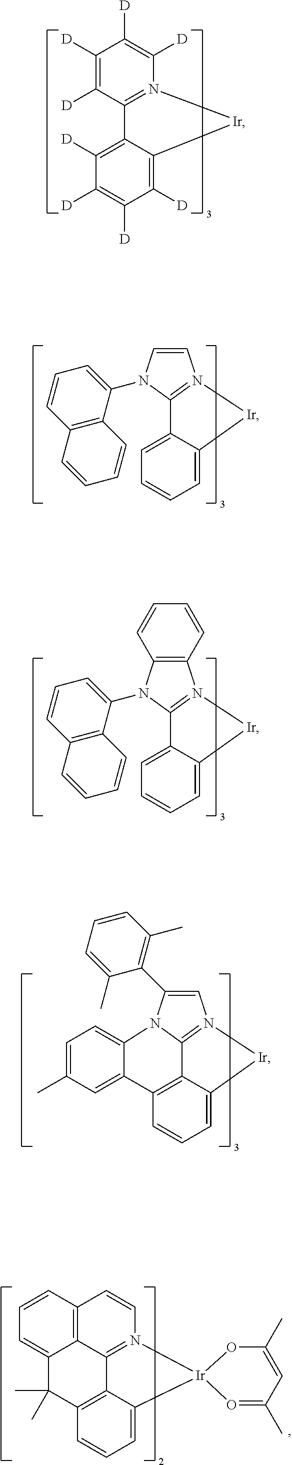

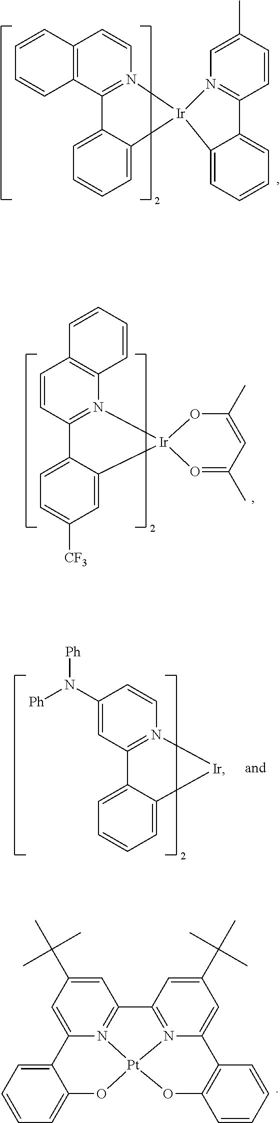

f) HBL:

A hole blocking layer (HBL) may be used to reduce the number of holes and/or excitons that leave the emissive layer. The presence of such a blocking layer in a device may result in substantially higher efficiencies and/or longer lifetime as compared to a similar device lacking a blocking layer. Also, a blocking layer may be used to confine emission to a desired region of an OLED. In some embodiments, the HBL material has a lower HOMO (further from the vacuum level) and/or higher triplet energy than the emitter closest to the HBL interface. In some embodiments, the HBL material has a lower HOMO (further from the vacuum level) and/or higher triplet energy than one or more of the hosts closest to the HBL interface.

In one aspect, compound used in HBL contains the same molecule or the same functional groups used as host described above.

In another aspect, compound used in HBL contains at least one of the following groups in the molecule:

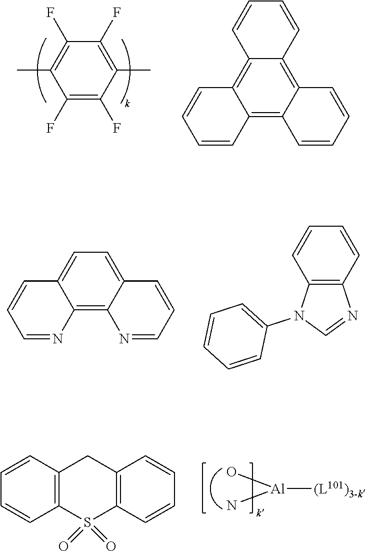

wherein k is an integer from 1 to 20; L¹⁰¹ is another ligand, k′ is an integer from 1 to 3.

g) ETL:

Electron transport layer (ETL) may include a material capable of transporting electrons. Electron transport layer may be intrinsic (undoped), or doped. Doping may be used to enhance conductivity. Examples of the ETL material are not particularly limited, and any metal complexes or organic compounds may be used as long as they are typically used to transport electrons.

In one aspect, compound used in ETL contains at least one of the following groups in the molecule:

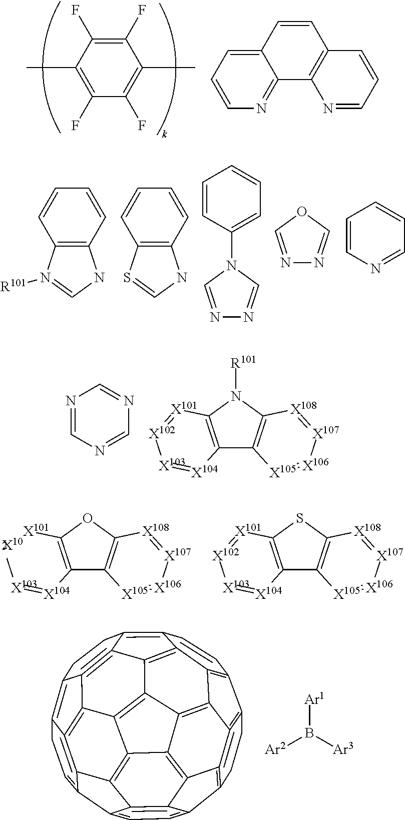

wherein R¹⁰¹ is selected from the group consisting of hydrogen, deuterium, halogen, alkyl, cycloalkyl, heteroalkyl, heterocycloalkyl, arylalkyl, alkoxy, aryloxy, amino, silyl, alkenyl, cycloalkenyl, heteroalkenyl, alkynyl, aryl, heteroaryl, acyl, carboxylic acids, ether, ester, nitrile, isonitrile, sulfanyl, sulfinyl, sulfonyl, phosphino, and combinations thereof, when it is aryl or heteroaryl, it has the similar definition as Ar's mentioned above. Ar¹ to Ar³ has the similar definition as Ar's mentioned above. k is an integer from 1 to 20. X¹⁰¹ to X¹⁰⁸ is selected from C (including CH) or N.

In another aspect, the metal complexes used in ETL contains, but not limit to the following general formula:

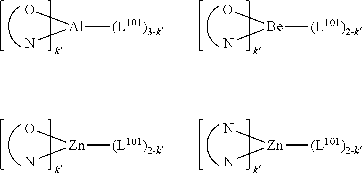

wherein (O—N) or (N—N) is a bidentate ligand, having metal coordinated to atoms O, N or N, N; L¹⁰¹ is another ligand; k′ is an integer value from 1 to the maximum number of ligands that may be attached to the metal.

Non-limiting examples of the ETL materials that may be used in an OLED in combination with materials disclosed herein are exemplified below together with references that disclose those materials: CN103508940, EP01602648, EP01734038, EP01956007, JP2004-022334, JP2005149918, JP2005-268199, KR0117693, KR20130108183, US20040036077, US20070104977, US2007018155, US20090101870, US20090115316, US20090140637, US20090179554, US2009218940, US2010108990, US2011156017, US2011210320, US2012193612, US2012214993, US2014014925, US2014014927, US20140284580, U.S. Pat. Nos. 6,656,612, 8,415,031, WO2003060956, WO2007111263, WO2009148269, WO2010067894, WO2010072300, WO2011074770, WO2011105373, WO2013079217, WO2013145667, WO2013180376, WO2014104499, WO2014104535,

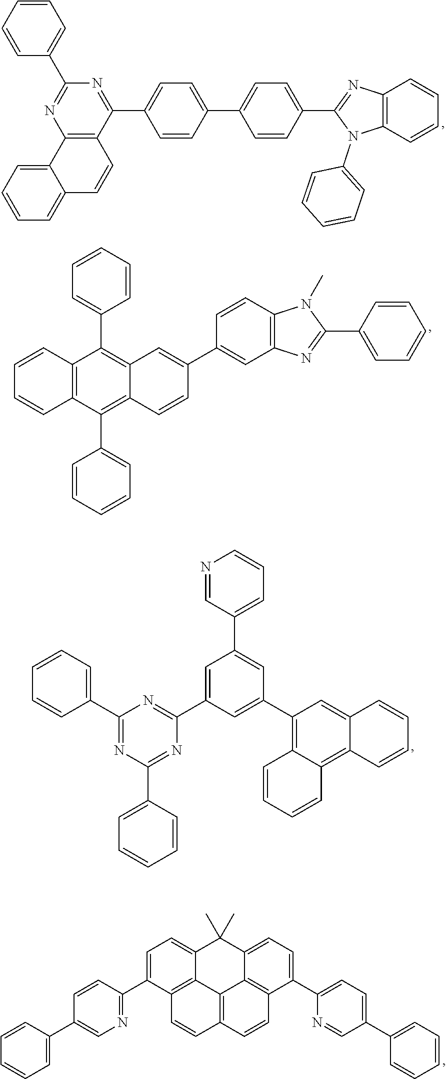 [SMILES: c1ccc(-c2nc(-c3ccc(-c4ccc(-c5nc6ccccc6n5-c5ccccc5)cc4)cc3)c3ccc4ccccc4c3n2)cc1 ; Cn1c(-c2ccccc2)nc2cc(-c3ccc4c(-c5ccccc5)c5ccccc5c(-c5ccccc5)c4c3)ccc21 ; c1ccc(-c2nc(-c3ccccc3)nc(-c3cc(-c4cccnc4)cc(-c4cc5ccccc5c5ccccc45)c3)n2)cc1 ; CC1(C)c2ccc(-c3ccc(-c4ccccc4)cn3)c3ccc4ccc5c(-c6ccc(-c7ccccc7)cn6)ccc1c5c4c23]

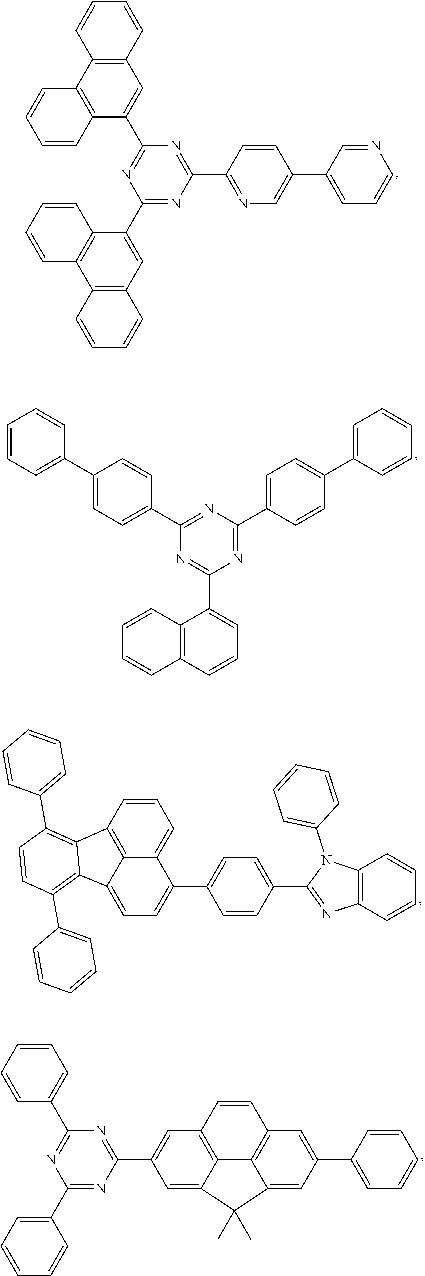 [SMILES: c1cncc(-c2ccc(-c3nc(-c4cc5ccccc5c5ccccc45)nc(-c4cc5ccccc5c5ccccc45)n3)nc2)c1 ; c1ccc(-c2ccc(-c3nc(-c4ccc(-c5ccccc5)cc4)nc(-c4cccc5ccccc45)n3)cc2)cc1 ; c1ccc(-c2ccc(-c3ccccc3)c3c2-c2cccc4c(-c5ccc(-c6nc7ccccc7n6-c6ccccc6)cc5)ccc-3c24)cc1 ; CC1(C)c2cc(-c3ccccc3)cc3ccc4cc(-c5nc(-c6ccccc6)nc(-c6ccccc6)n5)cc1c4c23]

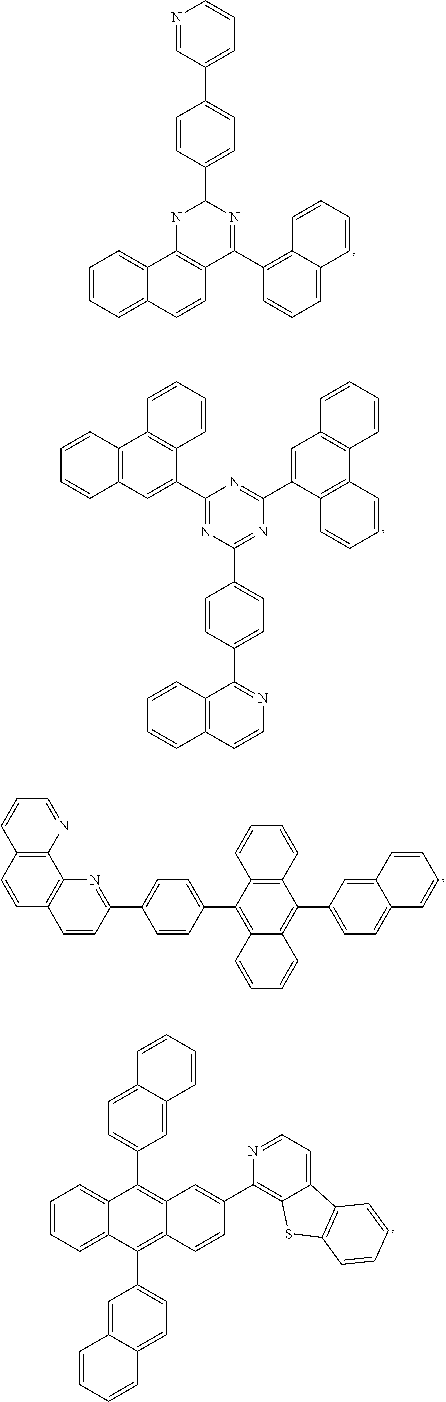 [SMILES: c1cncc(-c2ccc(C3N=C(c4cccc5ccccc45)c4ccc5ccccc5c4N3)cc2)c1 ; c1ccc2c(-c3ccc(-c4nc(-c5cc6ccccc6c6ccccc56)nc(-c5cc6ccccc6c6ccccc56)n4)cc3)nccc2c1 ; c1ccc2cc(-c3c4ccccc4c(-c4ccc(-c5ccc6ccc7cccnc7c6n5)cc4)c4ccccc34)ccc2c1 ; c1ccc2cc(-c3c4ccccc4c(-c4ccc5ccccc5c4)c4cc(-c5nccc6c5sc5ccccc56)ccc34)ccc2c1]

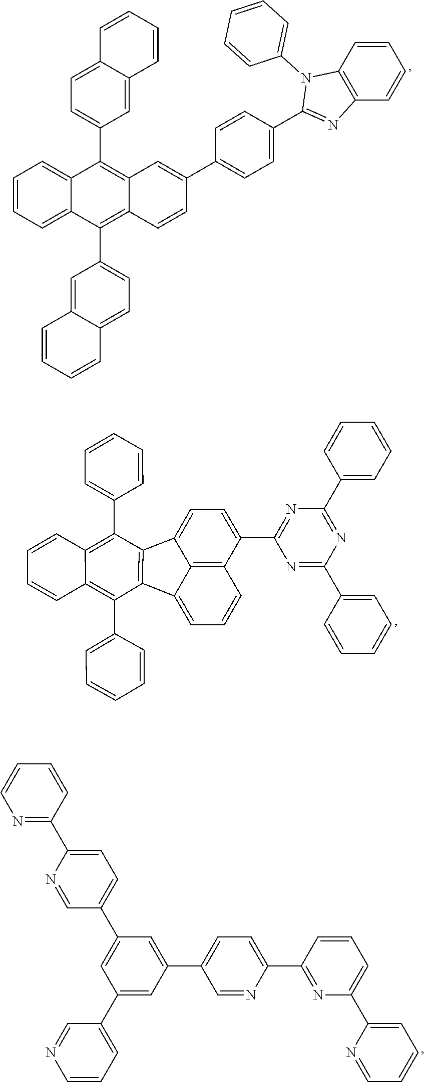 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2nc(-c3ccccc3)nc(-c3ccc4c5c(cccc35)-c3c-4c(-c4ccccc4)c4ccccc4c3-c3ccccc3)n2)cc1 ; c1ccc(-c2ccc(-c3cc(-c4cccnc4)cc(-c4ccc(-c5cccc(-c6ccccn6)n5)nc4)c3)cn2)nc1]

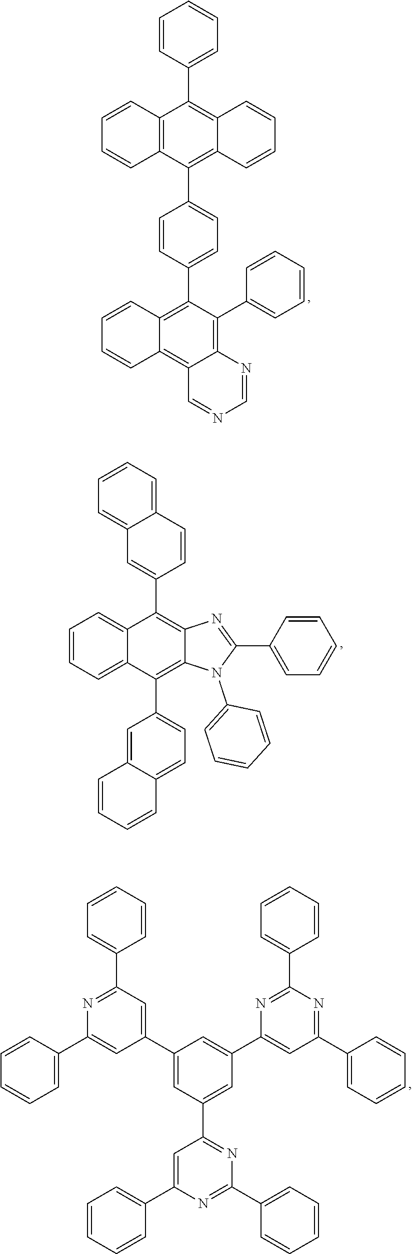 [SMILES: c1ccc(-c2c3ccccc3c(-c3ccc(-c4c(-c5ccccc5)c5ncncc5c5ccccc45)cc3)c3ccccc23)cc1 ; c1ccc(-c2nc3c(-c4ccc5ccccc5c4)c4ccccc4c(-c4ccc5ccccc5c4)c3n2-c2ccccc2)cc1 ; c1ccc(-c2cc(-c3cc(-c4cc(-c5ccccc5)nc(-c5ccccc5)n4)cc(-c4cc(-c5ccccc5)nc(-c5ccccc5)n4)c3)cc(-c3ccccc3)n2)cc1]

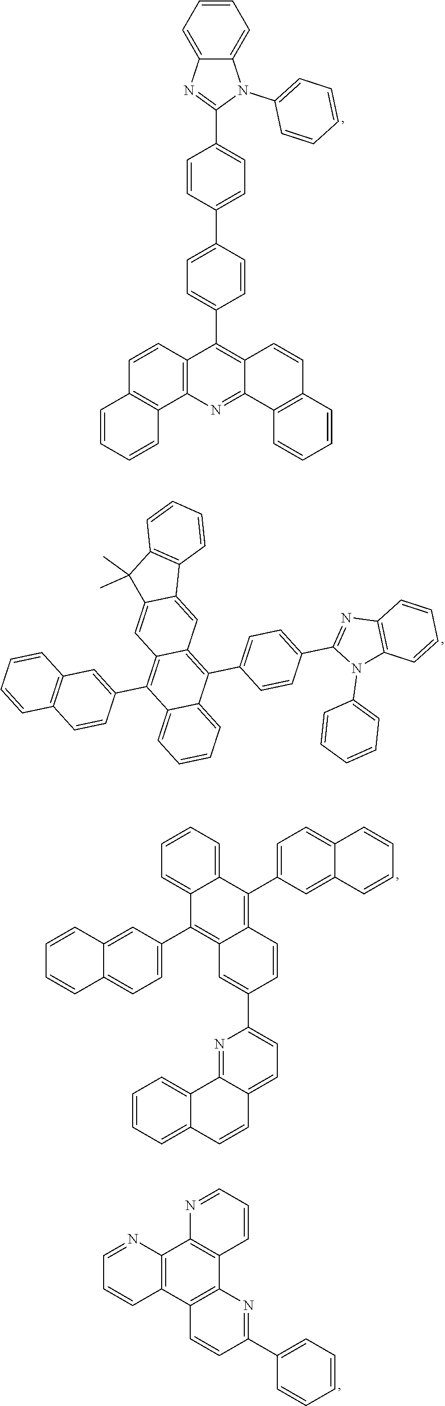

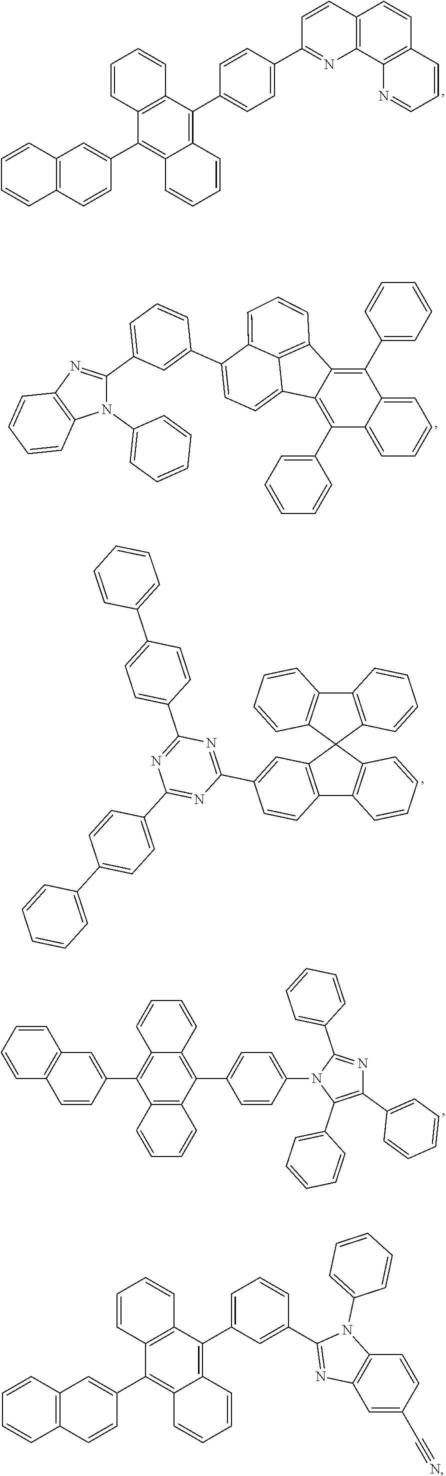 [SMILES: c1ccc2cc(-c3c4ccccc4c(-c4ccc(-c5ccc6ccc7cccnc7c6n5)cc4)c4ccccc34)ccc2c1 ; c1ccc(-c2c3c(c(-c4ccccc4)c4ccccc24)-c2ccc(-c4cccc(-c5nc6ccccc6n5-c5ccccc5)c4)c4cccc-3c24)cc1 ; c1ccc(-c2ccc(-c3nc(-c4ccc(-c5ccccc5)cc4)nc(-c4ccc5c(c4)C4(c6ccccc6-c6ccccc64)c4ccccc4-5)n3)cc2)cc1 ; c1ccc(-c2nc(-c3ccccc3)n(-c3ccc(-c4c5ccccc5c(-c5ccc6ccccc6c5)c5ccccc45)cc3)c2-c2ccccc2)cc1 ; N#Cc1ccc2c(c1)nc(-c1cccc(-c3c4ccccc4c(-c4ccc5ccccc5c4)c4ccccc34)c1)n2-c1ccccc1]

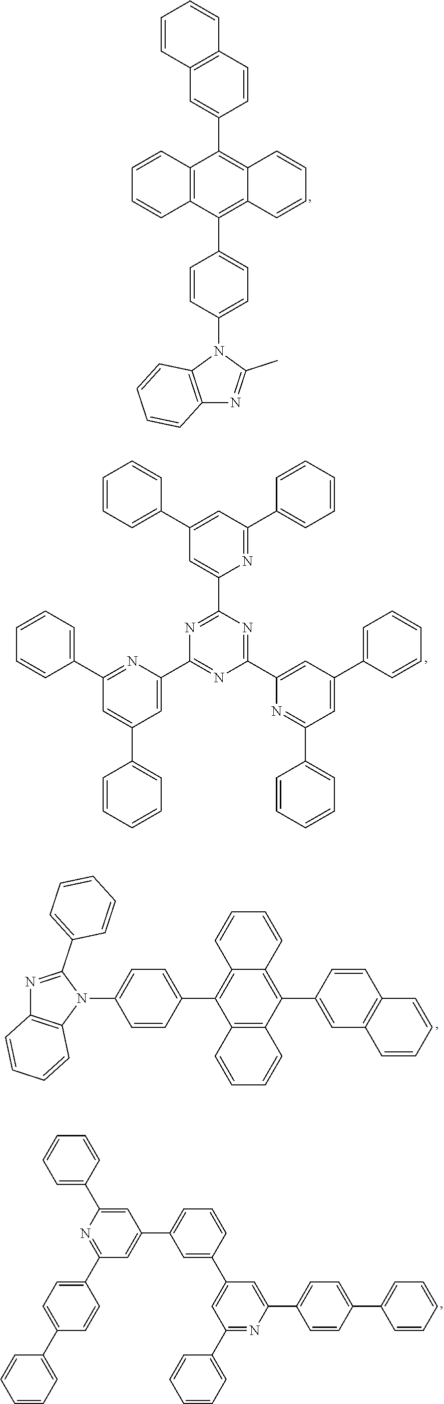

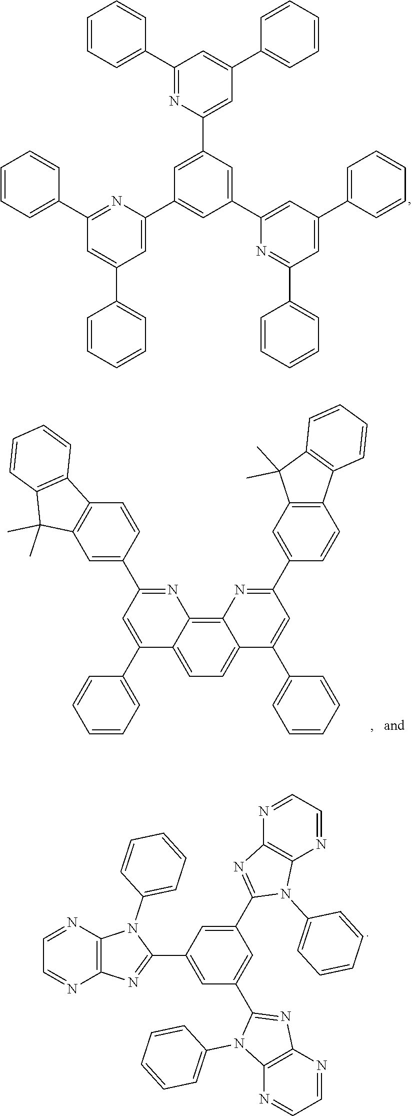 [SMILES: c1ccc(-c2cc(-c3ccccc3)nc(-c3cc(-c4cc(-c5ccccc5)cc(-c5ccccc5)n4)cc(-c4cc(-c5ccccc5)cc(-c5ccccc5)n4)c3)c2)cc1 ; CC1(C)c2ccccc2-c2ccc(-c3cc(-c4ccccc4)c4ccc5c(-c6ccccc6)cc(-c6ccc7c(c6)C(C)(C)c6ccccc6-7)nc5c4n3)cc21 ; c1ccc(-n2c(-c3cc(-c4nc5nccnc5n4-c4ccccc4)cc(-c4nc5nccnc5n4-c4ccccc4)c3)nc3nccnc32)cc1]

h) Charge Generation Layer (CGL)

In tandem or stacked OLEDs, the CGL plays an essential role in the performance, which is composed of an n-doped layer and a p-doped layer for injection of electrons and holes, respectively. Electrons and holes are supplied from the CGL and electrodes. The consumed electrons and holes in the CGL are refilled by the electrons and holes injected from the cathode and anode, respectively; then, the bipolar currents reach a steady state gradually. Typical CGL materials include n and p conductivity dopants used in the transport layers.

In any above-mentioned compounds used in each layer of the OLED device, the hydrogen atoms can be partially or fully deuterated. The minimum amount of hydrogen of the compound being deuterated is selected from the group consisting of 30%, 40%, 50%, 60%, 70%, 80%, 90%, 95%, 99%, and 100%. Thus, any specifically listed substituent, such as, without limitation, methyl, phenyl, pyridyl, etc. may be undeuterated, partially deuterated, and fully deuterated versions thereof. Similarly, classes of substituents such as, without limitation, alkyl, aryl, cycloalkyl, heteroaryl, etc. also may be undeuterated, partially deuterated, and fully deuterated versions thereof.

It is understood that the various embodiments described herein are by way of example only and are not intended to limit the scope of the invention. For example, many of the materials and structures described herein may be substituted with other materials and structures without deviating from the spirit of the invention. The present disclosure as claimed may therefore include variations from the particular examples and preferred embodiments described herein, as will be apparent to one of skill in the art. It is understood that various theories as to why the invention works are not intended to be limiting.

### D. Experimental Section

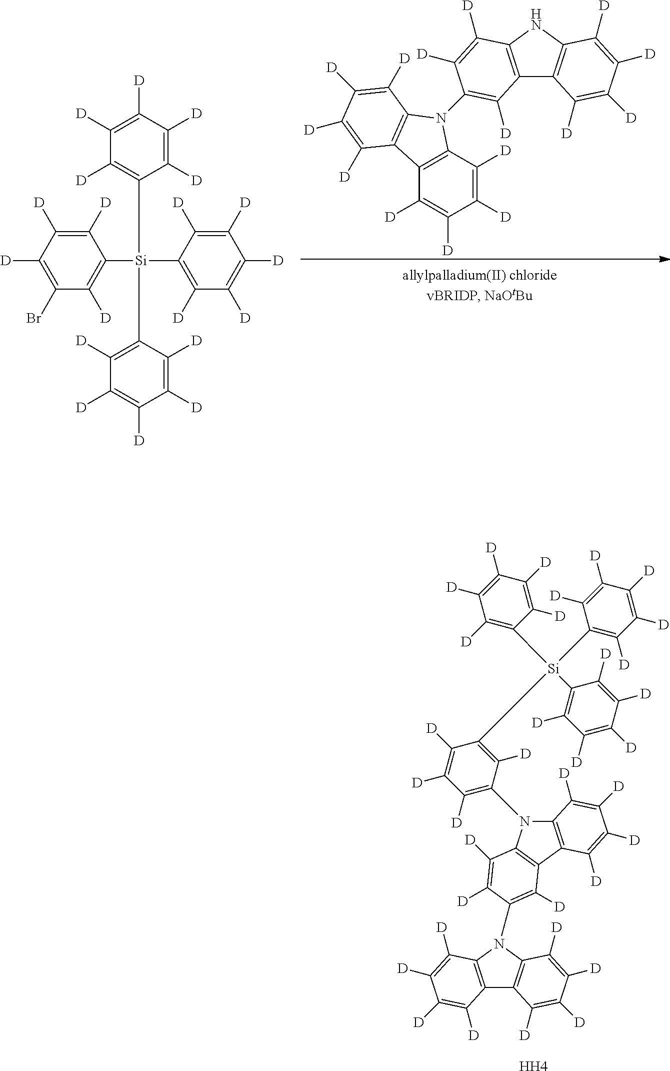

Sodium tert-butoxide (5.48 g, 57.0 mmol, 2.0 equiv) was added to dry xylene (158 mL) and the mixture was stirred at room temperature for 2 minutes. 9H-3,9′-bicarbazole-1,1′,2,2′,3′,4,4′,5,5′,6,6′,7,7′,8,8′-d₁₅ (9.9 g, 28.5 mmol, 1.0 equiv) was added, stirred for additional 2 minutes then nitrogen sparging was started. (3-Bromophenyl-2,4,5,6-d₄)tris(phenyl-d₅) (12.38 g, 28.5 mmol, 1.0 equiv) was added with continuous nitrogen sparging followed by the addition of di-tert-butyl(1-methyl-2,2-diphenylethenyl)phosphine, vBRIDP (0.58 g, 1.7 mmol, 0.06 mmol) and allylpalladium(II) chloride (0.63 g, 1.7 mmol, 0.06 mmol). The reaction mixture was then heated at 110° C. overnight. After cooling to room temperature, the reaction mixture was diluted with dichloromethane (600 mL) and water (150 mL). The layers were separated, and the organic layer was dried over sodium sulfate, filtered, and concentrated under reduced pressure. The residue was purified by column chromatography eluting with dichloromethane and hexanes to give HH4 (15.9 g, 80% yield) as a white solid.

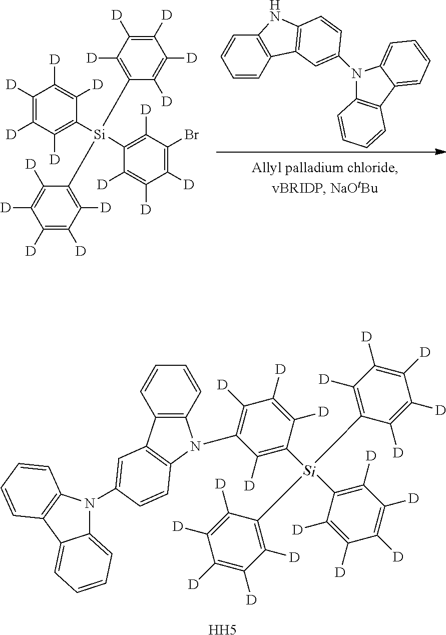

9H-3,9′-Bicarbazole (9.9 g, 29.8 mmol, 1.0 equiv) was added to a suspension of sodium tert-butoxide (5.72 g, 59.6 mmol, 2.0 equiv) in dry xylene (165 mL) and the resulting mixture was stirred at room temperature for 2 minutes. (3-Bromophenyl-2,4,5,6-d₄)tris(phenyl-d₅) (commercial) (12.94 g, 29.8 mmol, 1.0 equiv) was added and the mixture was sparged with nitrogen for 10 minutes. Bis(1,1-dimethylethyl)(1-methyl-2,2-diphenylethenyl)phosphine, vBRIDP (0.6 g, 1.79 mmol, 0.06 mmol) and allylpalladium(II) chloride (0.65 g, 1.79 mmol, 0.06 mmol) were added and the mixture was heated at 110° C. overnight. After cooling to room temperature, the reaction mixture was diluted with dichloromethane (600 mL) and water (150 mL). The layers were separated, and the organic layer was dried over sodium sulfate, filtered, and concentrated under reduced pressure. The residue was purified by column chromatography eluting with dichloromethane and hexanes to give HH5 (19.8 g, 97% yield) as white solid.

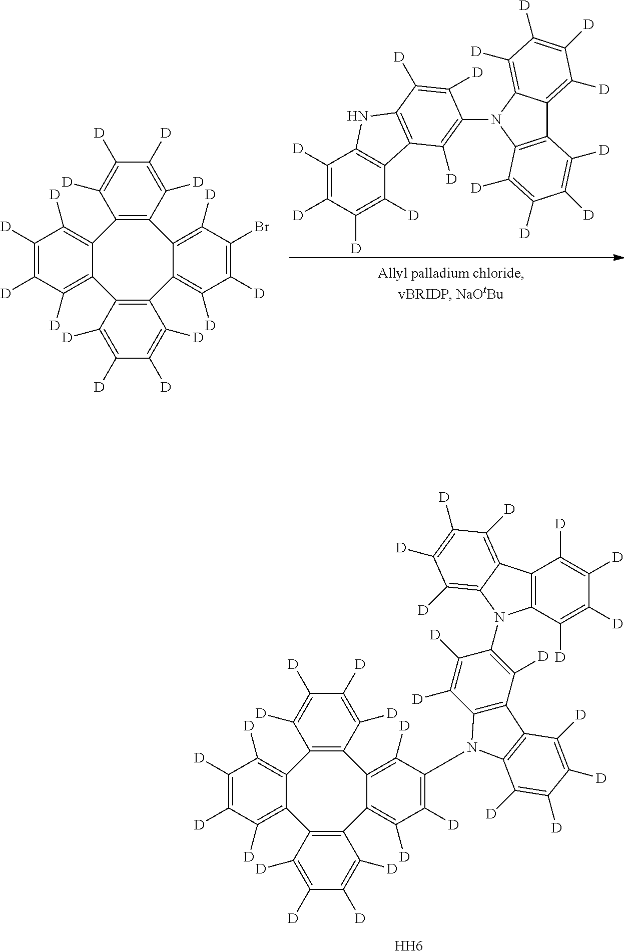

Following the above synthetic procedure with tetraphenylene in place of tetraphenyl silane will yield HH6.

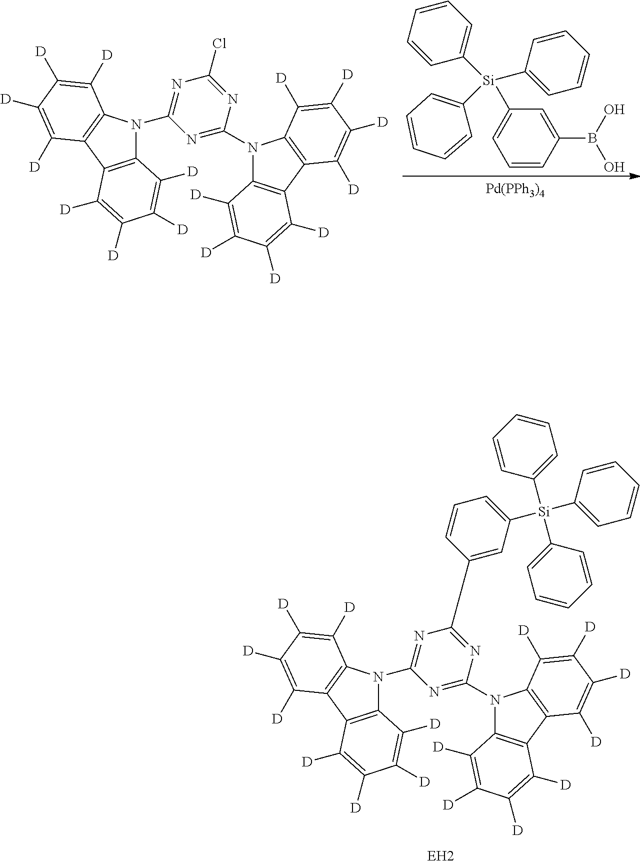

Potassium carbonate (4.31 g, 31.2 mmol, 2.0 equiv) in water (27 mL) was added to a solution of 9,9′-(6-chloro-1,3,5-triazine-2,4-diyl)bis(9H-carbazole-1,2,3,4,5,6,7,8-d₈) (7.2 g, 15.58 mmol, 1.0 equiv) and (3-(triphenylsilyl)phenyl)boronic acid (5.93 g, 15.58 mmol, 1.0 equiv) in THF (90 mL) and the mixture was sparged with nitrogen for 5 minutes. Tetrakis(triphenylphosphine)palladium(0) (1.81 g, 1.56 mmol, 0.1 equiv) was added and the mixture was heated at 72° C. overnight. The reaction mixture was quenched with water (300 mL) and extracted with dichloromethane (600 mL). The organic layer was separated, dried over anhydrous sodium sulfate, and concentrated under reduced pressure. The residue was purified by column chromatography eluting with dichloromethane and hexanes to give EH2 (5.89 g, 49% yield) as a white solid.

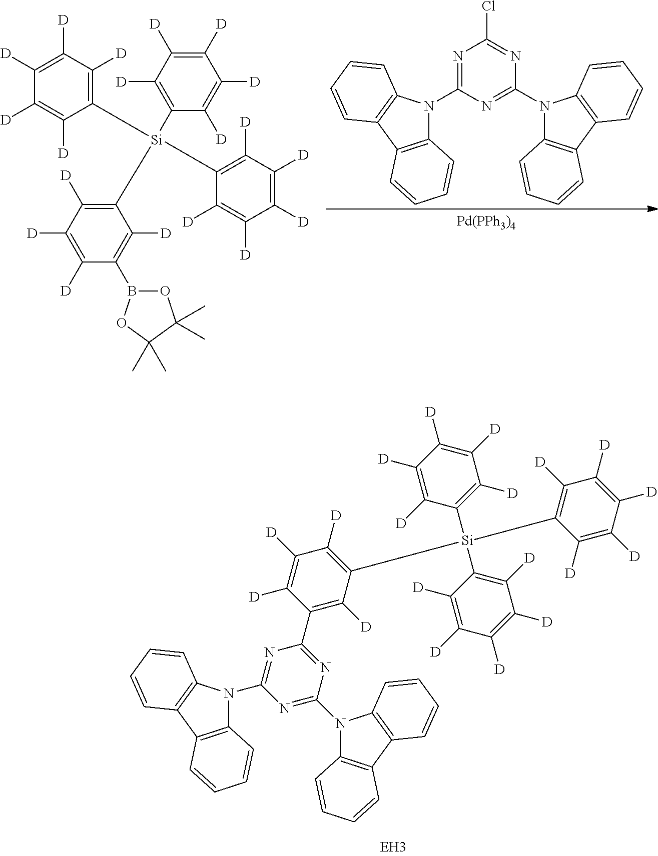 [SMILES: [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(B3OC(C)(C)C(C)(C)O3)c2[2H])c([2H])c1[2H] ; Clc1nc(-n2c3ccccc3c3ccccc32)nc(-n2c3ccccc3c3ccccc32)n1 ; [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(-c3nc(-n4c5ccccc5c5ccccc54)nc(-n4c5ccccc5c5ccccc54)n3)c2[2H])c([2H])c1[2H] ; "Pd(PPh3)4"]

Potassium carbonate (4.46 g, 32.3 mmol, 2.0 equiv) in water (41.4 mL) was added to a solution of 9,9′-(6-chloro-1,3,5-triazine-2,4-diyl)bis(9H-carbazole) (7.2 g, 16.15 mmol, 1.0 equiv) and tris(phenyl-d₈)(3-(4,4,5,5-tetramethyl-1,3,2-dioxaborolan-2-yl)phenyl-2,4,5,6-d₄)silane (7.78 g, 16.15 mmol, 1.0 equiv) in THF (138 mL) and the mixture was sparged with nitrogen for 5 minutes. Tetrakis(triphenylphosphine)palladium(0) (1.87 g, 1.6 mmol, 0.1 equiv) was added and the mixture was heated at 72° C. overnight. The reaction mixture was quenched with water (300 mL) and extracted with dichloromethane (600 mL). The organic layer was separated, dried over anhydrous sodium sulfate, and concentrated under reduced pressure. The residue was purified by column chromatography eluting with dichloromethane and hexanes to give EH3 (11 g, 14.38 mmol, 89% yield) as white solid.

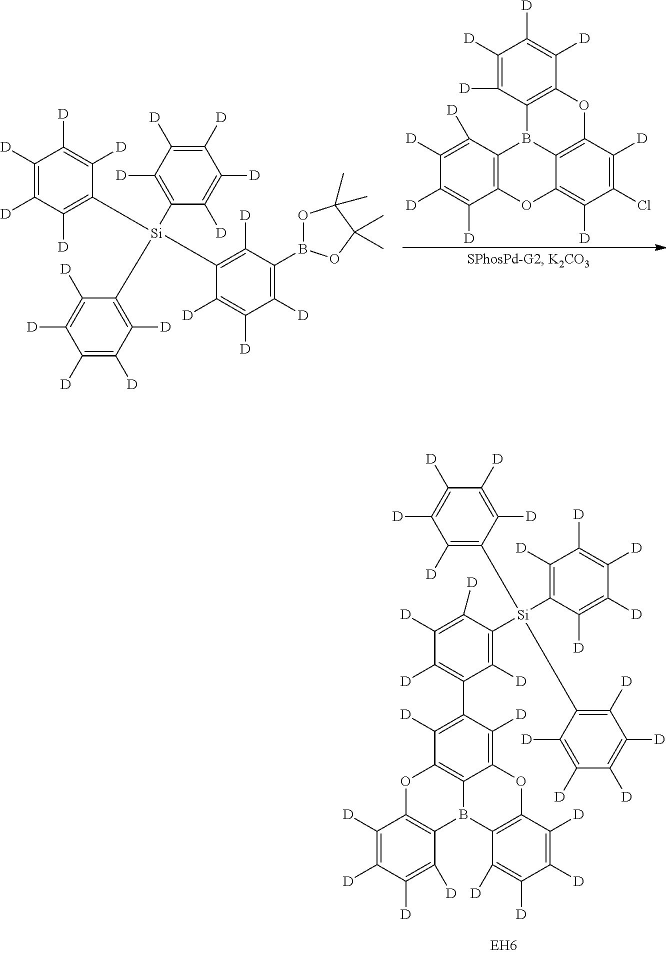

A mixture of tris(phenyl-d₅)(3-(4,4,5,5-tetramethyl-1,3,2-dioxaborolan-2-yl)phenyl-2,4,5,6-d₄)silane (12.0 g, 24.9 mmol, 1.0 equiv), 7-chloro-5,9-dioxa-13b-boranaphtho[3,2,1-de]anthracene-1,2,3,4,6,8,10,11,12,13-d₁₀ (8.6 g, 27.4 mmol, 1.1 equiv) and potassium carbonate (10.3 g, 74.8 mmol, 3 equiv) in 1,4-dioxane (120 mL) and water (30 mL) was degassed by sparging with nitrogen for 20 minutes. Chloro(2-dicyclohexylphosphino-2′,6′-dimethoxy-1,1′-biphenyl)[2-(2′-amino-1,1′-biphenyl)]palladium(II), SphosPd-G2 (1.8 g, 2.5 mmol, 0.10 equiv) was added and the mixture was degassed for another 10 minutes. The mixture was then heated to 85° C. and stirred overnight. The reaction mixture was cooled to room temperature and diluted with dichloromethane (1 L) and water (1 L). The layers were separated, and the aqueous layer was extracted with dichloromethane (300 mL). The combined organic layers were washed with saturated brine (500 mL), dried over sodium sulfate, and filtered. The filtrate was concentrated under reduced pressure and the residue was triturated with hexanes and dichloromethane at 35° C. for 1 hour to give EH6 (12.3 g, 78% yield) as a white solid.

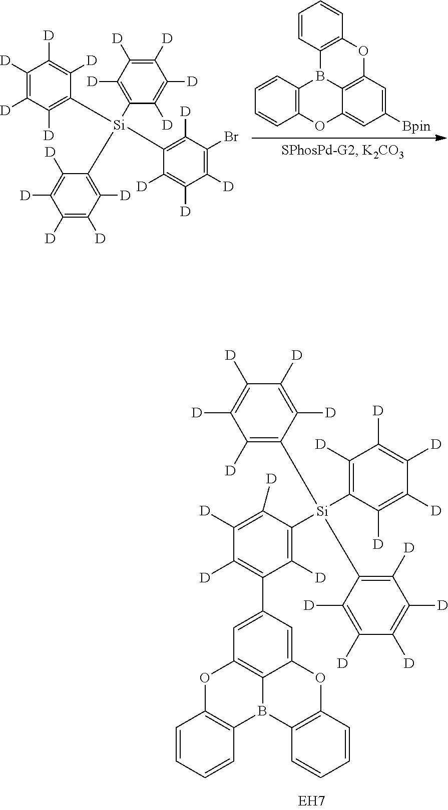

A mixture of (3-bromophenyl-2,4,5,6-d₄)tris(phenyl-d₅)silane (7.2 g, 16.6 mmol, 1.0 equiv), 7-(4,4,5,5-tetramethyl-1,3,2-dioxaborolan-2-yl)-5,9-dioxa-13b-boranaphtho[3,2,1-de]anthracene (7.2 g, 18.2 mmol, 1.1 equiv) and potassium carbonate (6.9 g, 50 mmol, 3 equiv) in 1,4-dioxane (100 mL) and water (25 mL) was degassed by sparging with nitrogen for 20 minutes. Chloro(2-dicyclohexylphosphino-2′,6′-dimethoxy-1,1′-biphenyl)[2-(2′-amino-1,1′-biphenyl)]palladium(II), SphosPd-G2 (1.2 g, 1.7 mmol, 0.10 equiv) was added and the mixture was degassed for another 10 minutes. The mixture was then heated to 80° C. and stirred overnight. The reaction mixture was cooled to room temperature and diluted with dichloromethane (1 L) and water (1 L). The layers were separated, and the aqueous layer was extracted with dichloromethane (300 mL). The combined organic layers were washed with saturated brine (500 mL), dried over sodium sulfate, and filtered. The filtrate was concentrated under reduced pressure and the residue was triturated with hexanes and dichloromethane at 35° C. for 1 hour to give EH7 (6.3 g, 61% yield) as a white solid.

OLED devices were fabricated using BD1 and BD2, blue phosphorescent emitters. The device results are shown in Table 1 and Table 2 respectively, where the EQE and voltage are taken at 10 mA/cm² and the lifetime (LT90) is the time to reduction of brightness to 90% of the initial luminance at a constant current density of 20 mA/cm².

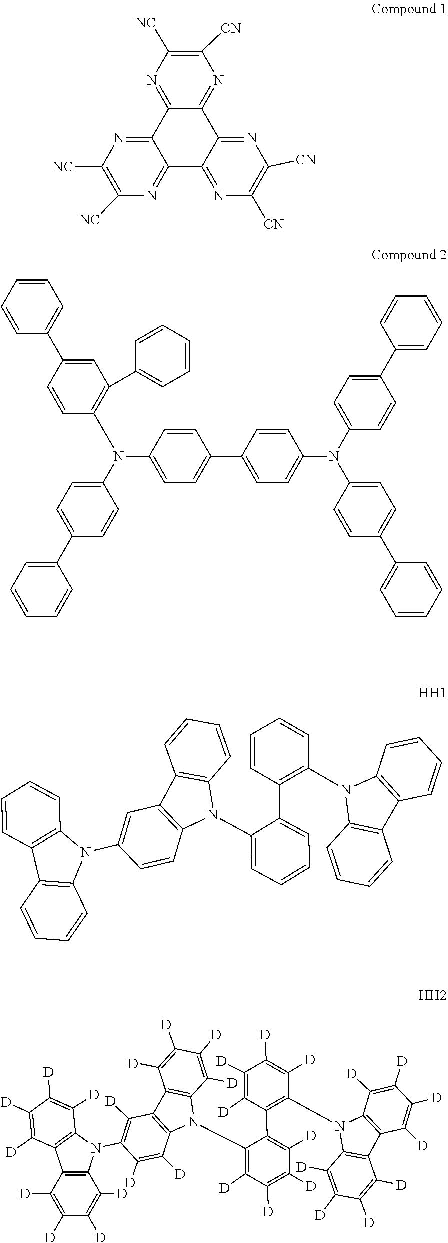

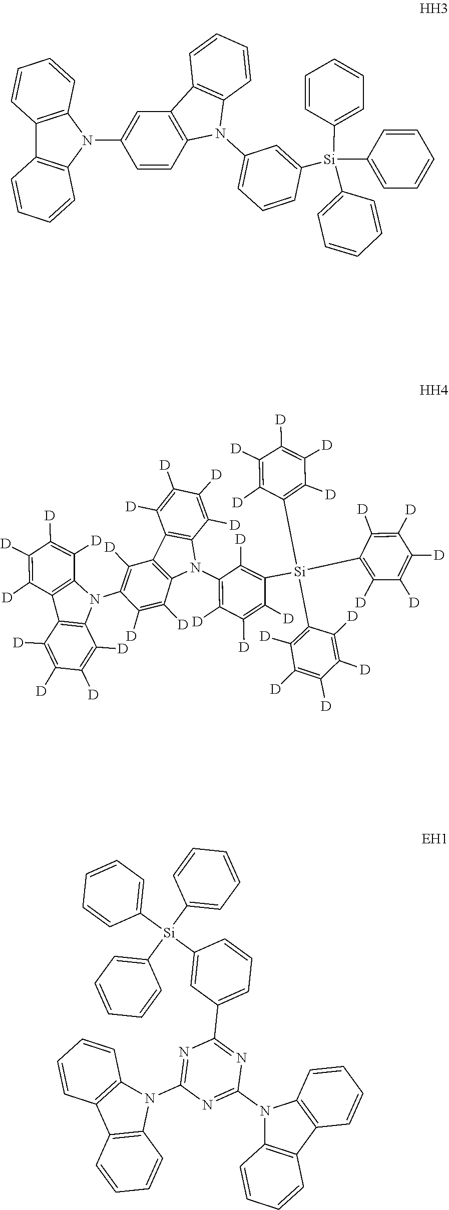 [SMILES: c1ccc([Si](c2ccccc2)(c2ccccc2)c2cccc(-n3c4ccccc4c4cc(-n5c6ccccc6c6ccccc65)ccc43)c2)cc1 ; [2H]c1c([2H])c([2H])c([Si](c2c([2H])c([2H])c([2H])c([2H])c2[2H])(c2c([2H])c([2H])c([2H])c([2H])c2[2H])c2c([2H])c([2H])c([2H])c(-n3c4c([2H])c([2H])c([2H])c([2H])c4c4c([2H])c(-n5c6c([2H])c([2H])c([2H])c([2H])c6c6c([2H])c([2H])c([2H])c([2H])c65)c([2H])c([2H])c43)c2[2H])c([2H])c1[2H] ; c1ccc([Si](c2ccccc2)(c2ccccc2)c2cccc(-c3nc(-n4c5ccccc5c5ccccc54)nc(-n4c5ccccc5c5ccccc54)n3)c2)cc1]

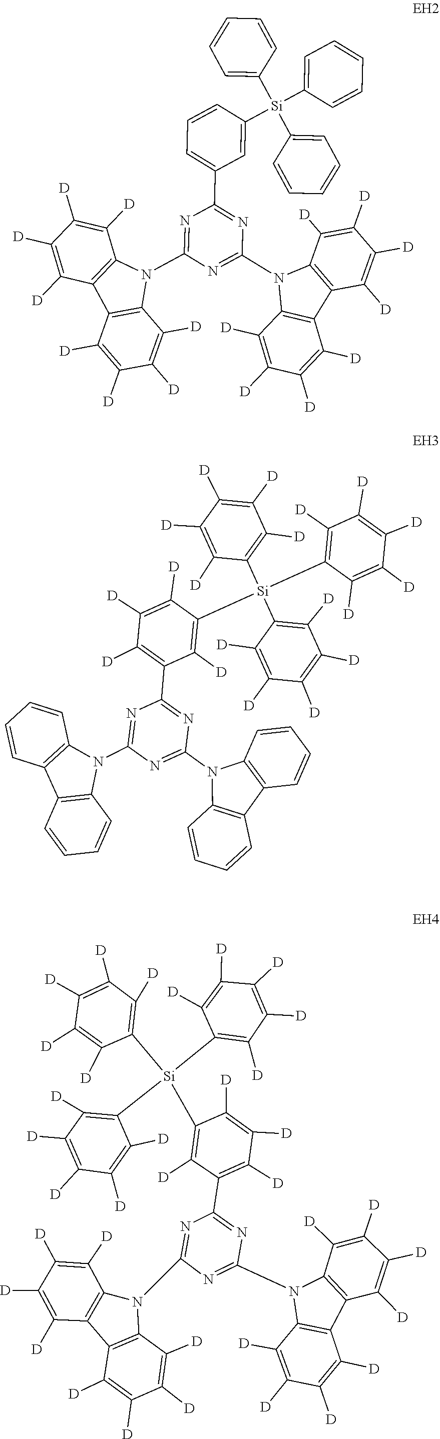

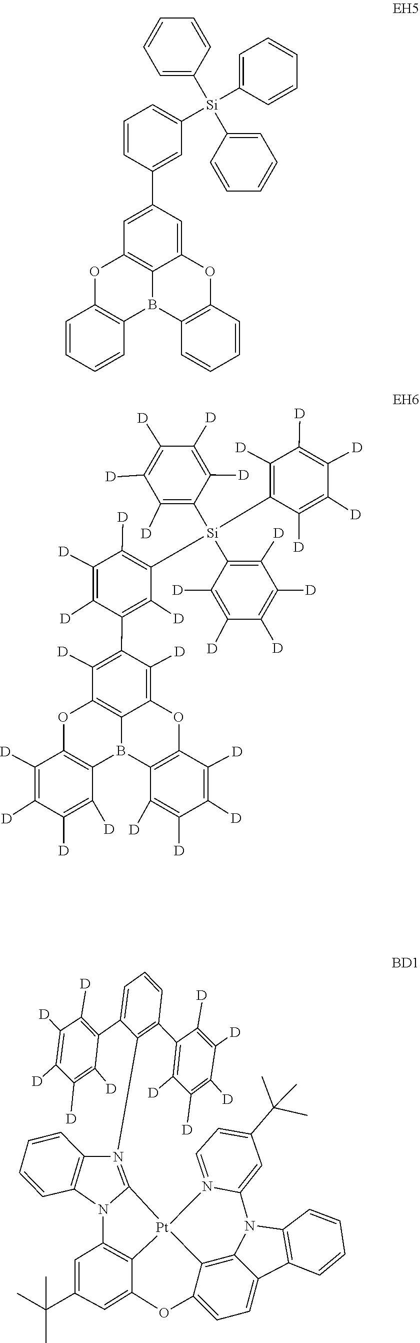

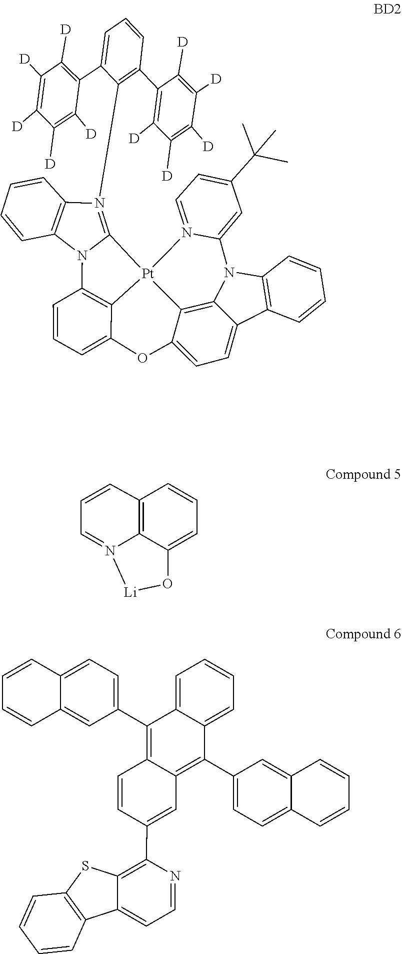

OLEDs were grown on a glass substrate pre-coated with an indium-tin-oxide (ITO) layer having a sheet resistance of 15-Ω/sq. Prior to any organic layer deposition or coating, the substrate was degreased with solvents and then treated with an oxygen plasma for 1.5 minutes with 50 W at 100 mTorr and with UV ozone for 5 minutes. The devices were fabricated in high vacuum (<10⁻⁶ Torr) by thermal evaporation. The anode electrode was 750 Å of indium tin oxide (ITO). All devices were encapsulated with a glass lid sealed with an epoxy resin in a nitrogen glove box (<1 ppm of H₂O and O₂) immediately after fabrication with a moisture getter incorporated inside the package. Doping percentages are in volume percent. The devices were grown in two different device structures.

The devices shown in Table 1 below had organic layers consisting of, sequentially, from the ITO surface, 100 Å of Compound 1 (HIL), 250 Å of Compound 2 (HTL), 50 Å EBL, 300 Å of Host 1 doped with X % of Host 2, and 12% of Dopant (EML), 50 Å of HBL, 300 Å of Compound 5 doped with 35% of Compound 6 (ETL), 10 Å of Compound 5 (EIL) followed by 1,000 Å of Al (Cathode), where EBL, Host 1, Host 2, X, Dopant, and HBL are as defined in Table 1. The color, EQE and LT₉₀ for the devices are reported in Table 2. The EQE and LT₉₀ for the devices Example 1, Example 2, and Comparison 1 are all reported relative to Comparison 2. The EQE and LT₉₀ for the devices Example 3 and Example 4 are reported relative to Comparison 3. The EQE and LT₉₀ for the device Example 5 is reported relative to Comparison 4. The EQE and LT₉₀ for the device Example 6 is reported relative to Comparison 5.

TABLE 1
Device composition

| | | | | X % of | | |
|---|---|---|---|---|---|---|
| Device | EBL | Host 1 | Host 2 | Host 2 | Dopant | HBL |
| Example 1 | HH1 | HH1 | EH4 | 50 | BD1 | EH1 |
| Example 2 | HH1 | HH1 | EH3 | 50 | BD1 | EH1 |
| Comparison 1 | HH1 | HH1 | EH2 | 50 | BD1 | EH1 |
| Comparison 2 | HH1 | HH1 | EH1 | 50 | BD1 | EH1 |
| Example 3 | HH1 | HH2 | EH4 | 50 | BD1 | EH1 |
| Example 4 | HH1 | HH2 | EH3 | 50 | BD1 | EH1 |
| Comparison 3 | HH1 | HH2 | EH1 | 50 | BD1 | EH1 |
| Example 5 | HH1 | HH1 | EH6 | 30 | BD2 | EH6 |
| Comparison 4 | HH1 | HH1 | EH5 | 30 | BD2 | EH5 |
| Example 6 | HH3 | HH4 | EH1 | 35 | BD2 | EH1 |
| Comparison 5 | HH3 | HH3 | EH1 | 35 | BD2 | EH1 |

TABLE 2
Device performance

| | CIE | | EQE | LT90 |
|---|---|---|---|---|
| Device | coordinates | λₘₐₓ | (relative) | (relative) |
| Example 1 | (0.140, 0.231) | 468 nm | 1.01 | 1.66 |
| Example 2 | (0.141, 0.231) | 468 nm | 1.02 | 1.38 |
| Comparison 1 | (0.139, 0.222) | 468 nm | 1.04 | 1.14 |
| Comparison 2 | (0.141, 0.230) | 468 nm | 1.00 | 1.00 |
| Example 3 | (0.140, 0.229) | 468 nm | 0.99 | 1.40 |
| Example 4 | (0.140, 0.229) | 468 nm | 1.00 | 1.27 |
| Comparison 3 | (0.139, 0.220) | 468 nm | 1.00 | 1.00 |
| Example 5 | (0.130, 0.155) | 463 nm | 0.98 | 1.43 |
| Comparison 4 | (0.130, 0.153) | 463 nm | 1.00 | 1.00 |
| Example 6 | (0.136, 0.155) | 462 nm | 0.99 | 1.43 |
| Comparison 5 | (0.136, 0.154) | 462 nm | 1.00 | 1.00 |

The above data shows that the device Example 1 and Example 2 exhibited a longer lifetime than the device without deuteration on Host 2, Comparison 2. The 38% to 66% longer lifetime for Example 1 and 2 are beyond any value that could be attributed to experimental error and the observed improvement is significant. Furthermore, the deuteration of just the tetraphenyl silane group for EH3 has a much larger impact on device lifetime than deuteration of just the bicarbazole triazine portion of the molecule in EH2, comparison 1. Based on the fact that the hosts have similar structures with the only difference being the portion of the molecule being deuterated, the significant performance improvement observed in the above data was unexpected. As shown in Table 3, EH3 and EH4 each have a first group which is a fully deuterated triphenyl silyl while maintaining nearly complete localization of the HOMO and LUMO orbitals on the second group comprising triazine and carbazole. Similarly, full deuteration of the triphenyl silane containing hosts EH6 and HH4 each resulted in 43% lifetime enhancement for Example 5 and Example 6 compared to Comparison 4 and Comparison 5, respectively, where the host was not deuterated. As with EH3 and EH4, HH4 and EH6 both had complete deuteration of the triphenyl silane while maintaining nearly complete localization of the HOMO and LUMO orbitals on the second groups of biscarbazole and phenyl-5,9-dioxa-13b-boranaphtho[3,2,1-de]anthracene respectively. Without being bound by any theories, this improvement may be attributed to preferential intermolecular reactions between excited molecules and the insulating groups on host materials. These insulating groups without any substantial HOMO or LUMO localization may be more susceptible to these intermolecular reactions due to the inability to share any charge or excited states across two or more molecules. Intermolecular reactions involving CH bonds on these insulating groups can have large kinetic isotope effects leading to lifetime enhancements from the deuteration of these insulating groups. Other insulating groups which are fully or partially deuterated and do not have much HOMO and LUMO electron density, such as tetraphenylene as in HH6, are expected to show similar enhancement.

TABLE 3
Molecular fragments and their HOMO and LUMO localizations

| | | % of sites | | | |
|---|---|---|---|---|---|
| Host | First group | deuterated | Second group | % HOMO | % LUMO |
| EH3, EH4 | 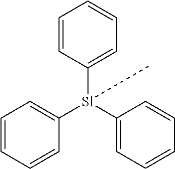 | 100% | 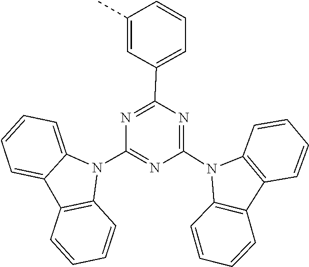 | 100% | 99% |
| EH1, EH2 | 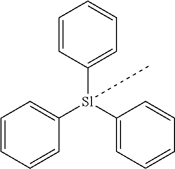 | 0% | 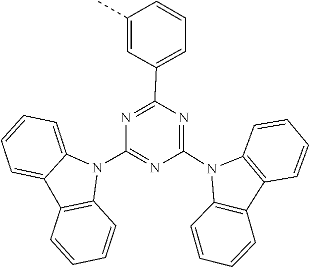 | 100% | 99% |
| HH4, HH5 | 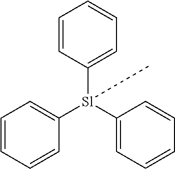 | 100% | 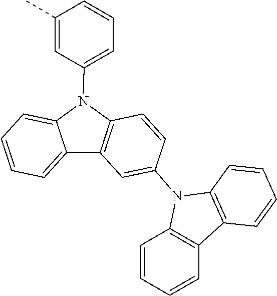 | 100% | 100% |
| HH4, HH5 | 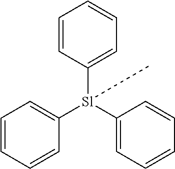 | 100% | 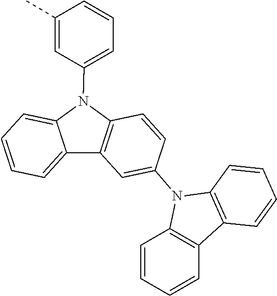 | 100% | 100% |
| EH6, EH7, | 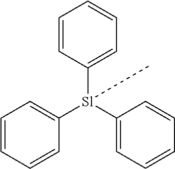 | 100% | 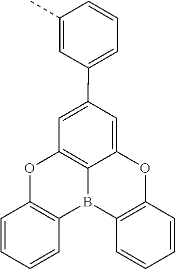 | 100% | 99% |
| EH5 | 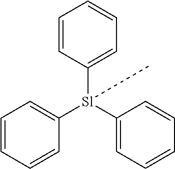 | 0% | 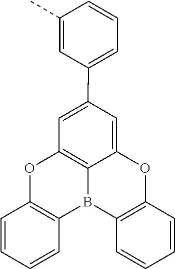 | 100% | 99% |
| HH6 | 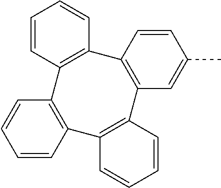 | 100 | 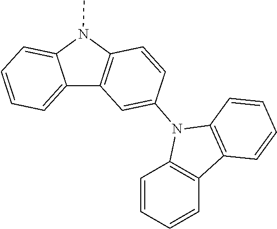 | 99% | 96% |

It should be understood that % D in the above table is meant the % of protons on a molecular structure which are exchanged in the average molecule. E.g. a sample of a compound which has each site deuterated 90% of the time would be called “fully Deuterated” or 100% Deuterated. However if all of the sites were at 100% isotopic purity but 1 site was a 0% D incorporation it would be considered partial Deuteration.

What is claimed is:

1. A compound consisting of a first group and a second group;
wherein,
each of the first group and the second group independently comprises at least one continuous moiety comprising at least three rings;
atoms of the first group do not overlap with atoms of the second group, and no fused ring system is part of both groups;
the compound has a highest occupied molecular orbital (HOMO) and a lowest unoccupied molecular orbital (LUMO);
the second group contains at least 70% of the electron density of the HOMO and at least 70% of the electron density of the LUMO; and
the first group is at least 30% deuterated.

2. The compound of claim 1, wherein the first group has a lowest triplet energy T₁ of at least 3.00 eV; and/or wherein the second group is not deuterated or wherein the second group has a maximum percentage of the deuteration of 20%.

3. A compound comprising or consisting of a first group and a second group;
wherein
each of the first group and the second group independently comprises at least one continuous moiety comprising at least three rings;
atoms of the first group do not overlap with atoms of the second group, and no fused ring system is part of both groups;
the first group comprises at least one first moiety selected from the group consisting of X—[Ar]ₙ, triphenylene, tetraphenylene, terphenyl, xanthene, and 9,10-dihydroanthracene;
X is selected from the group consisting of sp³ carbon atom, sp³ silicon atom, and sp³ germanium atom;
n is an integer of 1 to 4;
wherein, when n is an integer from 2 to 4, each Ar can be same or different; each Ar does not bond or fuse to each other;
wherein each Ar is independently a 5-membered or 6-membered carbocyclic or heterocyclic ring or a fused ring system comprising at least two rings that are independently 5-membered and/or 6-membered carbocyclic or heterocyclic rings, and each Ar can be partially or fully deuterated;
the second group comprises at least one second moiety selected from the group consisting of carbazole, indolocarbazole, dibenzothiophene, indolodibenzothiophene, dibenzofuran, indolodibenzofuran, dibenzoselenophene, indolodibenzoselenophene, 5,9-dioxa-13b-boranaphtho[3,2,1-de]anthracene, imidazole, pyridine, pyrimidine, pyrazine, pyridazine, triazine, benzo[d]benzo[4,5]imidazo[1,2-a]imidazole; and aza-variants thereof; and
wherein each benzene ring of the triphenylene, tetraphenylene, terphenyl, xanthene, and 9,10-dihydroanthracene of the first moiety comprises at least one D atom.

4. The compound of claim 3, wherein when Ar is present, each Ar has a minimum percentage of deuteration of 30%.

5. The compound of claim 3, wherein the second group is not deuterated or wherein the second group has a maximum percentage of the deuteration of 20%; and/or wherein the second group consists of a minimum percentage of the electron densities of each the HOMO and the LUMO of 80%.

6. The compound of claim 3, wherein the first group has a minimum percentage of deuteration of 30%; and/or wherein the first group has a lowest triplet energy T₁ of at least 3.00 eV.

7. The compound of claim 3, wherein the second group comprises at least one structure selected from the group consisting of: 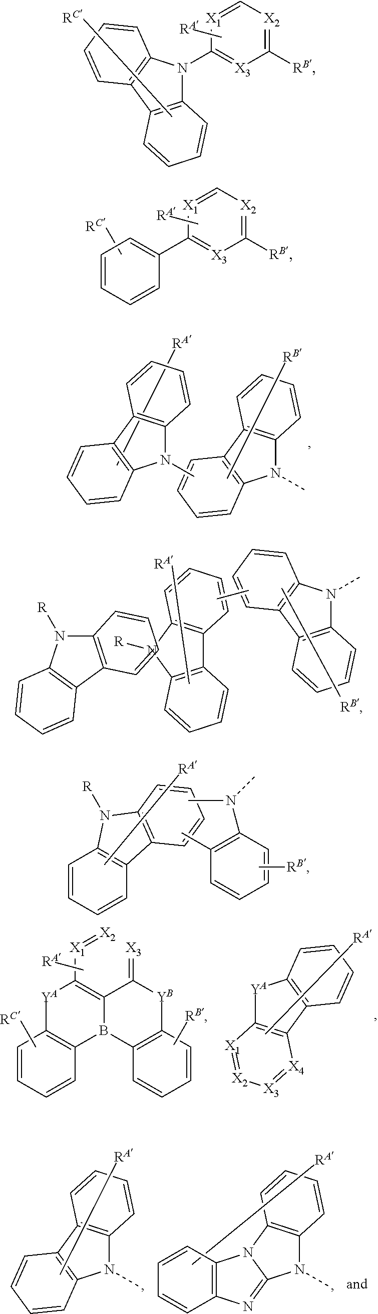 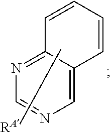
wherein:
each of X₁ to X₄ is independently selected from the group consisting of C and N;
each of Y^{A} and Y^{B} is independently selected from the group consisting of BR, NR, PR, O, S, Se, C═O, S═O, SO₂, CRR′, SiRR′, and GeRR′;
each of R^{A′}, R^{B′}, and R^{C′} is independently represent from mono to the maximum possible number of substitutions, or no substitution;
each R, R′, R^{A′}, R^{B′}, and R^{C′} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, selenyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof; and
wherein any two R, R′, R^{A′}, R^{B′}, and R^{C′} can be fused or joined to form a ring.

8. The compound of claim 3, wherein the first group comprises at least one structure selected from the group consisting of: 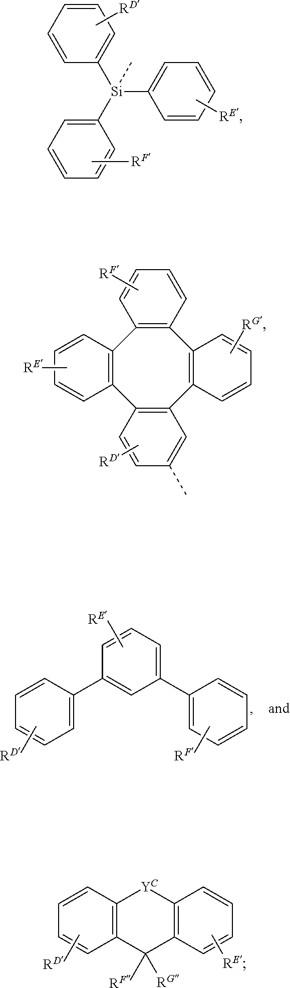
wherein:
Y^{C} is selected from the group consisting of BR, NR, PR, O, S, Se, C═O, S═O, SO₂, CRR′, SiRR′, and GeRR′;
each of R^{D′}, R^{E′}, R^{F′}, and R^{G′} independently represents from mono to the maximum possible number of substitutions, or no substitutions;
wherein each R, R′, R^{D′}, R^{E′}, R^{F′}, R^{F″}, R^{G′}, and R^{G″} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, boryl, selenyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, and combinations thereof;
wherein R, R′, R^{D′}, R^{E′}, R^{F′}, and R^{G′} are at least 30% deuterated.

9. The compound of claim 3, wherein the compound is selected from the group consisting of:

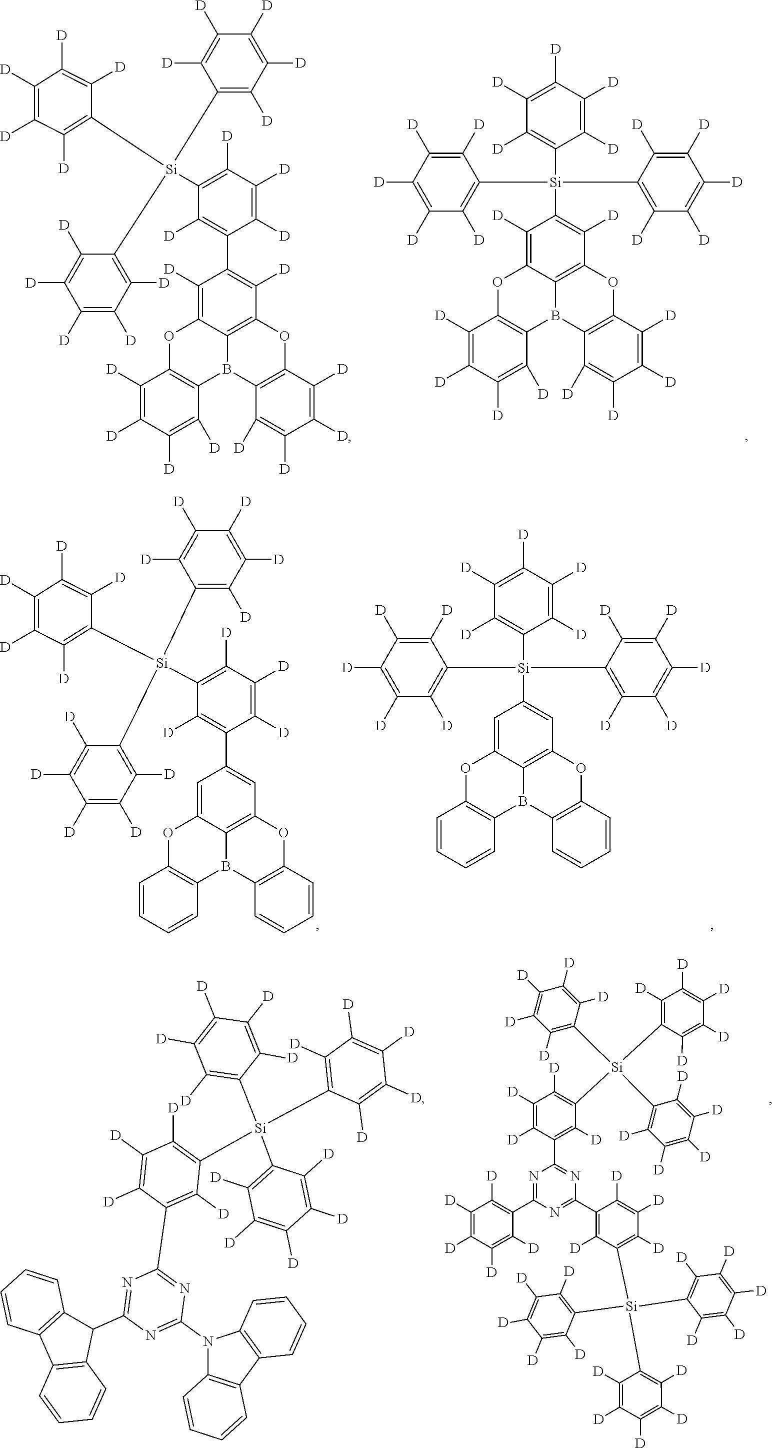

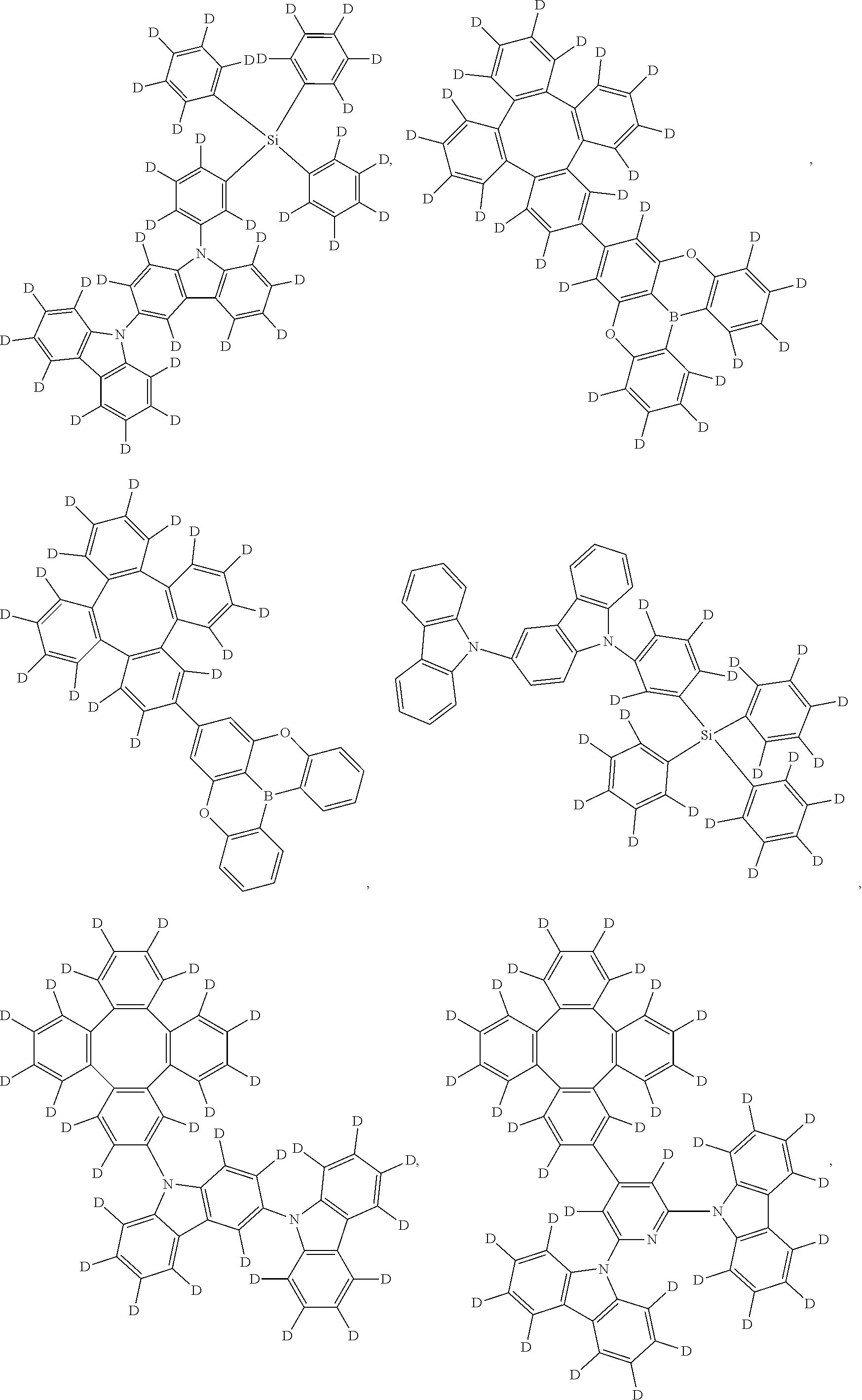

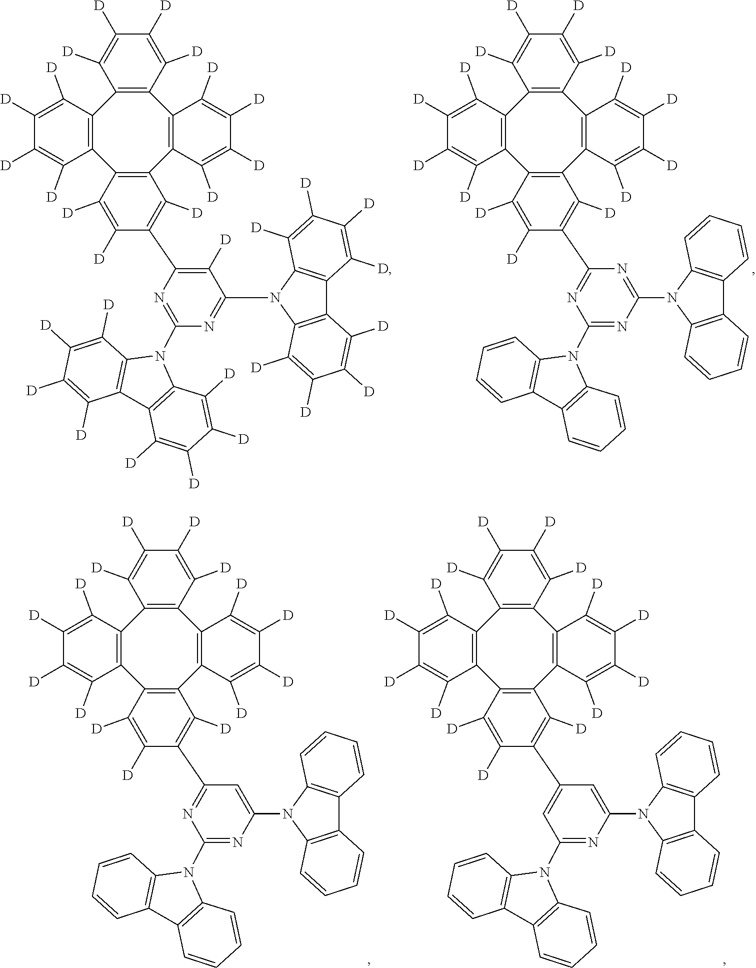

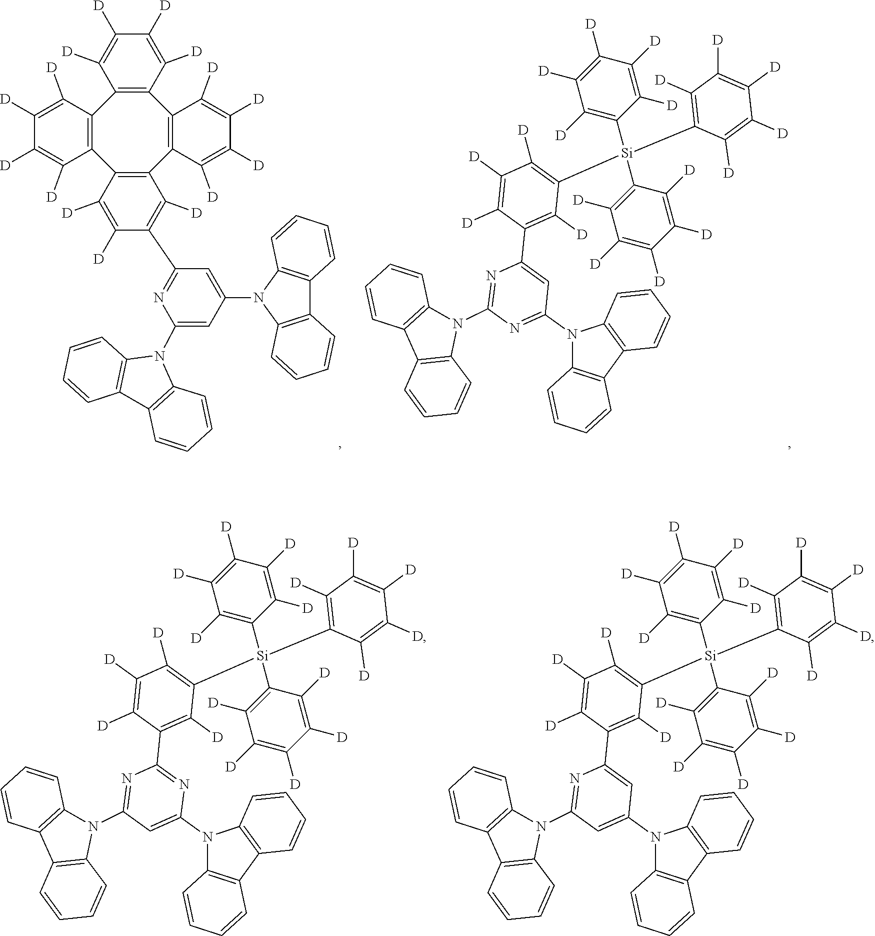

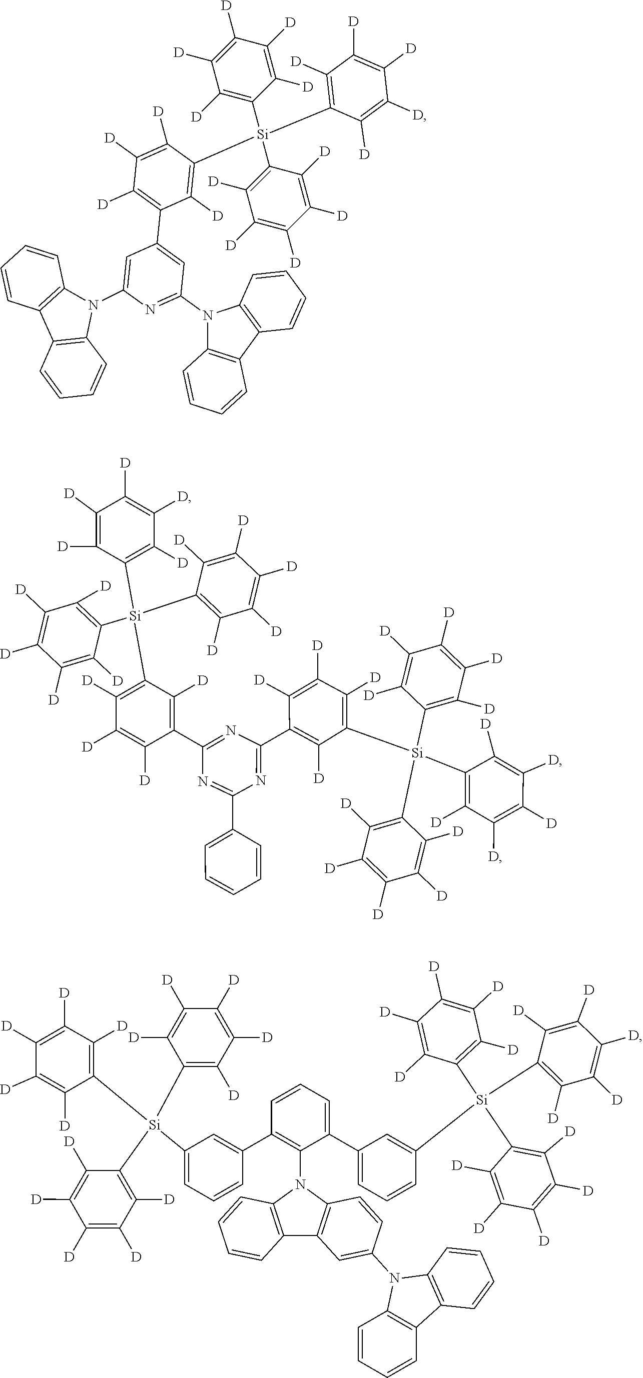

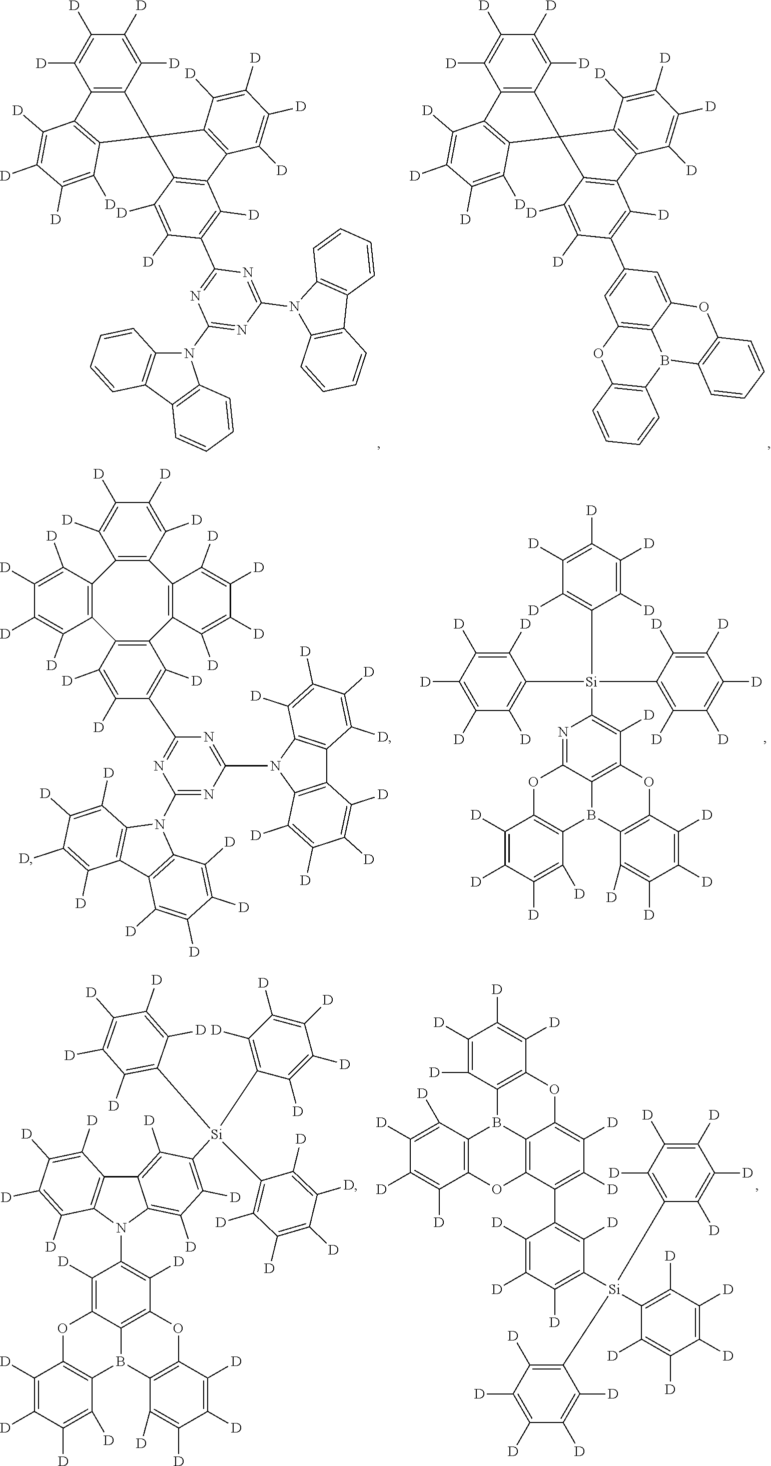

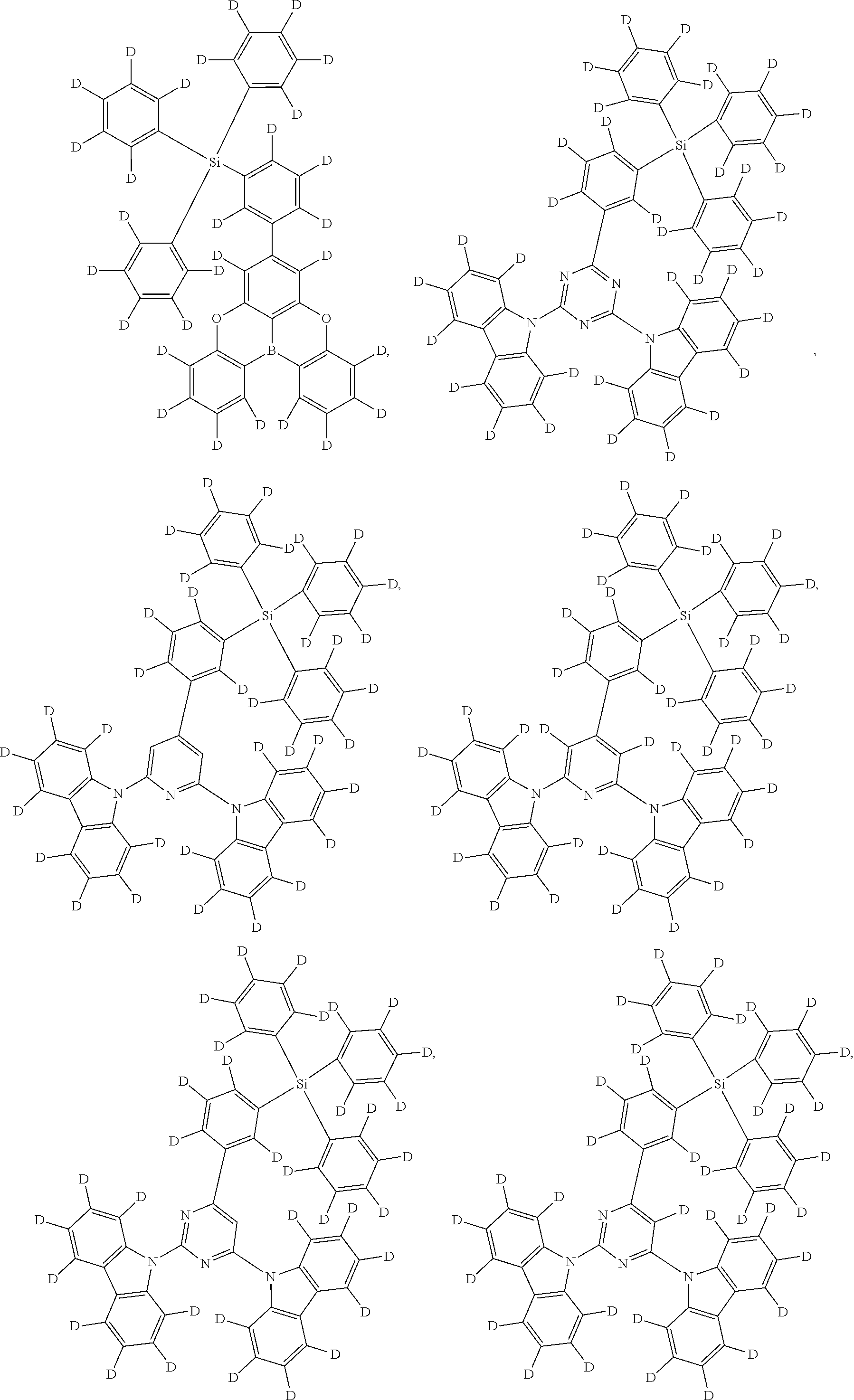

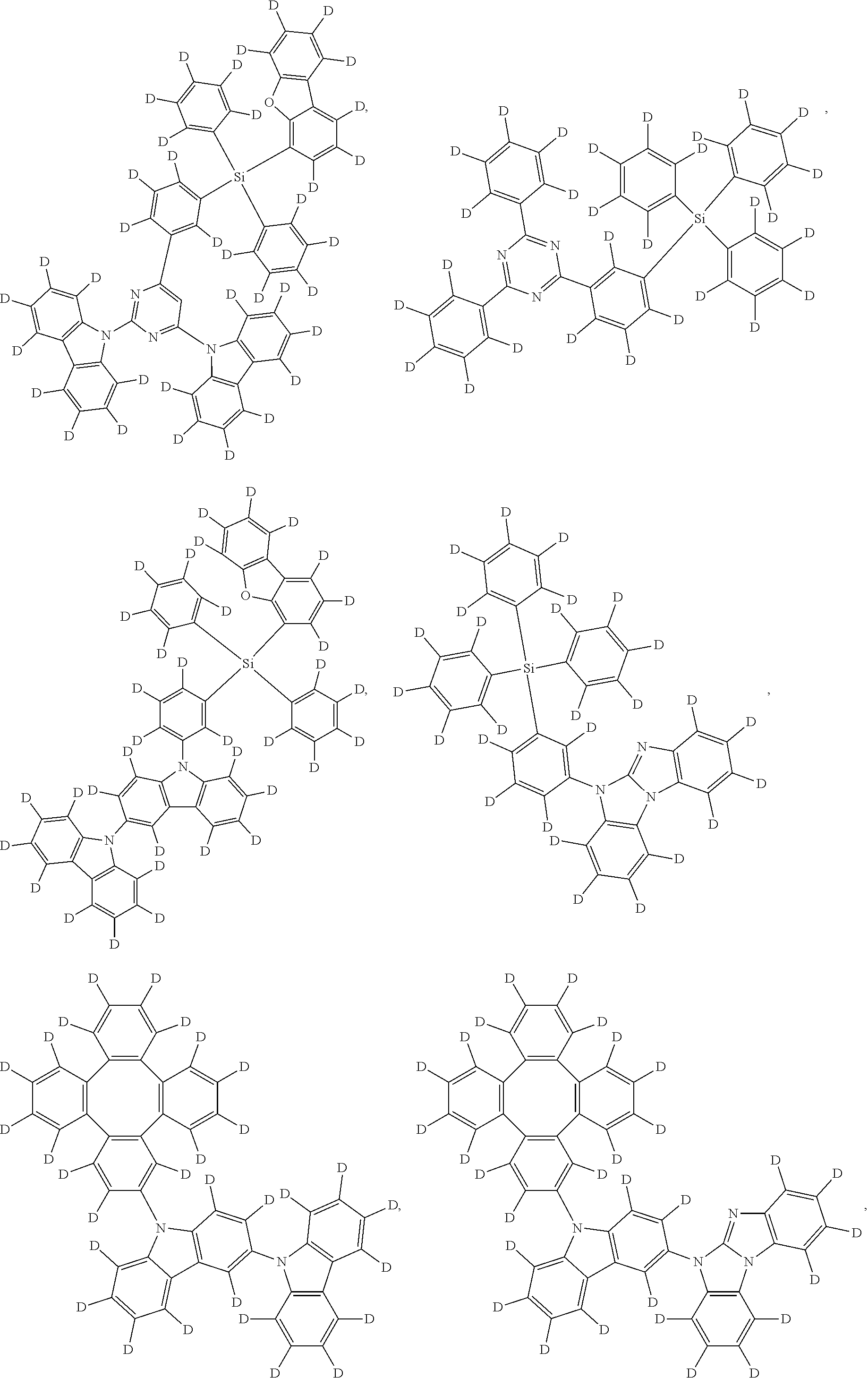

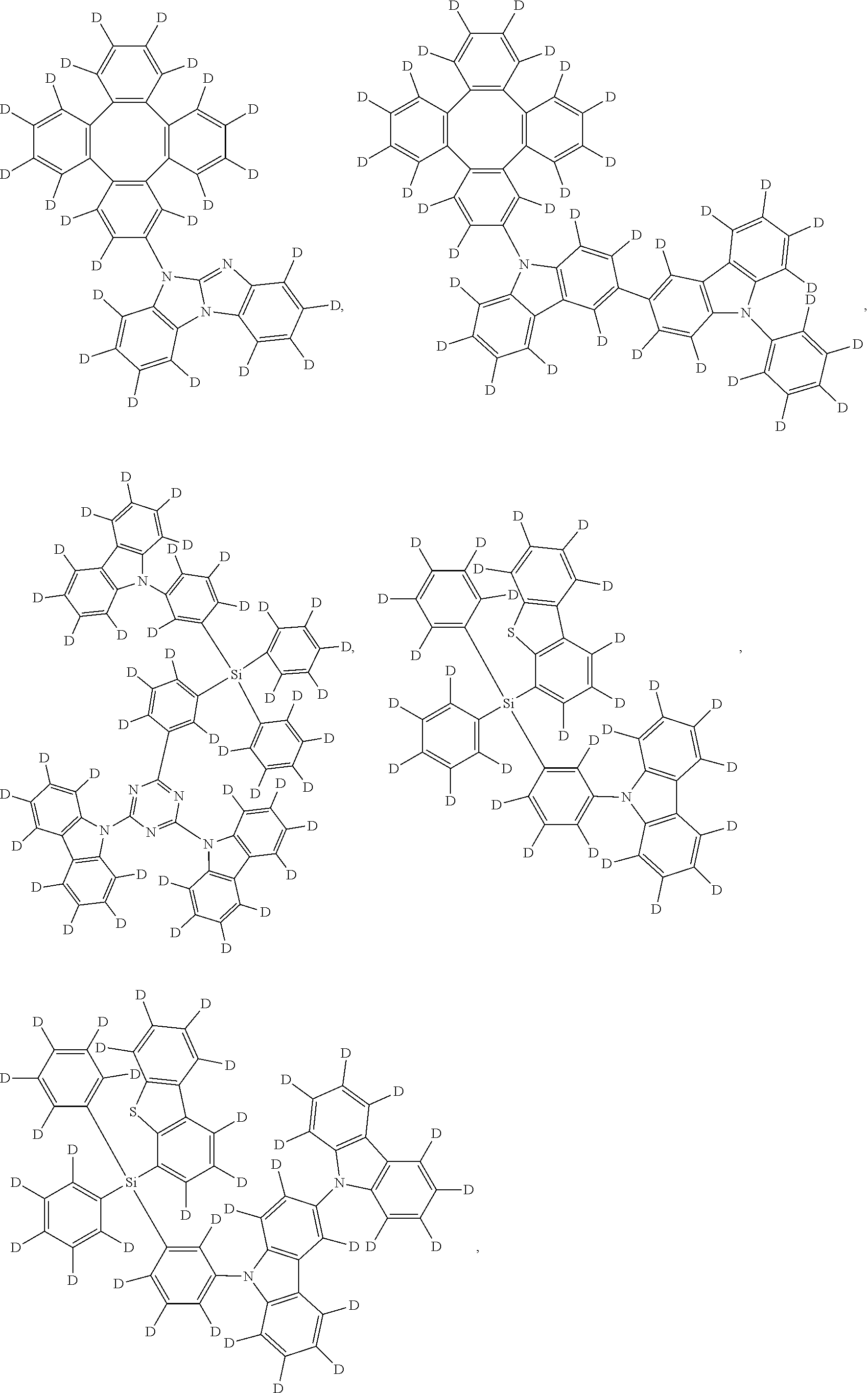

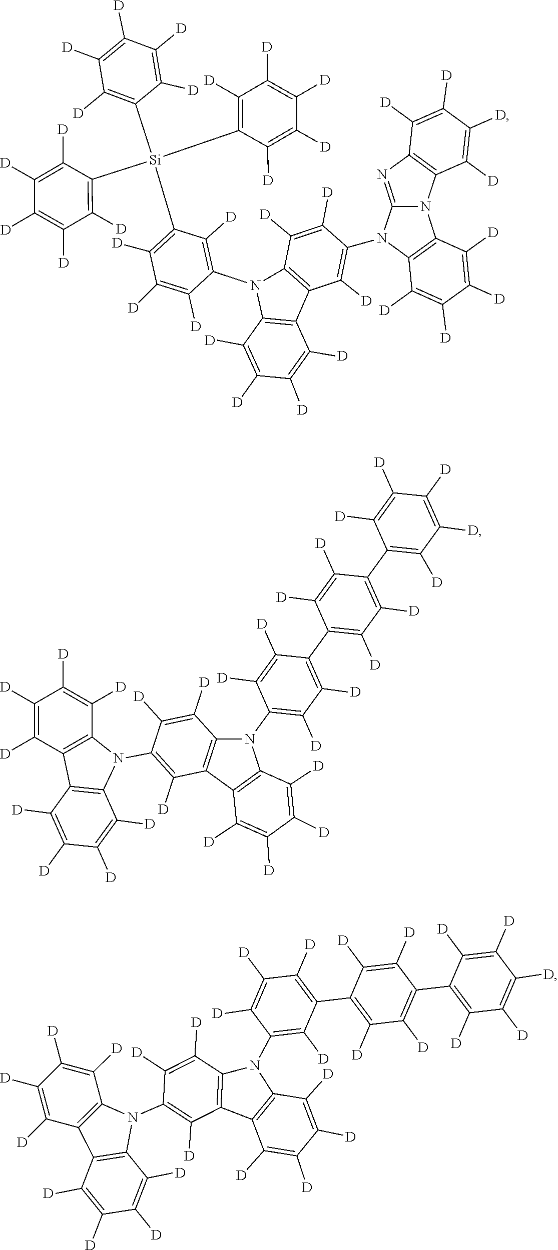

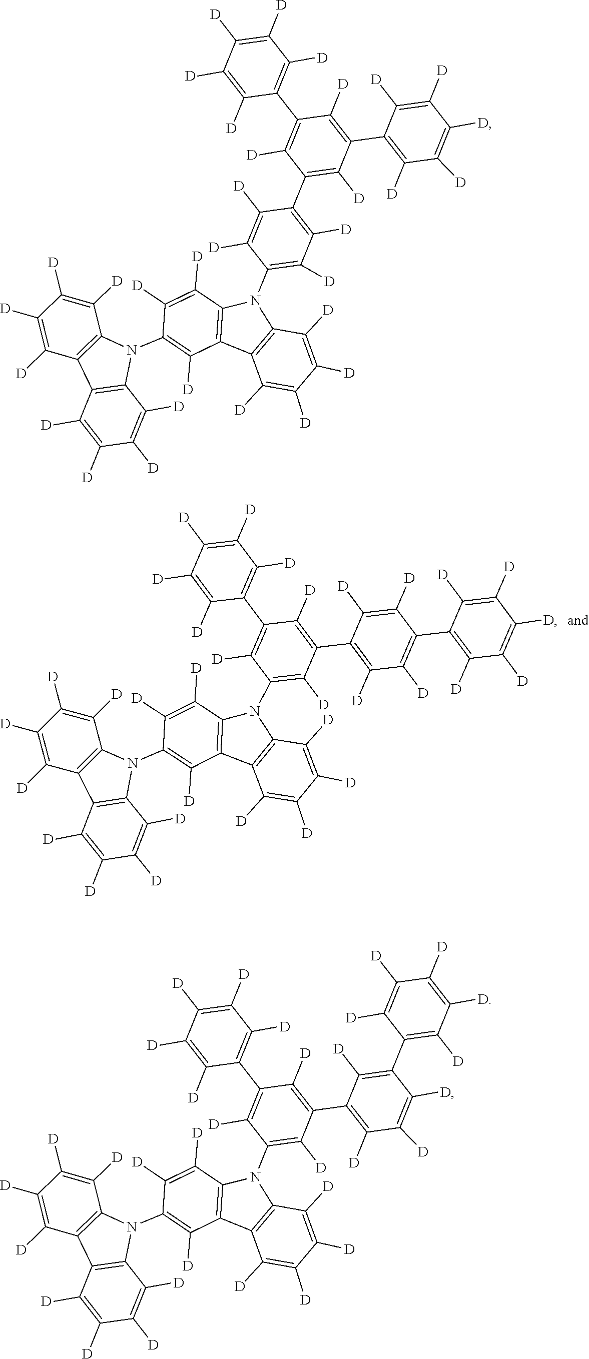

10. A compound comprising or consisting of a first group and a second group;
wherein
each of the first group and the second group independently comprises at least one continuous moiety comprising at least three rings;
atoms of the first group do not overlap with atoms of the second group, and no fused ring system is part of both groups;
wherein a device lifetime LT1 of an OLED in which the compound is used as a host is at least 30% higher than a device lifetime LT2 of a first test OLED in which a first comparative compound is used as a host; wherein the first comparative compound consists of a first group and a second group and has the same chemical structure as the compound except that the first group of the first comparative compound is not deuterated; and wherein the OLED and the first test OLED are different only in deuteration levels of the compound and the first comparative compound.

11. The compound of claim 10, wherein the second group is not deuterated or wherein the second group has a maximum percentage of deuteration of 20%; and/or wherein the second group consists of a minimum percentage of the electron densities of the HOMO and the LUMO of 70%.

12. The compound of claim 10, wherein the first group has a minimum percentage of deuteration of 30%; and/or wherein the first group has a lowest triplet energy T1 of at least 3.00 eV.

13. An organic light emitting device (OLED) comprising:
an anode;
a cathode; and
an organic layer disposed between the anode and the cathode,
wherein the organic layer comprises a compound according to claim 1.

14. The OLED of claim 13, wherein the compound is a host, and the organic layer is an emissive layer that comprises a phosphorescent material.

15. The OLED of claim 13, wherein the compound is a host and the OLED comprises an acceptor that is an emitter and a sensitizer selected from the group consisting of a delayed fluorescence material, a phosphorescent material, and combination thereof; wherein the sensitizer transfers energy to the acceptor.

16. The OLED of claim 13, wherein the phosphorescent material is a transition metal complex having at least one ligand or part of the ligand if the ligand is more than bidentate selected from the group consisting of: 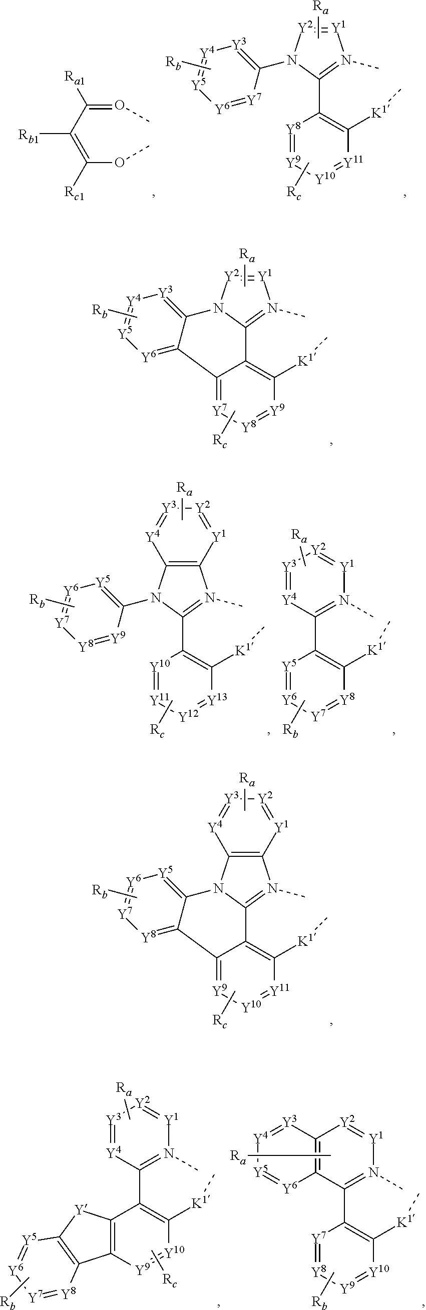 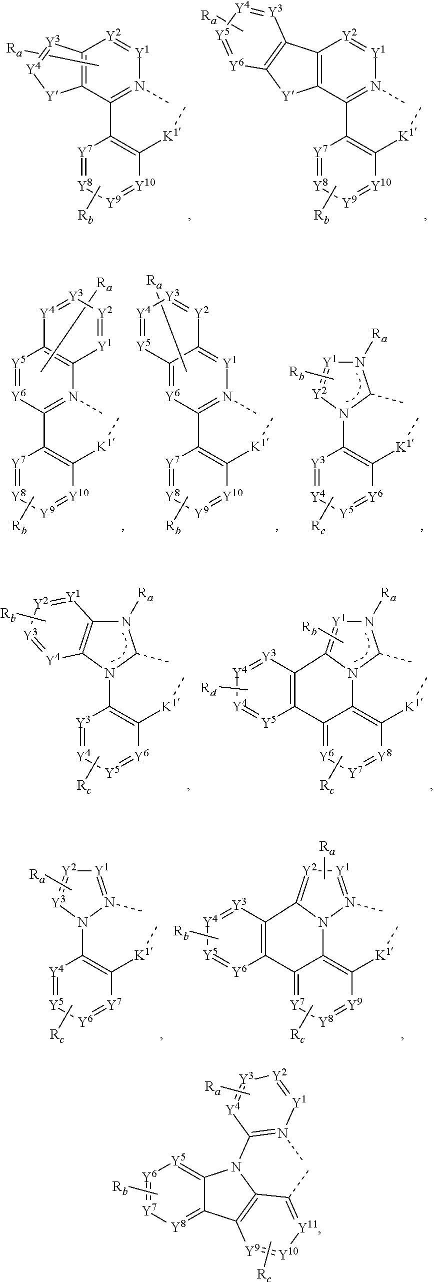 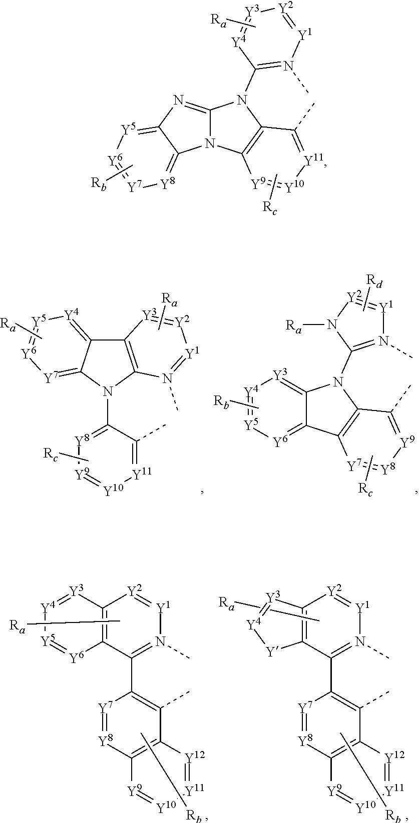 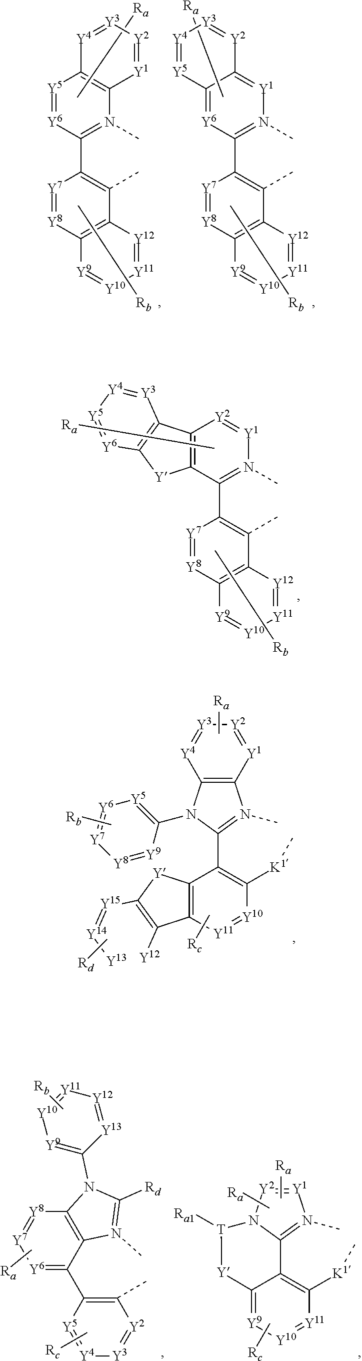 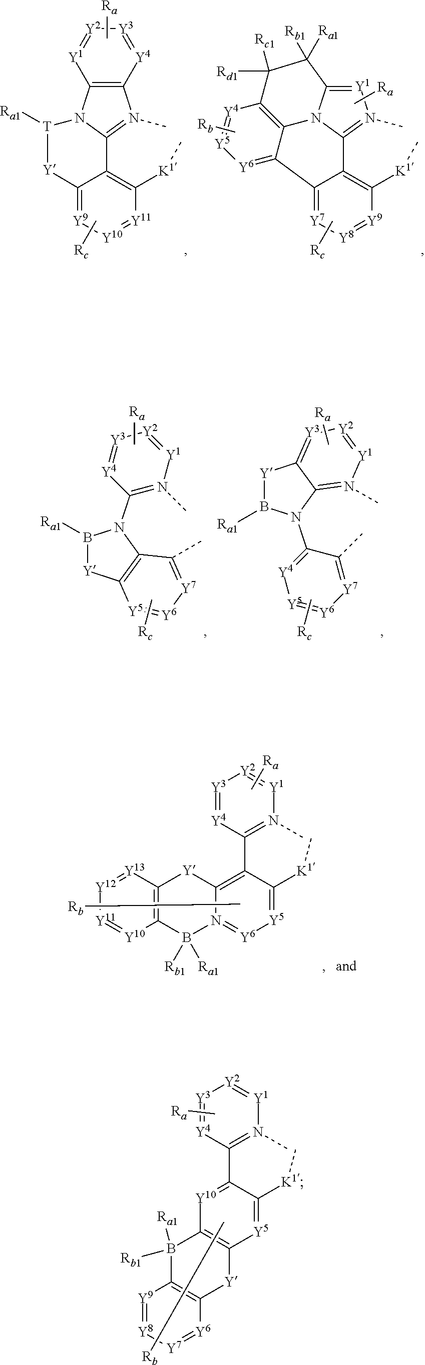
wherein:
T is selected from the group consisting of B, Al, Ga, and In;
K^{1′} is a direct bond or is selected from the group consisting of NRₑ, PRₑ, O, S, and Se;
each Y¹ to Y¹³ are independently selected from the group consisting of carbon and nitrogen;
Y′ is selected from the group consisting of B Rₑ, N Rₑ, P Rₑ, O, S, Se, C═O, S═O, SO₂, CRₑR_{f}, SiRₑR_{f}, and GeRₑR_{f};
Rₑ and R_{f} can be fused or joined to form a ring;
each Rₐ, R_{b}, R_{c}, and R_{d} can independently represent from mono to the maximum possible number of substitutions, or no substitution;
each Rₐ₁, R_{b1}, R_{c1}, R_{d1}, Rₐ, R_{b}, R_{c}, R_{d}, Rₑ, and R_{f} is independently a hydrogen or a substituent selected from the group consisting of the General Substituents as defined herein; and
any two adjacent substituents of Rₐ₁, R_{b1}, R_{c1}, R_{d1}, Rₐ, R_{b}, R_{c}, and R_{d} can be fused or joined to form a ring or form a multidentate ligand.

17. The OLED of claim 13, wherein the organic layer further comprises an additional host, wherein the additional host comprises at least one chemical moiety selected from the group consisting of triphenylene, carbazole, indolocarbazole, dibenzothiophene, dibenzofuran, dibenzoselenophene, 5λ²-benzo[d]benzo[4,5] imidazo[3,2-a]imidazole, 5,9-dioxa-13b-boranaphtho[3,2,1-de]anthracene, triazine, aza-triphenylene, aza-carbazole, aza-indolocarbazole, aza-dibenzothiophene, aza-dibenzofuran, aza-dibenzoselenophene, aza-5λ²-benzo[d]benzo[4,5]imidazo[3,2-a]imidazole, and aza-(5,9-dioxa-13b-boranaphtho[3,2,1-de]anthracene).

18. The OLED of claim 13, wherein the organic layer further comprises an additional host, wherein the additional host is selected from the group consisting of 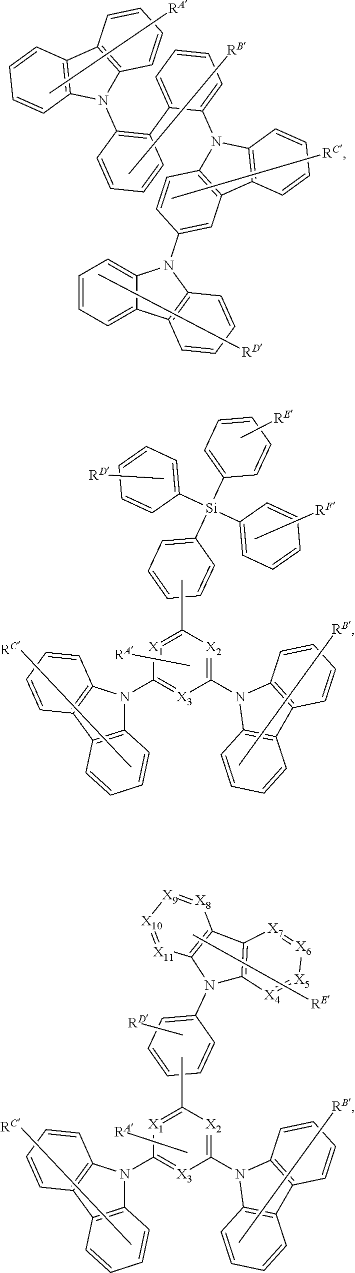 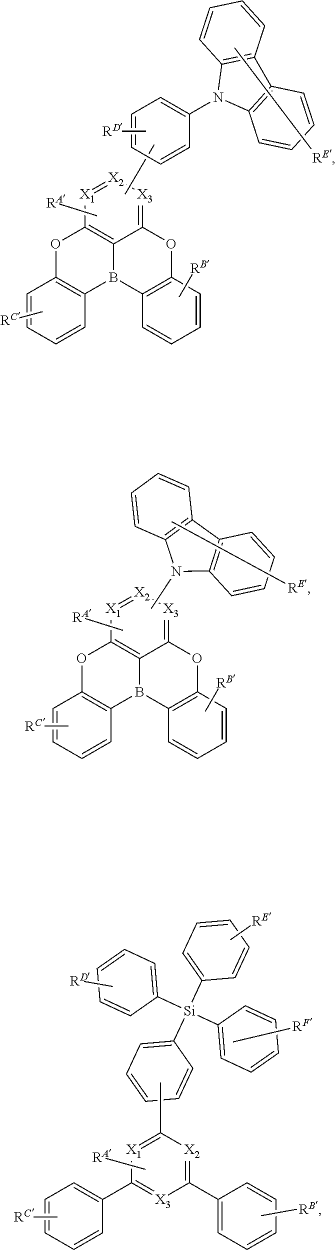 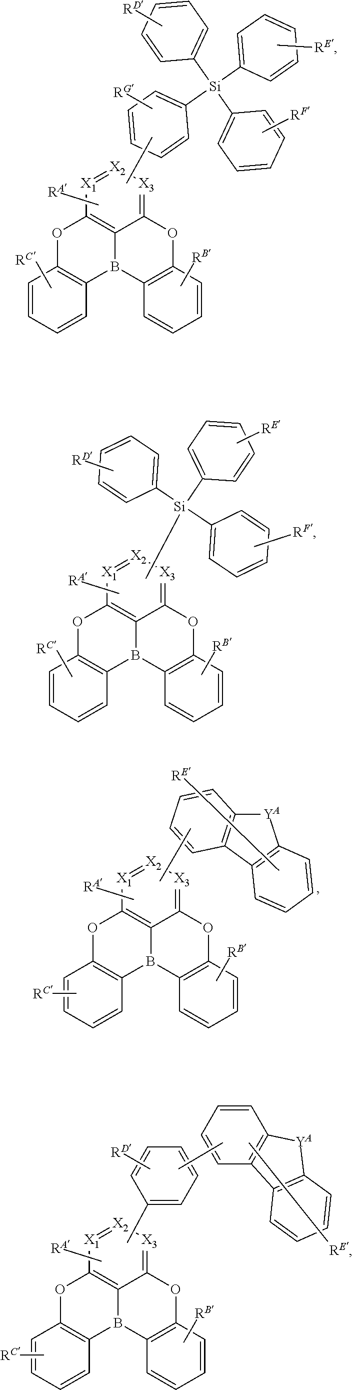 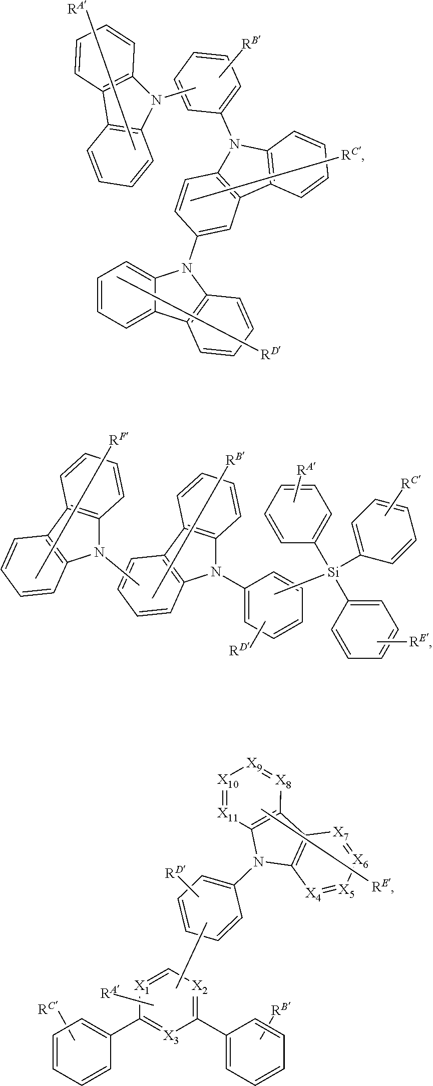 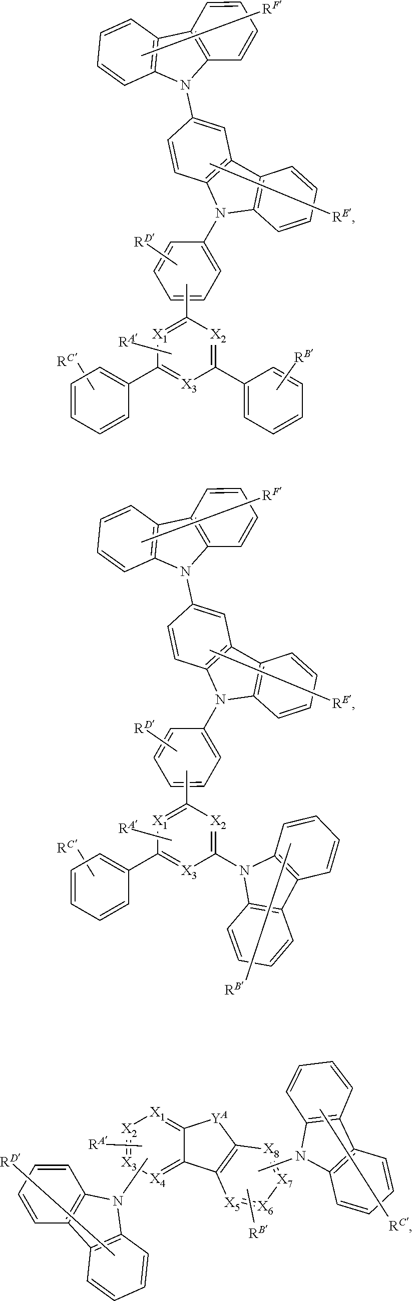 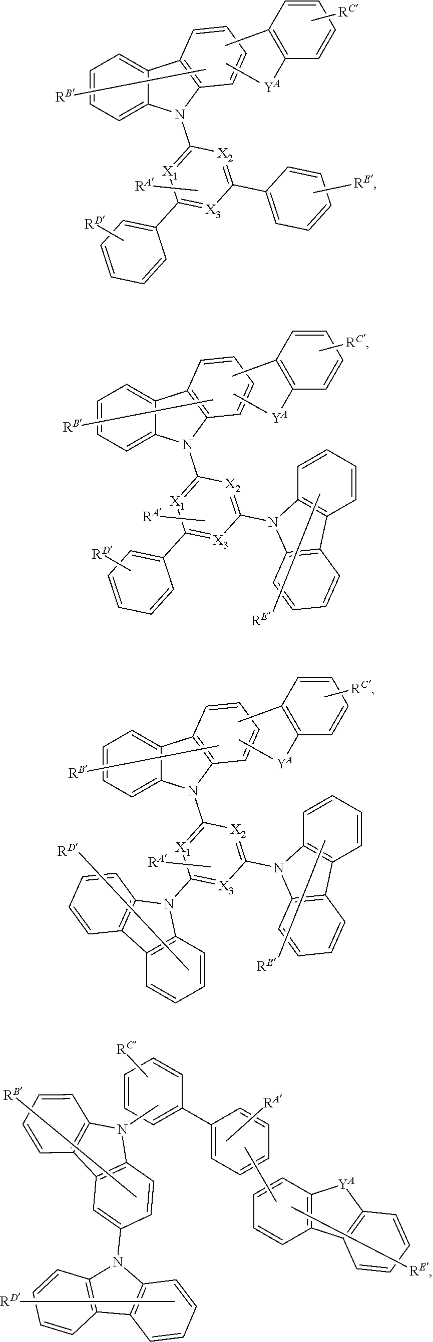 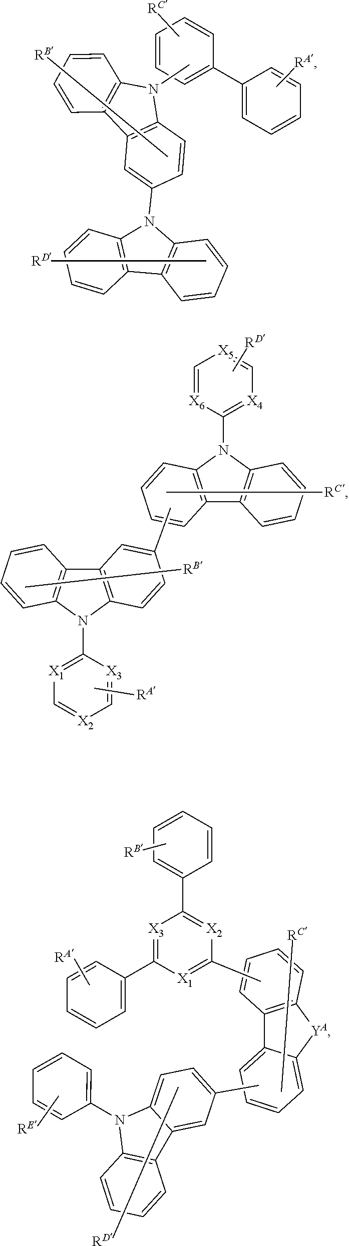 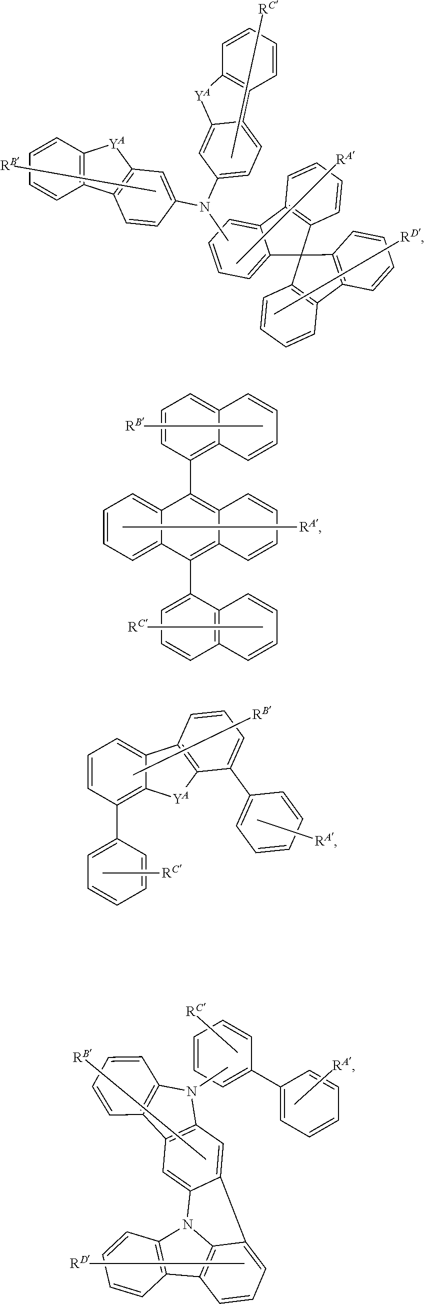 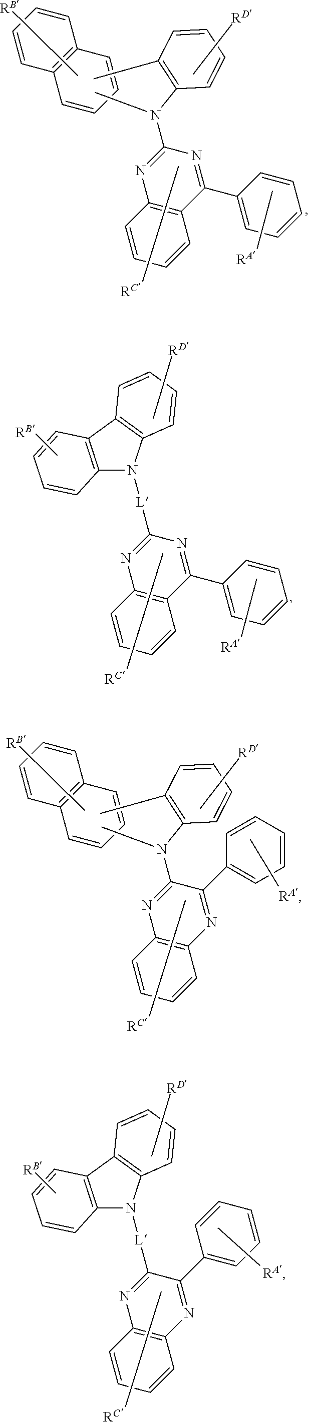 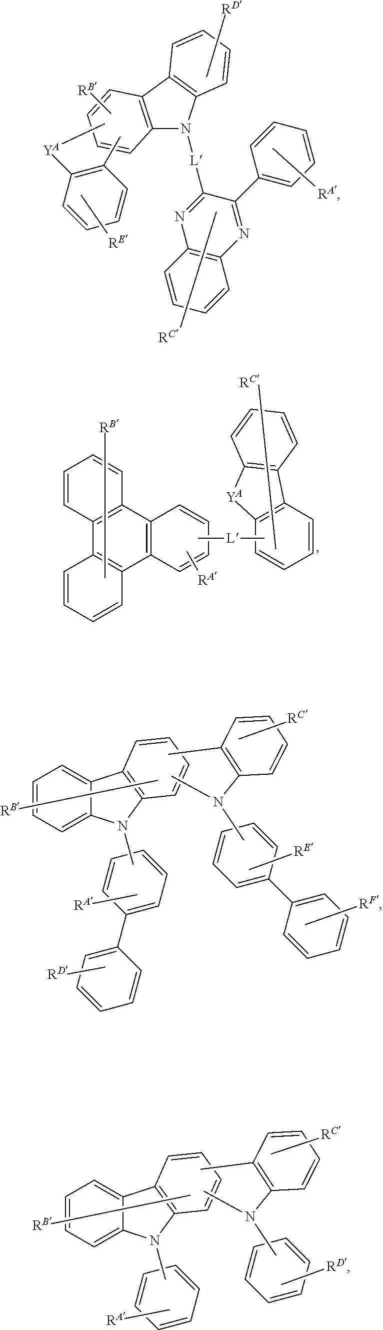 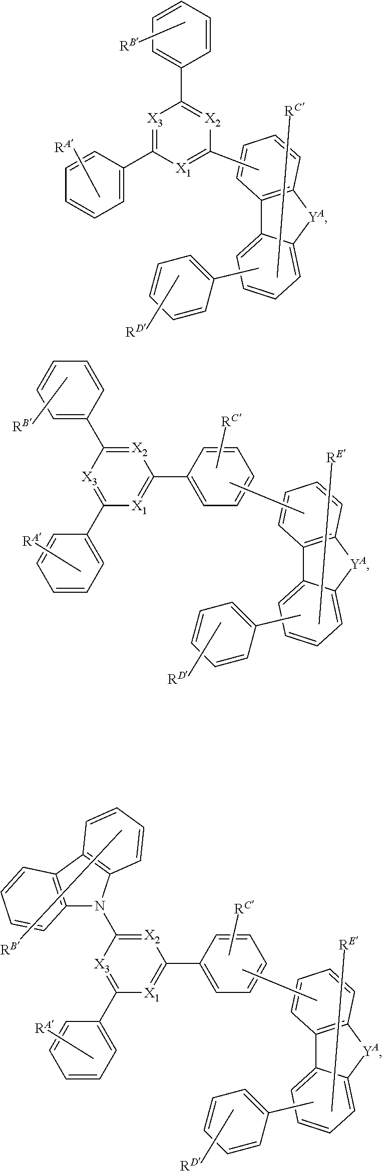 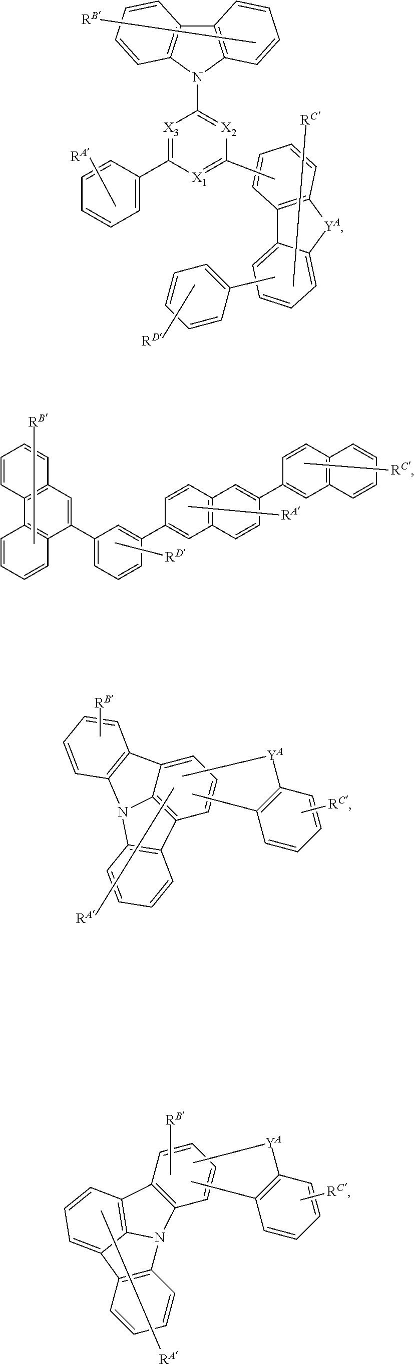 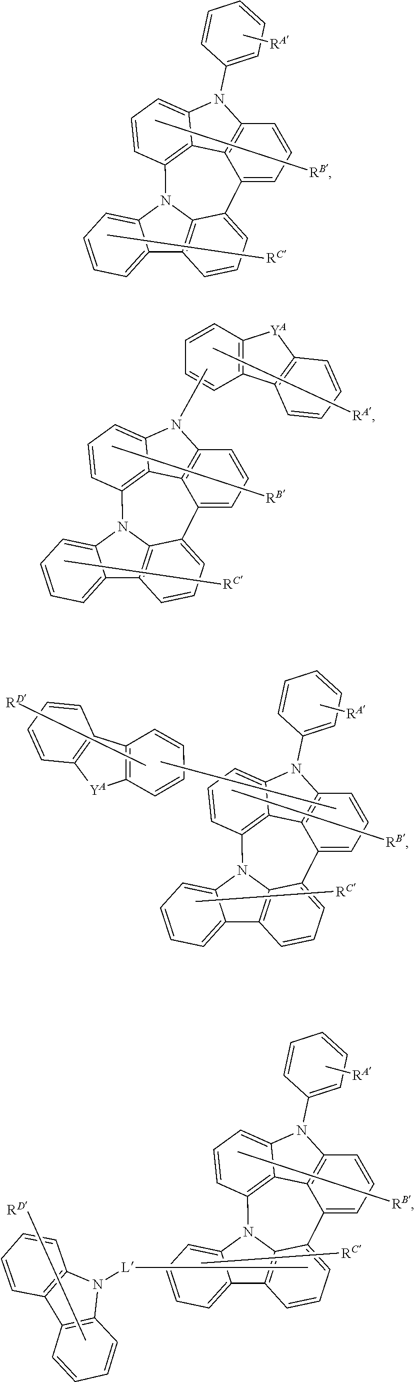 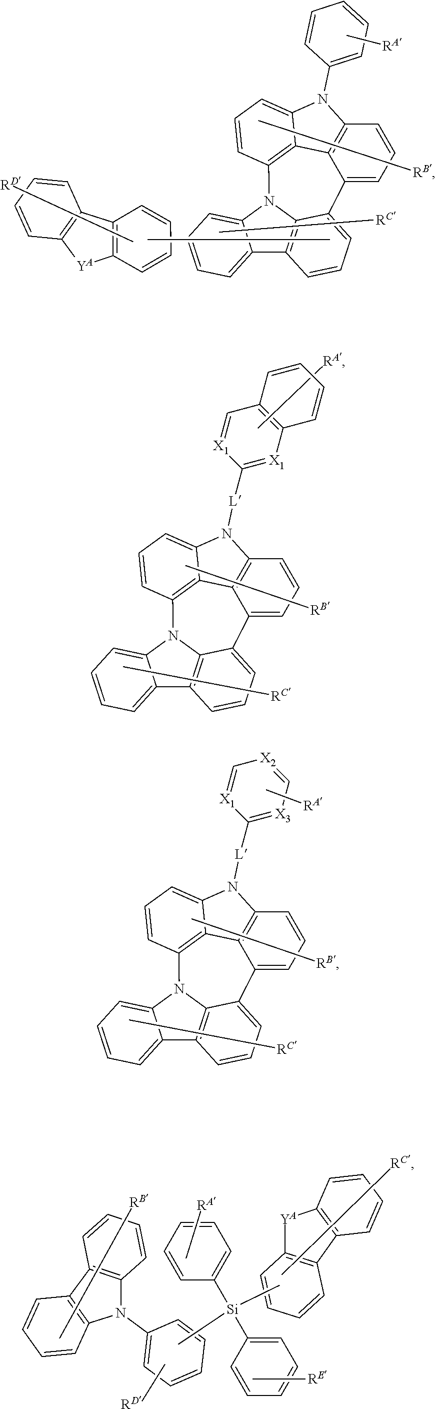 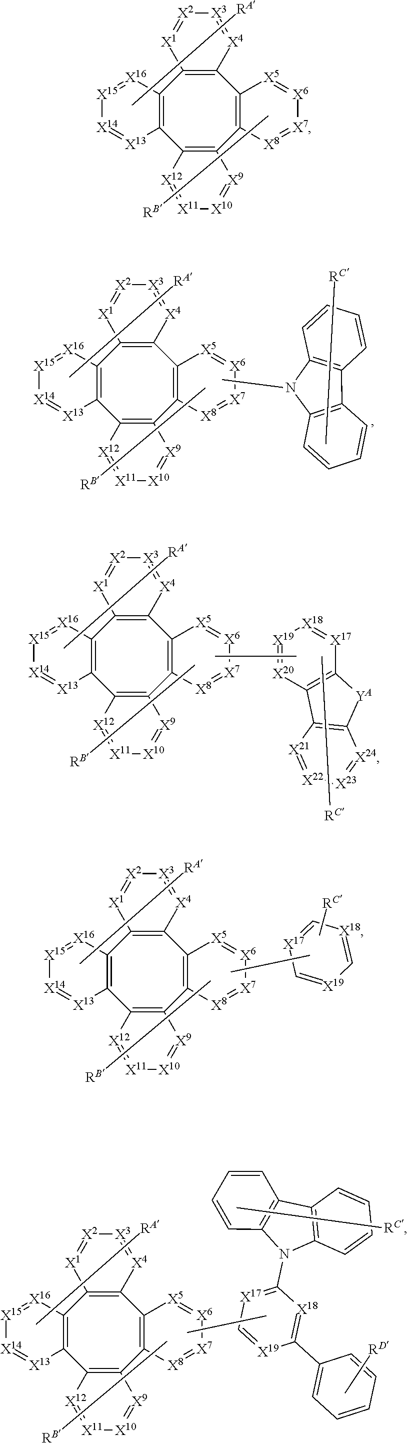 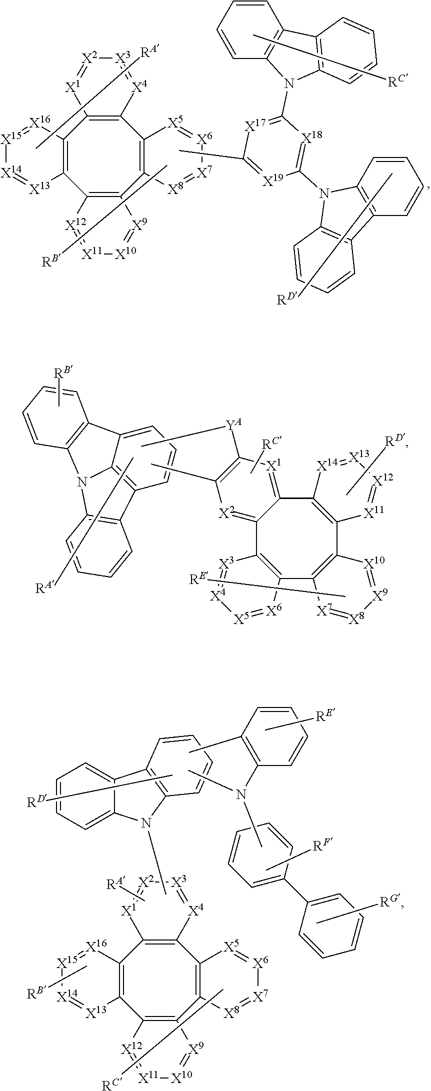 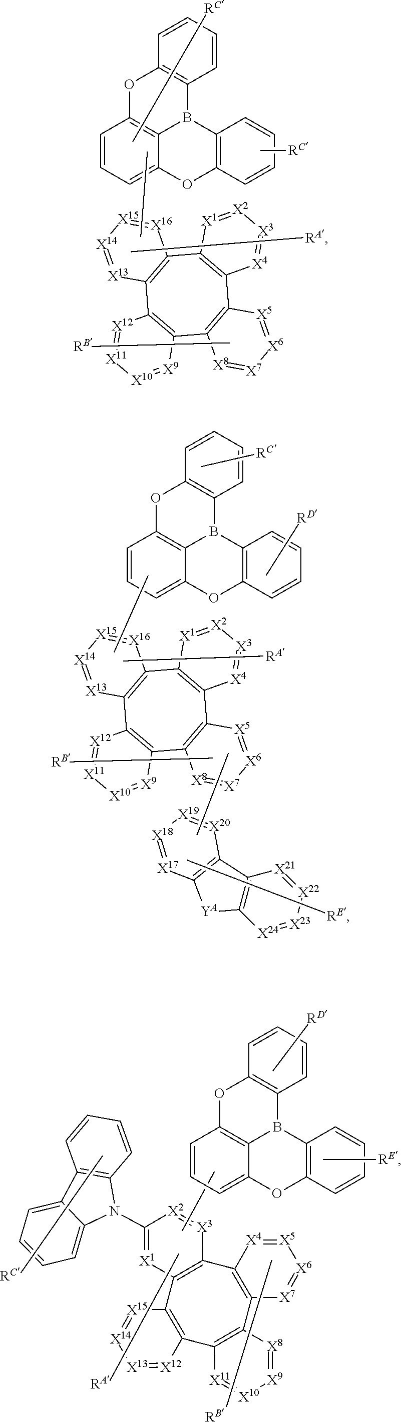 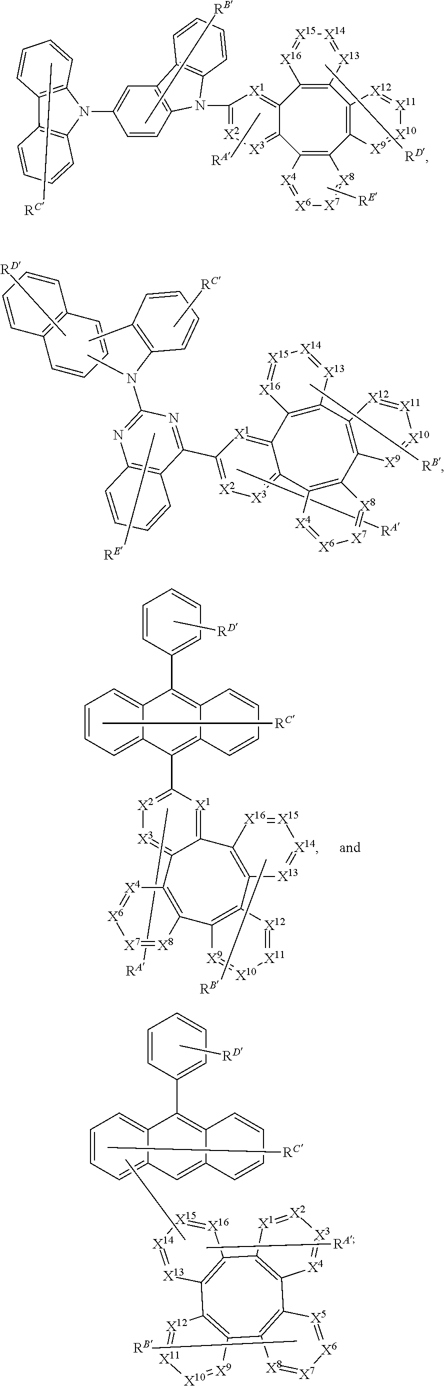
wherein:
each of X¹ to X²⁴ is independently C or N;
L′ is a direct bond or an organic linker;
each Y^{A} is independently selected from the group consisting of absent a bond, O, S, Se, CRR′, SiRR′, GeRR′, NR, BR, BRR′;
each of R^{A′}, R^{B′}, R^{C′}, R^{D′}, R^{E′}, R^{F′}, and R^{G′} independently represents mono, up to the maximum substitutions, or no substitutions;
each R, R′, R^{A′}, R^{B′}, R^{C′}, R^{D′}, R^{E′}, R^{F′}, and R^{G′} is independently a hydrogen or a substituent selected from the group consisting of deuterium, halogen, alkyl, cycloalkyl, heteroalkyl, heterocycloalkyl, arylalkyl, alkoxy, aryloxy, amino, silyl, germyl, selenyl, alkenyl, cycloalkenyl, heteroalkenyl, alkynyl, aryl, heteroaryl, acyl, carboxylic acid, ether, ester, nitrile, isonitrile, sulfanyl, sulfinyl, sulfonyl, phosphino, boryl, and combinations thereof;
two adjacent of R^{A′}, R^{B′}, R^{C′}, R^{D′}, R^{E′}, R^{F′}, and R^{G′} are optionally joined or fused to form a ring.

19. A consumer product comprising an organic light-emitting device (OLED) comprising:
an anode;
a cathode; and
an organic layer disposed between the anode and the cathode,
wherein the organic layer comprises a compound according to claim 1.

20. A formulation comprising a compound according to claim 1.
